(19) United States
(12) Patent Application Publication
(10) Pub. No.: US 2025/0376537 A1
(43) Pub. Date: Dec. 11, 2025

(54) METHODS OF TREATING CANCER USING TRISPECIFIC BINDING PROTEINS TARGETING HER2-EXPRESSING TUMORS

(71) Applicant: Sanofi, Paris (FR)

(72) Inventors: Giovanni ABBADESSA, Winchester, MA (US); Barbara Buday, Balatonfured (HU); Serena Masciari, Saugus, MA (US); Faiza Rharbaoui, Dossenheim (DE); Lei Wang, Belmont, MA (US); Ozlem Yildirim, Chelsea, MA (US)

(21) Appl. No.: 19/210,366

(22) Filed: May 16, 2025

Related U.S. Application Data
(60) Provisional application No. 63/648,948, filed on May 17, 2024.
(60) Provisional application No. 63/692,288, filed on Sep. 9, 2024.
(60) Provisional application No. 63/764,897, filed on Feb. 28, 2025.

(51) Int. Cl. C07K 16/32; A61K 39/00; A61P 35/00; A61P 37/04; C07K 16/28

(52) U.S. Cl. CPC C07K 16/32; A61P 35/00; A61P 37/04; C07K 16/2809; C07K 16/2818; A61K 2039/505; A61K 2039/54; A61K 2039/545; C07K 2317/31; C07K 2317/522; C07K 2317/524; C07K 2317/526; C07K 2317/53; C07K 2317/565

(57) ABSTRACT

The present disclosure provides method of treating a subject having a cancer comprising administering to the subject about 0.2 μg to about 4500 μg of a trispecific binding protein, wherein the trispecific binding protein comprises a first antigen binding site that binds a CD28 polypeptide, a second antigen binding site that binds a CD3 polypeptide, and a third antigen binding site that binds a HER2 polypeptide.

### CROSS-REFERENCE TO RELATED APPLICATIONS

This application claims the benefit of U.S. Provisional Application No. 63/648,948, filed May 17, 2024, U.S. Provisional Application No. 63/692,288, filed Sep. 9, 2024, and U.S. Provisional Application No. 63/764,897, filed Feb. 28, 2025, each of which is hereby incorporated by reference in its entirety.

### REFERENCE TO AN ELECTRONIC SEQUENCE LISTING

The contents of the electronic sequence listing (183952036200SEQLIST.xml; Size: 173,257 bytes; and Date of Creation: Apr. 21, 2025) is herein incorporated by reference in its entirety.

### FIELD

The disclosure relates generally to methods of treating cancer in a subject using trispecific binding proteins that bind to human epidermal growth factor receptor 2 (HER2), CD3, and CD28.

### BACKGROUND

HER2 is over-expressed in multiple cancers, including breast, gastric, lung, gastroesophageal, ovarian, bladder, colon, and others. HER2 is a clinically validated tumor target, and trastuzumab (Herceptin), a humanized HER2 antibody is used as the standard of care in combination with chemotherapy for participants with early stage HER2+ breast cancer as well as for participants with advanced-stage disease (Bartsch R. et al. Trastuzumab in the management of early and advanced stage breast cancer. Biologics. 2007; 1(1):19-31). Development of additional HER2 targeting agents, such as pertuzumab (Perjeta), a humanized HER2 antibody that binds to a domain distinct from trastuzumab, trastuzumab-DM (T-DM1) with cytotoxic payload coupled to trastuzumab, and small molecule inhibitors to disable HER2 kinase activity quickly followed to improve response rate and overall survival (Pernas S. et al. HER2-positive breast cancer: new therapeutic frontiers and overcoming resistance. Ther Adv Med Oncol. 2019; 11:1758835919833519; and Zhao D. et al. Progress and challenges in HER2-positive gastroesophageal adenocarcinoma. J Hematol Oncol. 2019; 12(1):50). Current HER2-targeting approaches in the clinic are focused against HER2 overexpressing tumors. Limited data are available on HER2 status in residual disease following HER2-targeted treatment.

Despite the multitude of potential treatment options, there is still an unmet need for safe and effective therapies for HER2+ cancers. Provided herein are trispecific binding proteins that meet such needs.

### BRIEF SUMMARY

The present disclosure provides methods for treating cancer in a subject by administering trispecific binding proteins that bind human epidermal growth factor receptor 2 (HER2) (expressed on tumor cells), CD3 (part of the T cell antigen receptor signaling complex on T cells) and CD28 (functions as a co-stimulatory molecule on T cells). HER2 amplification and overexpression can be found in molecular subtypes of breast cancer, and also in gastric, ovarian, lung and prostate carcinomas. Optimal activation of T cells requires two factors: 1. Antigen recognition and 2. Co-stimulation. Using the HER2/CD28×CD3 trispecific binding proteins described herein, Signal 1 is provided by an agonist anti-CD3 binding site, and Signal 2 is provided by an agonist anti-CD28 binding site. The trispecific binding protein recruits T cells to the tumor via HER2 and activates the engaged T cells via anti-CD3 and -CD28. The resulting activation induces the killing potential of the immune cells against the nearby tumor cells.

In some aspects, provided herein is a method of treating a subject having a cancer comprising administering to the subject about 0.2 μg to about 4500 μg of a trispecific binding protein, wherein the trispecific binding protein comprises a first antigen binding site that binds a CD28 polypeptide, a second antigen binding site that binds a CD3 polypeptide, and a third antigen binding site that binds a HER2 polypeptide.

In some embodiments, the trispecific binding protein comprises four polypeptide chains that form three antigen binding sites, wherein a first polypeptide chain comprises a structure represented by the formula:

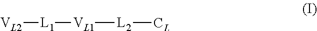

and a second polypeptide chain comprises a structure represented by the formula:

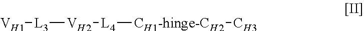

and a third polypeptide chain comprises a structure represented by the formula:

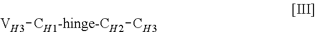

and a fourth polypeptide chain comprises a structure represented by the formula:

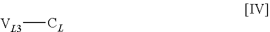

wherein:
V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain;
C_{H2} is an immunoglobulin C_{H2} heavy chain constant domain;
C_{H3} is an immunoglobulin C_{H3} heavy chain constant domain;
hinge is an immunoglobulin hinge region connecting the C_{H1} and C_{H2} domains; and
L₁, L₂, L₃ and L₄ are amino acid linkers;
wherein the polypeptide of formula I and the polypeptide of formula II form a cross-over light chain-heavy chain pair; and
wherein V_{H1} and V_{L1} form the first antigen binding site that binds a CD28 polypeptide, wherein the V_{H1} domain comprises a CDR-H1 sequence comprising the amino acid sequence of GYTFTSYY (SEQ ID NO: 49), a CDR-H2 sequence comprising the amino acid sequence of IYPGNVNT (SEQ ID NO:50), and a CDR-H3 sequence comprising the amino acid sequence of TRSHYGLDWNFDV (SEQ ID NO:51), and the V_{L1} domain comprises a CDR-L1 sequence comprising the amino acid sequence of QNIYVW (SEQ ID NO:52), a CDR-L2 sequence comprising the amino acid sequence of KAS (SEQ ID NO:53), and a CDR-L3 sequence comprising the amino acid sequence of QQGQTYPY (SEQ ID NO:54);
wherein V_{H2} and V_{L2} form the second antigen binding site that binds a CD3 polypeptide, wherein the V_{H2} domain comprises a CDR-H1 sequence comprising the amino acid sequence of GFTFTKAW (SEQ ID NO:55), a CDR-H2 sequence comprising the amino acid sequence of IKDKSNSYAT (SEQ ID NO:56), and a CDR-H3 sequence comprising the amino acid sequence of RGVYYALSPFDY (SEQ ID NO:57), and the V_{L2} domain comprises a CDR-L1 sequence comprising the amino acid sequence of QSLVHQNAQTY (SEQ ID NO:59), a CDR-L2 sequence comprising the amino acid sequence of KVS (SEQ ID NO:64), and a CDR-L3 sequence comprising the amino acid sequence of GQGTQYPFT (SEQ ID NO:65); and
wherein V_{H3} and V_{L3} form the third antigen binding site that binds a HER2 polypeptide, wherein the V_{H3} domain comprises a CDR-H1 sequence comprising the amino acid sequence of GFNIRDTY (SEQ ID NO:2), a CDR-H2 sequence comprising the amino acid sequence of IYPTQGYT (SEQ ID NO:4), and a CDR-H3 sequence comprising the amino acid sequence of SRWGGEGFYAMDY (SEQ ID NO:7), and the V_{L3} domain comprises a CDR-L1 sequence comprising the amino acid sequence of QDVNTA (SEQ ID NO:9), a CDR-L2 sequence comprising the amino acid sequence of SAS (SEQ ID NO:11), and a CDR-L3 sequence comprising the amino acid sequence of QQHYTTP (SEQ ID NO:12).

In some embodiments, the V_{H1} domain comprises the amino acid sequence of QVQLVQSGAEVVKPGASVKVSCKASGYTFTSYYIHWVRQAPGQGLEWIGSIYPGNVNT NYAQKFQGRATLTVDTSISTAYMELSRLRSDDTAVYYCTRSHYGLDWNFDVWGKGTT VTVSS (SEQ ID NO:91), and the V_{L1} domain comprises the amino acid sequence of DIQMTQSPSSLSASVGDRVTITCQASQNIYVWLNWYQQKPGKAPKLLIYKASNLHTGVP SRFSGSGSGTDFTLTISSLQPEDIATYYCQQGQTYPYTFGQGTKLEIK (SEQ ID NO:92).

In some embodiments, the V_{H2} domain comprises the amino acid sequence of QVQLVESGGGVVQPGRSLRLSCAASGFTFTKAWMHWVRQAPGKQLEWVAQIKDKSNS YATYYADSVKGRFTISRDDSKNTLYLQMNSLRAEDTAVYYCRGVYYALSPFDYWGQG TLVTVSS (SEQ ID NO:93), and the V_{L2} domain comprises the amino acid sequence of DIVMTQTPLSLSVTPGQPASISCKSSQSLVHQNAQTYLSWYLQKPGQSPQSLIYKVSNRF SGVPDRFSGSGSGTDFTLKISRVEAEDVGVYYCGQGTQYPFTFGSGTKVEIK (SEQ ID NO:95).

In some embodiments, the V_{H3} domain comprises the amino acid sequence of EVQLVESGGGLVQPGGSLRLSCAASGFNIRDTYIHWVRQAPGKGLEWVARIYPTQGYTR YADSVKGRFTISADTSKNTAYLQMNSLRAEDTAVYYCSRWGGEGFYAMDYWGQGTL VTVSS (SEQ ID NO:73), and the V_{L3} domain comprises the amino acid sequence of DIQMTQSPSSLSASVGDRVTITCRASQDVNTAVAWYQQKPGKAPKLLIYSASFLYSGVP SRFSGSRSGTDFTLTISSLQPEDFATYYCQQHYTTPPTFGQGTKVEIK (SEQ ID NO:77).

In some embodiments, the first polypeptide chain comprises the amino acid sequence of SEQ ID NO:104 or an amino acid sequence that is at least 95% identical to the amino acid sequence of SEQ ID NO:104; the second polypeptide chain comprises the amino acid sequence of SEQ ID NO:105 or an amino acid sequence that is at least 95% identical to the amino acid sequence of SEQ ID NO:105; the third polypeptide chain comprises the amino acid sequence of SEQ ID NO:106 or an amino acid sequence that is at least 95% identical to the amino acid sequence of SEQ ID NO:106; and the fourth polypeptide chain comprises the amino acid sequence of SEQ ID NO:107 or an amino acid sequence that is at least 95% identical to the amino acid sequence of SEQ ID NO:107.

In some embodiments, L₁, L₂, L₃ and L₄ each independently comprise the sequence DKTHT (SEQ ID NO:66).

In some embodiments, the hinge-C_{H2}-C_{H3} domains of the second and the third polypeptide chains are human IgG4 hinge-C_{H2}-C_{H3} domains, and wherein the hinge-C_{H2}-C_{H3} domains each comprise amino acid substitutions at positions corresponding to positions 234 and 235 of human IgG4 according to EU Index, wherein the amino acid substitutions are F234A and L235A.

In some embodiments, the hinge-C_{H2}-C_{H3} domains of the second and the third polypeptide chains are human IgG4 hinge-C_{H2}-C_{H3} domains, and wherein the hinge-C_{H2}-C_{H3} domains each comprise amino acid substitutions at positions corresponding to positions 228 and 409 of human IgG4 according to EU Index, wherein the amino acid substitutions are S228P and R409K.

In some embodiments, the hinge-C_{H2}-C_{H3} domain of the second polypeptide chain comprises amino acid substitutions at positions corresponding to positions 349, 366, 368, and 407 of human IgG1 or IgG4 according to EU Index, wherein the amino acid substitutions are Y349C, T366S, L368A, and Y407V; and wherein the hinge-C_{H2}-C_{H3} domain of the third polypeptide chain comprises amino acid substitutions at positions corresponding to positions 354 and 366 of human IgG1 or IgG4 according to EU Index, wherein the amino acid substitutions are S354C and T366W.

In some embodiments, the hinge-C_{H2}-C_{H3} domain of the second polypeptide chain comprises amino acid substitutions at positions corresponding to positions 354 and 366 of human IgG1 or IgG4 according to EU Index, wherein the amino acid substitutions are S354C and T366W; and wherein the hinge-C_{H2}-C_{H3} domain of the third polypeptide chain comprises amino acid substitutions at positions corresponding to positions 349, 366, 368, and 407 of human IgG1 or IgG4 according to EU Index, wherein the amino acid substitutions are Y349C, T366S, L368A, and Y407V.

In some embodiments, the trispecific binding protein is administered to the subject once per week, once every two weeks, once every three weeks, or once every four weeks. In some embodiments, the trispecific binding protein is administered to the subject for at least one 28-day cycle. In some embodiments, the trispecific binding protein is administered to the subject weekly at a dose of between about 10 μg to about 1500 μg during the at least one 28-day cycle. In some embodiments, each dose of the trispecific binding protein is about 18 μg, about 60 μg, about 180 μg, about 240 μg, about 360 μg, about 480 μg, about 720 μg, about 900 μg, about 1080 μg, about 1125 μg, or about 1440 μg. In some embodiments, the trispecific binding protein is administered to the subject on days 1, 8, 15, and 22 of the at least one 28-day cycle.

In some embodiments, the trispecific binding protein is administered to the subject during a lead-in phase prior to initiation of the at least one 28-day cycle. In some embodiments, the lead-in phase is 28 days, and wherein the trispecific binding protein is administered to the subject at a dose of between about 0.2 μg to about 1500 μg during the lead-in phase. In some embodiments, the lead-in phase comprises an escalating treatment regimen, wherein the escalating treatment regimen comprises administration of the trispecific binding protein to the subject at the following sequential doses: (i) about 0.6 μg, about 1.8 μg, about 6.0 μg, about 18 μg, and about 18 μg; (ii) about 1.8 μg, about 6.0 μg, about 18 μg, about 60 μg, and about 60 μg; (iii) about 6.0 μg, about 18 μg, about 60 μg, about 180 μg, and about 180 μg; (iv) about 9.0 μg, about 27 μg, about 90 μg, about 240 μg, and about 240 μg; (v) about 18 μg, about 60 μg, about 180 μg, about 360 μg, and about 360 μg; (vi) about 18 μg, about 60 μg, about 180 μg, about 720 μg, and about 720 μg; (vii) about 18 μg, about 60 μg, about 180 μg, about 1080 μg, and about 1080 μg; (viii) about 18 μg, about 60 μg, about 180 μg, about 1440 μg, and about 1440 μg; (ix) about 18 μg, about 60 μg, about 120 μg, about 240 μg, and about 480 μg; (x) about 18 μg, about 60 μg, about 120 μg, about 240 μg, and about 720 μg; (xi) about 18 μg, about 60 μg, about 150 μg, about 300 μg, and about 900 μg; or (xii) about 18 μg, about 60 μg, about 150 μg, about 300 μg, and about 1125 μg. In some embodiments, the lead-in phase comprises administration of the trispecific binding protein to the subject on days 1, 4, 8, 15, and 22 of the lead-in phase.

In some embodiments, the trispecific binding protein is administered to the subject intravenously.

In some embodiments, the trispecific binding protein is administered to the subject weekly at a dose of between about 300 μg to about 4500 μg. In some embodiments, each dose of the trispecific binding protein is about 360 μg, about 1100 μg, about 2200 μg, about 3300 μg, or about 4200 μg.

In some embodiments, the trispecific binding protein is administered to the subject during a lead-in phase prior to initiation of weekly administration of the trispecific binding protein. In some embodiments, the lead-in phase is 8 days, and wherein the trispecific binding protein is administered to the subject at a dose of between about 10 μg to about 600 μg during the lead-in phase. In some embodiments, the lead-in phase comprises an escalating treatment regimen, wherein the escalating treatment regimen comprises administration of the trispecific binding protein to the subject at the following sequential doses: (i) about 20 μg, about 60 μg, and about 180 μg; or (ii) about 60 μg, about 180 μg, and about 500 μg. In some embodiments, the lead-in phase comprises administration of the trispecific binding protein to the subject on days 1, 4, and 8 of the lead-in phase.

In some embodiments, the trispecific binding protein is administered to the subject subcutaneously.

In some embodiments, prior to initiation of the lead-in phase, the trispecific binding protein is administered to the subject intravenously in an additional escalating treatment regimen. In some embodiments, the additional escalating treatment regimen comprises administration of the trispecific binding protein to the subject within a 28-day period, and wherein the trispecific binding protein is administered to the subject at a dose of between about 0.2 μg to about 1500 μg during the additional escalating treatment regimen.

In some embodiments, the method does not comprise a lead-in phase.

In some embodiments, the cancer is a HER2-positive cancer. In some embodiments, the cancer has a HER2 immunohistochemistry (IHC) score of 1+, 2+, or 3+. In some embodiments, the cancer exhibits HER2 amplification. In some embodiments, the cancer does not exhibit HER2 amplification. In some embodiments, the cancer has at least one HER2 activating mutation. In some embodiments, the cancer comprises a solid tumor. In some embodiments, the cancer is advanced. In some embodiments, the cancer is metastatic. In some embodiments, the cancer is a relapsed and/or refractory cancer. In some embodiments, the cancer is breast cancer, gastric cancer, ovarian cancer, bladder cancer, colon cancer, or lung cancer. In some embodiments, the cancer is non-small cell lung cancer (NSCLC).

In some embodiments, the subject does not exhibit cytokine release syndrome (CRS), neurotoxicity, neutropenia, thrombocytopenia, hematologic toxicity, myocardial toxicity, pulmonary toxicity, hepatotoxicity, tumor lysis syndrome, or any combination thereof. In some embodiments, the subject does not exhibit transaminase and/or alanine aminotransferase elevation. In some embodiments, the subject does not exhibit pyrexia.

In some embodiments, administration of the trispecific binding protein to the subject results in an increase in the level of at least one cytokine in the peripheral blood of the subject compared to the level of the at least one cytokine in the peripheral blood of the subject prior to the administration of the trispecific binding protein. In some embodiments, the at least one cytokine is IL2, IFNγ, IL6, IL10, TNF-α, or a combination thereof.

In some embodiments, administration of the trispecific binding protein to the subject results in an increase in the level of at least one T cell activation marker in the peripheral blood of the subject compared to the level of the at least one T cell activation marker in the peripheral blood of the subject prior to the administration of the trispecific binding protein. In some embodiments, the at least one T cell activation marker is CD57, PD1, HLA-DR, or a combination thereof.

In some embodiments, administration of the trispecific binding protein to the subject results in an increase in the level of a cytotoxicity biomarker in the peripheral blood of the subject compared to the level of the cytotoxicity biomarker in the peripheral blood of the subject prior to the administration of the trispecific binding protein. In some embodiments, the cytotoxicity biomarker is granzyme B.

In some embodiments, administration of the trispecific binding protein to the subject results in an increase in the level of a proliferation biomarker in the peripheral blood of the subject compared to the level of the proliferation biomarker in the peripheral blood of the subject prior to the administration of the trispecific binding protein. In some embodiments, the proliferation biomarker is Ki67.

In some embodiments, the subject has received prior chemotherapy and/or radiation therapy. In some embodiments, the method delays progression of the cancer in the subject. In some embodiments, the method reduces the tumor size in the subject. In some embodiments, the method reduces the number and/or size of metastatic lesions in the subject. In some embodiments, the trispecific binding protein is administered until disease progression.

In some embodiments, the subject is a human.

In some aspects, provided herein is a trispecific binding protein for use in treating a subject having a cancer according to the method of any one of the preceding embodiments, wherein the trispecific binding protein comprises a first antigen binding site that binds a CD28 polypeptide, a second antigen binding site that binds a CD3 polypeptide, and a third antigen binding site that binds a HER2 polypeptide.

In some aspects, provided herein is a use of a trispecific binding protein for treating a subject having a cancer according to the method of any one of the preceding embodiments, wherein the trispecific binding protein comprises a first antigen binding site that binds a CD28 polypeptide, a second antigen binding site that binds a CD3 polypeptide, and a third antigen binding site that binds a HER2 polypeptide.

In some aspects, provided herein is a use of a trispecific binding protein for the manufacture of a medicament for treating a subject having a cancer according to the method of any one of the preceding embodiments, wherein the trispecific binding protein comprises a first antigen binding site that binds a CD28 polypeptide, a second antigen binding site that binds a CD3 polypeptide, and a third antigen binding site that binds a HER2 polypeptide.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE FIGURES

The present application can be understood by reference to the following description taken in conjunction with the accompanying figures.

FIG. 1 provides an exemplary schematic representation of a binding protein provided herein comprising four polypeptide chains that form three antigen binding sites that binds three target proteins: CD28, CD3, and HER2. A first pair of polypeptides possess dual variable domains having a cross-over orientation (VH1-VH2 and VL2-VL1) forming two antigen binding sites that recognize CD3 and CD28, and a second pair of polypeptides possess a single variable domain (VH3 and VL3) forming a single antigen binding site that recognizes HER2. The trispecific binding protein shown in FIG. 1 uses a constant region.

FIG. 2 provides a proposed mechanism of action for HER2/CD28×CD3 trispecific antibody-mediated T cell activation and HER2+ cancer cell killing.

FIGS. 3A-3B show Binding Protein #2 induces an increase in interleukin-2 (IL2) and interferon-T (IFNγ) levels after each infusion. Shown is median fold-change of IL2 (FIG. 3A) and IFNγ (FIG. 3B) in all patients over time. Abbreviations: C, cycle; D, day; T, time; H, hour.

FIGS. 4A-4D show the efficacy of Binding Protein #2 administered intravenously to patients with advanced solid tumors. Shown are tumor shrinkage percentage (FIG. 4A) and duration of disease control (FIG. 4B) for patients in the 2-week lead-in cohort. Also shown are tumor shrinkage percentage (FIG. 4C) and duration of disease control (FIG. 4D) for patients in the 3-week lead-in cohort. Abbreviations: AE, adverse event; BOR, best overall survival; DL, dose level; EOT, reason for discontinuation of treatment; NE, not evaluable; PD, progressive disease; SD, stable disease.

FIG. 5 shows the pharmacokinetic profile of Binding Protein #2 in patients of the 2-week lead-in cohort. Shown is mean Binding Protein #2 concentration (ng/mL) as a function of time (day) after intravenous administration of the binding protein on day 15 of cycle 1 in the dosing regimen. Abbreviations: C, cycle; D, day; IV, intravenous; PK, pharmacokinetics; T, time.

FIG. 6 shows a heatmap generated from a T cell flow cytometry panel, reflecting modulation of peripheral T cell activation in patients following administrations of Binding Protein #2 in a dosing regimen. Abbreviations: C, cycle; CD, cluster of differentiation; D, day; PD1, programmed cell death protein 1; HLA-DR, human leukocyte antigen-DR isotype; Tregs, regulatory T cells.

FIG. 7 shows an illustration of an in vitro system model. FIG. 7 shows interactions and processes included in the model at the molecular and cellular scale. There are three different cell types: CD8, CD4, and HER2-expressing target cell (denoted as X), as illustrated. Both CD8 and CD4 express CD3 and CD28 proteins, while the target cell X expresses HER2 protein. The number of each protein molecule at a given time depends on corresponding cell type and its expression level (copy number per cell). Upon addition of the tri-specific drug (Ab), it forms complexes via interaction with CD3, CD28, and HER2 molecules, as shown. Among these complexes, the trimeric complexes represent contributes to synapse formation between CD8 and X or CD4 and X. The model assumes that a synaptic formation can occur between two cells only considering steric hindrance. While in synapse, the T cell (CD8 or CD4) become activated leading to cell killing. The model assumes the T cell is freed immediately upon killing of the target cell (X). The activated T cells proliferate at a higher (induced) rate compared to its basal proliferation. Besides, the activated T cells are also subject to an induced rate of death relative to the basal apoptosis. Part of the activated T cells form memory T cells, as illustrated. The activated T cells release various cytokines.

FIGS. 8A-8C show T cell activation and cytotoxicity response profiles of Binding Protein #2 in vitro. The model was calibrated with data from HCC-1954 cell lines at 48 h after incubation with Binding Protein #2. In each panel, the points correspond to data and the dashed line represents model calibration. The dotted line and dash-dot line represent model-predicted responses at 10- and 100-fold lower HER2 expression than HCC-1954 cells (760,000 HER2/cell). Shown are data and predictions for CD4 activation (FIG. 8A), CD8 activation (FIG. 8B), and cytotoxicity response (FIG. 8C) profiles of Binding Protein #2, respectively, over the dose ranges indicated.

FIGS. 9A-9B show prediction of dose regiment for ex-vivo efficacy of Binding Protein #2. FIG. 9A shows model-predicted dose-response profiles for various trimer formation based on Binding Protein #2 interactions with the T cell and target cell surface proteins. The dashed line and dash-dot line represent trimeric complexes binding CD8 and target cell, while the dotted line and dash double-dot line represent trimeric complexes binding CD4 and target cell, as indicated in the figure label. The solid line represents sum of the four different trimeric complexes. FIG. 9B presents a 2D greyscale map showing trimer formation as a function of HER2 expression and Binding Protein #2 dosing.

FIG. 10 shows ex-vivo cytokine responses captured by the in vitro model. Dose dependence of four different cytokines (IL6, IL10, IFNγ and TNFα). The data corresponds to concentrations after 48 hours of incubation of primary T cells and HCC-1954 co-cultures with Binding Protein #2. The points represent data. The lines represent corresponding simulation after model calibration.

FIGS. 11A-11F show in vivo model-predicted T cell activation and IL-6 responses in cynomolgus monkeys based on target engagement in plasma. FIG. 11A shows Binding Protein #2 concentration in plasma for two different doses (10 μg/kg and 30 μg/kg) after infusion. Points represent data and lines represent model calibration. FIG. 11B shows predicted CD8 T cell activation in 500 virtual subjects in response to single dose of 10 μg/kg. FIG. 11C shows IL6 responses corresponding to the virtual subjects in FIG. 11B. FIG. 11D shows simulated repeat doses at 10 μg/kg infused at time, t=9, 72, 168, 240 hours. FIG. 11E shows predicted CD8 activation in 500 virtual subjects in response to the repeat doses in FIG. 11D. FIG. 11F shows IL-6 responses in virtual subjects corresponding to FIG. 11E. The virtual subjects were created by introducing log-normally distributed noise in blood T cell count.

### DETAILED DESCRIPTION

The present disclosure provides methods for treating cancer in an individual by administering a binding protein that binds to three distinct antigens, including HER2 on tumor cells, CD3 and CD28.

The following description sets forth exemplary methods, parameters and the like. It should be recognized, however, that such description is not intended as a limitation on the scope of the present disclosure but is instead provided as a description of exemplary embodiments.

The disclosures of all publications, patents, and patent applications referred to herein are each hereby incorporated by reference in their entireties. To the extent that any reference incorporated by reference conflicts with the instant disclosure, the instant disclosure shall control.

### I. Definitions

As utilized in accordance with the present disclosure, the following terms, unless otherwise indicated, shall be understood to have the following meanings. Unless otherwise required by context, singular terms shall include pluralities and plural terms shall include the singular.

It is understood that aspects and embodiments of the disclosure described herein include “comprising,” “consisting,” and “consisting essentially of” aspects and embodiments.

An “isolated polynucleotide” is a polynucleotide of genomic, cDNA, or synthetic origin or some combination thereof, which: (1) is not associated with all or a portion of a polynucleotide in which the isolated polynucleotide is found in nature, (2) is linked to a polynucleotide to which it is not linked in nature, or (3) does not occur in nature as part of a larger sequence.

An “isolated polypeptide” is one that: (1) is free of at least some other polypeptides with which it would normally be found, (2) is essentially free of other polypeptides from the same source, e.g., from the same species, (3) is expressed by a cell from a different species, (4) has been separated from at least about 50 percent of polynucleotides, lipids, carbohydrates, or other materials with which it is associated in nature, (5) is not associated (by covalent or noncovalent interaction) with portions of a polypeptide with which the “isolated polypeptide” is associated in nature, (6) is operably associated (by covalent or noncovalent interaction) with a polypeptide with which it is not associated in nature, or (7) does not occur in nature. Such an isolated polypeptide can be encoded by genomic DNA, cDNA, mRNA or other RNA, of synthetic origin, or any combination thereof. Preferably, the isolated polypeptide is substantially free from polypeptides or other contaminants that are found in its natural environment that would interfere with its use (therapeutic, diagnostic, prophylactic, research or otherwise).

Naturally occurring antibodies typically comprise a tetramer. Each such tetramer is typically composed of two identical pairs of polypeptide chains, each pair having one full-length “light” chain (typically having a molecular weight of about 25 kDa) and one full-length “heavy” chain (typically having a molecular weight of about 50-70 kDa). The terms “heavy chain” and “light chain” as used herein refer to any immunoglobulin polypeptide having sufficient variable domain sequence to confer specificity for a target antigen. The amino-terminal portion of each light and heavy chain typically includes a variable domain of about 100 to 110 or more amino acids that typically is responsible for antigen recognition. The carboxy-terminal portion of each chain typically defines a constant domain responsible for effector function. Thus, in a naturally occurring antibody, a full-length heavy chain immunoglobulin polypeptide includes a variable domain (V_{H}) and three constant domains (C_{H1}, C_{H2}, and C_{H3}), wherein the V_{H} domain is at the amino-terminus of the polypeptide and the C_{H3} domain is at the carboxyl-terminus, and a full-length light chain immunoglobulin polypeptide includes a variable domain (V_{L}) and a constant domain (C_{L}), wherein the V_{L} domain is at the amino-terminus of the polypeptide and the C_{L} domain is at the carboxyl-terminus.

Human light chains are typically classified as kappa and lambda light chains, and human heavy chains are typically classified as mu, delta, gamma, alpha, or epsilon, and define the antibody's isotype as IgM, IgD, IgG, IgA, and IgE, respectively. IgG has several subclasses, including, but not limited to, IgG1, IgG2, IgG3, and IgG4. IgM has subclasses including, but not limited to, IgM1 and IgM2. IgA is similarly subdivided into subclasses including, but not limited to, IgA1 and IgA2. Within full-length light and heavy chains, the variable and constant domains typically are joined by a “J” region of about 12 or more amino acids, with the heavy chain also including a “D” region of about 10 more amino acids. See, e.g., FUNDAMENTAL IMMUNOLOGY (Paul, W., ed., Raven Press, 2nd ed., 1989), which is incorporated by reference in its entirety for all purposes. The variable regions of each light/heavy chain pair typically form an antigen binding site. The variable domains of naturally occurring antibodies typically exhibit the same general structure of relatively conserved framework regions (FR) joined by three hypervariable regions, also called complementarity determining regions or CDRs. The CDRs from the two chains of each pair typically are aligned by the framework regions, which may enable binding to a specific epitope. From the amino-terminus to the carboxyl-terminus, both light and heavy chain variable domains typically comprise the domains FR1, CDR1, FR2, CDR2, FR3, CDR3, and FR4.

The term “CDR set” refers to a group of three CDRs that occur in a single variable region capable of binding the antigen. The exact boundaries of these CDRs have been defined differently according to different systems. The system described by Kabat (Kabat et al., SEQUENCES OF PROTEINS OF IMMUNOLOGICAL INTEREST (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia and Lesk, 1987, J. Mol. Biol. 196: 901-17; Chothia et al., 1989, Nature 342: 877-83) found that certain sub-portions within Kabat CDRs adopt nearly identical peptide backbone conformations, despite having great diversity at the level of amino acid sequence. These sub-portions were designated as L1, L2, and L3 or H1, H2, and H3 where the “L” and the “H” designates the light chain and the heavy chain regions, respectively. These regions may be referred to as Chothia CDRs, which have boundaries that overlap with Kabat CDRs. Other boundaries defining CDRs overlapping with the Kabat CDRs have been described by Padlan, 1995, FASEB J. 9: 133-39; MacCallum, 1996, J. Mol. Biol. 262(5): 732-45; and Lefranc, 2003, Dev. Comp. Immunol. 27: 55-77. Still other CDR boundary definitions may not strictly follow one of the herein systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat or Chothia defined CDRs. Identification of predicted CDRs using the amino acid sequence is well known in the field, such as in Martin, A. C. “Protein sequence and structure analysis of antibody variable domains,” In Antibody Engineering, Vol. 2. Kontermann R., Dübel S., eds. Springer-Verlag, Berlin, p. 33-51 (2010). The amino acid sequence of the heavy and/or light chain variable domain may be also inspected to identify the sequences of the CDRs by other conventional methods, e.g., by comparison to known amino acid sequences of other heavy and light chain variable regions to determine the regions of sequence hypervariability. The numbered sequences may be aligned by eye, or by employing an alignment program such as one of the CLUSTAL suite of programs, as described in Thompson, 1994, Nucleic Acids Res. 22: 4673-80. Molecular models are conventionally used to correctly delineate framework and CDR regions and thus correct the sequence-based assignments.

The term “Fc” as used herein refers to a molecule comprising the sequence of a non-antigen-binding fragment resulting from digestion of an antibody or produced by other means, whether in monomeric or multimeric form, and can contain the hinge region. The original immunoglobulin source of the native Fc is preferably of human origin and can be any of the immunoglobulins, although IgG1 and IgG2 are preferred. Fc molecules are made up of monomeric polypeptides that can be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, and IgE) or subclass (e.g., IgG1, IgG2, IgG3, IgA1, and IgGA2). One example of a Fc is a disulfide-bonded dimer resulting from papain digestion of an IgG. The term “native Fc” as used herein is generic to the monomeric, dimeric, and multimeric forms.

A F(ab) fragment typically includes one light chain and the V_{H} and C_{H1} domains of one heavy chain, wherein the V_{H}-C_{H1} heavy chain portion of the F(ab) fragment cannot form a disulfide bond with another heavy chain polypeptide. As used herein, a F(ab) fragment can also include one light chain containing two variable domains separated by an amino acid linker and one heavy chain containing two variable domains separated by an amino acid linker and a C_{H1} domain.

A F(ab′) fragment typically includes one light chain and a portion of one heavy chain that contains more of the constant region (between the C_{H1} and C_{H2} domains), such that an interchain disulfide bond can be formed between two heavy chains to form a F(ab′)2 molecule.

The term “binding protein” as used herein refers to a non-naturally occurring (or recombinant or engineered) molecule that specifically binds to three distinct target antigens. A trispecific binding protein of the present disclosure, unless otherwise specified, typically comprises four polypeptide chains that form at least three antigen binding sites, wherein a first polypeptide chain has a structure represented by the formula:

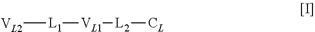

and a second polypeptide chain has a structure represented by the formula:

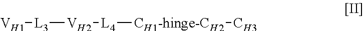

and a third polypeptide chain has a structure represented by the formula:

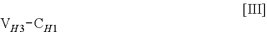

and a fourth polypeptide chain has a structure represented by the formula:

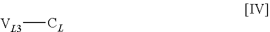

wherein:
VU is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is the immunoglobulin C_{H1} heavy chain constant domain; and
hinge is an immunoglobulin hinge region connecting the C_{H1} and C_{H2} domains;
L₁, L₂, L₃ and L₄ are amino acid linkers;
and wherein the polypeptide of formula I and the polypeptide of formula II form a cross-over light chain-heavy chain pair.

A “recombinant” molecule is one that has been prepared, expressed, created, or isolated by recombinant means.

The term “antigen” or “target antigen” or “antigen target” as used herein refers to a molecule or a portion of a molecule that is capable of being bound by a binding protein, and additionally is capable of being used in an animal to produce antibodies capable of binding to an epitope of that antigen. A target antigen may have one or more epitopes. With respect to each target antigen recognized by a binding protein, the binding protein is capable of competing with an intact antibody that recognizes the target antigen.

The term “HER2” refers to human epidermal growth factor receptor 2 which is a member of the epidermal growth factor receptor family.

“CD3” is cluster of differentiation factor 3 polypeptide and is a T-cell surface protein that is typically part of the T cell receptor (TCR) complex.

“CD28” is cluster of differentiation 28 polypeptide and is a T-cell surface protein that provides co-stimulatory signals for T-cell activation and survival.

The term “trispecific binding protein” refers to a binding protein that specifically binds to three different antigen targets.

The term “trivalent binding protein” refers to a binding protein that has three binding sites. Provided herein is a trivalent binding protein can bind to three antigen targets.

An “isolated” binding protein is one that has been identified and separated and/or recovered from a component of its natural environment. Contaminant components of its natural environment are materials that would interfere with diagnostic or therapeutic uses for the binding protein, and may include enzymes, hormones, and other proteinaceous or non-proteinaceous solutes. In some embodiments, the binding protein will be purified: (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated binding proteins include the binding protein in situ within recombinant cells since at least one component of the binding protein's natural environment will not be present.

The dissociation constant (K_{D}) of a binding protein can be determined, for example, by surface plasmon resonance. Generally, surface plasmon resonance analysis measures real-time binding interactions between ligand (a target antigen on a biosensor matrix) and analyte (a binding protein in solution) by surface plasmon resonance (SPR) using the BIAcore system (Pharmacia Biosensor; Piscataway, NJ). Surface plasmon analysis can also be performed by immobilizing the analyte (binding protein on a biosensor matrix) and presenting the ligand (target antigen). The term “K_{D},” as used herein refers to the dissociation constant of the interaction between a particular binding protein and a target antigen.

The term “specifically binds” as used herein refers to the ability of a binding protein or an antigen-binding fragment thereof to bind to an antigen containing an epitope with an Kd of at least about 1×10⁶M, 1×10⁷M, 1×10⁸M, 1×10⁹M, 1×10¹⁰M, 1×10¹¹M, 1×10¹²M, or more, and/or to bind to an epitope with an affinity that is at least two-fold greater than its affinity for a nonspecific antigen.

The term “linker” as used herein refers to one or more amino acid residues inserted between immunoglobulin domains to provide sufficient mobility for the domains of the light and heavy chains to fold into cross over dual variable region immunoglobulins. A linker is inserted at the transition between variable domains or between variable and constant domains, respectively, at the sequence level. The transition between domains can be identified because the approximate size of the immunoglobulin domains is well understood. The precise location of a domain transition can be determined by locating peptide stretches that do not form secondary structural elements such as beta-sheets or alpha-helices as demonstrated by experimental data or as can be assumed by techniques of modeling or secondary structure prediction. The linkers described herein are referred to as L₁, which is located on the light chain between the C-terminus of the V_{L2} and the N-terminus of the V_{L1} domain; and L₂, which is located on the light chain between the C-terminus of the V_{L1} and the N-terminus of the C_{L} domain. The heavy chain linkers are known as L₃, which is located between the C-terminus of the V_{H1} and the N-terminus of the V_{H2} domain; and L₄, which is located between the C-terminus of the V_{H2} and the N-terminus of the C_{H1} domain.

A skilled artisan will be able to determine suitable variants of the polypeptide chains of the binding proteins using well-known techniques. For example, one skilled in the art may identify suitable areas of a polypeptide chain that may be changed without destroying activity by targeting regions not believed to be important for activity. Alternatively, one skilled in the art can identify residues and portions of the molecules that are conserved among similar polypeptides. In addition, even areas that may be important for biological activity or for structure may be subject to conservative amino acid substitutions without destroying the biological activity or without adversely affecting the polypeptide structure.

The term “patient” or “subject” as used herein includes human and animal subjects.

The terms “treatment” or “treat” as used herein refer to both therapeutic treatment and prophylactic or preventative measures. Those in need of treatment include those having a disorder as well as those prone to have the disorder or those in which the disorder is to be prevented. In particular embodiments, binding proteins can be used to treat humans with cancer, or humans susceptible to cancer, in order to prevent, cure, delay, reduce the severity of, or ameliorate one or more symptoms of the cancer or recurring cancer, or in order to prolong the survival of a subject beyond that expected in the absence of such treatment. The binding proteins can also be used to prevent cancer in a human patient.

The terms “pharmaceutical composition” or “therapeutic composition” as used herein refer to a compound or composition capable of inducing a desired therapeutic effect when properly administered to a patient.

The phrase “gene amplification” refers generally to a process by which multiple copies of a gene or gene fragment (e.g., HER2) are formed in a particular cell or cell line (e.g., cells from a cancer). The duplicated region (a stretch of amplified DNA) is often referred to as “amplicon.”

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some embodiments, the term indicates deviation from the indicated numerical value by ±10%, ±5%, ±4%, ±3%, ±2%, ±1%, ±0.9%, ±0.8%, ±0.7%, ±0.6%, ±0.5%, ±0.4%, ±0.3%, ±0.2%, ±0.1%, ±0.05%, or 0.01%. In some embodiments, “about” indicates deviation from the indicated numerical value by ±10%. In some embodiments, “about” indicates deviation from the indicated numerical value by ±5%. In some embodiments, “about” indicates deviation from the indicated numerical value by ±4%. In some embodiments, “about” indicates deviation from the indicated numerical value by ±3%. In some embodiments, “about” indicates deviation from the indicated numerical value by ±2%. In some embodiments, “about” indicates deviation from the indicated numerical value by ±1%. In some embodiments, “about” indicates deviation from the indicated numerical value by ±0.9%. In some embodiments, “about” indicates deviation from the indicated numerical value by ±0.8%. In some embodiments, “about” indicates deviation from the indicated numerical value by ±0.7%. In some embodiments, “about” indicates deviation from the indicated numerical value by ±0.6%. In some embodiments, “about” indicates deviation from the indicated numerical value by ±0.5%. In some embodiments, “about” indicates deviation from the indicated numerical value by ±0.4%. In some embodiments, “about” indicates deviation from the indicated numerical value by ±0.3%. In some embodiments, “about” indicates deviation from the indicated numerical value by ±0.1%. In some embodiments, “about” indicates deviation from the indicated numerical value by ±0.05%. In some embodiments, “about” indicates deviation from the indicated numerical value by ±0.01%.

### II. Trispecific Binding Proteins

The present disclosure relates to methods of treating a subject having a cancer comprising administering to the subject about 0.2 μg to about 4500 μg of a trispecific binding protein, wherein the trispecific binding protein comprises a first antigen binding site that binds a CD28 polypeptide, a second antigen binding site that binds a CD3 polypeptide, and a third antigen binding site that binds a HER2 polypeptide. The binding proteins provided herein are trispecific antibodies capable of redirecting T cells to kill human epidermal growth factor receptor 2 (HER2)-expressing tumors, irrespective of T cell antigen receptor specificity. Without wishing to be bound by theory, the binding proteins function by co-engaging tumor cells via HER2 and T cells via CD3 and CD28, resulting in T cell activation and cytolysis of the tumors. HER2 is a tyrosine kinase receptor that is overexpressed in multiple cancers, including breast, gastric, lung, gastroesophageal, ovarian, bladder, colon, and others. HER2 is a clinically validated target for cancer treatment, with currently approved HER2-targeting antibodies and small molecule inhibitors of its kinase activity. As T cell engagers (TCEs), the binding proteins provided herein differentiate from available HER2 therapeutics by leveraging T cells to eradicate HER2-expressing cancers, harnessing the power of the immune system.

In some embodiments, the trispecific and trivalent binding protein comprises four polypeptide chains that form three antigen binding sites that specifically bind to a CD28 polypeptide, a CD3 polypeptide, and a HER2 polypeptide, wherein a first pair of polypeptides forming the binding protein possess dual variable domains having a cross-over orientation and wherein a second pair of polypeptides forming the binding protein possess a single variable domain. In some embodiments, the trispecific and trivalent binding protein is a trispecific and trivalent binding protein described in U.S. Ser. No. 11/613,576B2, the content of which is herein incorporated by reference in its entirety.

In some embodiments, the trispecific binding protein comprises four polypeptide chains that form the three antigen binding sites, wherein a first polypeptide chain comprises a structure represented by the formula:

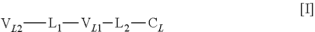

and a second polypeptide chain comprises a structure represented by the formula:

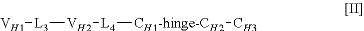

and a third polypeptide chain comprises a structure represented by the formula:

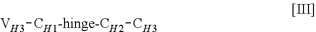

and a fourth polypeptide chain comprises a structure represented by the formula:

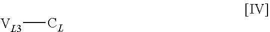

wherein:
V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain;
C_{H2} is an immunoglobulin C_{H2} heavy chain constant domain;
C_{H3} is an immunoglobulin C_{H3} heavy chain constant domain;
hinge is an immunoglobulin hinge region connecting the C_{H1} and C_{H2} domains; and
L₁, L₂, L₃ and L₄ are amino acid linkers;
   wherein the polypeptide of formula I and the polypeptide of formula II form a cross-over light chain-heavy chain pair.

In some embodiments, the term “T-cell engager” refers to binding proteins directed to a host's immune system, more specifically the T cells' cytotoxic activity as well as directed to a tumor target protein.

In some embodiments, a trispecific binding protein of the present disclosure comprises a V_{H1} and V_{L1} domain pair that form a first antigen binding site that binds a CD28 polypeptide, a V_{H2} and V_{L2} domain pair that form a second antigen binding site that binds a CD3 polypeptide, and a V_{H3} and V_{L3} domain pair that form a third antigen binding site that binds a HER2 polypeptide. Exemplary and non-limiting polypeptides that may find use in any of the trispecific binding proteins described herein are provided in Table 4.

In some embodiments, a binding protein of the present disclosure comprises four polypeptide chains that form three antigen binding sites, wherein the first polypeptide chain comprises the amino acid sequence of SEQ ID NO:100 or an amino acid sequence that is at least 95% identical to the amino acid sequence of SEQ ID NO:100; the second polypeptide chain comprises the amino acid sequence of SEQ ID NO:101 or an amino acid sequence that is at least 95% identical to the amino acid sequence of SEQ ID NO:101; the third polypeptide chain comprises the amino acid sequence of SEQ ID NO:102 or an amino acid sequence that is at least 95% identical to the amino acid sequence of SEQ ID NO:102; and the fourth polypeptide chain comprises the amino acid sequence of SEQ ID NO:103 or an amino acid sequence that is at least 95% identical to the amino acid sequence of SEQ ID NO:103.

In some embodiments, a binding protein of the present disclosure comprises four polypeptide chains that form three antigen binding sites, wherein the first polypeptide chain comprises the amino acid sequence of SEQ ID NO:104 or an amino acid sequence that is at least 95% identical to the amino acid sequence of SEQ ID NO:104; the second polypeptide chain comprises the amino acid sequence of SEQ ID NO:105 or an amino acid sequence that is at least 95% identical to the amino acid sequence of SEQ ID NO: 105; the third polypeptide chain comprises the amino acid sequence of SEQ ID NO:106 or an amino acid sequence that is at least 95% identical to the amino acid sequence of SEQ ID NO:106; and the fourth polypeptide chain comprises the amino acid sequence of SEQ ID NO:107 or an amino acid sequence that is at least 95% identical to the amino acid sequence of SEQ ID NO:107.

In some embodiments, a binding protein of the present disclosure comprises four polypeptide chains that form three antigen binding sites, wherein the first polypeptide chain comprises the amino acid sequence of SEQ ID NO:108 or an amino acid sequence that is at least 95% identical to the amino acid sequence of SEQ ID NO:108; the second polypeptide chain comprises the amino acid sequence of SEQ ID NO:109 or an amino acid sequence that is at least 95% identical to the amino acid sequence of SEQ ID NO: 109; the third polypeptide chain comprises the amino acid sequence of SEQ ID NO:110 or an amino acid sequence that is at least 95% identical to the amino acid sequence of SEQ ID NO:110; and the fourth polypeptide chain comprises the amino acid sequence of SEQ ID NO:111 or an amino acid sequence that is at least 95% identical to the amino acid sequence of SEQ ID NO:111.

In some embodiments, a binding protein of the present disclosure comprises four polypeptide chains that form three antigen binding sites, wherein the first polypeptide chain comprises the amino acid sequence of SEQ ID NO:112 or an amino acid sequence that is at least 95% identical to the amino acid sequence of SEQ ID NO:112; the second polypeptide chain comprises the amino acid sequence of SEQ ID NO:113 or an amino acid sequence that is at least 95% identical to the amino acid sequence of SEQ ID NO:113; the third polypeptide chain comprises the amino acid sequence of SEQ ID NO:114 or an amino acid sequence that is at least 95% identical to the amino acid sequence of SEQ ID NO:114; and the fourth polypeptide chain comprises the amino acid sequence of SEQ ID NO:115 or an amino acid sequence that is at least 95% identical to the amino acid sequence of SEQ ID NO:115.

In some embodiments, a binding protein of the present disclosure comprises four polypeptide chains that form three antigen binding sites, wherein the first polypeptide chain comprises the amino acid sequence of SEQ ID NO:116 or an amino acid sequence that is at least 95% identical to the amino acid sequence of SEQ ID NO:116; the second polypeptide chain comprises the amino acid sequence of SEQ ID NO:117 or an amino acid sequence that is at least 95% identical to the amino acid sequence of SEQ ID NO:117; the third polypeptide chain comprises the amino acid sequence of SEQ ID NO:118 or an amino acid sequence that is at least 95% identical to the amino acid sequence of SEQ ID NO:118; and the fourth polypeptide chain comprises the amino acid sequence of SEQ ID NO:119 or an amino acid sequence that is at least 95% identical to the amino acid sequence of SEQ ID NO:119.

In some embodiments, a binding protein of the present disclosure comprises four polypeptide chains that form three antigen binding sites, wherein the first polypeptide chain comprises the amino acid sequence of SEQ ID NO:120 or an amino acid sequence that is at least 95% identical to the amino acid sequence of SEQ ID NO:120; the second polypeptide chain comprises the amino acid sequence of SEQ ID NO:121 or an amino acid sequence that is at least 95% identical to the amino acid sequence of SEQ ID NO: 121; the third polypeptide chain comprises the amino acid sequence of SEQ ID NO:122 or an amino acid sequence that is at least 95% identical to the amino acid sequence of SEQ ID NO:122; and the fourth polypeptide chain comprises the amino acid sequence of SEQ ID NO:123 or an amino acid sequence that is at least 95% identical to the amino acid sequence of SEQ ID NO:123.

In some embodiments, a binding protein of the present disclosure comprises four polypeptide chains that form three antigen binding sites, wherein the first polypeptide chain comprises the amino acid sequence of SEQ ID NO:124 or an amino acid sequence that is at least 95% identical to the amino acid sequence of SEQ ID NO:124; the second polypeptide chain comprises the amino acid sequence of SEQ ID NO:125 or an amino acid sequence that is at least 95% identical to the amino acid sequence of SEQ ID NO: 125; the third polypeptide chain comprises the amino acid sequence of SEQ ID NO:126 or an amino acid sequence that is at least 95% identical to the amino acid sequence of SEQ ID NO:126; and the fourth polypeptide chain comprises the amino acid sequence of SEQ ID NO:127 or an amino acid sequence that is at least 95% identical to the amino acid sequence of SEQ ID NO:127.

In some embodiments, a binding protein of the present disclosure comprises four polypeptide chains that form three antigen binding sites, wherein the first polypeptide chain comprises the amino acid sequence of SEQ ID NO:128 or an amino acid sequence that is at least 95% identical to the amino acid sequence of SEQ ID NO:128; the second polypeptide chain comprises the amino acid sequence of SEQ ID NO:129 or an amino acid sequence that is at least 95% identical to the amino acid sequence of SEQ ID NO: 129; the third polypeptide chain comprises the amino acid sequence of SEQ ID NO:130 or an amino acid sequence that is at least 95% identical to the amino acid sequence of SEQ ID NO:130; and the fourth polypeptide chain comprises the amino acid sequence of SEQ ID NO:131 or an amino acid sequence that is at least 95% identical to the amino acid sequence of SEQ ID NO:131.

In some embodiments, a binding protein of the present disclosure comprises four polypeptide chains that form three antigen binding sites, wherein the first polypeptide chain comprises the amino acid sequence of SEQ ID NO:132 or an amino acid sequence that is at least 95% identical to the amino acid sequence of SEQ ID NO:132; the second polypeptide chain comprises the amino acid sequence of SEQ ID NO:133 or an amino acid sequence that is at least 95% identical to the amino acid sequence of SEQ ID NO: 133; the third polypeptide chain comprises the amino acid sequence of SEQ ID NO:134 or an amino acid sequence that is at least 95% identical to the amino acid sequence of SEQ ID NO:134; and the fourth polypeptide chain comprises the amino acid sequence of SEQ ID NO:135 or an amino acid sequence that is at least 95% identical to the amino acid sequence of SEQ ID NO:135.

In some embodiments, a binding protein of the present disclosure comprises four polypeptide chains that form three antigen binding sites, wherein the first polypeptide chain comprises the amino acid sequence of SEQ ID NO:136 or an amino acid sequence that is at least 95% identical to the amino acid sequence of SEQ ID NO:136; the second polypeptide chain comprises the amino acid sequence of SEQ ID NO:137 or an amino acid sequence that is at least 95% identical to the amino acid sequence of SEQ ID NO: 137; the third polypeptide chain comprises the amino acid sequence of SEQ ID NO:138 or an amino acid sequence that is at least 95% identical to the amino acid sequence of SEQ ID NO:138; and the fourth polypeptide chain comprises the amino acid sequence of SEQ ID NO:139 or an amino acid sequence that is at least 95% identical to the amino acid sequence of SEQ ID NO:139.

In some embodiments, a binding protein of the present disclosure comprises four polypeptide chains that form three antigen binding sites, wherein the first polypeptide chain comprises the amino acid sequence of SEQ ID NO:140 or an amino acid sequence that is at least 95% identical to the amino acid sequence of SEQ ID NO:140; the second polypeptide chain comprises the amino acid sequence of SEQ ID NO:141 or an amino acid sequence that is at least 95% identical to the amino acid sequence of SEQ ID NO: 141; the third polypeptide chain comprises the amino acid sequence of SEQ ID NO:142 or an amino acid sequence that is at least 95% identical to the amino acid sequence of SEQ ID NO:142; and the fourth polypeptide chain comprises the amino acid sequence of SEQ ID NO:143 or an amino acid sequence that is at least 95% identical to the amino acid sequence of SEQ ID NO:143.

In some embodiments, a binding protein of the present disclosure comprises four polypeptide chains that form three antigen binding sites, wherein the first polypeptide chain comprises the amino acid sequence of SEQ ID NO:144 or an amino acid sequence that is at least 95% identical to the amino acid sequence of SEQ ID NO:144; the second polypeptide chain comprises the amino acid sequence of SEQ ID NO:145 or an amino acid sequence that is at least 95% identical to the amino acid sequence of SEQ ID NO: 145; the third polypeptide chain comprises the amino acid sequence of SEQ ID NO:146 or an amino acid sequence that is at least 95% identical to the amino acid sequence of SEQ ID NO:146; and the fourth polypeptide chain comprises the amino acid sequence of SEQ ID NO:147 or an amino acid sequence that is at least 95% identical to the amino acid sequence of SEQ ID NO:147.

In some embodiments, a binding protein of the present disclosure comprises four polypeptide chains that form three antigen binding sites, wherein the first polypeptide chain comprises the amino acid sequence of SEQ ID NO:148 or an amino acid sequence that is at least 95% identical to the amino acid sequence of SEQ ID NO:148; the second polypeptide chain comprises the amino acid sequence of SEQ ID NO:149 or an amino acid sequence that is at least 95% identical to the amino acid sequence of SEQ ID NO: 149; the third polypeptide chain comprises the amino acid sequence of SEQ ID NO:150 or an amino acid sequence that is at least 95% identical to the amino acid sequence of SEQ ID NO:150; and the fourth polypeptide chain comprises the amino acid sequence of SEQ ID NO:151 or an amino acid sequence that is at least 95% identical to the amino acid sequence of SEQ ID NO:151.

In some embodiments, a binding protein of the present disclosure comprises four polypeptide chains that form three antigen binding sites, wherein the first polypeptide chain comprises the amino acid sequence of SEQ ID NO:152 or an amino acid sequence that is at least 95% identical to the amino acid sequence of SEQ ID NO:152; the second polypeptide chain comprises the amino acid sequence of SEQ ID NO:153 or an amino acid sequence that is at least 95% identical to the amino acid sequence of SEQ ID NO: 153; the third polypeptide chain comprises the amino acid sequence of SEQ ID NO:154 or an amino acid sequence that is at least 95% identical to the amino acid sequence of SEQ ID NO:154; and the fourth polypeptide chain comprises the amino acid sequence of SEQ ID NO:155 or an amino acid sequence that is at least 95% identical to the amino acid sequence of SEQ ID NO:155.

In some embodiments, a binding protein of the present disclosure comprises four polypeptide chains that form three antigen binding sites, wherein the first polypeptide chain comprises the amino acid sequence of SEQ ID NO:286 or an amino acid sequence that is at least 95% identical to the amino acid sequence of SEQ ID NO:286; the second polypeptide chain comprises the amino acid sequence of SEQ ID NO:287 or an amino acid sequence that is at least 95% identical to the amino acid sequence of SEQ ID NO:287; the third polypeptide chain comprises the amino acid sequence of SEQ ID NO:288 or an amino acid sequence that is at least 95% identical to the amino acid sequence of SEQ ID NO:288; and the fourth polypeptide chain comprises the amino acid sequence of SEQ ID NO:289 or an amino acid sequence that is at least 95% identical to the amino acid sequence of SEQ ID NO:289.

In some embodiments, a binding protein of the present disclosure comprises four polypeptide chains that form three antigen binding sites, wherein the first polypeptide chain comprises the amino acid sequence of SEQ ID NO:290 or an amino acid sequence that is at least 95% identical to the amino acid sequence of SEQ ID NO:290; the second polypeptide chain comprises the amino acid sequence of SEQ ID NO:291 or an amino acid sequence that is at least 95% identical to the amino acid sequence of SEQ ID NO:291; the third polypeptide chain comprises the amino acid sequence of SEQ ID NO:292 or an amino acid sequence that is at least 95% identical to the amino acid sequence of SEQ ID NO:292; and the fourth polypeptide chain comprises the amino acid sequence of SEQ ID NO:293 or an amino acid sequence that is at least 95% identical to the amino acid sequence of SEQ ID NO:293.

In some embodiments, a binding protein of the present disclosure comprises four polypeptide chains that form three antigen binding sites, wherein the first polypeptide chain comprises the amino acid sequence of SEQ ID NO:294 or an amino acid sequence that is at least 95% identical to the amino acid sequence of SEQ ID NO:294; the second polypeptide chain comprises the amino acid sequence of SEQ ID NO:295 or an amino acid sequence that is at least 95% identical to the amino acid sequence of SEQ ID NO:295; the third polypeptide chain comprises the amino acid sequence of SEQ ID NO:296 or an amino acid sequence that is at least 95% identical to the amino acid sequence of SEQ ID NO:296; and the fourth polypeptide chain comprises the amino acid sequence of SEQ ID NO:297 or an amino acid sequence that is at least 95% identical to the amino acid sequence of SEQ ID NO:297.

In some embodiments, a binding protein of the present disclosure comprises four polypeptide chains that form three antigen binding sites, wherein the first polypeptide chain comprises the amino acid sequence of SEQ ID NO:298 or an amino acid sequence that is at least 95% identical to the amino acid sequence of SEQ ID NO:298; the second polypeptide chain comprises the amino acid sequence of SEQ ID NO:299 or an amino acid sequence that is at least 95% identical to the amino acid sequence of SEQ ID NO:299; the third polypeptide chain comprises the amino acid sequence of SEQ ID NO:300 or an amino acid sequence that is at least 95% identical to the amino acid sequence of SEQ ID NO:300; and the fourth polypeptide chain comprises the amino acid sequence of SEQ ID NO:301 or an amino acid sequence that is at least 95% identical to the amino acid sequence of SEQ ID NO:301.

### A. Anti-HER2 Binding Site

In some embodiments, the HER2 polypeptide is a human HER2 polypeptide, also known as NEU, NGL, ERBB2, TKR1, CD340, HER-2, MLN19, and HER-2/neu. Human HER2 polypeptides are known in the art and include, without limitation, the polypeptides represented by NCBI Accession Numbers XP_024306411.1, XP_024306410.1, XP_024306409.1, NP_001276867.1, NP_001276866.1, NP 001276865.1, NP_001005862.1, or NP_004439.2, or a polypeptide produced from NCBI Gene ID Number 2064.

In some embodiments, an antigen binding site that binds HER2 comprises: an antibody heavy chain variable (VH) domain comprising a CDR-H1 sequence comprising the amino acid sequence of GFNIKDTY (SEQ ID NO:1) or GFNIRDTY (SEQ ID NO:2), a CDR-H2 sequence comprising the amino acid sequence of IYPTNGYT (SEQ ID NO:3), IYPTQGYT (SEQ ID NO:4), or IYPTNAYT (SEQ ID NO:5), and a CDR-H3 sequence comprising the amino acid sequence of SRWGGDGFYAMDY (SEQ ID NO:6), SRWGGEGFYAMDY (SEQ ID NO:7), or SRWGGSGFYAMDY (SEQ ID NO:8); and/or an antibody light chain variable (VL) domain comprising a CDR-L1 sequence comprising the amino acid sequence of QDVNTA (SEQ ID NO:9) or QDVQTA (SEQ ID NO:10), a CDR-L2 sequence comprising the amino acid sequence of SAS (SEQ ID NO:11), and a CDR-L3 sequence comprising the amino acid sequence of QQHYTTP (SEQ ID NO:12). In some embodiments, an antigen binding site that binds HER2 comprises: an antibody heavy chain variable (VH) domain comprising a CDR-H1 sequence comprising the amino acid sequence of GFNIKDTY (SEQ ID NO:1) or GFNIRDTY (SEQ ID NO:2), a CDR-H2 sequence comprising the amino acid sequence of IYPTNGYT (SEQ ID NO:3), IYPTQGYT (SEQ ID NO:4), or IYPTNAYT (SEQ ID NO:5), and a CDR-H3 sequence comprising the amino acid sequence of SRWGGDGFYAMDY (SEQ ID NO:6), SRWGGEGFYAMDY (SEQ ID NO:7), or SRWGGSGFYAMDY (SEQ ID NO:8); and an antibody light chain variable (VL) domain comprising a CDR-L1 sequence comprising the amino acid sequence of QDVNTA (SEQ ID NO:9) or QDVQTA (SEQ ID NO:10), a CDR-L2 sequence comprising the amino acid sequence of SAS (SEQ ID NO:11), and a CDR-L3 sequence comprising the amino acid sequence of QQHYTTP (SEQ ID NO:12).

In some embodiments, an antigen binding site that binds HER2 comprises: an antibody heavy chain variable (VH) domain comprising a CDR-H1 sequence comprising the amino acid sequence of GFNIKDTY (SEQ ID NO:1), a CDR-H2 sequence comprising the amino acid sequence of IYPTNGYT (SEQ ID NO:3), and a CDR-H3 sequence comprising the amino acid sequence of SRWGGDGFYAMDY (SEQ ID NO:6); and/or an antibody light chain variable (VL) domain comprising a CDR-L1 sequence comprising the amino acid sequence of QDVNTA (SEQ ID NO:9), a CDR-L2 sequence comprising the amino acid sequence of SAS (SEQ ID NO:11), and a CDR-L3 sequence comprising the amino acid sequence of QQHYTTP (SEQ ID NO:12). In some embodiments, an antigen binding site that binds HER2 comprises: an antibody heavy chain variable (VH) domain comprising a CDR-H1 sequence comprising the amino acid sequence of GFNIKDTY (SEQ ID NO:1), a CDR-H2 sequence comprising the amino acid sequence of IYPTNGYT (SEQ ID NO:3), and a CDR-H3 sequence comprising the amino acid sequence of SRWGGDGFYAMDY (SEQ ID NO:6); and an antibody light chain variable (VL) domain comprising a CDR-L1 sequence comprising the amino acid sequence of QDVNTA (SEQ ID NO:9), a CDR-L2 sequence comprising the amino acid sequence of SAS (SEQ ID NO:11), and a CDR-L3 sequence comprising the amino acid sequence of QQHYTTP (SEQ ID NO: 12).

In some embodiments, an antigen binding site that binds HER2 comprises: an antibody heavy chain variable (VH) domain comprising a CDR-H1 sequence comprising the amino acid sequence of GFNIRDTY (SEQ ID NO:2), a CDR-H2 sequence comprising the amino acid sequence of IYPTQGYT (SEQ ID NO:4), and a CDR-H3 sequence comprising the amino acid sequence of SRWGGEGFYAMDY (SEQ ID NO:7); and/or an antibody light chain variable (VL) domain comprising a CDR-L1 sequence comprising the amino acid sequence of QDVNTA (SEQ ID NO:9), a CDR-L2 sequence comprising the amino acid sequence of SAS (SEQ ID NO:11), and a CDR-L3 sequence comprising the amino acid sequence of QQHYTTP (SEQ ID NO:12). In some embodiments, an antigen binding site that binds HER2 comprises: an antibody heavy chain variable (VH) domain comprising a CDR-H1 sequence comprising the amino acid sequence of GFNIRDTY (SEQ ID NO:2), a CDR-H2 sequence comprising the amino acid sequence of IYPTQGYT (SEQ ID NO:4), and a CDR-H3 sequence comprising the amino acid sequence of SRWGGEGFYAMDY (SEQ ID NO:7); and an antibody light chain variable (VL) domain comprising a CDR-L1 sequence comprising the amino acid sequence of QDVNTA (SEQ ID NO:9), a CDR-L2 sequence comprising the amino acid sequence of SAS (SEQ ID NO:11), and a CDR-L3 sequence comprising the amino acid sequence of QQHYTTP (SEQ ID NO: 12).

In some embodiments, an antigen binding site that binds HER2 comprises: an antibody heavy chain variable (VH) domain comprising a CDR-H1 sequence comprising the amino acid sequence of GFNIRDTY (SEQ ID NO:2), a CDR-H2 sequence comprising the amino acid sequence of IYPTNAYT (SEQ ID NO:5), and a CDR-H3 sequence comprising the amino acid sequence of SRWGGSGFYAMDY (SEQ ID NO:8); and/or an antibody light chain variable (VL) domain comprising a CDR-L1 sequence comprising the amino acid sequence of QDVNTA (SEQ ID NO:9), a CDR-L2 sequence comprising the amino acid sequence of SAS (SEQ ID NO:11), and a CDR-L3 sequence comprising the amino acid sequence of QQHYTTP (SEQ ID NO:12). In some embodiments, an antigen binding site that binds HER2 comprises: an antibody heavy chain variable (VH) domain comprising a CDR-H1 sequence comprising the amino acid sequence of GFNIRDTY (SEQ ID NO:2), a CDR-H2 sequence comprising the amino acid sequence of IYPTNAYT (SEQ ID NO:5), and a CDR-H3 sequence comprising the amino acid sequence of SRWGGSGFYAMDY (SEQ ID NO:8); and an antibody light chain variable (VL) domain comprising a CDR-L1 sequence comprising the amino acid sequence of QDVNTA (SEQ ID NO:9), a CDR-L2 sequence comprising the amino acid sequence of SAS (SEQ ID NO:11), and a CDR-L3 sequence comprising the amino acid sequence of QQHYTTP (SEQ ID NO:12).

In some embodiments, an antigen binding site that binds HER2 comprises: an antibody heavy chain variable (VH) domain comprising a CDR-H1 sequence comprising the amino acid sequence of GFNIRDTY (SEQ ID NO:2), a CDR-H2 sequence comprising the amino acid sequence of IYPTQGYT (SEQ ID NO:4), and a CDR-H3 sequence comprising the amino acid sequence of SRWGGSGFYAMDY (SEQ ID NO:8); and/or an antibody light chain variable (VL) domain comprising a CDR-L1 sequence comprising the amino acid sequence of QDVNTA (SEQ ID NO:9), a CDR-L2 sequence comprising the amino acid sequence of SAS (SEQ ID NO:11), and a CDR-L3 sequence comprising the amino acid sequence of QQHYTTP (SEQ ID NO:12). In some embodiments, an antigen binding site that binds HER2 comprises: an antibody heavy chain variable (VH) domain comprising a CDR-H1 sequence comprising the amino acid sequence of GFNIRDTY (SEQ ID NO:2), a CDR-H2 sequence comprising the amino acid sequence of IYPTQGYT (SEQ ID NO:4), and a CDR-H3 sequence comprising the amino acid sequence of SRWGGSGFYAMDY (SEQ ID NO:8); and an antibody light chain variable (VL) domain comprising a CDR-L1 sequence comprising the amino acid sequence of QDVNTA (SEQ ID NO:9), a CDR-L2 sequence comprising the amino acid sequence of SAS (SEQ ID NO:11), and a CDR-L3 sequence comprising the amino acid sequence of QQHYTTP (SEQ ID NO: 12).

In some embodiments, an antigen binding site that binds HER2 comprises: an antibody heavy chain variable (VH) domain comprising a CDR-H1 sequence comprising the amino acid sequence of GFNIRDTY (SEQ ID NO:2), a CDR-H2 sequence comprising the amino acid sequence of IYPTNAYT (SEQ ID NO:5), and a CDR-H3 sequence comprising the amino acid sequence of SRWGGEGFYAMDY (SEQ ID NO:7); and/or an antibody light chain variable (VL) domain comprising a CDR-L1 sequence comprising the amino acid sequence of QDVNTA (SEQ ID NO:9), a CDR-L2 sequence comprising the amino acid sequence of SAS (SEQ ID NO:11), and a CDR-L3 sequence comprising the amino acid sequence of QQHYTTP (SEQ ID NO:12). In some embodiments, an antigen binding site that binds HER2 comprises: an antibody heavy chain variable (VH) domain comprising a CDR-H1 sequence comprising the amino acid sequence of GFNIRDTY (SEQ ID NO:2), a CDR-H2 sequence comprising the amino acid sequence of IYPTNAYT (SEQ ID NO:5), and a CDR-H3 sequence comprising the amino acid sequence of SRWGGEGFYAMDY (SEQ ID NO:7); and an antibody light chain variable (VL) domain comprising a CDR-L1 sequence comprising the amino acid sequence of QDVNTA (SEQ ID NO:9), a CDR-L2 sequence comprising the amino acid sequence of SAS (SEQ ID NO:11), and a CDR-L3 sequence comprising the amino acid sequence of QQHYTTP (SEQ ID NO:12).

In some embodiments, an antigen binding site that binds HER2 comprises: an antibody heavy chain variable (VH) domain comprising a CDR-H1 sequence comprising the amino acid sequence of GFNIKDTY (SEQ ID NO:1), a CDR-H2 sequence comprising the amino acid sequence of IYPTNGYT (SEQ ID NO:3), and a CDR-H3 sequence comprising the amino acid sequence of SRWGGDGFYAMDY (SEQ ID NO:6); and/or an antibody light chain variable (VL) domain comprising a CDR-L1 sequence comprising the amino acid sequence of QDVQTA (SEQ ID NO:10), a CDR-L2 sequence comprising the amino acid sequence of SAS (SEQ ID NO: 11), and a CDR-L3 sequence comprising the amino acid sequence of QQHYTTP (SEQ ID NO:12). In some embodiments, an antigen binding site that binds HER2 comprises: an antibody heavy chain variable (VH) domain comprising a CDR-H1 sequence comprising the amino acid sequence of GFNIKDTY (SEQ ID NO:1), a CDR-H2 sequence comprising the amino acid sequence of IYPTNGYT (SEQ ID NO:3), and a CDR-H3 sequence comprising the amino acid sequence of SRWGGDGFYAMDY (SEQ ID NO:6); and an antibody light chain variable (VL) domain comprising a CDR-L1 sequence comprising the amino acid sequence of QDVQTA (SEQ ID NO:10), a CDR-L2 sequence comprising the amino acid sequence of SAS (SEQ ID NO:11), and a CDR-L3 sequence comprising the amino acid sequence of QQHYTTP (SEQ ID NO:12).

In some embodiments, an antigen binding site that binds HER2 comprises: an antibody heavy chain variable (VH) domain comprising a CDR-H1 sequence comprising the amino acid sequence of GFNIRDTY (SEQ ID NO:2), a CDR-H2 sequence comprising the amino acid sequence of IYPTQGYT (SEQ ID NO:4), and a CDR-H3 sequence comprising the amino acid sequence of SRWGGEGFYAMDY (SEQ ID NO:7), and a CDR-H3 sequence comprising the amino acid sequence of SRWGGSGFYAMDY (SEQ ID NO:8); and/or an antibody light chain variable (VL) domain comprising a CDR-L1 sequence comprising the amino acid sequence of QDVQTA (SEQ ID NO:10), a CDR-L2 sequence comprising the amino acid sequence of SAS (SEQ ID NO:11), and a CDR-L3 sequence comprising the amino acid sequence of QQHYTTP (SEQ ID NO:12). In some embodiments, an antigen binding site that binds HER2 comprises: an antibody heavy chain variable (VH) domain comprising a CDR-H1 sequence comprising the amino acid sequence of GFNIRDTY (SEQ ID NO:2), a CDR-H2 sequence comprising the amino acid sequence of IYPTQGYT (SEQ ID NO:4), and a CDR-H3 sequence comprising the amino acid sequence of SRWGGEGFYAMDY (SEQ ID NO:7), and a CDR-H3 sequence comprising the amino acid sequence of SRWGGSGFYAMDY (SEQ ID NO:8); and an antibody light chain variable (VL) domain comprising a CDR-L1 sequence comprising the amino acid sequence of QDVQTA (SEQ ID NO:10), a CDR-L2 sequence comprising the amino acid sequence of SAS (SEQ ID NO:11), and a CDR-L3 sequence comprising the amino acid sequence of QQHYTTP (SEQ ID NO:12).

In some embodiments, an antigen binding site that binds HER2 comprises: an antibody heavy chain variable (VH) domain comprising a CDR-H1 sequence comprising the amino acid sequence of GFNIRDTY (SEQ ID NO:2), a CDR-H2 sequence comprising the amino acid sequence of IYPTNAYT (SEQ ID NO:5), and a CDR-H3 sequence comprising the amino acid sequence of SRWGGSGFYAMDY (SEQ ID NO:8); and/or an antibody light chain variable (VL) domain comprising a CDR-L1 sequence comprising the amino acid sequence of QDVQTA (SEQ ID NO:10), a CDR-L2 sequence comprising the amino acid sequence of SAS (SEQ ID NO: 11), and a CDR-L3 sequence comprising the amino acid sequence of QQHYTTP (SEQ ID NO:12). In some embodiments, an antigen binding site that binds HER2 comprises: an antibody heavy chain variable (VH) domain comprising a CDR-H1 sequence comprising the amino acid sequence of GFNIRDTY (SEQ ID NO:2), a CDR-H2 sequence comprising the amino acid sequence of IYPTNAYT (SEQ ID NO:5), and a CDR-H3 sequence comprising the amino acid sequence of SRWGGSGFYAMDY (SEQ ID NO:8); and an antibody light chain variable (VL) domain comprising a CDR-L1 sequence comprising the amino acid sequence of QDVQTA (SEQ ID NO:10), a CDR-L2 sequence comprising the amino acid sequence of SAS (SEQ ID NO:11), and a CDR-L3 sequence comprising the amino acid sequence of QQHYTTP (SEQ ID NO:12).

In some embodiments, an antigen binding site that binds HER2 comprises: an antibody heavy chain variable (VH) domain comprising a CDR-H1 sequence comprising the amino acid sequence of GFNIRDTY (SEQ ID NO:2), a CDR-H2 sequence comprising the amino acid sequence of IYPTQGYT (SEQ ID NO:4), and a CDR-H3 sequence comprising the amino acid sequence of SRWGGSGFYAMDY (SEQ ID NO:8); and/or an antibody light chain variable (VL) domain comprising a CDR-L1 sequence comprising the amino acid sequence of QDVQTA (SEQ ID NO:10), a CDR-L2 sequence comprising the amino acid sequence of SAS (SEQ ID NO:11), and a CDR-L3 sequence comprising the amino acid sequence of QQHYTTP (SEQ ID NO:12). In some embodiments, an antigen binding site that binds HER2 comprises: an antibody heavy chain variable (VH) domain comprising a CDR-H1 sequence comprising the amino acid sequence of GFNIRDTY (SEQ ID NO:2), a CDR-H2 sequence comprising the amino acid sequence of IYPTQGYT (SEQ ID NO:4), and a CDR-H3 sequence comprising the amino acid sequence of SRWGGSGFYAMDY (SEQ ID NO:8); and an antibody light chain variable (VL) domain comprising a CDR-L1 sequence comprising the amino acid sequence of QDVQTA (SEQ ID NO:10), a CDR-L2 sequence comprising the amino acid sequence of SAS (SEQ ID NO:11), and a CDR-L3 sequence comprising the amino acid sequence of QQHYTTP (SEQ ID NO:12).

In some embodiments, an antigen binding site that binds HER2 comprises: an antibody heavy chain variable (VH) domain comprising a CDR-H1 sequence comprising the amino acid sequence of GFNIRDTY (SEQ ID NO:2), a CDR-H2 sequence comprising the amino acid sequence of IYPTNAYT (SEQ ID NO:5), and a CDR-H3 sequence comprising the amino acid sequence of SRWGGEGFYAMDY (SEQ ID NO:7); and/or an antibody light chain variable (VL) domain comprising a CDR-L1 sequence comprising the amino acid sequence of QDVQTA (SEQ ID NO:10), a CDR-L2 sequence comprising the amino acid sequence of SAS (SEQ ID NO:11), and a CDR-L3 sequence comprising the amino acid sequence of QQHYTTP (SEQ ID NO:12). In some embodiments, an antigen binding site that binds HER2 comprises: an antibody heavy chain variable (VH) domain comprising a CDR-H1 sequence comprising the amino acid sequence of GFNIRDTY (SEQ ID NO:2), a CDR-H2 sequence comprising the amino acid sequence of IYPTNAYT (SEQ ID NO:5), and a CDR-H3 sequence comprising the amino acid sequence of SRWGGEGFYAMDY (SEQ ID NO:7); and an antibody light chain variable (VL) domain comprising a CDR-L1 sequence comprising the amino acid sequence of QDVQTA (SEQ ID NO:10), a CDR-L2 sequence comprising the amino acid sequence of SAS (SEQ ID NO:11), and a CDR-L3 sequence comprising the amino acid sequence of QQHYTTP (SEQ ID NO:12).

In some embodiments, an antigen binding site that binds HER2 comprises: an antibody heavy chain variable (VH) domain comprising an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of EVQLVESGGGLVQPGGSLRLSCAASGFNIKDTYIHWVRQAPGKGLEWVARIYPTNGYT RYADSVKGRFTISADTSKNTAYLQMNSLRAEDTAVYYCSRWGGDGFYAMDYWGQGT LVTVSS (SEQ ID NO:72), EVQLVESGGGLVQPGGSLRLSCAASGFNIRDTYIHWVRQAPGKGLEWVARIYPTQGYTR YADSVKGRFTISADTSKNTAYLQMNSLRAEDTAVYYCSRWGGEGFYAMDYWGQGTL VTVSS (SEQ ID NO:73), EVQLVESGGGLVQPGGSLRLSCAASGFNIRDTYIHWVRQAPGKGLEWVARIYPTQGYTR YADSVKGRFTISADTSKNTAYLQMNSLRAEDTAVYYCSRWGGSGFYAMDYWGQGTL VTVSS (SEQ ID NO:74), EVQLVESGGGLVQPGGSLRLSCAASGFNIRDTYIHWVRQAPGKGLEWVARIYPTNAYTR YADSVKGRFTISADTSKNTAYLQMNSLRAEDTAVYYCSRWGGSGFYAMDYWGQGTL VTVSS (SEQ ID NO:75), or EVQLVESGGGLVQPGGSLRLSCAASGFNIRDTYIHWVRQAPGKGLEWVARIYPTNAYTR YADSVKGRFTISADTSKNTAYLQMNSLRAEDTAVYYCSRWGGEGFYAMDYWGQGTL VTVSS (SEQ ID NO:76); and/or an antibody light chain variable (VL) domain comprising an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of DIQMTQSPSSLSASVGDRVTITCRASQDVNTAVAWYQQKPGKAPKLLIYSASFLYSGVP SRFSGSRSGTDFTLTISSLQPEDFATYYCQQHYTTPPTFGQGTKVEIK (SEQ ID NO:77) or DIQMTQSPSSLSASVGDRVTITCRASQDVQTAVAWYQQKPGKAPKLLIYSASFLYSGVP SRFSGSRSGTDFTLTISSLQPEDFATYYCQQHYTTPPTFGQGTKVEIK (SEQ ID NO:78). In some embodiments, an antigen binding site that binds HER2 comprises: an antibody heavy chain variable (VH) domain comprising the amino acid sequence of SEQ ID NO:72, SEQ ID NO:73, SEQ ID NO:74, SEQ ID NO:75, or SEQ ID NO:76; and/or an antibody light chain variable (VL) domain comprising the amino acid sequence of SEQ ID NO:77 or SEQ ID NO:78. In some embodiments, an antigen binding site that binds HER2 comprises: an antibody heavy chain variable (VH) domain comprising the amino acid sequence of SEQ ID NO:72, SEQ ID NO:73, SEQ ID NO:74, SEQ ID NO:75, or SEQ ID NO:76; and an antibody light chain variable (VL) domain comprising the amino acid sequence of SEQ ID NO:77 or SEQ ID NO:78.

In some embodiments, an antigen binding site that binds HER2 comprises: an antibody heavy chain variable (VH) domain comprising an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of EVQLVESGGGLVQPGGSLRLSCAASGFNIKDTYIHWVRQAPGKGLEWVARIYPTNGYT RYADSVKGRFTISADTSKNTAYLQMNSLRAEDTAVYYCSRWGGDGFYAMDYWGQGT LVTVSS (SEQ ID NO:72), and/or an antibody light chain variable (VL) domain comprising an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of DIQMTQSPSSLSASVGDRVTITCRASQDVNTAVAWYQQKPGKAPKLLIYSASFLYSGVP SRFSGSRSGTDFTLTISSLQPEDFATYYCQQHYTTPPTFGQGTKVEIK (SEQ ID NO:77). In some embodiments, an antigen binding site that binds HER2 comprises: an antibody heavy chain variable (VH) domain comprising the amino acid sequence of SEQ ID NO:72, and/or an antibody light chain variable (VL) domain comprising the amino acid sequence of SEQ ID NO:77. In some embodiments, an antigen binding site that binds HER2 comprises: an antibody heavy chain variable (VH) domain comprising the amino acid sequence of SEQ ID NO:72, and an antibody light chain variable (VL) domain comprising the amino acid sequence of SEQ ID NO:77.

In some embodiments, an antigen binding site that binds HER2 comprises: an antibody heavy chain variable (VH) domain comprising an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of EVQLVESGGGLVQPGGSLRLSCAASGFNIRDTYIHWVRQAPGKGLEWVARIYPTQGYTR YADSVKGRFTISADTSKNTAYLQMNSLRAEDTAVYYCSRWGGEGFYAMDYWGQGTL VTVSS (SEQ ID NO:73), and/or an antibody light chain variable (VL) domain comprising an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of DIQMTQSPSSLSASVGDRVTITCRASQDVNTAVAWYQQKPGKAPKLLIYSASFLYSGVP SRFSGSRSGTDFTLTISSLQPEDFATYYCQQHYTTPPTFGQGTKVEIK (SEQ ID NO:77). In some embodiments, an antigen binding site that binds HER2 comprises: an antibody heavy chain variable (VH) domain comprising the amino acid sequence of SEQ ID NO:73, and/or an antibody light chain variable (VL) domain comprising the amino acid sequence of SEQ ID NO:77. In some embodiments, an antigen binding site that binds HER2 comprises: an antibody heavy chain variable (VH) domain comprising the amino acid sequence of SEQ ID NO:73, and an antibody light chain variable (VL) domain comprising the amino acid sequence of SEQ ID NO:77.

In some embodiments, an antigen binding site that binds HER2 comprises: an antibody heavy chain variable (VH) domain comprising an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of EVQLVESGGGLVQPGGSLRLSCAASGFNIRDTYIHWVRQAPGKGLEWVARIYPTNAYTR YADSVKGRFTISADTSKNTAYLQMNSLRAEDTAVYYCSRWGGSGFYAMDYWGQGTL VTVSS (SEQ ID NO:75), and/or an antibody light chain variable (VL) domain comprising an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of DIQMTQSPSSLSASVGDRVTITCRASQDVNTAVAWYQQKPGKAPKLLIYSASFLYSGVP SRFSGSRSGTDFTLTISSLQPEDFATYYCQQHYTTPPTFGQGTKVEIK (SEQ ID NO:77). In some embodiments, an antigen binding site that binds HER2 comprises: an antibody heavy chain variable (VH) domain comprising the amino acid sequence of SEQ ID NO:75, and/or an antibody light chain variable (VL) domain comprising the amino acid sequence of SEQ ID NO:77. In some embodiments, an antigen binding site that binds HER2 comprises: an antibody heavy chain variable (VH) domain comprising the amino acid sequence of SEQ ID NO:75, and an antibody light chain variable (VL) domain comprising the amino acid sequence of SEQ ID NO:77.

In some embodiments, an antigen binding site that binds HER2 comprises: an antibody heavy chain variable (VH) domain comprising an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of EVQLVESGGGLVQPGGSLRLSCAASGFNIRDTYIHWVRQAPGKGLEWVARIYPTQGYTR YADSVKGRFTISADTSKNTAYLQMNSLRAEDTAVYYCSRWGGSGFYAMDYWGQGTL VTVSS (SEQ ID NO:74), and/or an antibody light chain variable (VL) domain comprising an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of DIQMTQSPSSLSASVGDRVTITCRASQDVNTAVAWYQQKPGKAPKLLIYSASFLYSGVP SRFSGSRSGTDFTLTISSLQPEDFATYYCQQHYTTPPTFGQGTKVEIK (SEQ ID NO:77). In some embodiments, an antigen binding site that binds HER2 comprises: an antibody heavy chain variable (VH) domain comprising the amino acid sequence of SEQ ID NO:74, and/or an antibody light chain variable (VL) domain comprising the amino acid sequence of SEQ ID NO:77. In some embodiments, an antigen binding site that binds HER2 comprises: an antibody heavy chain variable (VH) domain comprising the amino acid sequence of SEQ ID NO:74, and an antibody light chain variable (VL) domain comprising the amino acid sequence of SEQ ID NO:77.

In some embodiments, an antigen binding site that binds HER2 comprises: an antibody heavy chain variable (VH) domain comprising an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of EVQLVESGGGLVQPGGSLRLSCAASGFNIRDTYIHWVRQAPGKGLEWVARIYPTNAYTR YADSVKGRFTISADTSKNTAYLQMNSLRAEDTAVYYCSRWGGEGFYAMDYWGQGTL VTVSS (SEQ ID NO:76), and/or an antibody light chain variable (VL) domain comprising an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of DIQMTQSPSSLSASVGDRVTITCRASQDVNTAVAWYQQKPGKAPKLLIYSASFLYSGVP SRFSGSRSGTDFTLTISSLQPEDFATYYCQQHYTTPPTFGQGTKVEIK (SEQ ID NO:77). In some embodiments, an antigen binding site that binds HER2 comprises: an antibody heavy chain variable (VH) domain comprising the amino acid sequence of SEQ ID NO:76, and/or an antibody light chain variable (VL) domain comprising the amino acid sequence of SEQ ID NO:77. In some embodiments, an antigen binding site that binds HER2 comprises: an antibody heavy chain variable (VH) domain comprising the amino acid sequence of SEQ ID NO:76, and an antibody light chain variable (VL) domain comprising the amino acid sequence of SEQ ID NO:77.

In some embodiments, an antigen binding site that binds HER2 comprises: an antibody heavy chain variable (VH) domain comprising an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of EVQLVESGGGLVQPGGSLRLSCAASGFNIKDTYIHWVRQAPGKGLEWVARIYPTNGYT RYADSVKGRFTISADTSKNTAYLQMNSLRAEDTAVYYCSRWGGDGFYAMDYWGQGT LVTVSS (SEQ ID NO:72), and/or an antibody light chain variable (VL) domain comprising an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of DIQMTQSPSSLSASVGDRVTITCRASQDVQTAVAWYQQKPGKAPKLLIYSASFLYSGVP SRFSGSRSGTDFTLTISSLQPEDFATYYCQQHYTTPPTFGQGTKVEIK (SEQ ID NO:78). In some embodiments, an antigen binding site that binds HER2 comprises: an antibody heavy chain variable (VH) domain comprising the amino acid sequence of SEQ ID NO:72, and/or an antibody light chain variable (VL) domain comprising the amino acid sequence of SEQ ID NO:78. In some embodiments, an antigen binding site that binds HER2 comprises: an antibody heavy chain variable (VH) domain comprising the amino acid sequence of SEQ ID NO:72, and an antibody light chain variable (VL) domain comprising the amino acid sequence of SEQ ID NO:78.

In some embodiments, an anti-HER2 antigen binding site of the present disclosure comprises 1, 2, 3, 4, 5, or all 6 CDR sequences of anti-HER2 antibody trastuzumab, 30R/55Q/102E, 30R/56A/102S, 30R/55Q/102S, 30R/56A/102E, or 30Q. In some embodiments, an anti-HER2 antigen binding site of the present disclosure comprises a VH domain sequence and/or VL domain sequence of anti-HER2 antibody trastuzumab, 30R/55Q/102E, 30R/56A/102S, 30R/55Q/102S, 30R/56A/102E, or 30Q.

Sequences of exemplary anti-HER2 antigen binding sites are provided in Table 1. In some embodiments, an anti-HER2 antigen binding site of the present disclosure comprises 1, 2, 3, 4, 5, or all 6 CDR sequences of an anti-HER2 antibody described in Table 1. In some embodiments, an anti-HER2 antigen binding site of the present disclosure comprises a VH domain sequence and/or VL domain sequence of an anti-HER2 antibody described in Table 1.

TABLE 1
Anti-HER2 binding protein sequences.

| Sequence | | | SEQ ID | |
|---|---|---|---|---|
| Type | Molecule | Description | NO | Sequence |
| CDR | Anti-Her2 | CDR-H1 | 1 | GFNIKDTY |
| | (trastuzumab) | (original) | | |
| | Heavy chain | CDR-H1 | 2 | GFNIRDTY |
| | CDRs | 30R | | |
| | | CDR-H2 | 3 | IYPTNGYT |
| | | (original) | | |
| | | CDR-H2 | 4 | IYPTQGYT |
| | | 55Q | | |
| | | CDR-H2 | 5 | IYPTNAYT |
| | | 56A | | |
| | | CDR-H3 | 6 | SRWGGDGFYAMDY |
| | | (original) | | |
| | | CDR-H3 | 7 | SRWGGEGFYAMDY |
| | | 102E | | |
| | | CDR-H3 | 8 | SRWGGSGFYAMDY |
| | | 102S | | |
| | Anti-Her2 | CDR-L1 | 9 | QDVNTA |
| | (trastuzumab) | (original) | | |
| | Light chain | CDR-L1 | 10 | QDVQTA |
| | CDRs | 30Q | | |
| | | CDR-L2 | 11 | SAS |
| | | (original) | | |
| | | CDR-L3 | 12 | QQHYTTP |
| | | (original) | | |
| Variable | Anti-Her2 | VH wt | 72 | EVQLVESGGGLVQPGGSLRLSCAA |
| domain | Trastuzumab | | | SGFNIKDTYIHWVRQAPGKGLEW |
| | and variant | | | VARIYPTNGYTRYADSVKGRFTIS |
| | VH | | | ADTSKNTAYLQMNSLRAEDTAVY |
| | | | | YCSRWGGDGFYAMDYWGQGTLV |
| | | | | TVSS |
| | | VH | 73 | EVQLVESGGGLVQPGGSLRLSCAA |
| | | 30R/55Q/10 | | SGFNIRDTYIHWVRQAPGKGLEW |
| | | 2E | | VARIYPTQGYTRYADSVKGRFTIS |
| | | | | ADTSKNTAYLQMNSLRAEDTAVY |
| | | | | YCSRWGGEGFYAMDYWGQGTLV |
| | | | | TVSS |
| | | VH | 74 | EVQLVESGGGLVQPGGSLRLSCAA |
| | | 30R/55Q/10 | | SGFNIRDTYIHWVRQAPGKGLEW |
| | | 2S | | VARIYPTQGYTRYADSVKGRFTIS |
| | | | | ADTSKNTAYLQMNSLRAEDTAVY |
| | | | | YCSRWGGSGFYAMDYWGQGTLV |
| | | | | TVSS |
| | | VH | 75 | EVQLVESGGGLVQPGGSLRLSCAA |
| | | 30R/56A/10 | | SGFNIRDTYIHWVRQAPGKGLEW |
| | | 2S | | VARIYPTNAYTRYADSVKGRFTIS |
| | | | | ADTSKNTAYLQMNSLRAEDTAVY |
| | | | | YCSRWGGSGFYAMDYWGQGTLV |
| | | | | TVSS |
| | | VH | 76 | EVQLVESGGGLVQPGGSLRLSCAA |
| | | 30R/56A/10 | | SGFNIRDTYIHWVRQAPGKGLEW |
| | | 2E | | VARIYPTNAYTRYADSVKGRFTIS |
| | | | | ADTSKNTAYLQMNSLRAEDTAVY |
| | | | | YCSRWGGEGFYAMDYWGQGTLV |
| | | | | TVSS |
| | Anti-Her2 | VL wt | 77 | DIQMTQSPSSLSASVGDRVTITCRA |
| | Trastuzumab | | | SQDVNTAVAWYQQKPGKAPKLLI |
| | | | | YSASFLYSGVPSRFSGSRSGTDFTL |
| | | | | TISSLQPEDFATYYCQQHYTTPPTF |
| | | | | GQGTKVEIK |
| | and variant | VL 30Q | 78 | DIQMTQSPSSLSASVGDRVTITCRA |
| | VL | | | SQDVQTAVAWYQQKPGKAPKLLI |
| | | | | YSASFLYSGVPSRFSGSRSGTDFTL |
| | | | | TISSLQPEDFATYYCQQHYTTPPTF |
| | | | | GQGTKVEIK |

### B. Anti-CD28 Binding Site

In some embodiments, the CD28 polypeptide is a human CD28 polypeptide, also known as Tp44. Human CD28 polypeptides are known in the art and include, without limitation, the polypeptides represented by NCBI Accession Numbers XP_011510499.1, XP_011510497.1, XP_011510496.1, NP_001230007.1, NP_001230006.1, or NP_006130.1, or a polypeptide produced from NCBI Gene ID Number 940.

In some embodiments, an antigen binding site that binds CD28 comprises: an antibody heavy chain variable (VH) domain comprising a CDR-H1 sequence comprising the amino acid sequence of GYTFTSYY (SEQ ID NO:49), a CDR-H2 sequence comprising the amino acid sequence of IYPGNVNT (SEQ ID NO:50), and a CDR-H3 sequence comprising the amino acid sequence of TRSHYGLDWNFDV (SEQ ID NO:51) and/or an antibody light chain variable (VL) domain comprising a CDR-L1 sequence comprising the amino acid sequence of QNIYVW (SEQ ID NO:52), a CDR-L2 sequence comprising the amino acid sequence of KAS (SEQ ID NO:53), and a CDR-L3 sequence comprising the amino acid sequence of QQGQTYPY (SEQ ID NO:54). In some embodiments, an antigen binding site that binds CD28 comprises: an antibody heavy chain variable (VH) domain comprising a CDR-H1 sequence comprising the amino acid sequence of GYTFTSYY (SEQ ID NO:49), a CDR-H2 sequence comprising the amino acid sequence of IYPGNVNT (SEQ ID NO:50), and a CDR-H3 sequence comprising the amino acid sequence of TRSHYGLDWNFDV (SEQ ID NO:51); and an antibody light chain variable (VL) domain comprising a CDR-L1 sequence comprising the amino acid sequence of QNIYVW (SEQ ID NO:52), a CDR-L2 sequence comprising the amino acid sequence of KAS (SEQ ID NO:53), and a CDR-L3 sequence comprising the amino acid sequence of QQGQTYPY (SEQ ID NO:54).

In some embodiments, an antigen binding site that binds CD28 comprises: an antibody heavy chain variable (VH) domain comprising an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of QVQLVQSGAEVVKPGASVKVSCKASGYTFTSYYIHWVRQAPGQGLEWIGSIYPGNVNT NYAQKFQGRATLTVDTSISTAYMELSRLRSDDTAVYYCTRSHYGLDWNFDVWGKGTT VTVSS (SEQ ID NO:91), and/or an antibody light chain variable (VL) domain comprising an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of DIQMTQSPSSLSASVGDRVTITCQASQNIYVWLNWYQQKPGKAPKLLIYKASNLHTGVP SRFSGSGSGTDFTLTISSLQPEDIATYYCQQGQTYPYTFGQGTKLEIK (SEQ ID NO:92). In some embodiments, an antigen binding site that binds CD28 comprises: an antibody heavy chain variable (VH) domain comprising the amino acid sequence of SEQ ID NO:91, and/or an antibody light chain variable (VL) domain comprising the amino acid sequence of SEQ ID NO:92. In some embodiments, an antigen binding site that binds CD28 comprises: an antibody heavy chain variable (VH) domain comprising the amino acid sequence of SEQ ID NO:91, and an antibody light chain variable (VL) domain comprising the amino acid sequence of SEQ ID NO:92.

In some embodiments, the binding protein is a trispecific binding protein comprising four polypeptides comprising three antigen binding sites, wherein the polypeptide of formula I and the polypeptide of formula II form a cross-over light chain-heavy chain pair (e.g., as described herein). In some embodiments, the VH and VL domains of any of the anti-CD28 antigen binding sites described above represent V_{H1} and V_{L1} and form a first antigen binding site that binds a CD28 polypeptide. In some embodiments, the VH and VL domains of any of the anti-CD28 antigen binding sites described above and/or in Table 2 represent V_{H1} and V_{L1} and form a first antigen binding site that binds a CD28 polypeptide, V_{H2} and V_{L2} form a second antigen binding site that binds a CD3 polypeptide, and V_{H3} and V_{L3} and form a third antigen binding site that binds HER2.

Sequences of exemplary anti-CD28 antigen binding sites are provided in Table 2. In some embodiments, an anti-CD28 antigen binding site of the present disclosure comprises 1, 2, 3, 4, 5, or all 6 CDR sequences of an anti-CD28 antibody described in Table 2. In some embodiments, an anti-CD28 antigen binding site of the present disclosure comprises a VH domain sequence and/or VL domain sequence of an anti-CD28 antibody described in Table 2.

TABLE 2
Anti-CD28 binding protein sequences

| Sequence | | | SEQ | |
|---|---|---|---|---|
| Type | Molecule | Description | ID NO | Sequence |
| CDR | Anti-CD28 | CDR-H1 | 49 | GYTFTSYY |
| | (sup) | CDR-H2 | 50 | IYPGNVNT |
| | | CDR-H3 | 51 | TRSHYGLDWNFDV |
| | | CDR-L1 | 52 | QNIYVW |
| | | CDR-L2 | 53 | KAS |
| | | CDR-L3 | 54 | QQGQTYPY |
| Variable | Anti-CD28 | VH | 91 | QVQLVQSGAEVVKPGASVKVSCKASG |
| domain | (sup) | | | YTFTSYYIHWVRQAPGQGLEWIGSIY |
| | | | | PGNVNTNYAQKFQGRATLTVDTSIST |
| | | | | AYMELSRLRSDDTAVYYCTRSHYGLD |
| | | | | WNFDVWGKGTTVTVSS |
| | | VL | 92 | DIQMTQSPSSLSASVGDRVTITCQASQ |
| | | | | NIYVWLNWYQQKPGKAPKLLIYKASNL |
| | | | | HTGVPSRFSGSGSGTDFTLTISSLQPE |
| | | | | DIATYYCQQGQTYPYTFGQGTKLEIK |

### C. Anti-CD3 Binding Site

In some embodiments, the CD3 polypeptide is a human CD3 polypeptide, including CD3-delta (also known as T3D, IMD19, and CD3-DELTA), CD3-epsilon (also known as T3E, IMD18, and TCRE), and CD3-gamma (also known as T3G, IMD17, and CD3-GAMMA). Human CD3 polypeptides are known in the art and include, without limitation, the polypeptides represented by NCBI Accession Numbers XP_006510029.1 or NP_031674.1, or a polypeptide produced from NCBI Gene ID Numbers 915, 916, or 917.

In some embodiments, an antigen binding site that binds CD3 comprises: an antibody heavy chain variable (VH) domain comprising a CDR-H1 sequence comprising the amino acid sequence of GFTFTKAW (SEQ ID NO:55), a CDR-H2 sequence comprising the amino acid sequence of IKDKSNSYAT (SEQ ID NO:56), and a CDR-H3 sequence comprising the amino acid sequence of RGVYYALSPFDY (SEQ ID NO:57); and/or an antibody light chain variable (VL) domain comprising a CDR-L1 sequence comprising the amino acid sequence of QSLVHX₁NX₂X₃TY, wherein X₁ is E or Q, X₂ is A or L, and X₃ is Q, R, or F (SEQ ID NO:180), a CDR-L2 sequence comprising the amino acid sequence of KVS (SEQ ID NO:64), and a CDR-L3 sequence comprising the amino acid sequence of GQGTQYPFT (SEQ ID NO:65). In some embodiments, the CDR-L1 sequence of the V_{L2} domain comprises an amino acid sequence selected from the group consisting of QSLVHQNAQTY (SEQ ID NO:59), QSLVHENLQTY (SEQ ID NO:60), QSLVHENLFTY (SEQ ID NO:61), and QSLVHENLRTY (SEQ ID NO:62).

In some embodiments, an antigen binding site that binds CD3 comprises: an antibody heavy chain variable (VH) domain comprising a CDR-H1 sequence comprising the amino acid sequence of GFTFTKAW (SEQ ID NO:55), a CDR-H2 sequence comprising the amino acid sequence of IKDKSNSYAT (SEQ ID NO:56), and a CDR-H3 sequence comprising the amino acid sequence of RGVYYALSPFDY (SEQ ID NO:57); and/or an antibody light chain variable (VL) domain comprising a CDR-L1 sequence comprising the amino acid sequence of QSLVHQNAQTY (SEQ ID NO:59), a CDR-L2 sequence comprising the amino acid sequence of KVS (SEQ ID NO:64), and a CDR-L3 sequence comprising the amino acid sequence of GQGTQYPFT (SEQ ID NO:65). In some embodiments, an antigen binding site that binds CD3 comprises: an antibody heavy chain variable (VH) domain comprising a CDR-H1 sequence comprising the amino acid sequence of GFTFTKAW (SEQ ID NO:55), a CDR-H2 sequence comprising the amino acid sequence of IKDKSNSYAT (SEQ ID NO:56), and a CDR-H3 sequence comprising the amino acid sequence of RGVYYALSPFDY (SEQ ID NO:57); and an antibody light chain variable (VL) domain comprising a CDR-L1 sequence comprising the amino acid sequence of QSLVHQNAQTY (SEQ ID NO:59), a CDR-L2 sequence comprising the amino acid sequence of KVS (SEQ ID NO:64), and a CDR-L3 sequence comprising the amino acid sequence of GQGTQYPFT (SEQ ID NO:65).

In some embodiments, an antigen binding site that binds CD3 comprises: an antibody heavy chain variable (VH) domain comprising a CDR-H1 sequence comprising the amino acid sequence of GFTFTKAW (SEQ ID NO:55), a CDR-H2 sequence comprising the amino acid sequence of IKDKSNSYAT (SEQ ID NO:56), and a CDR-H3 sequence comprising the amino acid sequence of RGVYYALSPFDY (SEQ ID NO:57); and/or an antibody light chain variable (VL) domain comprising a CDR-L1 sequence comprising the amino acid sequence of QSLVHENLQTY (SEQ ID NO:60), a CDR-L2 sequence comprising the amino acid sequence of KVS (SEQ ID NO:64), and a CDR-L3 sequence comprising the amino acid sequence of GQGTQYPFT (SEQ ID NO:65). In some embodiments, an antigen binding site that binds CD3 comprises: an antibody heavy chain variable (VH) domain comprising a CDR-H1 sequence comprising the amino acid sequence of GFTFTKAW (SEQ ID NO:55), a CDR-H2 sequence comprising the amino acid sequence of IKDKSNSYAT (SEQ ID NO:56), and a CDR-H3 sequence comprising the amino acid sequence of RGVYYALSPFDY (SEQ ID NO:57); and an antibody light chain variable (VL) domain comprising a CDR-L1 sequence comprising the amino acid sequence of QSLVHENLQTY (SEQ ID NO:60), a CDR-L2 sequence comprising the amino acid sequence of KVS (SEQ ID NO:64), and a CDR-L3 sequence comprising the amino acid sequence of GQGTQYPFT (SEQ ID NO:65).

In some embodiments, an antigen binding site that binds CD3 comprises: an antibody heavy chain variable (VH) domain comprising a CDR-H1 sequence comprising the amino acid sequence of GFTFTKAW (SEQ ID NO:55), a CDR-H2 sequence comprising the amino acid sequence of IKDKSNSYAT (SEQ ID NO:56), and a CDR-H3 sequence comprising the amino acid sequence of RGVYYALSPFDY (SEQ ID NO:57); and/or an antibody light chain variable (VL) domain comprising a CDR-L1 sequence comprising the amino acid sequence of QSLVHENLFTY (SEQ ID NO:61), a CDR-L2 sequence comprising the amino acid sequence of KVS (SEQ ID NO:64), and a CDR-L3 sequence comprising the amino acid sequence of GQGTQYPFT (SEQ ID NO:65). In some embodiments, an antigen binding site that binds CD3 comprises: an antibody heavy chain variable (VH) domain comprising a CDR-H1 sequence comprising the amino acid sequence of GFTFTKAW (SEQ ID NO:55), a CDR-H2 sequence comprising the amino acid sequence of IKDKSNSYAT (SEQ ID NO:56), and a CDR-H3 sequence comprising the amino acid sequence of RGVYYALSPFDY (SEQ ID NO:57); and an antibody light chain variable (VL) domain comprising a CDR-L1 sequence comprising the amino acid sequence of QSLVHENLFTY (SEQ ID NO:61), a CDR-L2 sequence comprising the amino acid sequence of KVS (SEQ ID NO:64), and a CDR-L3 sequence comprising the amino acid sequence of GQGTQYPFT (SEQ ID NO:65).

In some embodiments, an antigen binding site that binds CD3 comprises: an antibody heavy chain variable (VH) domain comprising a CDR-H1 sequence comprising the amino acid sequence of GFTFTKAW (SEQ ID NO:55), a CDR-H2 sequence comprising the amino acid sequence of IKDKSNSYAT (SEQ ID NO:56), and a CDR-H3 sequence comprising the amino acid sequence of RGVYYALSPFDY (SEQ ID NO:57); and/or an antibody light chain variable (VL) domain comprising a CDR-L1 sequence comprising the amino acid sequence of QSLVHENLRTY (SEQ ID NO:62), a CDR-L2 sequence comprising the amino acid sequence of KVS (SEQ ID NO:64), and a CDR-L3 sequence comprising the amino acid sequence of GQGTQYPFT (SEQ ID NO:65). In some embodiments, an antigen binding site that binds CD3 comprises: an antibody heavy chain variable (VH) domain comprising a CDR-H1 sequence comprising the amino acid sequence of GFTFTKAW (SEQ ID NO:55), a CDR-H2 sequence comprising the amino acid sequence of IKDKSNSYAT (SEQ ID NO:56), and a CDR-H3 sequence comprising the amino acid sequence of RGVYYALSPFDY (SEQ ID NO:57); and an antibody light chain variable (VL) domain comprising a CDR-L1 sequence comprising the amino acid sequence of QSLVHENLRTY (SEQ ID NO:62), a CDR-L2 sequence comprising the amino acid sequence of KVS (SEQ ID NO:64), and a CDR-L3 sequence comprising the amino acid sequence of GQGTQYPFT (SEQ ID NO:65).

In some embodiments, an antigen binding site that binds CD3 comprises: an antibody heavy chain variable (VH) domain comprising an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of QVQLVESGGGVVQPGRSLRLSCAASGFTFTKAWMHWVRQAPGKQLEWVAQIKDKSNS YATYYADSVKGRFTISRDDSKNTLYLQMNSLRAEDTAVYYCRGVYYALSPFDYWGQG TLVTVSS (SEQ ID NO:93), and/or an antibody light chain variable (VL) domain comprising an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to an amino acid sequence selected from the group consisting of DIVMTQTPLSLSVTPGQPASISCKSSQSLVHQNAQTYLSWYLQKPGQSPQSLIYKVSNRF SGVPDRFSGSGSGTDFTLKISRVEAEDVGVYYCGQGTQYPFTFGSGTKVEIK (SEQ ID NO:95), DIVMTQTPLSLSVTPGQPASISCKSSQSLVHENLQTYLSWYLQKPGQSPQSLIYKVSNRFS GVPDRFSGSGSGTDFTLKISRVEAEDVGVYYCGQGTQYPFTFGSGTKVEIK (SEQ ID NO:96), DIVMTQTPLSLSVTPGQPASISCKSSQSLVHENLFTYLSWYLQKPGQSPQSLIYKVSNRFS GVPDRFSGSGSGTDFTLKISRVEAEDVGVYYCGQGTQYPFTFGSGTKVEIK (SEQ ID NO:97), and DIVMTQTPLSLSVTPGQPASISCKSSQSLVHENLRTYLSWYLQKPGQSPQSLIYKVSNRFS GVPDRFSGSGSGTDFTLKISRVEAEDVGVYYCGQGTQYPFTFGSGTKVEIK (SEQ ID NO:98). In some embodiments, an antigen binding site that binds CD3 comprises: an antibody heavy chain variable (VH) domain comprising the amino acid sequence of SEQ ID NO:93, and/or an antibody light chain variable (VL) domain comprising an amino acid sequence selected from the group consisting of SEQ ID NO:95, SEQ ID NO:96, SEQ ID NO:97, and SEQ ID NO:98. In some embodiments, an antigen binding site that binds CD3 comprises: an antibody heavy chain variable (VH) domain comprising the amino acid sequence of SEQ ID NO:93, and an antibody light chain variable (VL) domain comprising an amino acid sequence selected from the group consisting of SEQ ID NO:95, SEQ ID NO:96, SEQ ID NO:97, and SEQ ID NO:98.

In some embodiments, an antigen binding site that binds CD3 comprises: an antibody heavy chain variable (VH) domain comprising an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of QVQLVESGGGVVQPGRSLRLSCAASGFTFTKAWMHWVRQAPGKQLEWVAQIKDKSNS YATYYADSVKGRFTISRDDSKNTLYLQMNSLRAEDTAVYYCRGVYYALSPFDYWGQG TLVTVSS (SEQ ID NO:93), and/or an antibody light chain variable (VL) domain comprising an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of DIVMTQTPLSLSVTPGQPASISCKSSQSLVHQNAQTYLSWYLQKPGQSPQSLIYKVSNRF SGVPDRFSGSGSGTDFTLKISRVEAEDVGVYYCGQGTQYPFTFGSGTKVEIK (SEQ ID NO:95). In some embodiments, an antigen binding site that binds CD3 comprises: an antibody heavy chain variable (VH) domain comprising the amino acid sequence of SEQ ID NO:93, and/or an antibody light chain variable (VL) domain comprising the amino acid sequence of SEQ ID NO:95. In some embodiments, an antigen binding site that binds CD3 comprises: an antibody heavy chain variable (VH) domain comprising the amino acid sequence of SEQ ID NO:93, and an antibody light chain variable (VL) domain comprising the amino acid sequence of SEQ ID NO:95.

In some embodiments, an antigen binding site that binds CD3 comprises: an antibody heavy chain variable (VH) domain comprising an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of QVQLVESGGGVVQPGRSLRLSCAASGFTFTKAWMHWVRQAPGKQLEWVAQIKDKSNS YATYYADSVKGRFTISRDDSKNTLYLQMNSLRAEDTAVYYCRGVYYALSPFDYWGQG TLVTVSS (SEQ ID NO:93), and/or an antibody light chain variable (VL) domain comprising an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of DIVMTQTPLSLSVTPGQPASISCKSSQSLVHENLQTYLSWYLQKPGQSPQSLIYKVSNRFS GVPDRFSGSGSGTDFTLKISRVEAEDVGVYYCGQGTQYPFTFGSGTKVEIK (SEQ ID NO:96). In some embodiments, an antigen binding site that binds CD3 comprises: an antibody heavy chain variable (VH) domain comprising the amino acid sequence of SEQ ID NO:93, and/or an antibody light chain variable (VL) domain comprising the amino acid sequence of SEQ ID NO:96. In some embodiments, an antigen binding site that binds CD3 comprises: an antibody heavy chain variable (VH) domain comprising the amino acid sequence of SEQ ID NO:93, and an antibody light chain variable (VL) domain comprising the amino acid sequence of SEQ ID NO:96.

In some embodiments, an antigen binding site that binds CD3 comprises: an antibody heavy chain variable (VH) domain comprising an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of QVQLVESGGGVVQPGRSLRLSCAASGFTFTKAWMHWVRQAPGKQLEWVAQIKDKSNS YATYYADSVKGRFTISRDDSKNTLYLQMNSLRAEDTAVYYCRGVYYALSPFDYWGQG TLVTVSS (SEQ ID NO:93), and/or an antibody light chain variable (VL) domain comprising an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of DIVMTQTPLSLSVTPGQPASISCKSSQSLVHENLFTYLSWYLQKPGQSPQSLIYKVSNRFS GVPDRFSGSGSGTDFTLKISRVEAEDVGVYYCGQGTQYPFTFGSGTKVEIK (SEQ ID NO:97). In some embodiments, an antigen binding site that binds CD3 comprises: an antibody heavy chain variable (VH) domain comprising the amino acid sequence of SEQ ID NO:93, and/or an antibody light chain variable (VL) domain comprising the amino acid sequence of SEQ ID NO:97. In some embodiments, an antigen binding site that binds CD3 comprises: an antibody heavy chain variable (VH) domain comprising the amino acid sequence of SEQ ID NO:93, and an antibody light chain variable (VL) domain comprising the amino acid sequence of SEQ ID NO:97.

In some embodiments, an antigen binding site that binds CD3 comprises: an antibody heavy chain variable (VH) domain comprising an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of QVQLVESGGGVVQPGRSLRLSCAASGFTFTKAWMHWVRQAPGKQLEWVAQIKDKSNS YATYYADSVKGRFTISRDDSKNTLYLQMNSLRAEDTAVYYCRGVYYALSPFDYWGQG TLVTVSS (SEQ ID NO:93), and/or an antibody light chain variable (VL) domain comprising an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of DIVMTQTPLSLSVTPGQPASISCKSSQSLVHENLRTYLSWYLQKPGQSPQSLIYKVSNRFS GVPDRFSGSGSGTDFTLKISRVEAEDVGVYYCGQGTQYPFTFGSGTKVEIK (SEQ ID NO:98). In some embodiments, an antigen binding site that binds CD3 comprises: an antibody heavy chain variable (VH) domain comprising the amino acid sequence of SEQ ID NO:93, and/or an antibody light chain variable (VL) domain comprising the amino acid sequence of SEQ ID NO:98. In some embodiments, an antigen binding site that binds CD3 comprises: an antibody heavy chain variable (VH) domain comprising the amino acid sequence of SEQ ID NO:93, and an antibody light chain variable (VL) domain comprising the amino acid sequence of SEQ ID NO:98.

In some embodiments, an antigen binding site that binds CD3 comprises: an antibody heavy chain variable (VH) domain comprising an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of QVQLVESGGGVVQPGRSLRLSCAASGFTFTKAWMHWVRQAPGKQLEWVAQIKDKSNS YATYYASSVKGRFTISRDDSKNTLYLQMNSLRAEDTAVYYCRGVYYALSPFDYWGQG TLVTVSS (SEQ ID NO:302), and/or an antibody light chain variable (VL) domain comprising an amino acid sequence that is at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the amino acid sequence of DIVMTQTPLSLSVTPGQPASISCKSSQSLVHQNAQTYLSWYLQKPGQSPQSLIYKVSNRF SGVPDRFSGSGSGTDFTLKISRVEAEDVGVYYCGQGTQYPFTFGSGTKVEIK (SEQ ID NO:95). In some embodiments, an antigen binding site that binds CD3 comprises: an antibody heavy chain variable (VH) domain comprising the amino acid sequence of SEQ ID NO:302, and/or an antibody light chain variable (VL) domain comprising the amino acid sequence of SEQ ID NO:95. In some embodiments, an antigen binding site that binds CD3 comprises: an antibody heavy chain variable (VH) domain comprising the amino acid sequence of SEQ ID NO:302, and an antibody light chain variable (VL) domain comprising the amino acid sequence of SEQ ID NO:95.

In some embodiments, the binding protein is a trispecific binding protein comprising four polypeptides comprising three antigen binding sites, wherein the polypeptide of formula I and the polypeptide of formula II form a cross-over light chain-heavy chain pair (e.g., as described herein). In some embodiments, the VH and VL domains of any of the anti-CD3 antigen binding sites described above represent V_{H2} and V_{L2} and form a second antigen binding site that binds a CD3 polypeptide. In some embodiments, V_{H1} and V_{L1} form a first antigen binding site that binds a CD28 polypeptide, the VH and VL domains of any of the anti-CD3 antigen binding sites described above and/or in Table 3 represent V_{H2} and V_{L2} and form a second antigen binding site that binds a CD3 polypeptide, and V_{H3} and V_{L3} form a third antigen binding site that binds HER2.

Sequences of exemplary anti-CD3 antigen binding sites are provided in Table 3. In some embodiments, an anti-CD3 antigen binding site of the present disclosure comprises 1, 2, 3, 4, 5, or all 6 CDR sequences of an anti-CD3 antibody described in Table 3. In some embodiments, an anti-CD3 antigen binding site of the present disclosure comprises a VH domain sequence and/or VL domain sequence of an anti-CD3 antibody described in Table 3.

TABLE 3
Anti-CD3 binding protein sequences

| Sequence | | | SEQ ID | |
|---|---|---|---|---|
| Type | Molecule | Description | NO | Sequence |
| CDR | Anti-CD3 | CDR-H1 | 55 | GFTFTKAW |
| | (mid) | original | | |
| | | CDR-H2 | 56 | IKDKSNSYAT |
| | | original | | |
| | | CDR-H3 | 57 | RGVYYALSPFDY |
| | | original | | |
| | | CDR-L1 | 58 | QSLVHNNANTY |
| | | original | | |
| | | CDR-L1 | 59 | QSLVHQNAQTY |
| | | QQ | | |
| | | CDR-L1 | 60 | QSL VHENLQTY |
| | | ENLQ | | |
| | | CDR-L1 | 61 | QSLVHENLFTY |
| | | ENLF | | |
| | | CDR-L1 | 62 | QSL VHENLRTY |
| | | ENLR | | |
| | | CDR-L1 | 63 | QSLVHDNAQTY |
| | | DNAQ | | |
| | | CDR-L2 | 64 | KVS |
| | | original | | |
| | | CDR-L3 | 65 | GQGTQYPFT |
| | | Original | | |
| | | CD3mid | 180 | QSLVHX₁NX₂X₃TY, |
| | | consensus | | wherein X₁ is E or Q, |
| | | CDR-L1 | | X₂ is A or L, and |
| | | | | X₃ is Q, R, or F |
| Variable | Anti-CD3 | VH | 93 | QVQLVESGGGVVQPGRSLRLSCA |
| Domain | (mid) | | | ASGFTFTKAWMHWVRQAPGKQL |
| | | | | EWVAQIKDKSNSYATYYADSVK |
| | | | | GRFTISRDDSKNTLYLQMNSLRAE |
| | | | | DTAVYYCRGVYYALSPFDYWGQ |
| | | | | GTLVTVSS |
| | | VL | 94 | DIVMTQTPLSLSVTPGQPASISCKS |
| | | Original | | SQSLVHNNANTYLSWYLQKPGQS |
| | | | | PQSLIYKVSNRFSGVPDRFSGSGS |
| | | | | GTDFTLKISRVEAEDVGVYYCGQ |
| | | | | GTQYPFTFGSGTKVEIK |
| | | VL | 95 | DIVMTQTPLSLSVTPGQPASISCKS |
| | | 32/35 QQ | | SQSLVHQNAQTYLSWYLQKPGQS |
| | | | | PQSLIYKVSNRFSGVPDRFSGSGS |
| | | | | GTDFTLKISRVEAEDVGVYYCGQ |
| | | | | GTQYPFTFGSGTKVEIK |
| | | VL | 96 | DIVMTQTPLSLSVTPGQPASISCKS |
| | | ENLQ | | SQSLVHENLQTYLSWYLQKPGQS |
| | | | | PQSLIYKVSNRFSGVPDRFSGSGS |
| | | | | GTDFTLKISRVEAEDVGVYYCGQ |
| | | | | GTQYPFTFGSGTKVEIK |
| | | VL | 97 | DIVMTQTPLSLSVTPGQPASISCKS |
| | | ENLF | | SQSLVHENLFTYLSWYLQKPGQS |
| | | | | PQSLIYKVSNRFSGVPDRFSGSGS |
| | | | | GTDFTLKISRVEAEDVGVYYCGQ |
| | | | | GTQYPFTFGSGTKVEIK |
| | | VL | 98 | DIVMTQTPLSLSVTPGQPASISCKS |
| | | ENLR | | SQSLVHENLRTYLSWYLQKPGQS |
| | | | | PQSLIYKVSNRFSGVPDRFSGSGS |
| | | | | GTDFTLKISRVEAEDVGVYYCGQ |
| | | | | GTQYPFTFGSGTKVEIK |
| | | VL | 99 | DIVMTQTPLSLSVTPGQPASISCKS |
| | | DNAQ | | SQSLVHDNAQTYLSWYLQKPGQS |
| | | | | PQSLIYKVSNRFSGVPDRFSGSGS |
| | | | | GTDFTLKISRVEAEDVGVYYCGQ |
| | | | | GTQYPFTFGSGTKVEIK |
| | | VH 185S | 302 | QVQLVESGGGVVQPGRSLRLSCA |
| | | | | ASGFTFTKAWMHWVRQAPGKQL |
| | | | | EWVAQIKDKSNSYATYYASSVKG |
| | | | | RFTISRDDSKNTLYLQMNSLRAED |
| | | | | TAVYYCRGVYYALSPFDYWGQG |
| | | | | TLVTVSS |

### D. Linkers

In some embodiments, the linkers L₁, L₂, L₃, and L₄ range from no amino acids (length=0) to about 100 amino acids long, or less than 100, 50, 40, 30, 20, or 15 amino acids. The linkers can also be 10, 9, 8, 7, 6, 5, 4, 3, 2, or 1 amino acids long. L₁, L₂, L₃, and L₄ in one binding protein may all have the same amino acid sequence or may all have different amino acid sequences.

Examples of suitable linkers include, for example, GGGGSGGGGS (SEQ ID NO:69), GGGGSGGGGSGGGGS (SEQ ID NO: 70), S, RT, TKGPS (SEQ ID NO: 68), GQPKAAP (SEQ ID NO: 67), GGSGSSGSGG (SEQ ID NO: 71), and DKTHT (SEQ ID NO:66), as well as those disclosed in International Publication Nos. WO2017/074878 and WO2017/180913 and US20190054182A1 and US20170320967A1, all of which are herein incorporated by reference in their entireties. The examples listed above are not intended to limit the scope of the disclosure in any way, and linkers comprising randomly selected amino acids selected from the group consisting of valine, leucine, isoleucine, serine, threonine, lysine, arginine, histidine, aspartate, glutamate, asparagine, glutamine, glycine, and proline have been shown to be suitable in the binding proteins.

The identity and sequence of amino acid residues in the linker may vary depending on the type of secondary structural element necessary to achieve in the linker. For example, glycine, serine, and alanine are best for linkers having maximum flexibility. Some combination of glycine, proline, threonine, and serine are useful if a more rigid and extended linker is necessary. Any amino acid residue may be considered as a linker in combination with other amino acid residues to construct larger peptide linkers as necessary depending on the desired properties.

In some embodiments, the length of L₁ is at least twice the length of L₃. In some embodiments, the length of L₂ is at least twice the length of L₄. In some embodiments, the length of L₁ is at least twice the length of L₃, and the length of L₂ is at least twice the length of L₄. In some embodiments, L₁ is 3 to 12 amino acid residues in length, L₂ is 3 to 14 amino acid residues in length, L₃ is 1 to 8 amino acid residues in length, and L₄ is 1 to 3 amino acid residues in length. In some embodiments, L₁ is 5 to 10 amino acid residues in length, L₂ is 5 to 8 amino acid residues in length, L₃ is 1 to 5 amino acid residues in length, and L₄ is 1 to 2 amino acid residues in length. In some embodiments, L₁ is 7 amino acid residues in length, L₂ is 5 amino acid residues in length, L₃ is 1 amino acid residue in length, and L₄ is 2 amino acid residues in length.

In some embodiments, L₁, L₂, L₃ and L₄ each independently are zero amino acids in length or comprise a sequence selected from the group consisting of GGGGSGGGGS (SEQ ID NO:69), GGGGSGGGGSGGGGS (SEQ ID NO: 70), S, RT, TKGPS (SEQ ID NO: 68), GQPKAAP (SEQ ID NO: 67), and GGSGSSGSGG (SEQ ID NO: 71). In some embodiments, L₁, L₂, L₃ and L₄ each independently comprise a sequence selected from the group consisting of GGGGSGGGGS (SEQ ID NO:69), GGGGSGGGGSGGGGS (SEQ ID NO: 70), S, RT, TKGPS (SEQ ID NO: 68), GQPKAAP (SEQ ID NO: 67), and GGSGSSGSGG (SEQ ID NO: 71). In some embodiments, L₁ comprises the sequence GQPKAAP (SEQ ID NO: 67), L₂ comprises the sequence TKGPS (SEQ ID NO:68), L₃ comprises the sequence S, and L₄ comprises the sequence RT.

In some embodiments, at least one of L₁, L₂, L₃ or L₄ comprises the sequence DKTHT (SEQ ID NO:66). In some embodiments, L₁, L₂, L₃ and L₄ comprise the sequence DKTHT (SEQ ID NO: 66).

### E. Fc Regions and Constant Domains

In some embodiments, a binding protein of the present disclosure comprises a second polypeptide chain further comprising an Fc region linked to C_{H1}, the Fc region comprising an immunoglobulin hinge region and C_{H2} and C_{H3} immunoglobulin heavy chain constant domains. In some embodiments, a binding protein of the present disclosure comprises a third polypeptide chain further comprising an Fc region linked to C_{H1}, the Fc region comprising an immunoglobulin hinge region and C_{H2} and C_{H3} immunoglobulin heavy chain constant domains. In some embodiments, a binding protein of the present disclosure comprises a second polypeptide chain further comprising an Fc region linked to C_{H1}, the Fc region comprising an immunoglobulin hinge region and C_{H2} and C_{H3} immunoglobulin heavy chain constant domains, and a third polypeptide chain further comprising an Fc region linked to C_{H1}, the Fc region comprising an immunoglobulin hinge region and C_{H2} and C_{H3} immunoglobulin heavy chain constant domains.

In some embodiments, a binding protein of the present disclosure comprises a full-length antibody heavy chain or a polypeptide chain comprising an Fc region. In some embodiments, the Fc region is a human Fc region, e.g., a human IgG1, IgG2, IgG3, or IgG4 Fc region. In some embodiments, the Fc region includes an antibody hinge, C_{H1}, C_{H2}, C_{H3}, and optionally C_{H4} domains. In some embodiments, the Fc region is a human IgG1 Fc region. In some embodiments, the Fc region is a human IgG4 Fc region. In some embodiments, the Fc region includes one or more of the mutations described infra. In some embodiments, the Fc region is an Fc region of one of the heavy chain polypeptides (e.g., polypeptide 2 or 3) of a binding protein shown in Table 4. In some embodiments, the heavy chain constant region is a constant region of one of the heavy chain polypeptides (e.g., polypeptide 2 or 3) of a binding protein shown in Table 4. In some embodiments, the light chain constant region is a constant region of one of the light chain polypeptides (e.g., polypeptide 1 or 4) of a binding protein shown in Table 4.

In some embodiments, a binding protein of the present disclosure includes one or two Fc variants. The term “Fc variant” as used herein refers to a molecule or sequence that is modified from a native Fc but still comprises a binding site for the salvage receptor, FcRn (neonatal Fc receptor). Exemplary Fc variants, and their interaction with the salvage receptor, are known in the art. Thus, the term “Fc variant” can comprise a molecule or sequence that is humanized from a non-human native Fc. Furthermore, a native Fc comprises regions that can be removed because they provide structural features or biological activity that are not required for the antibody-like binding proteins of the invention. Thus, the term “Fc variant” comprises a molecule or sequence that lacks one or more native Fc sites or residues, or in which one or more Fc sites or residues has be modified, that affect or are involved in: (1) disulfide bond formation, (2) incompatibility with a selected host cell, (3) N-terminal heterogeneity upon expression in a selected host cell, (4) glycosylation, (5) interaction with complement, (6) binding to an Fc receptor other than a salvage receptor, or (7) antibody-dependent cellular cytotoxicity (ADCC).

In some embodiments, a binding protein of the present disclosure (e.g., a trispecific binding protein) comprises a “knob” mutation on the second polypeptide chain and a “hole” mutation on the third polypeptide chain. In some embodiments, a binding protein of the present disclosure comprises a “knob” mutation on the third polypeptide chain and a “hole” mutation on the second polypeptide chain. In some embodiments, the “knob” mutation comprises substitution(s) at positions corresponding to positions 354 and/or 366 of human IgG1 or IgG4 according to EU Index. In some embodiments, the amino acid substitutions are S354C, T366W, T366Y, S354C and T366W, or S354C and T366Y. In some embodiments, the “knob” mutation comprises substitutions at positions corresponding to positions 354 and 366 of human IgG1 or IgG4 according to EU Index. In some embodiments, the amino acid substitutions are S354C and T366W. In some embodiments, the “hole” mutation comprises substitution(s) at positions corresponding to positions 407 and, optionally, 349, 366, and/or 368 and of human IgG1 or IgG4 according to EU Index. In some embodiments, the amino acid substitutions are Y407V or Y407T and optionally Y349C, T366S, and/or L368A. In some embodiments, the “hole” mutation comprises substitutions at positions corresponding to positions 349, 366, 368, and 407 of human IgG1 or IgG4 according to EU Index. In some embodiments, the amino acid substitutions are Y349C, T366S, L368A, and Y407V.

In some embodiments, the second polypeptide chain further comprises a first Fc region linked to CH1, the first Fc region comprising an immunoglobulin hinge region and CH2 and CH3 immunoglobulin heavy chain constant domains, wherein the first Fc region comprises amino acid substitution(s) at positions corresponding to positions 366 and optionally 354 of human IgG1 or IgG4 according to EU Index, wherein the amino acid substitutions are T366W or T366Y and optionally S354C; and wherein the third polypeptide chain further comprises a second Fc region linked to CH1, the second Fc region comprising an immunoglobulin hinge region and CH2 and CH3 immunoglobulin heavy chain constant domains, wherein the second Fc region comprises amino acid substitution(s) at positions corresponding to positions 407 and optionally 349, 366, and/or 368 and of human IgG1 or IgG4 according to EU Index, wherein the amino acid substitutions are Y407V or Y407T and optionally Y349C, T366S, and/or L368A.

In some embodiments, the second polypeptide chain further comprises a first Fc region linked to CH1, the first Fc region comprising an immunoglobulin hinge region and CH2 and CH3 immunoglobulin heavy chain constant domains, wherein the first Fc region comprises amino acid substitution(s) at positions corresponding to positions 407 and optionally 349, 366, and/or 368 and of human IgG1 or IgG4 according to EU Index, wherein the amino acid substitutions are Y407V or Y407T and optionally Y349C, T366S, and/or L368A; and wherein the third polypeptide chain further comprises a second Fc region linked to CH1, the second Fc region comprising an immunoglobulin hinge region and CH2 and CH3 immunoglobulin heavy chain constant domains, wherein the second Fc region comprises amino acid substitution(s) at positions corresponding to positions 366 and optionally 354 of human IgG1 or IgG4 according to EU Index, wherein the amino acid substitutions are T366W or T366Y and optionally S354C.

In some embodiments, the second polypeptide chain further comprises a first Fc region linked to CH1, the first Fc region comprising an immunoglobulin hinge region and CH2 and CH3 immunoglobulin heavy chain constant domains, wherein the first Fc region comprises amino acid substitution at position corresponding to position 366 of human IgG1 or IgG4 according to EU Index, wherein the amino acid substitution is T366W; and wherein the third polypeptide chain further comprises a second Fc region linked to CH1, the second Fc region comprising an immunoglobulin hinge region and CH2 and CH3 immunoglobulin heavy chain constant domains, wherein the second Fc region comprises amino acid substitution(s) at positions corresponding to positions 366, 368, and/or 407 and of human IgG1 or IgG4 according to EU Index, wherein the amino acid substitutions are T366S, L368A, and/or Y407V.

In some embodiments, the second polypeptide chain further comprises a first Fc region linked to CH1, the first Fc region comprising an immunoglobulin hinge region and CH2 and CH3 immunoglobulin heavy chain constant domains, wherein the first Fc region comprises amino acid substitution(s) at positions corresponding to positions 366, 368, and/or 407 and of human IgG1 or IgG4 according to EU Index, wherein the amino acid substitutions are T366S, L368A, and/or Y407V; and wherein the third polypeptide chain further comprises a second Fc region linked to CH1, the second Fc region comprising an immunoglobulin hinge region and CH2 and CH3 immunoglobulin heavy chain constant domains, wherein the second Fc region comprises amino acid substitution at position corresponding to position 366 of human IgG1 or IgG4 according to EU Index, wherein the amino acid substitution is T366W.

In some embodiments, the second polypeptide chain further comprises a first Fc region linked to CH1, the first Fc region comprising an immunoglobulin hinge region and CH2 and CH3 immunoglobulin heavy chain constant domains, wherein the first Fc region comprises amino acid substitutions at positions corresponding to positions 354 and 366 of human IgG1 or IgG4 according to EU Index, wherein the amino acid substitutions are S354C and T366W; and wherein the third polypeptide chain further comprises a second Fc region linked to CH1, the second Fc region comprising an immunoglobulin hinge region and CH2 and CH3 immunoglobulin heavy chain constant domains, wherein the second Fc region comprises amino acid substitutions at positions corresponding to positions 349, 366, 368, and 407 of human IgG1 or IgG4 according to EU Index, wherein the amino acid substitutions are Y349C, T366S, L368A, and Y407V. In some embodiments, the second polypeptide chain further comprises a first Fc region linked to CH1, the first Fc region comprising an immunoglobulin hinge region and CH2 and CH3 immunoglobulin heavy chain constant domains, wherein the first Fc region comprises amino acid substitutions at positions corresponding to positions 349, 366, 368, and 407 of human IgG1 or IgG4 according to EU Index, wherein the amino acid substitutions are Y349C, T366S, L368A, and Y407V; and wherein the third polypeptide chain further comprises a second Fc region linked to CH1, the second Fc region comprising an immunoglobulin hinge region and CH2 and CH3 immunoglobulin heavy chain constant domains, wherein the second Fc region comprises amino acid substitutions at positions corresponding to positions 354 and 366 of human IgG1 or IgG4 according to EU Index, wherein the amino acid substitutions are S354C and T366W. In some embodiments, the first and/or second Fc regions are human IgG1 Fc regions. In some embodiments, the first and/or second Fc regions are human IgG4 Fc regions.

In some embodiments, the second polypeptide chain further comprises a first Fc region linked to CH1, wherein the first Fc region is a human IgG4 Fc region comprising an immunoglobulin hinge region and CH2 and CH3 immunoglobulin heavy chain constant domains, wherein the first Fc region comprises amino acid substitutions at positions corresponding to positions 228, 354, 366, and 409 of human IgG4 according to EU Index, wherein the amino acid substitutions are S228P, S354C, T366W, and R409K; and wherein the third polypeptide chain further comprises a second Fc region linked to CH1, wherein the second Fc region is a human IgG4 Fc region comprising an immunoglobulin hinge region and CH2 and CH3 immunoglobulin heavy chain constant domains, wherein the second Fc region comprises amino acid substitutions at positions corresponding to positions 228, 349, 366, 368, 407, and 409 of human IgG4 according to EU Index, wherein the amino acid substitutions are S228P, Y349C, T366S, L368A, Y407V, and R409K. In some embodiments, the second polypeptide chain further comprises a first Fc region linked to CH1, wherein the first Fc region is a human IgG4 Fc region comprising an immunoglobulin hinge region and CH2 and CH3 immunoglobulin heavy chain constant domains, wherein the first Fc region comprises amino acid substitutions at positions corresponding to positions 228, 349, 366, 368, 407, and 409 of human IgG4 according to EU Index, wherein the amino acid substitutions are S228P, Y349C, T366S, L368A, Y407V, and R409K; and wherein the third polypeptide chain further comprises a second Fc region linked to CH1, wherein the second Fc region is a human IgG4 Fc region comprising an immunoglobulin hinge region and CH2 and CH3 immunoglobulin heavy chain constant domains, wherein the second Fc region comprises amino acid substitutions at positions corresponding to positions 228, 354, 366, and 409 of human IgG4 according to EU Index, wherein the amino acid substitutions are S228P, S354C, T366W, and R409K.

In some embodiments, the second polypeptide chain further comprises a first Fc region linked to CH1, wherein the first Fc region is a human IgG4 Fc region comprising an immunoglobulin hinge region and CH2 and CH3 immunoglobulin heavy chain constant domains, wherein the first Fc region comprises amino acid substitutions at positions corresponding to positions 234, 235, 354, and 366 of human IgG4 according to EU Index, wherein the amino acid substitutions are F234A, L235A, S354C, and T366W; and wherein the third polypeptide chain further comprises a second Fc region linked to CH1, wherein the second Fc region is a human IgG4 Fc region comprising an immunoglobulin hinge region and CH2 and CH3 immunoglobulin heavy chain constant domains, wherein the second Fc region comprises amino acid substitutions at positions corresponding to positions 234, 235, 349, 366, 368, and 407 of human IgG4 according to EU Index, wherein the amino acid substitutions are F234A, L235A, Y349C, T366S, L368A, and Y407V. In some embodiments, the second polypeptide chain further comprises a first Fc region linked to CH1, wherein the first Fc region is a human IgG4 Fc region comprising an immunoglobulin hinge region and CH2 and CH3 immunoglobulin heavy chain constant domains, wherein the first Fc region comprises amino acid substitutions at positions corresponding to positions 234, 235, 349, 366, 368, and 407 of human IgG4 according to EU Index, wherein the amino acid substitutions are F234A, L235A, Y349C, T366S, L368A, and Y407V; and wherein the third polypeptide chain further comprises a second Fc region linked to CH1, wherein the second Fc region is a human IgG4 Fc region comprising an immunoglobulin hinge region and CH2 and CH3 immunoglobulin heavy chain constant domains, wherein the second Fc region comprises amino acid substitutions at positions corresponding to positions 234, 235, 354, and 366 of human IgG4 according to EU Index, wherein the amino acid substitutions are F234A, L235A, S354C, and T366W.

In some embodiments, a binding protein of the present disclosure comprises one or more mutations to reduce effector function, e.g., Fc receptor-mediated antibody-dependent cellular phagocytosis (ADCP), complement-dependent cytotoxicity (CDC), and/or antibody-dependent cellular cytotoxicity (ADCC). In some embodiments, the second polypeptide chain further comprises a first Fc region linked to C_{H1}, the first Fc region comprising an immunoglobulin hinge region and C_{H2} and C_{H3} immunoglobulin heavy chain constant domains; wherein the third polypeptide chain further comprises a second Fc region linked to C_{H1}, the second Fc region comprising an immunoglobulin hinge region and C_{H2} and C_{H3} immunoglobulin heavy chain constant domains; wherein the first and second Fc regions are human IgG1 Fc regions; and wherein the first and the second Fc regions each comprise amino acid substitutions at positions corresponding to positions 234 and 235 of human IgG1 according to EU Index, wherein the amino acid substitutions are L234A and L235A. In some embodiments, the Fc regions of the second and the third polypeptide chains are human IgG1 Fc regions, and wherein the Fc regions each comprise amino acid substitutions at positions corresponding to positions 234 and 235 of human IgG1 according to EU Index, wherein the amino acid substitutions are L234A and L235A. In some embodiments, the second polypeptide chain further comprises a first Fc region linked to C_{H1}, the first Fc region comprising an immunoglobulin hinge region and C_{H2} and C_{H3} immunoglobulin heavy chain constant domains; wherein the third polypeptide chain further comprises a second Fc region linked to C_{H1}, the second Fc region comprising an immunoglobulin hinge region and C_{H2} and C_{H3} immunoglobulin heavy chain constant domains; wherein the first and second Fc regions are human IgG1 Fc regions; and wherein the first and the second Fc regions each comprise amino acid substitutions at positions corresponding to positions 234, 235, and 329 of human IgG1 according to EU Index, wherein the amino acid substitutions are L234A, L235A, and P329A. In some embodiments, the Fc regions of the second and the third polypeptide chains are human IgG1 Fc regions, and wherein the Fc regions each comprise amino acid substitutions at positions corresponding to positions 234, 235, and 329 of human IgG1 according to EU Index, wherein the amino acid substitutions are L234A, L235A, and P329A. In some embodiments, the Fc regions of the second and the third polypeptide chains are human IgG4 Fc regions, and the Fc regions each comprise amino acid substitutions at positions corresponding to positions 234 and 235 of human IgG4 according to EU Index, wherein the amino acid substitutions are F234A and L235A. In some embodiments, the binding protein comprises a second polypeptide chain further comprising a first Fc region linked to C_{H1}, the first Fc region comprising an immunoglobulin hinge region and C_{H2} and C_{H3} immunoglobulin heavy chain constant domains, and a third polypeptide chain further comprising a second Fc region linked to C_{H1}, the second Fc region comprising an immunoglobulin hinge region and C_{H2} and C_{H3} immunoglobulin heavy chain constant domains; and wherein the first and the second Fc regions each comprise amino acid substitutions at positions corresponding to positions 234 and 235 of human IgG4 according to EU Index, wherein the amino acid substitutions are F234A and L235A.

In some embodiments, the second polypeptide chain further comprises a first Fc region linked to CH1, wherein the first Fc region is a human IgG4 Fc region comprising an immunoglobulin hinge region and CH2 and CH3 immunoglobulin heavy chain constant domains, wherein the first Fc region comprises amino acid substitutions at positions corresponding to positions 228, 234, 235, 354, 366, and 409 of human IgG4 according to EU Index, wherein the amino acid substitutions are S228P, F234A, L235A, S354C, T366W, and R409K; and wherein the third polypeptide chain further comprises a second Fc region linked to CH1, wherein the second Fc region is a human IgG4 Fc region comprising an immunoglobulin hinge region and CH2 and CH3 immunoglobulin heavy chain constant domains, wherein the second Fc region comprises amino acid substitutions at positions corresponding to positions 228, 234, 235, 349, 366, 368, 407, and 409 of human IgG4 according to EU Index, wherein the amino acid substitutions are S228P, F234A, L235A, Y349C, T366S, L368A, Y407V, and R409K. In some embodiments, the second polypeptide chain further comprises a first Fc region linked to CH1, wherein the first Fc region is a human IgG4 Fc region comprising an immunoglobulin hinge region and CH2 and CH3 immunoglobulin heavy chain constant domains, wherein the first Fc region comprises amino acid substitutions at positions corresponding to positions 228, 234, 235, 349, 366, 368, 407, and 409 of human IgG4 according to EU Index, wherein the amino acid substitutions are S228P, F234A, L235A, Y349C, T366S, L368A, Y407V, and R409K; and wherein the third polypeptide chain further comprises a second Fc region linked to CH1, wherein the second Fc region is a human IgG4 Fc region comprising an immunoglobulin hinge region and CH2 and CH3 immunoglobulin heavy chain constant domains, wherein the second Fc region comprises amino acid substitutions at positions corresponding to positions 228, 234, 235, 354, 366, and 409 of human IgG4 according to EU Index, wherein the amino acid substitutions are S228P, F234A, L235A, S354C, T366W, and R409K.

In some embodiments, the Fc region is a human IgG4 Fc region comprising one or more mutations that reduce or eliminate FcγI and/or FcγII binding. In some embodiments, the Fc region is a human IgG4 Fc region comprising one or more mutations that reduce or eliminate FcγI and/or FcγII binding but do not affect FcRn binding. In some embodiments, the Fc region is a human IgG4 Fc region comprising amino acid substitutions at positions corresponding to positions 228 and/or 409 of human IgG4 according to EU Index. In some embodiments, the amino acid substitutions are S228P and/or R409K. In some embodiments, the Fc region is a human IgG4 Fc region comprising amino acid substitutions at positions corresponding to positions 234 and/or 235 of human IgG4 according to EU Index. In some embodiments, the amino acid substitutions are F234A and/or L235A. In some embodiments, the Fc region is a human IgG4 Fc region comprising amino acid substitutions at positions corresponding to positions 228, 234, 235, and/or 409 of human IgG4 according to EU Index. In some embodiments, the amino acid substitutions are S228P, F234A, L235A, and/or R409K. In some embodiments, the Fc region is a human IgG4 Fc region comprising amino acid substitutions at positions corresponding to positions 233-236 of human IgG4 according to EU Index. In some embodiments, the amino acid substitutions are E233P, F234V, L235A, and a deletion at 236. In some embodiments, the Fc region is a human IgG4 Fc region comprising amino acid mutations at substitutions corresponding to positions 228, 233-236, and/or 409 of human IgG4 according to EU Index. In some embodiments, the amino acid mutations are S228P; E233P, F234V, L235A, and a deletion at 236; and/or R409K.

In some embodiments, the Fc region comprises one or more mutations that reduce or eliminate Fc receptor binding and/or effector function of the Fc region (e.g., Fc receptor-mediated antibody-dependent cellular phagocytosis (ADCP), complement-dependent cytotoxicity (CDC), and/or antibody-dependent cellular cytotoxicity (ADCC)).

In some embodiments, the Fc region is a human IgG1 Fc region comprising one or more amino acid substitutions at positions corresponding to positions 234, 235, and/or 329 of human IgG1 according to EU Index. In some embodiments, the amino acid substitutions are L234A, L235A, and/or P329A. In some embodiments, the Fc region is a human IgG1 Fc region comprising amino acid substitutions at positions corresponding to positions 298, 299, and/or 300 of human IgG1 according to EU Index. In some embodiments, the amino acid substitutions are S298N, T299A, and/or Y300S.

In some embodiments, a binding protein of the present disclosure comprises one or more mutations to improve stability, e.g., of the hinge region and/or dimer interface of IgG4 (See e.g., Spiess, C. et al. (2013) J. Biol. Chem. 288:26583-26593, the content of which is herein incorporated by reference in its entirety). In some embodiments, the mutation comprises substitutions at positions corresponding to positions 228 and 409 of human IgG4 according to EU Index, wherein the amino acid substitutions are S228P and R409K. In some embodiments, the binding protein comprises a second polypeptide chain further comprising a first Fc region linked to C_{H1}, the first Fc region comprising an immunoglobulin hinge region and C_{H2} and C_{H3} immunoglobulin heavy chain constant domains, and a third polypeptide chain further comprising a second Fc region linked to C_{H1}, the second Fc region comprising an immunoglobulin hinge region and C_{H2} and C_{H3} immunoglobulin heavy chain constant domains; wherein the first and second Fc regions are human IgG4 Fc regions; and wherein the first and the second Fc regions each comprise amino acid substitutions at positions corresponding to positions 228 and 409 of human IgG4 according to EU Index, wherein the amino acid substitutions are S228P and R409K. In some embodiments, a binding protein of the present disclosure comprises knob and hole mutations and one or more mutations to improve stability. In some embodiments, the first and/or second Fc regions are human IgG4 Fc regions.

In some embodiments, the Fc region is a human IgG1 Fc region comprising one or more amino acid substitutions at positions corresponding to positions 234, 235, and/or 329 of human IgG1 according to EU Index. In some embodiments, the amino acid substitutions are L234A, L235A, and/or P329A. In some embodiments, the Fc region is a human IgG1 Fc region comprising amino acid substitutions at positions corresponding to positions 298, 299, and/or 300 of human IgG1 according to EU Index. In some embodiments, the amino acid substitutions are S298N, T299A, and/or Y300S.

### F. Sequences

TABLE 4
Trispecific binding protein polypeptide sequences

| | Polypeptide | | |
|---|---|---|---|
| | Number | SEQ | |
| | (acc. to | ID | |
| Molecule | formula) | NO | Sequence |
| HER2 (WT- | 1 | 100 | DIVMTQTPLSLSVTPGQPASISCKSSQSLVHQNAQTYLSWYLQ |
| trastuzumab)/ | | | KPGQSPQSLIYKVSNRFSGVPDRFSGSGSGTDFTLKISRVEAE |
| CD28supxCD3m | | | DVGVYYCGQGTQYPFTFGSGTKVEIKDKTHTDIQMTQSPSSLS |
| id (32/35 QQ | | | ASVGDRVTITCQASQNIYVWLNWYQQKPGKAPKLLIYKASNLH |
| (LC); DKTHT | | | TGVPSRFSGSGSGTDFTLTISSLQPEDIATYYCQQGQTYPYTF |
| linkers on | | | GQGTKLEIKDKTHTRTVAAPSVFIFPPSDEQLKSGTASVVCLL |
| HC/LC) IgG4 | | | NNFYPREAKVQWKVDNALQSGNSQESVTEQDSKDSTYSLSSTL |
| FALA | | | TLSKADYEKHKVYACEVTHQGLSSPVTKSFNRGEC |
| Binding Protein | | | |
| (BP) # 1 | | | |
| | 2 | 101 | QVQLVQSGAEVVKPGASVKVSCKASGYTFTSYYIHWVRQAPG |
| | | | QGLEWIGSIYPGNVNTNYAQKFQGRATLTVDTSISTAYMELS |
| | | | RLRSDDTAVYYCTRSHYGLDWNFDVWGKGTTVTVSSDKTHT |
| | | | QVQLVESGGGVVQPGRSLRLSCAASGFTFTKAWMHWVRQAP |
| | | | GKQLEWVAQIKDKSNSYATYYADSVKGRFTISRDDSKNTLYL |
| | | | QMNSLRAEDTAVYYCRGVYYALSPFDYWGQGTLVTVSSDKTH |
| | | | TASTKGPSVFPLAPCSRSTSESTAALGCLVKDYFPEPVTVSW |
| | | | NSGALTSGVHTFPAVLQSSGLYSLSSVVTVPSSSLGTKTYTC |
| | | | NVDHKPSNTKVDKRVESKYGPPCPPCPAPEAAGGPSVFLFPP |
| | | | KPKDTLMISRTPEVTCVVVDVSQEDPEVQFNWYVDGVEVHNA |
| | | | KTKPREEQFNSTYRVVSVLTVLHQDWLNGKEYKCKVSNKGLP |
| | | | SSIEKTISKAKGQPREPQVCTLPPSQEEMTKNQVSLSCAVKG |
| | | | FYPSDIAVEWESNGQPENNYKTTPPVLDSDGSFFLVSKLTVD |
| | | | KSRWQEGNVFSCSVMHEALHNHYTQKSLSLSLG |
| | 3 | 102 | EVQLVESGGGLVQPGGSLRLSCAASGFNIKDTYIHWVRQAPG |
| | | | KGLEWVARIYPTNGYTRYADSVKGRFTISADTSKNTAYLQMN |
| | | | SLRAEDTAVYYCSRWGGDGFYAMDYWGQGTLVTVSSASTKGP |
| | | | SVFPLAPCSRSTSESTAALGCLVKDYFPEPVTVSWNSGALTS |
| | | | GVHTFPAVLQSSGLYSLSSVVTVPSSSLGTKTYTCNVDHKPS |
| | | | NTKVDKRVESKYGPPCPPCPAPEAAGGPSVFLFPPKPKDTLM |
| | | | ISRTPEVTCVVVDVSQEDPEVQFNWYVDGVEVHNAKTKPREE |
| | | | QFNSTYRVVSVLTVLHQDWLNGKEYKCKVSNKGLPSSIEKTI |
| | | | SKAKGQPREPQVYTLPPCQEEMTKNQVSLWCLVKGFYPSDIA |
| | | | VEWESNGQPENNYKTTPPVLDSDGSFFLYSKLTVDKSRWQEG |
| | | | NVFSCSVMHEALHNHYTQKSLSLSLG |
| | 4 | 103 | DIQMTQSPSSLSASVGDRVTITCRASQDVNTAVAWYQQKPGKA |
| | | | PKLLIYSASFLYSGVPSRFSGSRSGTDFTLTISSLQPEDFATY |
| | | | YCQQHYTTPPTFGQGTKVEIKRTVAAPSVFIFPPSDEQLKSGT |
| | | | ASVVCLLNNFYPREAKVQWKVDNALQSGNSQESVTEQDSKDST |
| | | | YSLSSTLTLSKADYEKHKVYACEVTHQGLSSPVTKSFNRGEC |
| HER2 | 1 | 104 | DIVMTQTPLSLSVTPGQPASISCKSSQSLVHQNAQTYLSWYLQ |
| (30R/55Q/102E | | | KPGQSPQSLIYKVSNRFSGVPDRFSGSGSGTDFTLKISRVEAE |
| + LC-WT- | | | DVGVYYCGQGTQYPFTFGSGTKVEIKDKTHTDIQMTQSPSSLS |
| trastuzumab)/ | | | ASVGDRVTITCQASQNIYVWLNWYQQKPGKAPKLLIYKASNLH |
| CD28supxCD3mid | | | TGVPSRFSGSGSGTDFTLTISSLQPEDIATYYCQQGQTYPYTF |
| (32/35 QQ | | | GQGTKLEIKDKTHTRTVAAPSVFIFPPSDEQLKSGTASVVCLL |
| (LC); DKTHT | | | NNFYPREAKVQWKVDNALQSGNSQESVTEQDSKDSTYSLSSTL |
| linkers on | | | TLSKADYEKHKVYACEVTHQGLSSPVTKSFNRGEC |
| HC/LC) IgG4 | | | |
| FALA | | | |
| BP # 2 | | | |
| | 2 | 105 | QVQLVQSGAEVVKPGASVKVSCKASGYTFTSYYIHWVRQAPG |
| | | | QGLEWIGSIYPGNVNTNYAQKFQGRATLTVDTSISTAYMELS |
| | | | RLRSDDTAVYYCTRSHYGLDWNFDVWGKGTTVTVSSDKTHT |
| | | | QVQLVESGGGVVQPGRSLRLSCAASGFTFTKAWMHWVRQAP |
| | | | GKQLEWVAQIKDKSNSYATYYADSVKGRFTISRDDSKNTLYL |
| | | | QMNSLRAEDTAVYYCRGVYYALSPFDYWGQGTLVTVSSDKTH |
| | | | TASTKGPSVFPLAPCSRSTSESTAALGCLVKDYFPEPVTVSW |
| | | | NSGALTSGVHTFPAVLQSSGLYSLSSVVTVPSSSLGTKTYTC |
| | | | NVDHKPSNTKVDKRVESKYGPPCPPCPAPEAAGGPSVFLFPP |
| | | | KPKDTLMISRTPEVTCVVVDVSQEDPEVQFNWYVDGVEVHNA |
| | | | KTKPREEQFNSTYRVVSVLTVLHQDWLNGKEYKCKVSNKGLP |
| | | | SSIEKTISKAKGQPREPQVCTLPPSQEEMTKNQVSLSCAVKG |
| | | | FYPSDIAVEWESNGQPENNYKTTPPVLDSDGSFFLVSKLTVD |
| | | | KSRWQEGNVFSCSVMHEALHNHYTQKSLSLSLG |
| | 3 | 106 | EVQLVESGGGLVQPGGSLRLSCAASGFNIRDTYIHWVRQAPG |
| | | | KGLEWVARIYPTQGYTRYADSVKGRFTISADTSKNTAYLQMN |
| | | | SLRAEDTAVYYCSRWGGEGFYAMDYWGQGTLVTVSSASTKGP |
| | | | SVFPLAPCSRSTSESTAALGCLVKDYFPEPVTVSWNSGALTS |
| | | | GVHTFPAVLQSSGLYSLSSVVTVPSSSLGTKTYTCNVDHKPS |
| | | | NTKVDKRVESKYGPPCPPCPAPEAAGGPSVFLFPPKPKDTLM |
| | | | ISRTPEVTCVVVDVSQEDPEVQFNWYVDGVEVHNAKTKPREE |
| | | | QFNSTYRVVSVLTVLHQDWLNGKEYKCKVSNKGLPSSIEKTI |
| | | | SKAKGQPREPQVYTLPPCQEEMTKNQVSLWCLVKGFYPSDIA |
| | | | VEWESNGQPENNYKTTPPVLDSDGSFFLYSKLTVDKSRWQEG |
| | | | NVFSCSVMHEALHNHYTQKSLSLSLG |
| | 4 | 107 | DIQMTQSPSSLSASVGDRVTITCRASQDVNTAVAWYQQKPGK |
| | | | APKLLIYSASFLYSGVPSRFSGSRSGTDFTLTISSLQPEDFA |
| | | | TYYCQQHYTTPPTFGQGTKVEIKRTVAAPSVFIFPPSDEQLK |
| | | | SGTASVVCLLNNFYPREAKVQWKVDNALQSGNSQESVTEQDS |
| | | | KDSTYSLSSTLTLSKADYEKHKVYACEVTHQGLSSPVTKSFN |
| | | | RGEC |
| HER2 | 1 | 108 | DIVMTQTPLSLSVTPGQPASISCKSSQSLVHDNAQTYLSWYLQK |
| (30R/55Q/102E + | | | PGQSPQSLIYKVSNRFSGVPDRFSGSGSGTDFTLKISRVEAED |
| LC-WT- | | | VGVYYCGQGTQYPFTFGSGTKVEIKDKTHTDIQMTQSPSSLSA |
| trastuzumab)/ | | | SVGDRVTITCQASQNIYVWLNWYQQKPGKAPKLLIYKASNLHT |
| CD28ₛᵤₚxCD3_{mid} | | | GVPSRFSGSGSGTDFTLTISSLQPEDIATYYCQQGQTYPYTFG |
| (DNAQ (LC); | | | QGTKLEIKDKTHTRTVAAPSVFIFPPSDEQLKSGTASVVCLLN |
| DKTHT linkers | | | NFYPREAKVQWKVDNALQSGNSQESVTEQDSKDSTYSLSSTLT |
| on HC/LC) IgG4 | | | LSKADYEKHKVYACEVTHQGLSSPVTKSFNRGEC |
| FALA | | | |
| BP # 8 | | | |
| | 2 | 109 | QVQLVQSGAEVVKPGASVKVSCKASGYTFTSYYIHWVRQAP |
| | | | GQGLEWIGSIYPGNVNTNYAQKFQGRATLTVDTSISTAYMELS |
| | | | RLRSDDTAVYYCTRSHYGLDWNFDVWGKGTTVTVSSDKTHT |
| | | | QVQLVESGGGVVQPGRSLRLSCAASGFTFTKAWMHWVRQAP |
| | | | GKQLEWVAQIKDKSNSYATYYADSVKGRFTISRDDSKNTLYL |
| | | | QMNSLRAEDTAVYYCRGVYYALSPFDYWGQGTLVTVSSDKT |
| | | | HTASTKGPSVFPLAPCSRSTSESTAALGCLVKDYFPEPVTVSW |
| | | | NSGALTSGVHTFPAVLQSSGLYSLSSVVTVPSSSLGTKTYTCN |
| | | | VDHKPSNTKVDKRVESKYGPPCPPCPAPEAAGGPSVFLFPPKP |
| | | | KDTLMISRTPEVTCVVVDVSQEDPEVQFNWYVDGVEVHNAK |
| | | | TKPREEQFNSTYRVVSVLTVLHQDWLNGKEYKCKVSNKGLPS |
| | | | SIEKTISKAKGQPREPQVCTLPPSQEEMTKNQVSLSCAVKGFY |
| | | | PSDIAVEWESNGQPENNYKTTPPVLDSDGSFFLVSKLTVDKSR |
| | | | WQEGNVFSCSVMHEALHNHYTQKSLSLSLG |
| | 3 | 110 | EVQLVESGGGLVQPGGSLRLSCAASGFNIRDTYIHWVRQAPG |
| | | | KGLEWVARIYPTQGYTRYADSVKGRFTISADTSKNTAYLQMN |
| | | | SLRAEDTAVYYCSRWGGEGFYAMDYWGQGTLVTVSSASTKG |
| | | | PSVFPLAPCSRSTSESTAALGCLVKDYFPEPVTVSWNSGALTS |
| | | | GVHTFPAVLQSSGLYSLSSVVTVPSSSLGTKTYTCNVDHKPSN |
| | | | TKVDKRVESKYGPPCPPCPAPEAAGGPSVFLFPPKPKDTLMIS |
| | | | RTPEVTCVVVDVSQEDPEVQFNWYVDGVEVHNAKTKPREEQ |
| | | | FNSTYRVVSVLTVLHQDWLNGKEYKCKVSNKGLPSSIEKTISK |
| | | | AKGQPREPQVYTLPPCQEEMTKNQVSLWCLVKGFYPSDIAVE |
| | | | WESNGQPENNYKTTPPVLDSDGSFFLYSKLTVDKSRWQEGNV |
| | | | FSCSVMHEALHNHYTQKSLSLSLG |
| | 4 | 111 | DIQMTQSPSSLSASVGDRVTITCRASQDVNTAVAWYQQKPGKA |
| | | | PKLLIYSASFLYSGVPSRFSGSRSGTDFTLTISSLQPEDFATYY |
| | | | CQQHYTTPPTFGQGTKVEIKRTVAAPSVFIFPPSDEQLKSGTAS |
| | | | VVCLLNNFYPREAKVQWKVDNALQSGNSQESVTEQDSKDST |
| | | | YSLSSTLTLSKADYEKHKVYACEVTHQGLSSPVTKSFNRGEC |
| HER2V | 1 | 286 | DIVMTQTPLSLSVTPGQPASISCKSSQSLVHQNAQTYLSWYLQ |
| (30R/56A/102S | | | KPGQSPQSLIYKVSNRFSGVPDRFSGSGSGTDFTLKISRVEAED |
| + LC-WT- | | | VGVYYCGQGTQYPFTFGSGTKVEIKDKTHTDIQMTQSPSSLSA |
| trastuzumab)/ | | | SVGDRVTITCQASQNIYVWLNWYQQKPGKAPKLLIYKASNLH |
| CD28supxCD3m | | | TGVPSRFSGSGSGTDFTLTISSLQPEDIATYYCQQGQTYPYTFG |
| id | | | QGTKLEIKDKTHTRTVAAPSVFIFPPSDEQLKSGTASVVCLLNN |
| (32/35QQ185E) | | | FYPREAKVQWKVDNALQSGNSQESVTEQDSKDSTYSLSSTLT |
| IgG4 FALA | | | LSKADYEKHKVYACEVTHQGLSSPVTKSFNRGEC |
| BP # 3 | | | |
| | 2 | 287 | QVQLVQSGAEVVKPGASVKVSCKASGYTFTSYYIHWVRQAP |
| | | | GQGLEWIGSIYPGNVNTNYAQKFQGRATLTVDTSISTAYMELS |
| | | | RLRSDDTAVYYCTRSHYGLDWNFDVWGKGTTVTVSSDKTHT |
| | | | QVQLVESGGGVVQPGRSLRLSCAASGFTFTKAWMHWVRQAP |
| | | | GKQLEWVAQIKDKSNSYATYYADSVKGRFTISRDDSKNTLYL |
| | | | QMNSLRAEDTAVYYCRGVYYALSPFDYWGQGTLVTVSSDKT |
| | | | HTASTKGPSVFPLAPCSRSTSESTAALGCLVKDYFPEPVTVSW |
| | | | NSGALTSGVHTFPAVLQSSGLYSLSSVVTVPSSSLGTKTYTCN |
| | | | VDHKPSNTKVDKRVESKYGPPCPPCPAPEAAGGPSVFLFPPKP |
| | | | KDTLMISRTPEVTCVVVDVSQEDPEVQFNWYVDGVEVHNAK |
| | | | TKPREEQFNSTYRVVSVLTVLHQDWLNGKEYKCKVSNKGLPS |
| | | | SIEKTISKAKGQPREPQVCTLPPSQEEMTKNQVSLSCAVKGFY |
| | | | PSDIAVEWESNGQPENNYKTTPPVLDSDGSFFLVSKLTVDKSR |
| | | | WQEGNVFSCSVMHEALHNHYTQKSLSLSLG |
| | 3 | 288 | EVQLVESGGGLVQPGGSLRLSCAASGFNIRDTYIHWVRQAPG |
| | | | KGLEWVARIYPTNAYTRYADSVKGRFTISADTSKNTAYLQMN |
| | | | SLRAEDTAVYYCSRWGGSGFYAMDYWGQGTLVTVSSASTKG |
| | | | PSVFPLAPCSRSTSESTAALGCLVKDYFPEPVTVSWNSGALTS |
| | | | GVHTFPAVLQSSGLYSLSSVVTVPSSSLGTKTYTCNVDHKPSN |
| | | | TKVDKRVESKYGPPCPPCPAPEFLGGPSVFLFPPKPKDTLMISR |
| | | | TPEVTCVVVDVSQEDPEVQFNWYVDGVEVHNAKTKPREEQF |
| | | | NSTYRVVSVLTVLHQDWLNGKEYKCKVSNKGLPSSIEKTISK |
| | | | AKGQPREPQVYTLPPCQEEMTKNQVSLWCLVKGFYPSDIAVE |
| | | | WESNGQPENNYKTTPPVLDSDGSFFLYSKLTVDKSRWQEGNV |
| | | | FSCSVMHEALHNHYTQKSLSLSLG |
| | 4 | 289 | DIQMTQSPSSLSASVGDRVTITCRASQDVNTAVAWYQQKPGKA |
| | | | PKLLIYSASFLYSGVPSRFSGSRSGTDFTLTISSLQPEDFATYY |
| | | | CQQHYTTPPTFGQGTKVEIKRTVAAPSVFIFPPSDEQLKSGTAS |
| | | | VVCLLNNFYPREAKVQWKVDNALQSGNSQESVTEQDSKDST |
| | | | YSLSSTLTLSKADYEKHKVYACEVTHQGLSSPVTKSFNRGEC |
| HER2 | −1 | 290 | DIVMTQTPLSLSVTPGQPASISCKSSQSLVHQNAQTYLSWYLQ |
| (30R/55Q/102E | | | KPGQSPQSLIYKVSNRFSGVPDRFSGSGSGTDFTLKISRVEAED |
| + LC-WT- | | | VGVYYCGQGTQYPFTFGSGTKVEIKGQPKAAPDIQMTQSPSSL |
| trastuzumab)/ | | | SASVGDRVTITCQASQNIYVWLNWYQQKPGKAPKLLIYKASN |
| CD28supxCD3m | | | LHTGVPSRFSGSGSGTDFTLTISSLQPEDIATYYCQQGQTYPYT |
| id | | | FGQGTKLEIKTKGPSRTVAAPSVFIFPPSDEQLKSGTASVVCLL |
| (32/35QQ185E) | | | NNFYPREAKVQWKVDNALQSGNSQESVTEQDSKDSTYSLSST |
| IgG4 FALA | | | LTLSKADYEKHKVYACEVTHQGLSSPVTKSFNRGEC |
| BP # 4 | | | |
| | 2 | 291 | QVQLVQSGAEVVKPGASVKVSCKASGYTFTSYYIHWVRQAP |
| | | | GQGLEWIGSIYPGNVNTNYAQKFQGRATLTVDTSISTAYMELS |
| | | | RLRSDDTAVYYCTRSHYGLDWNFDVWGKGTTVTVSSDKTHT |
| | | | QVQLVESGGGVVQPGRSLRLSCAASGFTFTKAWMHWVRQAP |
| | | | GKQLEWVAQIKDKSNSYATYYAESVKGRFTISRDDSKNTLYL |
| | | | QMNSLRAEDTAVYYCRGVYYALSPFDYWGQGTLVTVSSDKT |
| | | | HTASTKGPSVFPLAPCSRSTSESTAALGCLVKDYFPEPVTVSW |
| | | | NSGALTSGVHTFPAVLQSSGLYSLSSVVTVPSSSLGTKTYTCN |
| | | | VDHKPSNTKVDKRVESKYGPPCPPCPAPEAAGGPSVFLFPPKP |
| | | | KDTLMISRTPEVTCVVVDVSQEDPEVQFNWYVDGVEVHNAK |
| | | | TKPREEQFNSTYRVVSVLTVLHQDWLNGKEYKCKVSNKGLPS |
| | | | SIEKTISKAKGQPREPQVCTLPPSQEEMTKNQVSLSCAVKGFY |
| | | | PSDIAVEWESNGQPENNYKTTPPVLDSDGSFFLVSKLTVDKSR |
| | | | WQEGNVFSCSVMHEALHNHYTQKSLSLSLG |
| | 3 | 292 | EVQLVESGGGLVQPGGSLRLSCAASGFNIRDTYIHWVRQAPG |
| | | | KGLEWVARIYPTQGYTRYADSVKGRFTISADTSKNTAYLQMN |
| | | | SLRAEDTAVYYCSRWGGEGFYAMDYWGQGTLVTVSSASTKG |
| | | | PSVFPLAPCSRSTSESTAALGCLVKDYFPEPVTVSWNSGALTS |
| | | | GVHTFPAVLQSSGLYSLSSVVTVPSSSLGTKTYTCNVDHKPSN |
| | | | TKVDKRVESKYGPPCPPCPAPEAAGGPSVFLFPPKPKDTLMIS |
| | | | RTPEVTCVVVDVSQEDPEVQFNWYVDGVEVHNAKTKPREEQ |
| | | | FNSTYRVVSVLTVLHQDWLNGKEYKCKVSNKGLPSSIEKTISK |
| | | | AKGQPREPQVYTLPPCQEEMTKNQVSLWCLVKGFYPSDIAVE |
| | | | WESNGQPENNYKTTPPVLDSDGSFFLYSKLTVDKSRWQEGNV |
| | | | FSCSVMHEALHNHYTQKSLSLSLG |
| | 4 | 293 | DIQMTQSPSSLSASVGDRVTITCRASQDVNTAVAWYQQKPGK |
| | | | APKLLIYSASFLYSGVPSRFSGSRSGTDFTLTISSLQPEDFATYY |
| | | | CQQHYTTPPTFGQGTKVEIKRTVAAPSVFIFPPSDEQLKSGTAS |
| | | | VVCLLNNFYPREAKVQWKVDNALQSGNSQESVTEQDSKDST |
| | | | YSLSSTLTLSKADYEKHKVYACEVTHQGLSSPVTKSFNRGEC |
| HER2 | 1 | 294 | DIVMTQTPLSLSVTPGQPASISCKSSQSLVHQNAQTYLSWYLQ |
| (30R/55Q/102E | | | KPGQSPQSLIYKVSNRFSGVPDRFSGSGSGTDFTLKISRVEAED |
| + LC-WT- | | | VGVYYCGQGTQYPFTFGSGTKVEIKGQPKAAPDIQMTQSPSSL |
| trastuzumab)/ | | | SASVGDRVTITCQASQNIYVWLNWYQQKPGKAPKLLIYKASN |
| CD28supxCD3m | | | LHTGVPSRFSGSGSGTDFTLTISSLQPEDIATYYCQQGQTYPYT |
| id | | | FGQGTKLEIKTKGPSRTVAAPSVFIFPPSDEQLKSGTASVVCLL |
| (32/35QQ185S) | | | NNFYPREAKVQWKVDNALQSGNSQESVTEQDSKDSTYSLSST |
| IgG4 FALA | | | LTLSKADYEKHKVYACEVTHQGLSSPVTKSFNRGEC |
| BP # 5 | | | |
| | 2 | 295 | QVQLVQSGAEVVKPGASVKVSCKASGYTFTSYYIHWVRQAP |
| | | | GQGLEWIGSIYPGNVNTNYAQKFQGRATLTVDTSISTAYMELS |
| | | | RLRSDDTAVYYCTRSHYGLDWNFDVWGKGTTVTVSSSQVQL |
| | | | VESGGGVVQPGRSLRLSCAASGFTFTKAWMHWVRQAPGKQL |
| | | | EWVAQIKDKSNSYATYYASSVKGRFTISRDDSKNTLYLQMNS |
| | | | LRAEDTAVYYCRGVYYALSPFDYWGQGTLVTVSSRTASTKGP |
| | | | SVFPLAPCSRSTSESTAALGCLVKDYFPEPVTVSWNSGALTSG |
| | | | VHTFPAVLQSSGLYSLSSVVTVPSSSLGTKTYTCNVDHKPSNT |
| | | | KVDKRVESKYGPPCPPCPAPEAAGGPSVFLFPPKPKDTLMISR |
| | | | TPEVTCVVVDVSQEDPEVQFNWYVDGVEVHNAKTKPREEQF |
| | | | NSTYRVVSVLTVLHQDWLNGKEYKCKVSNKGLPSSIEKTISK |
| | | | AKGQPREPQVCTLPPSQEEMTKNQVSLSCAVKGFYPSDIAVE |
| | | | WESNGQPENNYKTTPPVLDSDGSFFLVSKLTVDKSRWQEGNV |
| | | | FSCSVMHEALHNHYTQKSLSLSLG |
| | 3 | 296 | EVQLVESGGGLVQPGGSLRLSCAASGFNIRDTYIHWVRQAPG |
| | | | KGLEWVARIYPTQGYTRYADSVKGRFTISADTSKNTAYLQMN |
| | | | SLRAEDTAVYYCSRWGGEGFYAMDYWGQGTLVTVSSASTKG |
| | | | PSVFPLAPCSRSTSESTAALGCLVKDYFPEPVTVSWNSGALTS |
| | | | GVHTFPAVLQSSGLYSLSSVVTVPSSSLGTKTYTCNVDHKPSN |
| | | | TKVDKRVESKYGPPCPPCPAPEAAGGPSVFLFPPKPKDTLMIS |
| | | | RTPEVTCVVVDVSQEDPEVQFNWYVDGVEVHNAKTKPREEQ |
| | | | FNSTYRVVSVLTVLHQDWLNGKEYKCKVSNKGLPSSIEKTISK |
| | | | AKGQPREPQVYTLPPCQEEMTKNQVSLWCLVKGFYPSDIAVE |
| | | | WESNGQPENNYKTTPPVLDSDGSFFLYSKLTVDKSRWQEGNV |
| | | | FSCSVMHEALHNHYTQKSLSLSLG |
| | 4 | 297 | DIQMTQSPSSLSASVGDRVTITCRASQDVNTAVAWYQQKPGK |
| | | | APKLLIYSASFLYSGVPSRFSGSRSGTDFTLTISSLQPEDFATYY |
| | | | CQQHYTTPPTFGQGTKVEIKRTVAAPSVFIFPPSDEQLKSGTAS |
| | | | VVCLLNNFYPREAKVQWKVDNALQSGNSQESVTEQDSKDST |
| | | | YSLSSTLTLSKADYEKHKVYACEVTHQGLSSPVTKSFNRGEC |
| HER2 | −1 | 298 | DIVMTQTPLSLSVTPGQPASISCKSSQSLVHQSAQTYLSWYLQ |
| (30R/55Q/102E | | | KPGQSPQSLIYKVSNRFSGVPDRFSGSGSGTDFTLKISRVEAED |
| + LC-WT- | | | VGVYYCGQGTQYPFTFGSGTKVEIKGQPKAAPDIQMTQSPSSL |
| trastuzumab)/ | | | SASVGDRVTITCQASQNIYVWLNWYQQKPGKAPKLLIYKASN |
| CD28supxCD3m | | | LHTGVPSRFSGSGSGTDFTLTISSLQPEDIATYYCQQGQTYPYT |
| id | | | FGQGTKLEIKTKGPSRTVAAPSVFIFPPSDEQLKSGTASVVCLL |
| (32/33/35QSQ18 | | | NNFYPREAKVQWKVDNALQSGNSQESVTEQDSKDSTYSLSST |
| 5S) IgG4 FALA | | | LTLSKADYEKHKVYACEVTHQGLSSPVTKSFNRGEC |
| BP # 6 | | | |
| | 2 | 299 | QVQLVQSGAEVVKPGASVKVSCKASGYTFTSYYIHWVRQAP |
| | | | GQGLEWIGSIYPGNVNTNYAQKFQGRATLTVDTSISTAYMELS |
| | | | RLRSDDTAVYYCTRSHYGLDWNFDVWGKGTTVTVSSSQVQL |
| | | | VESGGGVVQPGRSLRLSCAASGFTFTKAWMHWVRQAPGKQL |
| | | | EWVAQIKDKSNSYATYYASSVKGRFTISRDDSKNTLYLQMNS |
| | | | LRAEDTAVYYCRGVYYALSPFDYWGQGTLVTVSSRTASTKGP |
| | | | SVFPLAPCSRSTSESTAALGCLVKDYFPEPVTVSWNSGALTSG |
| | | | VHTFPAVLQSSGLYSLSSVVTVPSSSLGTKTYTCNVDHKPSNT |
| | | | KVDKRVESKYGPPCPPCPAPEAAGGPSVFLFPPKPKDTLMISR |
| | | | TPEVTCVVVDVSQEDPEVQFNWYVDGVEVHNAKTKPREEQF |
| | | | NSTYRVVSVLTVLHQDWLNGKEYKCKVSNKGLPSSIEKTISK |
| | | | AKGQPREPQVCTLPPSQEEMTKNQVSLSCAVKGFYPSDIAVE |
| | | | WESNGQPENNYKTTPPVLDSDGSFFLVSKLTVDKSRWQEGNV |
| | | | FSCSVMHEALHNHYTQKSLSLSLG |
| | 3 | 300 | EVQLVESGGGLVQPGGSLRLSCAASGFNIRDTYIHWVRQAPG |
| | | | KGLEWVARIYPTQGYTRYADSVKGRFTISADTSKNTAYLQMN |
| | | | SLRAEDTAVYYCSRWGGEGFYAMDYWGQGTLVTVSSASTKG |
| | | | PSVFPLAPCSRSTSESTAALGCLVKDYFPEPVTVSWNSGALTS |
| | | | GVHTFPAVLQSSGLYSLSSVVTVPSSSLGTKTYTCNVDHKPSN |
| | | | TKVDKRVESKYGPPCPPCPAPEAAGGPSVFLFPPKPKDTLMIS |
| | | | RTPEVTCVVVDVSQEDPEVQFNWYVDGVEVHNAKTKPREEQ |
| | | | FNSTYRVVSVLTVLHQDWLNGKEYKCKVSNKGLPSSIEKTISK |
| | | | AKGQPREPQVYTLPPCQEEMTKNQVSLWCLVKGFYPSDIAVE |
| | | | WESNGQPENNYKTTPPVLDSDGSFFLYSKLTVDKSRWQEGNV |
| | | | FSCSVMHEALHNHYTQKSLSLSLG |
| | 4 | 301 | DIQMTQSPSSLSASVGDRVTITCRASQDVNTAVAWYQQKPGK |
| | | | APKLLIYSASFLYSGVPSRFSGSRSGTDFTLTISSLQPEDFATYY |
| | | | CQQHYTTPPTFGQGTKVEIKRTVAAPSVFIFPPSDEQLKSGTAS |
| | | | VVCLLNNFYPREAKVQWKVDNALQSGNSQESVTEQDSKDST |
| | | | YSLSSTLTLSKADYEKHKVYACEVTHQGLSSPVTKSFNRGEC |
| HER2 | 1 | 112 | DIVMTQTPLSLSVTPGQPASISCKSSQSLVHQNAQTYLSWYLQ |
| (30R/55Q/102E | | | KPGQSPQSLIYKVSNRFSGVPDRFSGSGSGTDFTLKISRVEAED |
| + LC-WT- | | | VGVYYCGQGTQYPFTFGSGTKVEIKGQPKAAPDIQMTQSPSSL |
| trastuzumab)/ | | | SASVGDRVTITCQASQNIYVWLNWYQQKPGKAPKLLIYKASN |
| CD28ₛᵤₚxCD3_{mid} | | | LHTGVPSRFSGSGSGTDFTLTISSLQPEDIATYYCQQGQTYPYT |
| (32/35QQ (LC); | | | FGQGTKLEIKTKGPSRTVAAPSVFIFPPSDEQLKSGTASVVCLL |
| L1 linker) IgG4 | | | NNFYPREAKVQWKVDNALQSGNSQESVTEQDSKDSTYSLSST |
| FALA | | | LTLSKADYEKHKVYACEVTHQGLSSPVTKSFNRGEC |
| BP # 9 | | | |
| | 2 | 113 | QVQLVQSGAEVVKPGASVKVSCKASGYTFTSYYIHWVRQAP |
| | | | GQGLEWIGSIYPGNVNTNYAQKFQGRATLTVDTSISTAYMELS |
| | | | RLRSDDTAVYYCTRSHYGLDWNFDVWGKGTTVTVSSSQVQL |
| | | | VESGGGVVQPGRSLRLSCAASGFTFTKAWMHWVRQAPGKQL |
| | | | EWVAQIKDKSNSYATYYADSVKGRFTISRDDSKNTLYLQMNS |
| | | | LRAEDTAVYYCRGVYYALSPFDYWGQGTLVTVSSRTASTKGP |
| | | | SVFPLAPCSRSTSESTAALGCLVKDYFPEPVTVSWNSGALTSG |
| | | | VHTFPAVLQSSGLYSLSSVVTVPSSSLGTKTYTCNVDHKPSNT |
| | | | KVDKRVESKYGPPCPPCPAPEAAGGPSVFLFPPKPKDTLMISR |
| | | | TPEVTCVVVDVSQEDPEVQFNWYVDGVEVHNAKTKPREEQF |
| | | | NSTYRVVSVLTVLHQDWLNGKEYKCKVSNKGLPSSIEKTISK |
| | | | AKGQPREPQVCTLPPSQEEMTKNQVSLSCAVKGFYPSDIAVE |
| | | | WESNGQPENNYKTTPPVLDSDGSFFLVSKLTVDKSRWQEGNV |
| | | | FSCSVMHEALHNHYTQKSLSLSLG |
| | 3 | 114 | EVQLVESGGGLVQPGGSLRLSCAASGFNIRDTYIHWVRQAPG |
| | | | KGLEWVARIYPTQGYTRYADSVKGRFTISADTSKNTAYLQMN |
| | | | SLRAEDTAVYYCSRWGGEGFYAMDYWGQGTLVTVSSASTKG |
| | | | PSVFPLAPCSRSTSESTAALGCLVKDYFPEPVTVSWNSGALTS |
| | | | GVHTFPAVLQSSGLYSLSSVVTVPSSSLGTKTYTCNVDHKPSN |
| | | | TKVDKRVESKYGPPCPPCPAPEAAGGPSVFLFPPKPKDTLMIS |
| | | | RTPEVTCVVVDVSQEDPEVQFNWYVDGVEVHNAKTKPREEQ |
| | | | FNSTYRVVSVLTVLHQDWLNGKEYKCKVSNKGLPSSIEKTISK |
| | | | AKGQPREPQVYTLPPCQEEMTKNQVSLWCLVKGFYPSDIAVE |
| | | | WESNGQPENNYKTTPPVLDSDGSFFLYSKLTVDKSRWQEGNV |
| | | | FSCSVMHEALHNHYTQKSLSLSLG |
| | 4 | 115 | DIQMTQSPSSLSASVGDRVTITCRASQDVNTAVAWYQQKPGK |
| | | | APKLLIYSASFLYSGVPSRFSGSRSGTDFTLTISSLQPEDFATYY |
| | | | CQQHYTTPPTFGQGTKVEIKRTVAAPSVFIFPPSDEQLKSGTAS |
| | | | VVCLLNNFYPREAKVQWKVDNALQSGNSQESVTEQDSKDST |
| | | | YSLSSTLTLSKADYEKHKVYACEVTHQGLSSPVTKSFNRGEC |
| HER2-30R/ | 1 | 116 | DIVMTQTPLSLSVTPGQPASISCKSSQSLVHNNANTYLSWYLQ |
| 55Q/102S + | | | KPGQSPQSLIYKVSNRFSGVPDRFSGSGSGTDFTLKISRVEAED |
| LC-WT- | | | VGVYYCGQGTQYPFTFGSGTKVEIKGQPKAAPDIQMTQSPSSL |
| trastuzumab/ | | | SASVGDRVTITCQASQNIYVWLNWYQQKPGKAPKLLIYKASN |
| CD28supxCD3m | | | LHTGVPSRFSGSGSGTDFTLTISSLQPEDIATYYCQQGQTYPYT |
| id L 1 linker | | | FGQGTKLEIKTKGPSRTVAAPSVFIFPPSDEQLKSGTASVVCLL |
| IgG4 FALA | | | NNFYPREAKVQWKVDNALQSGNSQESVTEQDSKDSTYSLSST |
| BP # 10 | | | LTLSKADYEKHKVYACEVTHQGLSSPVTKSFNRGEC |
| | 2 | 117 | QVQLVQSGAEVVKPGASVKVSCKASGYTFTSYYIHWVRQAP |
| | | | GQGLEWIGSIYPGNVNTNYAQKFQGRATLTVDTSISTAYMELS |
| | | | RLRSDDTAVYYCTRSHYGLDWNFDVWGKGTTVTVSSSQVQL |
| | | | VESGGGVVQPGRSLRLSCAASGFTFTKAWMHWVRQAPGKQL |
| | | | EWVAQIKDKSNSYATYYADSVKGRFTISRDDSKNTLYLQMNS |
| | | | LRAEDTAVYYCRGVYYALSPFDYWGQGTLVTVSSRTASTKGP |
| | | | SVFPLAPCSRSTSESTAALGCLVKDYFPEPVTVSWNSGALTSG |
| | | | VHTFPAVLQSSGLYSLSSVVTVPSSSLGTKTYTCNVDHKPSNT |
| | | | KVDKRVESKYGPPCPPCPAPEAAGGPSVFLFPPKPKDTLMISR |
| | | | TPEVTCVVVDVSQEDPEVQFNWYVDGVEVHNAKTKPREEQF |
| | | | NSTYRVVSVLTVLHQDWLNGKEYKCKVSNKGLPSSIEKTISK |
| | | | AKGQPREPQVCTLPPSQEEMTKNQVSLSCAVKGFYPSDIAVE |
| | | | WESNGQPENNYKTTPPVLDSDGSFFLVSKLTVDKSRWQEGNV |
| | | | FSCSVMHEALHNHYTQKSLSLSLG |
| | 3 | 118 | EVQLVESGGGLVQPGGSLRLSCAASGFNIRDTYIHWVRQAPG |
| | | | KGLEWVARIYPTQGYTRYADSVKGRFTISADTSKNTAYLQMN |
| | | | SLRAEDTAVYYCSRWGGSGFYAMDYWGQGTLVTVSSASTKG |
| | | | PSVFPLAPCSRSTSESTAALGCLVKDYFPEPVTVSWNSGALTS |
| | | | GVHTFPAVLQSSGLYSLSSVVTVPSSSLGTKTYTCNVDHKPSN |
| | | | TKVDKRVESKYGPPCPPCPAPEFLGGPSVFLFPPKPKDTLMISR |
| | | | TPEVTCVVVDVSQEDPEVQFNWYVDGVEVHNAKTKPREEQF |
| | | | NSTYRVVSVLTVLHQDWLNGKEYKCKVSNKGLPSSIEKTISK |
| | | | AKGQPREPQVYTLPPCQEEMTKNQVSLWCLVKGFYPSDIAVE |
| | | | WESNGQPENNYKTTPPVLDSDGSFFLYSKLTVDKSRWQEGNV |
| | | | FSCSVMHEALHNHYTQKSLSLSLG |
| | 4 | 119 | DIQMTQSPSSLSASVGDRVTITCRASQDVNTAVAWYQQKPGK |
| | | | APKLLIYSASFLYSGVPSRFSGSRSGTDFTLTISSLQPEDFATYY |
| | | | CQQHYTTPPTFGQGTKVEIKRTVAAPSVFIFPPSDEQLKSGTAS |
| | | | VVCLLNNFYPREAKVQWKVDNALQSGNSQESVTEQDSKDST |
| | | | YSLSSTLTLSKADYEKHKVYACEVTHQGLSSPVTKSFNRGEC |
| HER2-30R/ | 1 | 120 | DIVMTQTPLSLSVTPGQPASISCKSSQSLVHNNANTYLSWYLQ |
| 56A/102S + | | | KPGQSPQSLIYKVSNRFSGVPDRFSGSGSGTDFTLKISRVEAED |
| LC-WT- | | | VGVYYCGQGTQYPFTFGSGTKVEIKGQPKAAPDIQMTQSPSSL |
| trastuzumab/ | | | SASVGDRVTITCQASQNIYVWLNWYQQKPGKAPKLLIYKASN |
| CD28supxCD3m | | | LHTGVPSRFSGSGSGTDFTLTISSLQPEDIATYYCQQGQTYPYT |
| id L1 linker | | | FGQGTKLEIKTKGPSRTVAAPSVFIFPPSDEQLKSGTASVVCLL |
| IgG4 FALA | | | NNFYPREAKVQWKVDNALQSGNSQESVTEQDSKDSTYSLSST |
| BP # 11 | | | LTLSKADYEKHKVYACEVTHQGLSSPVTKSFNRGEC |
| | 2 | 121 | QVQLVQSGAEVVKPGASVKVSCKASGYTFTSYYIHWVRQAP |
| | | | GQGLEWIGSIYPGNVNTNYAQKFQGRATLTVDTSISTAYMELS |
| | | | RLRSDDTAVYYCTRSHYGLDWNFDVWGKGTTVTVSSSQVQL |
| | | | VESGGGVVQPGRSLRLSCAASGFTFTKAWMHWVRQAPGKQL |
| | | | EWVAQIKDKSNSYATYYADSVKGRFTISRDDSKNTLYLQMNS |
| | | | LRAEDTAVYYCRGVYYALSPFDYWGQGTLVTVSSRTASTKGP |
| | | | SVFPLAPCSRSTSESTAALGCLVKDYFPEPVTVSWNSGALTSG |
| | | | VHTFPAVLQSSGLYSLSSVVTVPSSSLGTKTYTCNVDHKPSNT |
| | | | KVDKRVESKYGPPCPPCPAPEAAGGPSVFLFPPKPKDTLMISR |
| | | | TPEVTCVVVDVSQEDPEVQFNWYVDGVEVHNAKTKPREEQF |
| | | | NSTYRVVSVLTVLHQDWLNGKEYKCKVSNKGLPSSIEKTISK |
| | | | AKGQPREPQVCTLPPSQEEMTKNQVSLSCAVKGFYPSDIAVE |
| | | | WESNGQPENNYKTTPPVLDSDGSFFLVSKLTVDKSRWQEGNV |
| | | | FSCSVMHEALHNHYTQKSLSLSLG |
| | 3 | 122 | EVQLVESGGGLVQPGGSLRLSCAASGFNIRDTYIHWVRQAPG |
| | | | KGLEWVARIYPTNAYTRYADSVKGRFTISADTSKNTAYLQMN |
| | | | SLRAEDTAVYYCSRWGGSGFYAMDYWGQGTLVTVSSASTKG |
| | | | PSVFPLAPCSRSTSESTAALGCLVKDYFPEPVTVSWNSGALTS |
| | | | GVHTFPAVLQSSGLYSLSSVVTVPSSSLGTKTYTCNVDHKPSN |
| | | | TKVDKRVESKYGPPCPPCPAPEFLGGPSVFLFPPKPKDTLMISR |
| | | | TPEVTCVVVDVSQEDPEVQFNWYVDGVEVHNAKTKPREEQF |
| | | | NSTYRVVSVLTVLHQDWLNGKEYKCKVSNKGLPSSIEKTISK |
| | | | AKGQPREPQVYTLPPCQEEMTKNQVSLWCLVKGFYPSDIAVE |
| | | | WESNGQPENNYKTTPPVLDSDGSFFLYSKLTVDKSRWQEGNV |
| | | | FSCSVMHEALHNHYTQKSLSLSLG |
| | 4 | 123 | DIQMTQSPSSLSASVGDRVTITCRASQDVNTAVAWYQQKPGK |
| | | | APKLLIYSASFLYSGVPSRFSGSRSGTDFTLTISSLQPEDFATYY |
| | | | CQQHYTTPPTFGQGTKVEIKRTVAAPSVFIFPPSDEQLKSGTAS |
| | | | VVCLLNNFYPREAKVQWKVDNALQSGNSQESVTEQDSKDST |
| | | | YSLSSTLTLSKADYEKHKVYACEVTHQGLSSPVTKSFNRGEC |
| HER2-30R/ | 1 | 124 | DIVMTQTPLSLSVTPGQPASISCKSSQSLVHNNANTYLSWYLQ |
| 56A/102E/ | | | KPGQSPQSLIYKVSNRFSGVPDRFSGSGSGTDFTLKISRVEAED |
| CD28supxCD3m | | | VGVYYCGQGTQYPFTFGSGTKVEIKGQPKAAPDIQMTQSPSSL |
| id L1 linker | | | SASVGDRVTITCQASQNIYVWLNWYQQKPGKAPKLLIYKASN |
| IgG4 FALA | | | LHTGVPSRFSGSGSGTDFTLTISSLQPEDIATYYCQQGQTYPYT |
| BP # 12 | | | FGQGTKLEIKTKGPSRTVAAPSVFIFPPSDEQLKSGTASVVCLL |
| | | | NNFYPREAKVQWKVDNALQSGNSQESVTEQDSKDSTYSLSST |
| | | | LTLSKADYEKHKVYACEVTHQGLSSPVTKSFNRGEC |
| | 2 | 125 | QVQLVQSGAEVVKPGASVKVSCKASGYTFTSYYIHWVRQAP |
| | | | GQGLEWIGSIYPGNVNTNYAQKFQGRATLTVDTSISTAYMELS |
| | | | RLRSDDTAVYYCTRSHYGLDWNFDVWGKGTTVTVSSSQVQL |
| | | | VESGGGVVQPGRSLRLSCAASGFTFTKAWMHWVRQAPGKQL |
| | | | EWVAQIKDKSNSYATYYADSVKGRFTISRDDSKNTLYLQMNS |
| | | | LRAEDTAVYYCRGVYYALSPFDYWGQGTLVTVSSRTASTKGP |
| | | | SVFPLAPCSRSTSESTAALGCLVKDYFPEPVTVSWNSGALTSG |
| | | | VHTFPAVLQSSGLYSLSSVVTVPSSSLGTKTYTCNVDHKPSNT |
| | | | KVDKRVESKYGPPCPPCPAPEAAGGPSVFLFPPKPKDTLMISR |
| | | | TPEVTCVVVDVSQEDPEVQFNWYVDGVEVHNAKTKPREEQF |
| | | | NSTYRVVSVLTVLHQDWLNGKEYKCKVSNKGLPSSIEKTISK |
| | | | AKGQPREPQVCTLPPSQEEMTKNQVSLSCAVKGFYPSDIAVE |
| | | | WESNGQPENNYKTTPPVLDSDGSFFLVSKLTVDKSRWQEGNV |
| | | | FSCSVMHEALHNHYTQKSLSLSLG |
| | 3 | 126 | EVQLVESGGGLVQPGGSLRLSCAASGFNIRDTYIHWVRQAPG |
| | | | KGLEWVARIYPTNAYTRYADSVKGRFTISADTSKNTAYLQMN |
| | | | SLRAEDTAVYYCSRWGGEGFYAMDYWGQGTLVTVSSASTKG |
| | | | PSVFPLAPCSRSTSESTAALGCLVKDYFPEPVTVSWNSGALTS |
| | | | GVHTFPAVLQSSGLYSLSSVVTVPSSSLGTKTYTCNVDHKPSN |
| | | | TKVDKRVESKYGPPCPPCPAPEFLGGPSVFLFPPKPKDTLMISR |
| | | | TPEVTCVVVDVSQEDPEVQFNWYVDGVEVHNAKTKPREEQF |
| | | | NSTYRVVSVLTVLHQDWLNGKEYKCKVSNKGLPSSIEKTISK |
| | | | AKGQPREPQVYTLPPCQEEMTKNQVSLWCLVKGFYPSDIAVE |
| | | | WESNGQPENNYKTTPPVLDSDGSFFLYSKLTVDKSRWQEGNV |
| | | | FSCSVMHEALHNHYTQKSLSLSLG |
| | 4 | 127 | DIQMTQSPSSLSASVGDRVTITCRASQDVNTAVAWYQQKPGK |
| | | | APKLLIYSASFLYSGVPSRFSGSRSGTDFTLTISSLQPEDFATYY |
| | | | CQQHYTTPPTFGQGTKVEIKRTVAAPSVFIFPPSDEQLKSGTAS |
| | | | VVCLLNNFYPREAKVQWKVDNALQSGNSQESVTEQDSKDST |
| | | | YSLSSTLTLSKADYEKHKVYACEVTHQGLSSPVTKSFNRGEC |
| HER2-WT + | 1 | 128 | DIVMTQTPLSLSVTPGQPASISCKSSQSLVHQNAQTYLSWYLQ |
| trastuzumab/ | | | KPGQSPQSLIYKVSNRFSGVPDRFSGSGSGTDFTLKISRVEAED |
| CD28supxCD3m | | | VGVYYCGQGTQYPFTFGSGTKVEIKGQPKAAPDIQMTQSPSSL |
| id (32/35QQ) L1 | | | SASVGDRVTITCQASQNIYVWLNWYQQKPGKAPKLLIYKASN |
| linker IgG4 | | | LHTGVPSRFSGSGSGTDFTLTISSLQPEDIATYYCQQGQTYPYT |
| FALA | | | FGQGTKLEIKTKGPSRTVAAPSVFIFPPSDEQLKSGTASVVCLL |
| BP # 15 | | | NNFYPREAKVQWKVDNALQSGNSQESVTEQDSKDSTYSLSST |
| | | | LTLSKADYEKHKVYACEVTHQGLSSPVTKSFNRGEC |
| | 2 | 129 | QVQLVQSGAEVVKPGASVKVSCKASGYTFTSYYIHWVRQAP |
| | | | GQGLEWIGSIYPGNVNTNYAQKFQGRATLTVDTSISTAYMELS |
| | | | RLRSDDTAVYYCTRSHYGLDWNFDVWGKGTTVTVSSSQVQL |
| | | | VESGGGVVQPGRSLRLSCAASGFTFTKAWMHWVRQAPGKQL |
| | | | EWVAQIKDKSNSYATYYADSVKGRFTISRDDSKNTLYLQMNS |
| | | | LRAEDTAVYYCRGVYYALSPFDYWGQGTLVTVSSRTASTKGP |
| | | | SVFPLAPCSRSTSESTAALGCLVKDYFPEPVTVSWNSGALTSG |
| | | | VHTFPAVLQSSGLYSLSSVVTVPSSSLGTKTYTCNVDHKPSNT |
| | | | KVDKRVESKYGPPCPPCPAPEAAGGPSVFLFPPKPKDTLMISR |
| | | | TPEVTCVVVDVSQEDPEVQFNWYVDGVEVHNAKTKPREEQF |
| | | | NSTYRVVSVLTVLHQDWLNGKEYKCKVSNKGLPSSIEKTISK |
| | | | AKGQPREPQVCTLPPSQEEMTKNQVSLSCAVKGFYPSDIAVE |
| | | | WESNGQPENNYKTTPPVLDSDGSFFLVSKLTVDKSRWQEGNV |
| | | | FSCSVMHEALHNHYTQKSLSLSLG |
| | | 130 | EVQLVESGGGLVQPGGSLRLSCAASGFNIKDTYIHWVRQAPG |
| | | | KGLEWVARIYPTNGYTRYADSVKGRFTISADTSKNTAYLQMN |
| | | | SLRAEDTAVYYCSRWGGDGFYAMDYWGQGTLVTVSSASTK |
| | | | GPSVFPLAPCSRSTSESTAALGCLVKDYFPEPVTVSWNSGALT |
| | | | SGVHTFPAVLQSSGLYSLSSVVTVPSSSLGTKTYTCNVDHKPS |
| | | | NTKVDKRVESKYGPPCPPCPAPEAAGGPSVFLFPPKPKDTLMI |
| | | | SRTPEVTCVVVDVSQEDPEVQFNWYVDGVEVHNAKTKPREE |
| | | | QFNSTYRVVSVLTVLHQDWLNGKEYKCKVSNKGLPSSIEKTIS |
| | | | KAKGQPREPQVYTLPPCQEEMTKNQVSLWCLVKGFYPSDIAV |
| | | | EWESNGQPENNYKTTPPVLDSDGSFFLYSKLTVDKSRWQEGN |
| | | | VFSCSVMHEALHNHYTQKSLSLSLG |
| | 4 | 131 | DIQMTQSPSSLSASVGDRVTITCRASQDVNTAVAWYQQKPGK |
| | | | APKLLIYSASFLYSGVPSRFSGSRSGTDFTLTISSLQPEDFATYY |
| | | | CQQHYTTPPTFGQGTKVEIKRTVAAPSVFIFPPSDEQLKSGTAS |
| | | | VVCLLNNFYPREAKVQWKVDNALQSGNSQESVTEQDSKDST |
| | | | YSLSSTLTLSKADYEKHKVYACEVTHQGLSSPVTKSFNRGEC |
| HER2/ | 1 | 132 | DIVMTQTPLSLSVTPGQPASISCKSSQSLVHNNANTYLSWYLQ |
| CD28ₛᵤₚxCD3_{mid} | | | KPGQSPQSLIYKVSNRFSGVPDRFSGSGSGTDFTLKISRVEAED |
| DKTHT linkers | | | VGVYYCGQGTQYPFTFGSGTKVEIKDKTHTDIQMTQSPSSLSA |
| on HC/LC) IgG4 | | | SVGDRVTITCQASQNIYVWLNWYQQKPGKAPKLLIYKASNLH |
| FALA | | | TGVPSRFSGSGSGTDFTLTISSLQPEDIATYYCQQGQTYPYTFG |
| BP # 25 | | | QGTKLEIKDKTHTRTVAAPSVFIFPPSDEQLKSGTASVVCLLNN |
| | | | FYPREAKVQWKVDNALQSGNSQESVTEQDSKDSTYSLSSTLT |
| | | | LSKADYEKHKVYACEVTHQGLSSPVTKSFNRGEC |
| | 2 | 133 | QVQLVQSGAEVVKPGASVKVSCKASGYTFTSYYIHWVRQAP |
| | | | GQGLEWIGSIYPGNVNTNYAQKFQGRATLTVDTSISTAYMELS |
| | | | RLRSDDTAVYYCTRSHYGLDWNFDVWGKGTTVTVSSDKTHT |
| | | | QVQLVESGGGVVQPGRSLRLSCAASGFTFTKAWMHWVRQAP |
| | | | GKQLEWVAQIKDKSNSYATYYADSVKGRFTISRDDSKNTLYL |
| | | | QMNSLRAEDTAVYYCRGVYYALSPFDYWGQGTLVTVSSDKT |
| | | | HTASTKGPSVFPLAPCSRSTSESTAALGCLVKDYFPEPVTVSW |
| | | | NSGALTSGVHTFPAVLQSSGLYSLSSVVTVPSSSLGTKTYTCN |
| | | | VDHKPSNTKVDKRVESKYGPPCPPCPAPEAAGGPSVFLFPPKP |
| | | | KDTLMISRTPEVTCVVVDVSQEDPEVQFNWYVDGVEVHNAK |
| | | | TKPREEQFNSTYRVVSVLTVLHQDWLNGKEYKCKVSNKGLPS |
| | | | SIEKTISKAKGQPREPQVCTLPPSQEEMTKNQVSLSCAVKGFY |
| | | | PSDIAVEWESNGQPENNYKTTPPVLDSDGSFFLVSKLTVDKSR |
| | | | WQEGNVFSCSVMHEALHNHYTQKSLSLSLG |
| | 3 | 134 | EVQLVESGGGLVQPGGSLRLSCAASGFNIKDTYIHWVRQAPG |
| | | | KGLEWVARIYPTNGYTRYADSVKGRFTISADTSKNTAYLQMN |
| | | | SLRAEDTAVYYCSRWGGDGFYAMDYWGQGTLVTVSSASTK |
| | | | GPSVFPLAPCSRSTSESTAALGCLVKDYFPEPVTVSWNSGALT |
| | | | SGVHTFPAVLQSSGLYSLSSVVTVPSSSLGTKTYTCNVDHKPS |
| | | | NTKVDKRVESKYGPPCPPCPAPEAAGGPSVFLFPPKPKDTLMI |
| | | | SRTPEVTCVVVDVSQEDPEVQFNWYVDGVEVHNAKTKPREE |
| | | | QFNSTYRVVSVLTVLHQDWLNGKEYKCKVSNKGLPSSIEKTIS |
| | | | KAKGQPREPQVYTLPPCQEEMTKNQVSLWCLVKGFYPSDIAV |
| | | | EWESNGQPENNYKTTPPVLDSDGSFFLYSKLTVDKSRWQEGN |
| | | | VFSCSVMHEALHNHYTQKSLSLSLG |
| | 4 | 135 | DIQMTQSPSSLSASVGDRVTITCRASQDVNTAVAWYQQKPGK |
| | | | APKLLIYSASFLYSGVPSRFSGSRSGTDFTLTISSLQPEDFATYY |
| | | | CQQHYTTPPTFGQGTKVEIKRTVAAPSVFIFPPSDEQLKSGTAS |
| | | | VVCLLNNFYPREAKVQWKVDNALQSGNSQESVTEQDSKDST |
| | | | YSLSSTLTLSKADYEKHKVYACEVTHQGLSSPVTKSFNRGEC |
| HER2/ | 1 | 136 | DIVMTQTPLSLSVTPGQPASISCKSSQSLVHENLRTYLSWYLQ |
| CD28ₛᵤₚxCD3_{mid} | | | KPGQSPQSLIYKVSNRFSGVPDRFSGSGSGTDFTLKISRVEAED |
| (32/33/3435 | | | VGVYYCGQGTQYPFTFGSGTKVEIKDKTHTDIQMTQSPSSLSA |
| ENLR (LC); | | | SVGDRVTITCQASQNIYVWLNWYQQKPGKAPKLLIYKASNLH |
| DKTHT linkers | | | TGVPSRFSGSGSGTDFTLTISSLQPEDIATYYCQQGQTYPYTFG |
| on HC/LC) IgG4 | | | QGTKLEIKDKTHTRTVAAPSVFIFPPSDEQLKSGTASVVCLLNN |
| FALA | | | FYPREAKVQWKVDNALQSGNSQESVTEQDSKDSTYSLSSTLT |
| BP # 26 | | | LSKADYEKHKVYACEVTHQGLSSPVTKSFNRGEC |
| | 2 | 137 | QVQLVQSGAEVVKPGASVKVSCKASGYTFTSYYIHWVRQAP |
| | | | GQGLEWIGSIYPGNVNTNYAQKFQGRATLTVDTSISTAYMELS |
| | | | RLRSDDTAVYYCTRSHYGLDWNFDVWGKGTTVTVSSDKTHT |
| | | | QVQLVESGGGVVQPGRSLRLSCAASGFTFTKAWMHWVRQAP |
| | | | GKQLEWVAQIKDKSNSYATYYADSVKGRFTISRDDSKNTLYL |
| | | | QMNSLRAEDTAVYYCRGVYYALSPFDYWGQGTLVTVSSDKT |
| | | | HTASTKGPSVFPLAPCSRSTSESTAALGCLVKDYFPEPVTVSW |
| | | | NSGALTSGVHTFPAVLQSSGLYSLSSVVTVPSSSLGTKTYTCN |
| | | | VDHKPSNTKVDKRVESKYGPPCPPCPAPEAAGGPSVFLFPPKP |
| | | | KDTLMISRTPEVTCVVVDVSQEDPEVQFNWYVDGVEVHNAK |
| | | | TKPREEQFNSTYRVVSVLTVLHQDWLNGKEYKCKVSNKGLPS |
| | | | SIEKTISKAKGQPREPQVCTLPPSQEEMTKNQVSLSCAVKGFY |
| | | | PSDIAVEWESNGQPENNYKTTPPVLDSDGSFFLVSKLTVDKSR |
| | | | WQEGNVFSCSVMHEALHNHYTQKSLSLSLG |
| | 3 | 138 | EVQLVESGGGLVQPGGSLRLSCAASGFNIKDTYIHWVRQAPG |
| | | | KGLEWVARIYPTNGYTRYADSVKGRFTISADTSKNTAYLQMN |
| | | | SLRAEDTAVYYCSRWGGDGFYAMDYWGQGTLVTVSSASTK |
| | | | GPSVFPLAPCSRSTSESTAALGCLVKDYFPEPVTVSWNSGALT |
| | | | SGVHTFPAVLQSSGLYSLSSVVTVPSSSLGTKTYTCNVDHKPS |
| | | | NTKVDKRVESKYGPPCPPCPAPEAAGGPSVFLFPPKPKDTLMI |
| | | | SRTPEVTCVVVDVSQEDPEVQFNWYVDGVEVHNAKTKPREE |
| | | | QFNSTYRVVSVLTVLHQDWLNGKEYKCKVSNKGLPSSIEKTIS |
| | | | KAKGQPREPQVYTLPPCQEEMTKNQVSLWCLVKGFYPSDIAV |
| | | | EWESNGQPENNYKTTPPVLDSDGSFFLYSKLTVDKSRWQEGN |
| | | | VFSCSVMHEALHNHYTQKSLSLSLG |
| | 4 | 139 | DIQMTQSPSSLSASVGDRVTITCRASQDVNTAVAWYQQKPGK |
| | | | APKLLIYSASFLYSGVPSRFSGSRSGTDFTLTISSLQPEDFATYY |
| | | | CQQHYTTPPTFGQGTKVEIKRTVAAPSVFIFPPSDEQLKSGTAS |
| | | | VVCLLNNFYPREAKVQWKVDNALQSGNSQESVTEQDSKDST |
| | | | YSLSSTLTLSKADYEKHKVYACEVTHQGLSSPVTKSFNRGEC |
| HER2/ | 1 | 140 | DIVMTQTPLSLSVTPGQPASISCKSSQSLVHENLQTYLSWYLQ |
| CD28ₛᵤₚxCD3_{mid} | | | KPGQSPQSLIYKVSNRFSGVPDRFSGSGSGTDFTLKISRVEAED |
| (32/33/3435 | | | VGVYYCGQGTQYPFTFGSGTKVEIKDKTHTDIQMTQSPSSLSA |
| ENLQ (LC); | | | SVGDRVTITCQASQNIYVWLNWYQQKPGKAPKLLIYKASNLH |
| DKTHT linkers | | | TGVPSRFSGSGSGTDFTLTISSLQPEDIATYYCQQGQTYPYTFG |
| on HC/LC) IgG4 | | | QGTKLEIKDKTHTRTVAAPSVFIFPPSDEQLKSGTASVVCLLNN |
| FALA | | | FYPREAKVQWKVDNALQSGNSQESVTEQDSKDSTYSLSSTLT |
| BP # 27 | | | LSKADYEKHKVYACEVTHQGLSSPVTKSFNRGEC |
| | 2 | 141 | QVQLVQSGAEVVKPGASVKVSCKASGYTFTSYYIHWVRQAP |
| | | | GQGLEWIGSIYPGNVNTNYAQKFQGRATLTVDTSISTAYMELS |
| | | | RLRSDDTAVYYCTRSHYGLDWNFDVWGKGTTVTVSSDKTHT |
| | | | QVQLVESGGGVVQPGRSLRLSCAASGFTFTKAWMHWVRQAP |
| | | | GKQLEWVAQIKDKSNSYATYYADSVKGRFTISRDDSKNTLYL |
| | | | QMNSLRAEDTAVYYCRGVYYALSPFDYWGQGTLVTVSSDKT |
| | | | HTASTKGPSVFPLAPCSRSTSESTAALGCLVKDYFPEPVTVSW |
| | | | NSGALTSGVHTFPAVLQSSGLYSLSSVVTVPSSSLGTKTYTCN |
| | | | VDHKPSNTKVDKRVESKYGPPCPPCPAPEAAGGPSVFLFPPKP |
| | | | KDTLMISRTPEVTCVVVDVSQEDPEVQFNWYVDGVEVHNAK |
| | | | TKPREEQFNSTYRVVSVLTVLHQDWLNGKEYKCKVSNKGLPS |
| | | | SIEKTISKAKGQPREPQVCTLPPSQEEMTKNQVSLSCAVKGFY |
| | | | PSDIAVEWESNGQPENNYKTTPPVLDSDGSFFLVSKLTVDKSR |
| | | | WQEGNVFSCSVMHEALHNHYTQKSLSLSLG |
| | 3 | 142 | EVQLVESGGGLVQPGGSLRLSCAASGFNIKDTYIHWVRQAPG |
| | | | KGLEWVARIYPTNGYTRYADSVKGRFTISADTSKNTAYLQMN |
| | | | SLRAEDTAVYYCSRWGGDGFYAMDYWGQGTLVTVSSASTK |
| | | | GPSVFPLAPCSRSTSESTAALGCLVKDYFPEPVTVSWNSGALT |
| | | | SGVHTFPAVLQSSGLYSLSSVVTVPSSSLGTKTYTCNVDHKPS |
| | | | NTKVDKRVESKYGPPCPPCPAPEAAGGPSVFLFPPKPKDTLMI |
| | | | SRTPEVTCVVVDVSQEDPEVQFNWYVDGVEVHNAKTKPREE |
| | | | QFNSTYRVVSVLTVLHQDWLNGKEYKCKVSNKGLPSSIEKTIS |
| | | | KAKGQPREPQVYTLPPCQEEMTKNQVSLWCLVKGFYPSDIAV |
| | | | EWESNGQPENNYKTTPPVLDSDGSFFLYSKLTVDKSRWQEGN |
| | | | VFSCSVMHEALHNHYTQKSLSLSLG |
| | 4 | 143 | DIQMTQSPSSLSASVGDRVTITCRASQDVNTAVAWYQQKPGK |
| | | | APKLLIYSASFLYSGVPSRFSGSRSGTDFTLTISSLQPEDFATYY |
| | | | CQQHYTTPPTFGQGTKVEIKRTVAAPSVFIFPPSDEQLKSGTAS |
| | | | VVCLLNNFYPREAKVQWKVDNALQSGNSQESVTEQDSKDST |
| | | | YSLSSTLTLSKADYEKHKVYACEVTHQGLSSPVTKSFNRGEC |
| HER2/ | 1 | 144 | DIVMTQTPLSLSVTPGQPASISCKSSQSLVHENLFTYLSWYLQK |
| CD28ₛᵤₚxCD3_{mid} | | | PGQSPQSLIYKVSNRFSGVPDRFSGSGSGTDFTLKISRVEAEDV |
| (32/33/3435 | | | GVYYCGQGTQYPFTFGSGTKVEIKDKTHTDIQMTQSPSSLSAS |
| ENLF (LC); | | | VGDRVTITCQASQNIYVWLNWYQQKPGKAPKLLIYKASNLHT |
| DKTHT linkers | | | GVPSRFSGSGSGTDFTLTISSLQPEDIATYYCQQGQTYPYTFGQ |
| on HC/LC) IgG4 | | | GTKLEIKDKTHTRTVAAPSVFIFPPSDEQLKSGTASVVCLLNNF |
| FALA | | | YPREAKVQWKVDNALQSGNSQESVTEQDSKDSTYSLSSTLTL |
| BP # 28 | | | SKADYEKHKVYACEVTHQGLSSPVTKSFNRGEC |
| | 2 | 145 | QVQLVQSGAEVVKPGASVKVSCKASGYTFTSYYIHWVRQAP |
| | | | GQGLEWIGSIYPGNVNTNYAQKFQGRATLTVDTSISTAYMELS |
| | | | RLRSDDTAVYYCTRSHYGLDWNFDVWGKGTTVTVSSDKTHT |
| | | | QVQLVESGGGVVQPGRSLRLSCAASGFTFTKAWMHWVRQAP |
| | | | GKQLEWVAQIKDKSNSYATYYADSVKGRFTISRDDSKNTLYL |
| | | | QMNSLRAEDTAVYYCRGVYYALSPFDYWGQGTLVTVSSDKT |
| | | | HTASTKGPSVFPLAPCSRSTSESTAALGCLVKDYFPEPVTVSW |
| | | | NSGALTSGVHTFPAVLQSSGLYSLSSVVTVPSSSLGTKTYTCN |
| | | | VDHKPSNTKVDKRVESKYGPPCPPCPAPEAAGGPSVFLFPPKP |
| | | | KDTLMISRTPEVTCVVVDVSQEDPEVQFNWYVDGVEVHNAK |
| | | | TKPREEQFNSTYRVVSVLTVLHQDWLNGKEYKCKVSNKGLPS |
| | | | SIEKTISKAKGQPREPQVCTLPPSQEEMTKNQVSLSCAVKGFY |
| | | | PSDIAVEWESNGQPENNYKTTPPVLDSDGSFFLVSKLTVDKSR |
| | | | WQEGNVFSCSVMHEALHNHYTQKSLSLSLG |
| | 3 | 146 | EVQLVESGGGLVQPGGSLRLSCAASGFNIKDTYIHWVRQAPG |
| | | | KGLEWVARIYPTNGYTRYADSVKGRFTISADTSKNTAYLQMN |
| | | | SLRAEDTAVYYCSRWGGDGFYAMDYWGQGTLVTVSSASTK |
| | | | GPSVFPLAPCSRSTSESTAALGCLVKDYFPEPVTVSWNSGALT |
| | | | SGVHTFPAVLQSSGLYSLSSVVTVPSSSLGTKTYTCNVDHKPS |
| | | | NTKVDKRVESKYGPPCPPCPAPEAAGGPSVFLFPPKPKDTLMI |
| | | | SRTPEVTCVVVDVSQEDPEVQFNWYVDGVEVHNAKTKPREE |
| | | | QFNSTYRVVSVLTVLHQDWLNGKEYKCKVSNKGLPSSIEKTIS |
| | | | KAKGQPREPQVYTLPPCQEEMTKNQVSLWCLVKGFYPSDIAV |
| | | | EWESNGQPENNYKTTPPVLDSDGSFFLYSKLTVDKSRWQEGN |
| | | | VFSCSVMHEALHNHYTQKSLSLSLG |
| | 4 | 147 | DIQMTQSPSSLSASVGDRVTITCRASQDVNTAVAWYQQKPGK |
| | | | APKLLIYSASFLYSGVPSRFSGSRSGTDFTLTISSLQPEDFATYY |
| | | | CQQHYTTPPTFGQGTKVEIKRTVAAPSVFIFPPSDEQLKSGTAS |
| | | | VVCLLNNFYPREAKVQWKVDNALQSGNSQESVTEQDSKDST |
| | | | YSLSSTLTLSKADYEKHKVYACEVTHQGLSSPVTKSFNRGEC |
| anti- | 1 | 148 | DIVMTQTPLSLSVTPGQPASISCKSSQSLVHNNANTYLSWYLQ |
| Her2/CD3/3CD28 | | | KPGQSPQSLIYKVSNRFSGVPDRFSGSGSGTDFTLKISRVEAED |
| IgG4 FALA | | | VGVYYCGQGTQYPFTFGSGTKVEIKGQPKAAPDIQMTQSPSSL |
| BP # 29 | | | SASVGDRVTITCQASQNIYVWLNWYQQKPGKAPKLLIYKASN |
| | | | LHTGVPSRFSGSGSGTDFTLTISSLQPEDIATYYCQQGQTYPYT |
| | | | FGQGTKLEIKTKGPSRTVAAPSVFIFPPSDEQLKSGTASVVCLL |
| | | | NNFYPREAKVQWKVDNALQSGNSQESVTEQDSKDSTYSLSST |
| | | | LTLSKADYEKHKVYACEVTHQGLSSPVTKSFNRGEC |
| | 2 | 149 | QVQLVQSGAEVVKPGASVKVSCKASGYTFTSYYIHWVRQAP |
| | | | GQGLEWIGSIYPGNVNTNYAQKFQGRATLTVDTSISTAYMELS |
| | | | RLRSDDTAVYYCTRSHYGLDWNFDVWGKGTTVTVSSSQVQL |
| | | | VESGGGVVQPGRSLRLSCAASGFTFTKAWMHWVRQAPGKQL |
| | | | EWVAQIKDKSNSYATYYADSVKGRFTISRDDSKNTLYLQMNS |
| | | | LRAEDTAVYYCRGVYYALSPFDYWGQGTLVTVSSRTASTKGP |
| | | | SVFPLAPCSRSTSESTAALGCLVKDYFPEPVTVSWNSGALTSG |
| | | | VHTFPAVLQSSGLYSLSSVVTVPSSSLGTKTYTCNVDHKPSNT |
| | | | KVDKRVESKYGPPCPPCPAPEAAGGPSVFLFPPKPKDTLMISR |
| | | | TPEVTCVVVDVSQEDPEVQFNWYVDGVEVHNAKTKPREEQF |
| | | | NSTYRVVSVLTVLHQDWLNGKEYKCKVSNKGLPSSIEKTISK |
| | | | AKGQPREPQVCTLPPSQEEMTKNQVSLSCAVKGFYPSDIAVE |
| | | | WESNGQPENNYKTTPPVLDSDGSFFLVSKLTVDKSRWQEGNV |
| | | | FSCSVMHEALHNHYTQKSLSLSLG |
| | 3 | 150 | EVQLVESGGGLVQPGGSLRLSCAASGFNIKDTYIHWVRQAPG |
| | | | KGLEWVARIYPTNGYTRYADSVKGRFTISADTSKNTAYLQMN |
| | | | SLRAEDTAVYYCSRWGGDGFYAMDYWGQGTLVTVSSASTK |
| | | | GPSVFPLAPCSRSTSESTAALGCLVKDYFPEPVTVSWNSGALT |
| | | | SGVHTFPAVLQSSGLYSLSSVVTVPSSSLGTKTYTCNVDHKPS |
| | | | NTKVDKRVESKYGPPCPPCPAPEAAGGPSVFLFPPKPKDTLMI |
| | | | SRTPEVTCVVVDVSQEDPEVQFNWYVDGVEVHNAKTKPREE |
| | | | QFNSTYRVVSVLTVLHQDWLNGKEYKCKVSNKGLPSSIEKTIS |
| | | | KAKGQPREPQVYTLPPCQEEMTKNQVSLWCLVKGFYPSDIAV |
| | | | EWESNGQPENNYKTTPPVLDSDGSFFLYSKLTVDKSRWQEGN |
| | | | VFSCSVMHEALHNHYTQKSLSLSLG |
| | 4 | 151 | DIQMTQSPSSLSASVGDRVTITCRASQDVNTAVAWYQQKPGK |
| | | | APKLLIYSASFLYSGVPSRFSGSRSGTDFTLTISSLQPEDFATYY |
| | | | CQQHYTTPPTFGQGTKVEIKRTVAAPSVFIFPPSDEQLKSGTAS |
| | | | VVCLLNNFYPREAKVQWKVDNALQSGNSQESVTEQDSKDST |
| | | | YSLSSTLTLSKADYEKHKVYACEVTHQGLSSPVTKSFNRGEC |
| HER230R/55Q/1 | 1 | 152 | DIVMTQTPLSLSVTPGQPASISCKSSQSLVHENLRTYLSWYLQ |
| 02E/ | | | KPGQSPQSLIYKVSNRFSGVPDRFSGSGSGTDFTLKISRVEAED |
| CD28ₛᵤₚxCD3_{mid} | | | VGVYYCGQGTQYPFTFGSGTKVEIKDKTHTDIQMTQSPSSLSA |
| (32/33/3435 | | | SVGDRVTITCQASQNIYVWLNWYQQKPGKAPKLLIYKASNLH |
| ENLR (LC); | | | TGVPSRFSGSGSGTDFTLTISSLQPEDIATYYCQQGQTYPYTFG |
| DKTHT linkers | | | QGTKLEIKDKTHTRTVAAPSVFIFPPSDEQLKSGTASVVCLLNN |
| on HC/LC) IgG4 | | | FYPREAKVQWKVDNALQSGNSQESVTEQDSKDSTYSLSSTLT |
| FALA | | | LSKADYEKHKVYACEVTHQGLSSPVTKSFNRGEC |
| BP # 31 | | | |
| | 2 | 153 | QVQLVQSGAEVVKPGASVKVSCKASGYTFTSYYIHWVRQAP |
| | | | GQGLEWIGSIYPGNVNTNYAQKFQGRATLTVDTSISTAYMELS |
| | | | RLRSDDTAVYYCTRSHYGLDWNFDVWGKGTTVTVSSDKTHT |
| | | | QVQLVESGGGVVQPGRSLRLSCAASGFTFTKAWMHWVRQAP |
| | | | GKQLEWVAQIKDKSNSYATYYADSVKGRFTISRDDSKNTLYL |
| | | | QMNSLRAEDTAVYYCRGVYYALSPFDYWGQGTLVTVSSDKT |
| | | | HTASTKGPSVFPLAPCSRSTSESTAALGCLVKDYFPEPVTVSW |
| | | | NSGALTSGVHTFPAVLQSSGLYSLSSVVTVPSSSLGTKTYTCN |
| | | | VDHKPSNTKVDKRVESKYGPPCPPCPAPEAAGGPSVFLFPPKP |
| | | | KDTLMISRTPEVTCVVVDVSQEDPEVQFNWYVDGVEVHNAK |
| | | | TKPREEQFNSTYRVVSVLTVLHQDWLNGKEYKCKVSNKGLPS |
| | | | SIEKTISKAKGQPREPQVCTLPPSQEEMTKNQVSLSCAVKGFY |
| | | | PSDIAVEWESNGQPENNYKTTPPVLDSDGSFFLVSKLTVDKSR |
| | | | WQEGNVFSCSVMHEALHNHYTQKSLSLSLG |
| | 3 | 154 | EVQLVESGGGLVQPGGSLRLSCAASGFNIRDTYIHWVRQAPG |
| | | | KGLEWVARIYPTQGYTRYADSVKGRFTISADTSKNTAYLQMN |
| | | | SLRAEDTAVYYCSRWGGEGFYAMDYWGQGTLVTVSSASTKG |
| | | | PSVFPLAPCSRSTSESTAALGCLVKDYFPEPVTVSWNSGALTS |
| | | | GVHTFPAVLQSSGLYSLSSVVTVPSSSLGTKTYTCNVDHKPSN |
| | | | TKVDKRVESKYGPPCPPCPAPEAAGGPSVFLFPPKPKDTLMIS |
| | | | RTPEVTCVVVDVSQEDPEVQFNWYVDGVEVHNAKTKPREEQ |
| | | | FNSTYRVVSVLTVLHQDWLNGKEYKCKVSNKGLPSSIEKTISK |
| | | | AKGQPREPQVYTLPPCQEEMTKNQVSLWCLVKGFYPSDIAVE |
| | | | WESNGQPENNYKTTPPVLDSDGSFFLYSKLTVDKSRWQEGNV |
| | | | FSCSVMHEALHNHYTQKSLSLSLG |
| | 4 | 155 | DIQMTQSPSSLSASVGDRVTITCRASQDVNTAVAWYQQKPGK |
| | | | APKLLIYSASFLYSGVPSRFSGSRSGTDFTLTISSLQPEDFATYY |
| | | | CQQHYTTPPTFGQGTKVEIKRTVAAPSVFIFPPSDEQLKSGTAS |
| | | | VVCLLNNFYPREAKVQWKVDNALQSGNSQESVTEQDSKDST |
| | | | YSLSSTLTLSKADYEKHKVYACEVTHQGLSSPVTKSFNRGEC |

### III. Methods of Treating Cancer Using Trispecific Binding Proteins

The present disclosure relates to methods of treating a subject having a cancer comprising administering to the subject about 0.2 μg to about 4500 μg of a trispecific binding protein, wherein the trispecific binding protein comprises a first antigen binding site that binds a CD28 polypeptide, a second antigen binding site that binds a CD3 polypeptide, and a third antigen binding site that binds a HER2 polypeptide. In some embodiments, the present disclosure relates to a trispecific binding protein provided herein for use in treating a subject having a cancer, wherein the trispecific binding protein is administered to the subject at a dose of about 0.2 μg to about 4500 μg, and wherein the trispecific binding protein comprises a first antigen binding site that binds a CD28 polypeptide, a second antigen binding site that binds a CD3 polypeptide, and a third antigen binding site that binds a HER2 polypeptide. In some embodiments, the present disclosure relates to use of a trispecific binding protein provided herein for treating a subject having a cancer, wherein the trispecific binding protein is administered to the subject at a dose of about 0.2 μg to about 4500 μg, and wherein the trispecific binding protein comprises a first antigen binding site that binds a CD28 polypeptide, a second antigen binding site that binds a CD3 polypeptide, and a third antigen binding site that binds a HER2 polypeptide. In some embodiments, the present disclosure relates to use of a trispecific binding protein provided herein for the manufacture of a medicament for treating a subject having a cancer, wherein the trispecific binding protein is administered to the subject at a dose of about 0.2 μg to about 4500 μg, and wherein the trispecific binding protein comprises a first antigen binding site that binds a CD28 polypeptide, a second antigen binding site that binds a CD3 polypeptide, and a third antigen binding site that binds a HER2 polypeptide. In some embodiments, the patient is a human.

In some embodiments, the binding protein is administered (or is to be administered) in combination with one or more anti-cancer therapies (e.g., any anti-cancer therapy known in the art, such as a chemotherapeutic agent). In some embodiments, the binding protein is administered (or is to be administered) before the one or more anti-cancer therapies. In some embodiments, the binding protein is administered (or is to be administered) concurrently with the one or more anti-cancer therapies. In some embodiments, the binding protein is administered (or is to be administered) after the one or more anti-cancer therapies.

In some embodiments, the binding protein comprises two antigen binding sites that binds a T-cell surface protein and another antigen binding site that binds the extracellular domain of a human HER2 polypeptide. In some embodiments, the binding protein comprises an antigen binding site that binds the extracellular domain of a human HER2 polypeptide, an antigen binding site that binds a human CD28 polypeptide, and an antigen binding site that binds a human CD3 polypeptide.

In some embodiments, the cancer is a HER2-positive cancer. In some embodiments, the subject is selected for treatment on the basis that cells of the cancer express a human HER2 polypeptide. In some embodiments, HER2 is detected and/or assessed using any suitable method known in the art. For example, HER2 may be detected and/or assessed using an immunohistochemistry (IHC) assay and/or a fluorescence in situ hybridization (FISH) assay. In some embodiments, a HER2-positive breast cancer is defined according to the HER2 Testing in Breast Cancer Guideline: 2018 Focused Update (Wolff et al. J Clin Oncol 2018, 36(20):2105-2122, the content of which is herein incorporated by reference in its entirety).

In some embodiments, the cancer is assessed using a HER2 immunohistochemistry (IHC) score of 0 to 3+. In some embodiments, a HER2 IHC score of the cancer is assessed according to the American Society of Clinical Oncology (ASCO) and College of American Pathologists (CAP) guidelines (see J Clin Oncol 2018, 36(20):2105-2122, the content of which is herein incorporated by reference in its entirety). In some embodiments, a HER2 IHC score of 0 to 1+ is classified as HER2 negative. In some embodiments, a HER2 IHC score of 2+ is classified as uncertain (e.g., further testing is needed). In some embodiments, a HER2 IHC score of 3+ is classified as HER2 positive. In some embodiments, the cancer has a HER2 IHC score of 1+, 2+, or 3+.

In some embodiments, the cancer exhibits HER2 gene amplification. In some embodiments, the cancer does not exhibit HER2 amplification.

In some embodiments, the cancer has at least one HER2 activating mutation. In some embodiments, the cancer has at least one HER2 activating mutation and does not exhibit HER2 amplification. In some embodiments, a HER2 activating mutation is a mutation (e.g., point mutations, deletions, insertions, inversions, etc.) that leads to an increase in at least one biological activity of HER2, such as cell proliferation. In some embodiments, the presence or absence of a HER2 activating mutation is detected and/or assessed using any suitable method known in the art. In some embodiments, the at least one HER2 activating mutation is in the tyrosine kinase domain or the extracellular dimerization domain of HER2. For example, the presence or absence of a HER2 activating mutation detected and/or assessed by collecting tumor tissue from a subject with cancer and subjecting a formalin-fixed paraffin-embedded specimen (FFPE) to real-time quantitative PCR (qRT-PCR), or microarray analysis. In some embodiments, the presence or absence of a HER2 activating mutation is detected and/or assessed by isolating DNA from a tumor sample from a subject with cancer and subjecting the DNA to PCR-based next generation sequencing (NGS). In some embodiments, the presence or absence of a HER2 activating mutation is detected and/or assessed by collecting cell-free circulating tumor DNA (ctDNA) from a subject with cancer and subjecting it to next generation sequencing (NGS) (see, e.g., J Clin Oncol 2013; 31:1997-2003, Clin Cancer Res 2012; 18:4910-8, J Thorac Oncol 2012; 7:85-9, Lung Cancer 2011; 74:139-44, Cancer Res 2005; 65:1642-6, Cancer Sci 2006; 97:753-9, ESMO Open 2017; 2:e000279, and Annals of Oncology 26:1421-1427, 2015).

In some embodiments, the cancer comprises a solid tumor. In some embodiments, the cancer is advanced. In some embodiments, the cancer is metastatic. In some embodiments, the cancer is a relapsed and/or refractory cancer. In some embodiments, the cancer is breast cancer, gastric cancer, gastroesophageal cancer, ovarian cancer, bladder cancer, colon cancer, osteosarcoma, or lung cancer. In some embodiments, the cancer is non-small cell lung cancer (NSCLC).

In some embodiments, the methods of treatment described herein generally reduce or prevent the expansion or burden of the cancer in the subject. For example, in some embodiments, the methods generally reduce tumor size, bulk, metastasis, and/or improve prognosis or survival or other symptom associated with the cancer. In some embodiments, the methods of treatment described herein reduce the tumor size in the subject. In some embodiments, the methods of treatment described herein reduce the number and/or size of metastatic lesions in the subject.

In some embodiments, the subject does not exhibit cytokine release syndrome (CRS), neurotoxicity, neutropenia, thrombocytopenia, hematologic toxicity, myocardial toxicity, pulmonary toxicity, hepatoxicity, tumor lysis syndrome, or any combination thereof following administration of the binding protein. In some embodiments, the subject does not exhibit transaminase elevation after administration of the binding protein. In some embodiments, the subject does not exhibit alanine aminotransferase elevation after administration of the binding protein. In some embodiments, the subject does not exhibit pyrexia after administration of the binding protein. In some embodiments, the subject does not exhibit immunogenicity after administration of the binding protein.

In some embodiments, the method further comprises measuring and/or monitoring one or more inflammatory cytokines and/or chemokines before, during, or after administration of the binding protein. In some embodiments, the one or more cytokines or chemokines include IFN-7, TNF-α, IL-2, IL-1β, IL-6, IL-7, IL-8, IL-10, IL-12, sIL-2Rα, granulocyte macrophage colony stimulating factor (GM-CSF), or macrophage inflammatory protein (MIP). In some embodiments, IFN-γ and IL-2 are measured and/or monitored. In some embodiments, measuring and/or monitoring one or more inflammatory cytokines and/or chemokines before, during, or after administration of the binding protein is performed using any suitable method known in the art, e.g., enzyme linked immunosorbent assay (ELISA) or polymerase chain reaction (PCR). In some embodiments, administration of the trispecific binding protein to the subject results in an increase in the level of at least one cytokine in the peripheral blood of the subject compared to the level of the at least one cytokine in the peripheral blood of the subject prior to the administration of the trispecific binding protein. In some embodiments, the at least one cytokine is IL2, IFNγ, IL6, IL10, TNF-α, or a combination thereof.

In some embodiments, the method further comprises measuring and/or monitoring at least one T cell activation marker before, during, or after administration of the trispecific binding protein. In some embodiments, measuring and/or monitoring the at least one T cell activation marker before, during, or after administration of the trispecific binding protein is performed using any suitable method known in the art, for example flow cytometry or mass cytometry. In some embodiments, administration of the trispecific binding protein to the subject results in an increase in the level of at least one T cell activation marker in the peripheral blood of the subject as compared to the level of the at least one T cell activation marker in the peripheral blood of the subject prior to the administration of the trispecific binding protein. In some embodiments, the at least one T cell activation marker is cluster of differentiation 57 (CD57), programmed cell death protein 1 (PD1), human leukocyte antigen-DR isotype (HLA-DR), or any combination thereof.

In some embodiments, the method further comprises measuring and/or monitoring at least one immune marker before, during, or after administration of the trispecific binding protein. In some embodiments, measuring and/or monitoring the at least one immune marker before, during, or after administration of the trispecific binding protein is performed using any suitable method known in the art, for example immunohistochemistry. In some embodiments, administration of the trispecific binding protein to the subject results in an increase in the level of at least one immune marker in the tumor tissue of the subject as compared to the level of the at least one immune marker in the tumor tissue of the subject prior to the administration of the trispecific binding protein. In some embodiments, the at least one immune marker is programmed cell death protein 1 (PD1), programmed death ligand 1 (PDL1), Ki67, or any combination thereof.

In some embodiments, the method further comprises measuring and/or monitoring at least one cytotoxicity biomarker before, during, or after administration of the trispecific binding protein. In some embodiments, measuring and/or monitoring the at least one cytotoxicity biomarker before, during, or after administration of the trispecific binding protein is performed using any suitable method known in the art, for example flow cytometry or mass cytometry. In some embodiments, administration of the trispecific binding protein to the subject results in an increase in the level of at least one cytotoxicity biomarker in the peripheral blood of the subject as compared to the level of the at least one cytotoxicity biomarker in the peripheral blood of the subject prior to the administration of the trispecific binding protein. In some embodiments, the cytotoxicity biomarker is granzyme B.

In some embodiments, the method further comprises measuring and/or monitoring at least one proliferation biomarker before, during, or after administration of the trispecific binding protein. In some embodiments, measuring and/or monitoring the at least one proliferation biomarker before, during, or after administration of the trispecific binding protein is performed using any suitable method known in the art, for example flow cytometry or mass cytometry. In some embodiments, administration of the trispecific binding protein to the subject results in an increase in the level of at least one proliferation biomarker in the peripheral blood of the subject compared to the level of the at least one proliferation biomarker in the peripheral blood of the subject prior to the administration of the trispecific binding protein. In some embodiments, the proliferation biomarker is Ki67.

In some embodiments, the method further comprises measuring and/or monitoring the level of regulatory T cells (Tregs) before, during, or after administration of the trispecific binding protein. In some embodiments, measuring and/or monitoring the level of Tregs before, during, or after administration of the trispecific binding protein is performed using any suitable method known in the art, for example flow cytometry or mass cytometry. In some embodiments, administration of the trispecific binding protein to the subject results in a decrease in the level of Tregs in the peripheral blood of the subject compared to the level of Tregs in the peripheral blood of the subject prior to the administration of the trispecific binding protein.

In some embodiments, the method further comprises measuring and/or monitoring the level of lymphocytes before, during, or after administration of the trispecific binding protein. In some embodiments, measuring and/or monitoring the level of lymphocytes before, during, or after administration of the trispecific binding protein is performed using any suitable method known in the art, for example flow cytometry or mass cytometry. In some embodiments, administration of the trispecific binding protein to the subject results in a decrease in the level of lymphocytes in the peripheral blood of the subject compared to the level of lymphocytes in the peripheral blood of the subject prior to the administration of the trispecific binding protein.

In some embodiments, the method further comprises measuring and/or monitoring the level of NK cells before, during, or after administration of the trispecific binding protein. In some embodiments, measuring and/or monitoring the level of NK cells before, during, or after administration of the trispecific binding protein is performed using any suitable method known in the art, for example flow cytometry or mass cytometry. In some embodiments, administration of the trispecific binding protein to the subject results in an increase in the level of NK cells in the peripheral blood of the subject compared to the level of NK cells in the peripheral blood of the subject prior to the administration of the trispecific binding protein.

In some embodiments, the method further comprises measuring and/or monitoring the level of T cells before, during, or after administration of the trispecific binding protein. In some embodiments, measuring and/or monitoring the level of T cells before, during, or after administration of the trispecific binding protein is performed using any suitable method known in the art, for example flow cytometry or mass cytometry. In some embodiments, administration of the trispecific binding protein to the subject results in an increase in the level of T cells in the peripheral blood of the subject compared to the level of T cells in the peripheral blood of the subject prior to the administration of the trispecific binding protein.

In some embodiments, the subject has received prior chemotherapy and/or radiation therapy. In some embodiments, the cancer is refractory to a prior therapy. In some embodiments, the cancer relapsed while the subject was being treated with the prior chemotherapy and/or radiation therapy.

Any of the binding proteins described herein may find use in the methods of the present disclosure.

For diagnostic applications, in certain embodiments, binding proteins can be labeled with a detectable moiety. The detectable moiety can be any one that is capable of producing, either directly or indirectly, a detectable signal. For example, the detectable moiety can be a radioisotope, such as ³H, ¹⁴C, ³²P, ³⁵S, ¹²⁵I, ⁹⁹Tc, ¹¹¹In, or ⁶⁷Ga; a fluorescent or chemiluminescent compound, such as fluorescein isothiocyanate, rhodamine, or luciferin; or an enzyme, such as alkaline phosphatase, β-galactosidase, or horseradish peroxidase.

The binding proteins are also useful for in vivo imaging. A binding protein labeled with a detectable moiety can be administered to an animal, preferably into the bloodstream, and the presence and location of the labeled antibody in the host assayed. The binding protein can be labeled with any moiety that is detectable in an animal, whether by nuclear magnetic resonance, radiology, or other detection means known in the art.

For clinical or research applications, in certain embodiments, binding proteins can be conjugated to a cytotoxic agent. A variety of antibodies coupled to cytotoxic agents (i.e., antibody-drug conjugates) have been used to target cytotoxic payloads to specific tumor cells. Cytotoxic agents and linkers that conjugate the agents to an antibody are known in the art; see, e.g., Parslow, A. C. et al. (2016) Biomedicines 4:14 and Kalim, M. et al. (2017) Drug Des. Devel. Ther. 11:2265-2276, both of which are herein incorporated by reference in their entireties.

### A. Dosing of Trispecific Binding Proteins

The present disclosure provides methods of treating a subject having a cancer comprising administering to the subject about 0.2 μg to about 4500 μg of a trispecific binding protein, wherein the trispecific binding protein comprises a first antigen binding site that binds a CD28 polypeptide, a second antigen binding site that binds a CD3 polypeptide, and a third antigen binding site that binds a HER2 polypeptide. In some embodiments, the dose of the binding protein is between 0.2 μg and 4500 μg.

In some embodiments, the binding protein provided is administered to the subject at a dose of about 0.2 μg to about 4500 μg per administration (e.g., injection). In some embodiments, the binding protein provided is administered to the subject at a dose of 0.2 μg to 4500 μg per administration (e.g., injection).

In some embodiments, the binding protein is administered to the subject at a dose of 10 μg to 1500 μg. In some embodiments, the binding protein is administered to the subject at a dose between about 10 μg to about 25 μg, about 20 μg to about 50 μg, about 40 μg to about 70 μg, about 60 μg to about 100 μg, about 90 μg to about 150 μg, about 140 μg to about 200 μg, about 190 μg to about 250 μg, about 240 μg to about 300 μg, about 290 μg to about 350 μg, about 340 μg to about 400 μg, about 390 μg to about 460 μg, about 450 μg to about 500 μg, about 490 μg to about 600 μg, about 590 μg to about 700 μg, about 690 μg to about 750 μg, about 740 μg to about 800 μg, about 790 μg to about 880 μg, about 870 μg to about 950 μg, about 940 μg to about 1000 μg, about 990 μg to about 1100 μg, about 1090 μg to about 1200 μg, about 1190 μg to about 1300 μg, about 1290 μg to about 1400 μg, or about 1390 μg to about 1500 μg. In some embodiments, the binding protein is administered to the subject at a dose between 10 μg to 25 μg, 20 μg to 50 μg, 40 μg to 70 μg, 60 μg to 100 μg, 90 μg to 150 μg, 140 μg to 200 μg, 190 μg to 250 μg, 240 μg to 300 μg, 290 μg to 350 μg, 340 μg to 400 μg, 390 μg to 460 μg, 450 μg to 500 μg, 490 μg to 600 μg, 590 μg to 700 μg, 690 μg to 750 μg, 740 μg to 800 μg, 790 μg to 880 μg, 870 μg to 950 μg, 940 μg to 1000 μg, 990 μg to 1100 μg, 1090 μg to 1200 μg, 1190 μg to 1300 μg, 1290 μg to 1400 μg, or 1390 μg to 1500 μg.

In some embodiments, the binding protein is administered to the subject at a dose of about 10 μg, about 11 μg, about 12 μg, about 13 μg, about 14 μg, about 15 μg, about 16 μg, about 17 μg, about 18 μg, about 19 μg, about 20 μg, about 30 μg, about 40 μg, about 50 μg, about 60 μg, about 70 μg, about 80 μg, about 90 μg, about 100 μg, about 110 μg, about 120 μg, about 130 μg, about 140 μg, about 150 μg, about 160 μg, about 170 μg, about 180 μg, about 190 μg, about 200 μg, about 220 μg, about 240 μg, about 260 μg, about 280 μg, about 300 μg, about 320 μg, about 340 μg, about 360 μg, about 380 μg, about 400 μg, about 420 μg, about 440 μg, about 460 μg, about 480 μg, about 500 μg, about 520 μg, about 540 μg, about 560 μg, about 580 μg, about 600 μg, about 620 μg, about 640 μg, about 660 μg, about 680 μg, about 700 μg, about 720 μg, about 740 μg, about 760 μg, about 780 μg, about 800 μg, about 820 μg, about 840 μg, about 860 μg, about 880 μg, about 900 μg, about 920 μg, about 940 μg, about 960 μg, about 980 μg, about 1000 μg, about 1020 μg, about 1040 μg, about 1060 μg, about 1080 μg, about 1100 μg, about 1120 μg, about 1125 μg, about 1140 μg, about 1160 μg, about 1180 μg, about 1200 μg, about 1220 μg, about 1240 μg, about 1260 μg, about 1280 μg, about 1300 μg, about 1320 μg, about 1340 μg, about 1360 μg, about 1380 μg, about 1400 μg, about 1420 μg, or about 1440 μg, about 1460 μg, about 1480 μg, about 1500 μg.

In some embodiments, the binding protein is administered to the subject at a dose of 10 μg, 11 μg, 12 μg, 13 μg, 14 μg, 15 μg, 16 μg, 17 μg, 18 μg, 19 μg, 20 μg, 30 μg, 40 μg, 50 μg, 60 μg, 70 μg, 80 μg, 90 μg, 100 μg, 110 μg, 120 μg, 130 μg, 140 μg, 150 μg, 160 μg, 170 μg, 180 μg, 190 μg, 200 μg, 220 μg, 240 μg, 260 μg, 280 μg, 300 μg, 320 μg, 340 μg, 360 μg, 380 μg, 400 μg, 420 μg, 440 μg, 460 μg, 480 μg, 500 μg, 520 μg, 540 μg, 560 μg, 580 μg, 600 μg, 620 μg, 640 μg, 660 μg, 680 μg, 700 μg, 720 μg, 740 μg, 760 μg, 780 μg, 800 μg, 820 μg, 840 μg, 860 μg, 880 μg, 900 μg, 920 μg, 940 μg, 960 μg, 980 μg, 1000 μg, 1020 μg, 1040 μg, 1060 μg, 1080 μg, 1100 μg, 1120 μg, 1125 μg, 1140 μg, 1160 μg, 1180 μg, 1200 μg, 1220 μg, 1240 μg, 1260 μg, 1280 μg, 1300 μg, 1320 μg, 1340 μg, 1360 μg, 1380 μg, 1400 μg, 1420 μg, or 1440 μg, 1460 μg, 1480 μg, 1500 μg.

In some embodiments, the binding protein is administered to the subject at a dose of 18 μg, 60 μg, 180 μg, 240 μg, 360 μg, 480 μg, 720 μg, 900 μg, 1080 μg, 1125 μg, or 1440 μg.

In some embodiments, the binding protein is administered to the subject intermittently, e.g., at a frequency of about three times per week, twice per week, once per week, once every two weeks, once every three weeks, once every four weeks, once every five weeks, once every six weeks, once every seven weeks, or once every eight weeks. In some embodiments, the binding protein is administered to the subject intermittently, e.g., at a frequency of three times per week, twice per week, once per week, once every two weeks, once every three weeks, once every four weeks, once every five weeks, once every six weeks, once every seven weeks, or once every eight weeks.

In some embodiments, the binding protein is administered to the subject at a dose of 10 μg to 1500 μg at a frequency of three times per week. In some embodiments, the binding protein is administered to the subject at a dose between about 10 μg to about 25 μg, about 20 μg to about 50 μg, about 40 μg to about 70 μg, about 60 μg to about 100 μg, about 90 μg to about 150 μg, about 140 μg to about 200 μg, about 190 μg to about 250 μg, about 240 μg to about 300 μg, about 290 μg to about 350 μg, about 340 μg to about 400 μg, about 390 μg to about 460 μg, about 450 μg to about 500 μg, about 490 μg to about 600 μg, about 590 μg to about 700 μg, about 690 μg to about 750 μg, about 740 μg to about 800 μg, about 790 μg to about 880 μg, about 870 μg to about 950 μg, about 940 μg to about 1000 μg, about 990 μg to about 1100 μg, about 1090 μg to about 1200 μg, about 1190 μg to about 1300 μg, about 1290 μg to about 1400 μg, or about 1390 μg to about 1500 μg at a frequency of three times per week. In some embodiments, the binding protein is administered to the subject at a dose between 10 μg to 25 μg, 20 μg to 50 μg, 40 μg to 70 μg, 60 μg to 100 μg, 90 μg to 150 μg, 140 μg to 200 μg, 190 μg to 250 μg, 240 μg to 300 μg, 290 μg to 350 μg, 340 μg to 400 μg, 390 μg to 460 μg, 450 μg to 500 μg, 490 μg to 600 μg, 590 μg to 700 μg, 690 μg to 750 μg, 740 μg to 800 μg, 790 μg to 880 μg, 870 μg to 950 μg, 940 μg to 1000 μg, 990 μg to 1100 μg, 1090 μg to 1200 μg, 1190 μg to 1300 μg, 1290 μg to 1400 μg, or 1390 μg to 1500 μg at a frequency of three times per week.

In some embodiments, the binding protein is administered to the subject at a dose of about 10 μg, about 11 μg, about 12 μg, about 13 μg, about 14 μg, about 15 μg, about 16 μg, about 17 μg, about 18 μg, about 19 μg, about 20 μg, about 30 μg, about 40 μg, about 50 μg, about 60 μg, about 70 μg, about 80 μg, about 90 μg, about 100 μg, about 110 μg, about 120 μg, about 130 μg, about 140 μg, about 150 μg, about 160 μg, about 170 μg, about 180 μg, about 190 μg, about 200 μg, about 220 μg, about 240 μg, about 260 μg, about 280 μg, about 300 μg, about 320 μg, about 340 μg, about 360 μg, about 380 μg, about 400 μg, about 420 μg, about 440 μg, about 460 μg, about 480 μg, about 500 μg, about 520 μg, about 540 μg, about 560 μg, about 580 μg, about 600 μg, about 620 μg, about 640 μg, about 660 μg, about 680 μg, about 700 μg, about 720 μg, about 740 μg, about 760 μg, about 780 μg, about 800 μg, about 820 μg, about 840 μg, about 860 μg, about 880 μg, about 900 μg, about 920 μg, about 940 μg, about 960 μg, about 980 μg, about 1000 μg, about 1020 μg, about 1040 μg, about 1060 μg, about 1080 μg, about 1100 μg, about 1120 μg, about 1125 μg, about 1140 μg, about 1160 μg, about 1180 μg, about 1200 μg, about 1220 μg, about 1240 μg, about 1260 μg, about 1280 μg, about 1300 μg, about 1320 μg, about 1340 μg, about 1360 μg, about 1380 μg, about 1400 μg, about 1420 μg, or about 1440 μg, about 1460 μg, about 1480 μg, about 1500 μg at a frequency of three times per week.

In some embodiments, the binding protein is administered to the subject at a dose of 10 μg, 11 μg, 12 μg, 13 μg, 14 μg, 15 μg, 16 μg, 17 μg, 18 μg, 19 μg, 20 μg, 30 μg, 40 μg, 50 μg, 60 μg, 70 μg, 80 μg, 90 μg, 100 μg, 110 μg, 120 μg, 130 μg, 140 μg, 150 μg, 160 μg, 170 μg, 180 μg, 190 μg, 200 μg, 220 μg, 240 μg, 260 μg, 280 μg, 300 μg, 320 μg, 340 μg, 360 μg, 380 μg, 400 μg, 420 μg, 440 μg, 460 μg, 480 μg, 500 μg, 520 μg, 540 μg, 560 μg, 580 μg, 600 μg, 620 μg, 640 μg, 660 μg, 680 μg, 700 μg, 720 μg, 740 μg, 760 μg, 780 μg, 800 μg, 820 μg, 840 μg, 860 μg, 880 μg, 900 μg, 920 μg, 940 μg, 960 μg, 980 μg, 1000 μg, 1020 μg, 1040 μg, 1060 μg, 1080 μg, 1100 μg, 1120 μg, 1125 μg, 1140 μg, 1160 μg, 1180 μg, 1200 μg, 1220 μg, 1240 μg, 1260 μg, 1280 μg, 1300 μg, 1320 μg, 1340 μg, 1360 μg, 1380 μg, 1400 μg, 1420 μg, or 1440 μg, 1460 μg, 1480 μg, 1500 μg at a frequency of three times per week.

In some embodiments, the binding protein is administered to the subject at a dose of 18 μg, 60 μg, 180 μg, 240 μg, 360 μg, 480 μg, 720 μg, 900 μg, 1080 μg, 1125 μg, or 1440 μg at a frequency of three times per week.

In some embodiments, the binding protein is administered to the subject at a dose of 10 μg to 1500 μg at a frequency of twice per week. In some embodiments, the binding protein is administered to the subject at a dose between about 10 μg to about 25 μg, about 20 μg to about 50 μg, about 40 μg to about 70 μg, about 60 μg to about 100 μg, about 90 μg to about 150 μg, about 140 μg to about 200 μg, about 190 μg to about 250 μg, about 240 μg to about 300 μg, about 290 μg to about 350 μg, about 340 μg to about 400 μg, about 390 μg to about 460 μg, about 450 μg to about 500 μg, about 490 μg to about 600 μg, about 590 μg to about 700 μg, about 690 μg to about 750 μg, about 740 μg to about 800 μg, about 790 μg to about 880 μg, about 870 μg to about 950 μg, about 940 μg to about 1000 μg, about 990 μg to about 1100 μg, about 1090 μg to about 1200 μg, about 1190 μg to about 1300 μg, about 1290 μg to about 1400 μg, or about 1390 μg to about 1500 μg at a frequency of twice per week. In some embodiments, the binding protein is administered to the subject at a dose between 10 μg to 25 μg, 20 μg to 50 μg, 40 μg to 70 μg, 60 μg to 100 μg, 90 μg to 150 μg, 140 μg to 200 μg, 190 μg to 250 μg, 240 μg to 300 μg, 290 μg to 350 μg, 340 μg to 400 μg, 390 μg to 460 μg, 450 μg to 500 μg, 490 μg to 600 μg, 590 μg to 700 μg, 690 μg to 750 μg, 740 μg to 800 μg, 790 μg to 880 μg, 870 μg to 950 μg, 940 μg to 1000 μg, 990 μg to 1100 μg, 1090 μg to 1200 μg, 1190 μg to 1300 μg, 1290 μg to 1400 μg, or 1390 μg to 1500 μg at a frequency of twice per week.

In some embodiments, the binding protein is administered to the subject at a dose of about 10 μg, about 11 μg, about 12 μg, about 13 μg, about 14 μg, about 15 μg, about 16 μg, about 17 μg, about 18 μg, about 19 μg, about 20 μg, about 30 μg, about 40 μg, about 50 μg, about 60 μg, about 70 μg, about 80 μg, about 90 μg, about 100 μg, about 110 μg, about 120 μg, about 130 μg, about 140 μg, about 150 μg, about 160 μg, about 170 μg, about 180 μg, about 190 μg, about 200 μg, about 220 μg, about 240 μg, about 260 μg, about 280 μg, about 300 μg, about 320 μg, about 340 μg, about 360 μg, about 380 μg, about 400 μg, about 420 μg, about 440 μg, about 460 μg, about 480 μg, about 500 μg, about 520 μg, about 540 μg, about 560 μg, about 580 μg, about 600 μg, about 620 μg, about 640 μg, about 660 μg, about 680 μg, about 700 μg, about 720 μg, about 740 μg, about 760 μg, about 780 μg, about 800 μg, about 820 μg, about 840 μg, about 860 μg, about 880 μg, about 900 μg, about 920 μg, about 940 μg, about 960 μg, about 980 μg, about 1000 μg, about 1020 μg, about 1040 μg, about 1060 μg, about 1080 μg, about 1100 μg, about 1120 μg, about 1125 μg, about 1140 μg, about 1160 μg, about 1180 μg, about 1200 μg, about 1220 μg, about 1240 μg, about 1260 μg, about 1280 μg, about 1300 μg, about 1320 μg, about 1340 μg, about 1360 μg, about 1380 μg, about 1400 μg, about 1420 μg, or about 1440 μg, about 1460 μg, about 1480 μg, about 1500 μg at a frequency of twice per week.

In some embodiments, the binding protein is administered to the subject at a dose of 10 μg, 11 μg, 12 μg, 13 μg, 14 μg, 15 μg, 16 μg, 17 μg, 18 μg, 19 μg, 20 μg, 30 μg, 40 μg, 50 μg, 60 μg, 70 μg, 80 μg, 90 μg, 100 μg, 110 μg, 120 μg, 130 μg, 140 μg, 150 μg, 160 μg, 170 μg, 180 μg, 190 μg, 200 μg, 220 μg, 240 μg, 260 μg, 280 μg, 300 μg, 320 μg, 340 μg, 360 μg, 380 μg, 400 μg, 420 μg, 440 μg, 460 μg, 480 μg, 500 μg, 520 μg, 540 μg, 560 μg, 580 μg, 600 μg, 620 μg, 640 μg, 660 μg, 680 μg, 700 μg, 720 μg, 740 μg, 760 μg, 780 μg, 800 μg, 820 μg, 840 μg, 860 μg, 880 μg, 900 μg, 920 μg, 940 μg, 960 μg, 980 μg, 1000 μg, 1020 μg, 1040 μg, 1060 μg, 1080 μg, 1100 μg, 1120 μg, 1125 μg, 1140 μg, 1160 μg, 1180 μg, 1200 μg, 1220 μg, 1240 μg, 1260 μg, 1280 μg, 1300 μg, 1320 μg, 1340 μg, 1360 μg, 1380 μg, 1400 μg, 1420 μg, or 1440 μg, 1460 μg, 1480 μg, 1500 μg at a frequency of twice per week.

In some embodiments, the binding protein is administered to the subject at a dose of 18 μg, 60 μg, 180 μg, 240 μg, 360 μg, 480 μg, 720 μg, 900 μg, 1080 μg, 1125 μg, or 1440 μg at a frequency of twice per week.

In some embodiments, the binding protein is administered to the subject at a dose of 10 μg to 1500 μg at a frequency of once per week. In some embodiments, the binding protein is administered to the subject at a dose between about 10 μg to about 25 μg, about 20 μg to about 50 μg, about 40 μg to about 70 μg, about 60 μg to about 100 μg, about 90 μg to about 150 μg, about 140 μg to about 200 μg, about 190 μg to about 250 μg, about 240 μg to about 300 μg, about 290 μg to about 350 μg, about 340 μg to about 400 μg, about 390 μg to about 460 μg, about 450 μg to about 500 μg, about 490 μg to about 600 μg, about 590 μg to about 700 μg, about 690 μg to about 750 μg, about 740 μg to about 800 μg, about 790 μg to about 880 μg, about 870 μg to about 950 μg, about 940 μg to about 1000 μg, about 990 μg to about 1100 μg, about 1090 μg to about 1200 μg, about 1190 μg to about 1300 μg, about 1290 μg to about 1400 μg, or about 1390 μg to about 1500 μg at a frequency of once per week. In some embodiments, the binding protein is administered to the subject at a dose between 10 μg to 25 μg, 20 μg to 50 μg, 40 μg to 70 μg, 60 μg to 100 μg, 90 μg to 150 μg, 140 μg to 200 μg, 190 μg to 250 μg, 240 μg to 300 μg, 290 μg to 350 μg, 340 μg to 400 μg, 390 μg to 460 μg, 450 μg to 500 μg, 490 μg to 600 μg, 590 μg to 700 μg, 690 μg to 750 μg, 740 μg to 800 μg, 790 μg to 880 μg, 870 μg to 950 μg, 940 μg to 1000 μg, 990 μg to 1100 μg, 1090 μg to 1200 μg, 1190 μg to 1300 μg, 1290 μg to 1400 μg, or 1390 μg to 1500 μg at a frequency of once per week.

In some embodiments, the binding protein is administered to the subject at a dose of about 10 μg, about 11 μg, about 12 μg, about 13 μg, about 14 μg, about 15 μg, about 16 μg, about 17 μg, about 18 μg, about 19 μg, about 20 μg, about 30 μg, about 40 μg, about 50 μg, about 60 μg, about 70 μg, about 80 μg, about 90 μg, about 100 μg, about 110 μg, about 120 μg, about 130 μg, about 140 μg, about 150 μg, about 160 μg, about 170 μg, about 180 μg, about 190 μg, about 200 μg, about 220 μg, about 240 μg, about 260 μg, about 280 μg, about 300 μg, about 320 μg, about 340 μg, about 360 μg, about 380 μg, about 400 μg, about 420 μg, about 440 μg, about 460 μg, about 480 μg, about 500 μg, about 520 μg, about 540 μg, about 560 μg, about 580 μg, about 600 μg, about 620 μg, about 640 μg, about 660 μg, about 680 μg, about 700 μg, about 720 μg, about 740 μg, about 760 μg, about 780 μg, about 800 μg, about 820 μg, about 840 μg, about 860 μg, about 880 μg, about 900 μg, about 920 μg, about 940 μg, about 960 μg, about 980 μg, about 1000 μg, about 1020 μg, about 1040 μg, about 1060 μg, about 1080 μg, about 1100 μg, about 1120 μg, about 1125 μg, about 1140 μg, about 1160 μg, about 1180 μg, about 1200 μg, about 1220 μg, about 1240 μg, about 1260 μg, about 1280 μg, about 1300 μg, about 1320 μg, about 1340 μg, about 1360 μg, about 1380 μg, about 1400 μg, about 1420 μg, or about 1440 μg, about 1460 μg, about 1480 μg, about 1500 μg at a frequency of once per week.

In some embodiments, the binding protein is administered to the subject at a dose of 10 μg, 11 μg, 12 μg, 13 μg, 14 μg, 15 μg, 16 μg, 17 μg, 18 μg, 19 μg, 20 μg, 30 μg, 40 μg, 50 μg, 60 μg, 70 μg, 80 μg, 90 μg, 100 μg, 110 μg, 120 μg, 130 μg, 140 μg, 150 μg, 160 μg, 170 μg, 180 μg, 190 μg, 200 μg, 220 μg, 240 μg, 260 μg, 280 μg, 300 μg, 320 μg, 340 μg, 360 μg, 380 μg, 400 μg, 420 μg, 440 μg, 460 μg, 480 μg, 500 μg, 520 μg, 540 μg, 560 μg, 580 μg, 600 μg, 620 μg, 640 μg, 660 μg, 680 μg, 700 μg, 720 μg, 740 μg, 760 μg, 780 μg, 800 μg, 820 μg, 840 μg, 860 μg, 880 μg, 900 μg, 920 μg, 940 μg, 960 μg, 980 μg, 1000 μg, 1020 μg, 1040 μg, 1060 μg, 1080 μg, 1100 μg, 1120 μg, 1125 μg, 1140 μg, 1160 μg, 1180 μg, 1200 μg, 1220 μg, 1240 μg, 1260 μg, 1280 μg, 1300 μg, 1320 μg, 1340 μg, 1360 μg, 1380 μg, 1400 μg, 1420 μg, or 1440 μg, 1460 μg, 1480 μg, 1500 μg at a frequency of once per week.

In some embodiments, the binding protein is administered to the subject at a dose of 18 μg, 60 μg, 180 μg, 240 μg, 360 μg, 480 μg, 720 μg, 900 μg, 1080 μg, 1125 μg, or 1440 μg at a frequency of once per week.

In some embodiments, the binding protein is administered to the subject at a dose of 10 μg to 1500 μg at a frequency of once every two weeks. In some embodiments, the binding protein is administered to the subject at a dose between about 10 μg to about 25 μg, about 20 μg to about 50 μg, about 40 μg to about 70 μg, about 60 μg to about 100 μg, about 90 μg to about 150 μg, about 140 μg to about 200 μg, about 190 μg to about 250 μg, about 240 μg to about 300 μg, about 290 μg to about 350 μg, about 340 μg to about 400 μg, about 390 μg to about 460 μg, about 450 μg to about 500 μg, about 490 μg to about 600 μg, about 590 μg to about 700 μg, about 690 μg to about 750 μg, about 740 μg to about 800 μg, about 790 μg to about 880 μg, about 870 μg to about 950 μg, about 940 μg to about 1000 μg, about 990 μg to about 1100 μg, about 1090 μg to about 1200 μg, about 1190 μg to about 1300 μg, about 1290 μg to about 1400 μg, or about 1390 μg to about 1500 μg at a frequency of once every two weeks. In some embodiments, the binding protein is administered to the subject at a dose between 10 μg to 25 μg, 20 μg to 50 μg, 40 μg to 70 μg, 60 μg to 100 μg, 90 μg to 150 μg, 140 μg to 200 μg, 190 μg to 250 μg, 240 μg to 300 μg, 290 μg to 350 μg, 340 μg to 400 μg, 390 μg to 460 μg, 450 μg to 500 μg, 490 μg to 600 μg, 590 μg to 700 μg, 690 μg to 750 μg, 740 μg to 800 μg, 790 μg to 880 μg, 870 μg to 950 μg, 940 μg to 1000 μg, 990 μg to 1100 μg, 1090 μg to 1200 μg, 1190 μg to 1300 μg, 1290 μg to 1400 μg, or 1390 μg to 1500 μg at a frequency of once every two weeks.

In some embodiments, the binding protein is administered to the subject at a dose of about 10 μg, about 11 μg, about 12 μg, about 13 μg, about 14 μg, about 15 μg, about 16 μg, about 17 μg, about 18 μg, about 19 μg, about 20 μg, about 30 μg, about 40 μg, about 50 μg, about 60 μg, about 70 μg, about 80 μg, about 90 μg, about 100 μg, about 110 μg, about 120 μg, about 130 μg, about 140 μg, about 150 μg, about 160 μg, about 170 μg, about 180 μg, about 190 μg, about 200 μg, about 220 μg, about 240 μg, about 260 μg, about 280 μg, about 300 μg, about 320 μg, about 340 μg, about 360 μg, about 380 μg, about 400 μg, about 420 μg, about 440 μg, about 460 μg, about 480 μg, about 500 μg, about 520 μg, about 540 μg, about 560 μg, about 580 μg, about 600 μg, about 620 μg, about 640 μg, about 660 μg, about 680 μg, about 700 μg, about 720 μg, about 740 μg, about 760 μg, about 780 μg, about 800 μg, about 820 μg, about 840 μg, about 860 μg, about 880 μg, about 900 μg, about 920 μg, about 940 μg, about 960 μg, about 980 μg, about 1000 μg, about 1020 μg, about 1040 μg, about 1060 μg, about 1080 μg, about 1100 μg, about 1120 μg, about 1125 μg, about 1140 μg, about 1160 μg, about 1180 μg, about 1200 μg, about 1220 μg, about 1240 μg, about 1260 μg, about 1280 μg, about 1300 μg, about 1320 μg, about 1340 μg, about 1360 μg, about 1380 μg, about 1400 μg, about 1420 μg, or about 1440 μg, about 1460 μg, about 1480 μg, about 1500 μg at a frequency of once every two weeks.

In some embodiments, the binding protein is administered to the subject at a dose of 10 μg, 11 μg, 12 μg, 13 μg, 14 μg, 15 μg, 16 μg, 17 μg, 18 μg, 19 μg, 20 μg, 30 μg, 40 μg, 50 μg, 60 μg, 70 μg, 80 μg, 90 μg, 100 μg, 110 μg, 120 μg, 130 μg, 140 μg, 150 μg, 160 μg, 170 μg, 180 μg, 190 μg, 200 μg, 220 μg, 240 μg, 260 μg, 280 μg, 300 μg, 320 μg, 340 μg, 360 μg, 380 μg, 400 μg, 420 μg, 440 μg, 460 μg, 480 μg, 500 μg, 520 μg, 540 μg, 560 μg, 580 μg, 600 μg, 620 μg, 640 μg, 660 μg, 680 μg, 700 μg, 720 μg, 740 μg, 760 μg, 780 μg, 800 μg, 820 μg, 840 μg, 860 μg, 880 μg, 900 μg, 920 μg, 940 μg, 960 μg, 980 μg, 1000 μg, 1020 μg, 1040 μg, 1060 μg, 1080 μg, 1100 μg, 1120 μg, 1125 μg, 1140 μg, 1160 μg, 1180 μg, 1200 μg, 1220 μg, 1240 μg, 1260 μg, 1280 μg, 1300 μg, 1320 μg, 1340 μg, 1360 μg, 1380 μg, 1400 μg, 1420 μg, or 1440 μg, 1460 μg, 1480 μg, 1500 μg at a frequency of once every two weeks.

In some embodiments, the binding protein is administered to the subject at a dose of 18 μg, 60 μg, 180 μg, 240 μg, 360 μg, 480 μg, 720 μg, 900 μg, 1080 μg, 1125 μg, or 1440 μg at a frequency of once every two weeks.

In some embodiments, the binding protein is administered to the subject at a dose of 10 μg to 1500 μg at a frequency of once every three weeks. In some embodiments, the binding protein is administered to the subject at a dose between about 10 μg to about 25 μg, about 20 μg to about 50 μg, about 40 μg to about 70 μg, about 60 μg to about 100 μg, about 90 μg to about 150 μg, about 140 μg to about 200 μg, about 190 μg to about 250 μg, about 240 μg to about 300 μg, about 290 μg to about 350 μg, about 340 μg to about 400 μg, about 390 μg to about 460 μg, about 450 μg to about 500 μg, about 490 μg to about 600 μg, about 590 μg to about 700 μg, about 690 μg to about 750 μg, about 740 μg to about 800 μg, about 790 μg to about 880 μg, about 870 μg to about 950 μg, about 940 μg to about 1000 μg, about 990 μg to about 1100 μg, about 1090 μg to about 1200 μg, about 1190 μg to about 1300 μg, about 1290 μg to about 1400 μg, or about 1390 μg to about 1500 μg at a frequency of once every three weeks. In some embodiments, the binding protein is administered to the subject at a dose between 10 μg to 25 μg, 20 μg to 50 μg, 40 μg to 70 μg, 60 μg to 100 μg, 90 μg to 150 μg, 140 μg to 200 μg, 190 μg to 250 μg, 240 μg to 300 μg, 290 μg to 350 μg, 340 μg to 400 μg, 390 μg to 460 μg, 450 μg to 500 μg, 490 μg to 600 μg, 590 μg to 700 μg, 690 μg to 750 μg, 740 μg to 800 μg, 790 μg to 880 μg, 870 μg to 950 μg, 940 μg to 1000 μg, 990 μg to 1100 μg, 1090 μg to 1200 μg, 1190 μg to 1300 μg, 1290 μg to 1400 μg, or 1390 μg to 1500 μg at a frequency of once every three weeks.

In some embodiments, the binding protein is administered to the subject at a dose of about 10 μg, about 11 μg, about 12 μg, about 13 μg, about 14 μg, about 15 μg, about 16 μg, about 17 μg, about 18 μg, about 19 μg, about 20 μg, about 30 μg, about 40 μg, about 50 μg, about 60 μg, about 70 μg, about 80 μg, about 90 μg, about 100 μg, about 110 μg, about 120 μg, about 130 μg, about 140 μg, about 150 μg, about 160 μg, about 170 μg, about 180 μg, about 190 μg, about 200 μg, about 220 μg, about 240 μg, about 260 μg, about 280 μg, about 300 μg, about 320 μg, about 340 μg, about 360 μg, about 380 μg, about 400 μg, about 420 μg, about 440 μg, about 460 μg, about 480 μg, about 500 μg, about 520 μg, about 540 μg, about 560 μg, about 580 μg, about 600 μg, about 620 μg, about 640 μg, about 660 μg, about 680 μg, about 700 μg, about 720 μg, about 740 μg, about 760 μg, about 780 μg, about 800 μg, about 820 μg, about 840 μg, about 860 μg, about 880 μg, about 900 μg, about 920 μg, about 940 μg, about 960 μg, about 980 μg, about 1000 μg, about 1020 μg, about 1040 μg, about 1060 μg, about 1080 μg, about 1100 μg, about 1120 μg, about 1125 μg, about 1140 μg, about 1160 μg, about 1180 μg, about 1200 μg, about 1220 μg, about 1240 μg, about 1260 μg, about 1280 μg, about 1300 μg, about 1320 μg, about 1340 μg, about 1360 μg, about 1380 μg, about 1400 μg, about 1420 μg, or about 1440 μg, about 1460 μg, about 1480 μg, about 1500 μg at a frequency of once every three weeks.

In some embodiments, the binding protein is administered to the subject at a dose of 10 μg, 11 μg, 12 μg, 13 μg, 14 μg, 15 μg, 16 μg, 17 μg, 18 μg, 19 μg, 20 μg, 30 μg, 40 μg, 50 μg, 60 μg, 70 μg, 80 μg, 90 μg, 100 μg, 110 μg, 120 μg, 130 μg, 140 μg, 150 μg, 160 μg, 170 μg, 180 μg, 190 μg, 200 μg, 220 μg, 240 μg, 260 μg, 280 μg, 300 μg, 320 μg, 340 μg, 360 μg, 380 μg, 400 μg, 420 μg, 440 μg, 460 μg, 480 μg, 500 μg, 520 μg, 540 μg, 560 μg, 580 μg, 600 μg, 620 μg, 640 μg, 660 μg, 680 μg, 700 μg, 720 μg, 740 μg, 760 μg, 780 μg, 800 μg, 820 μg, 840 μg, 860 μg, 880 μg, 900 μg, 920 μg, 940 μg, 960 μg, 980 μg, 1000 μg, 1020 μg, 1040 μg, 1060 μg, 1080 μg, 1100 μg, 1120 μg, 1125 μg, 1140 μg, 1160 μg, 1180 μg, 1200 μg, 1220 μg, 1240 μg, 1260 μg, 1280 μg, 1300 μg, 1320 μg, 1340 μg, 1360 μg, 1380 μg, 1400 μg, 1420 μg, or 1440 μg, 1460 μg, 1480 μg, 1500 μg at a frequency of once every three weeks.

In some embodiments, the binding protein is administered to the subject at a dose of 18 μg, 60 μg, 180 μg, 240 μg, 360 μg, 480 μg, 720 μg, 900 μg, 1080 μg, 1125 μg, or 1440 μg at a frequency of once every three weeks.

In some embodiments, the binding protein is administered to the subject at a dose of 10 μg to 1500 μg at a frequency of once every four weeks. In some embodiments, the binding protein is administered to the subject at a dose between about 10 μg to about 25 μg, about 20 μg to about 50 μg, about 40 μg to about 70 μg, about 60 μg to about 100 μg, about 90 μg to about 150 μg, about 140 μg to about 200 μg, about 190 μg to about 250 μg, about 240 μg to about 300 μg, about 290 μg to about 350 μg, about 340 μg to about 400 μg, about 390 μg to about 460 μg, about 450 μg to about 500 μg, about 490 μg to about 600 μg, about 590 μg to about 700 μg, about 690 μg to about 750 μg, about 740 μg to about 800 μg, about 790 μg to about 880 μg, about 870 μg to about 950 μg, about 940 μg to about 1000 μg, about 990 μg to about 1100 μg, about 1090 μg to about 1200 μg, about 1190 μg to about 1300 μg, about 1290 μg to about 1400 μg, or about 1390 μg to about 1500 μg at a frequency of once every four weeks. In some embodiments, the binding protein is administered to the subject at a dose between 10 μg to 25 μg, 20 μg to 50 μg, 40 μg to 70 μg, 60 μg to 100 μg, 90 μg to 150 μg, 140 μg to 200 μg, 190 μg to 250 μg, 240 μg to 300 μg, 290 μg to 350 μg, 340 μg to 400 μg, 390 μg to 460 μg, 450 μg to 500 μg, 490 μg to 600 μg, 590 μg to 700 μg, 690 μg to 750 μg, 740 μg to 800 μg, 790 μg to 880 μg, 870 μg to 950 μg, 940 μg to 1000 μg, 990 μg to 1100 μg, 1090 μg to 1200 μg, 1190 μg to 1300 μg, 1290 μg to 1400 μg, or 1390 μg to 1500 μg at a frequency of once every four weeks.

In some embodiments, the binding protein is administered to the subject at a dose of about 10 μg, about 11 μg, about 12 μg, about 13 μg, about 14 μg, about 15 μg, about 16 μg, about 17 μg, about 18 μg, about 19 μg, about 20 μg, about 30 μg, about 40 μg, about 50 μg, about 60 μg, about 70 μg, about 80 μg, about 90 μg, about 100 μg, about 110 μg, about 120 μg, about 130 μg, about 140 μg, about 150 μg, about 160 μg, about 170 μg, about 180 μg, about 190 μg, about 200 μg, about 220 μg, about 240 μg, about 260 μg, about 280 μg, about 300 μg, about 320 μg, about 340 μg, about 360 μg, about 380 μg, about 400 μg, about 420 μg, about 440 μg, about 460 μg, about 480 μg, about 500 μg, about 520 μg, about 540 μg, about 560 μg, about 580 μg, about 600 μg, about 620 μg, about 640 μg, about 660 μg, about 680 μg, about 700 μg, about 720 μg, about 740 μg, about 760 μg, about 780 μg, about 800 μg, about 820 μg, about 840 μg, about 860 μg, about 880 μg, about 900 μg, about 920 μg, about 940 μg, about 960 μg, about 980 μg, about 1000 μg, about 1020 μg, about 1040 μg, about 1060 μg, about 1080 μg, about 1100 μg, about 1120 μg, about 1125 μg, about 1140 μg, about 1160 μg, about 1180 μg, about 1200 μg, about 1220 μg, about 1240 μg, about 1260 μg, about 1280 μg, about 1300 μg, about 1320 μg, about 1340 μg, about 1360 μg, about 1380 μg, about 1400 μg, about 1420 μg, or about 1440 μg, about 1460 μg, about 1480 μg, about 1500 μg at a frequency of once every four weeks.

In some embodiments, the binding protein is administered to the subject at a dose of 10 μg, 11 μg, 12 μg, 13 μg, 14 μg, 15 μg, 16 μg, 17 μg, 18 μg, 19 μg, 20 μg, 30 μg, 40 μg, 50 μg, 60 μg, 70 μg, 80 μg, 90 μg, 100 μg, 110 μg, 120 μg, 130 μg, 140 μg, 150 μg, 160 μg, 170 μg, 180 μg, 190 μg, 200 μg, 220 μg, 240 μg, 260 μg, 280 μg, 300 μg, 320 μg, 340 μg, 360 μg, 380 μg, 400 μg, 420 μg, 440 μg, 460 μg, 480 μg, 500 μg, 520 μg, 540 μg, 560 μg, 580 μg, 600 μg, 620 μg, 640 μg, 660 μg, 680 μg, 700 μg, 720 μg, 740 μg, 760 μg, 780 μg, 800 μg, 820 μg, 840 μg, 860 μg, 880 μg, 900 μg, 920 μg, 940 μg, 960 μg, 980 μg, 1000 μg, 1020 μg, 1040 μg, 1060 μg, 1080 μg, 1100 μg, 1120 μg, 1125 μg, 1140 μg, 1160 μg, 1180 μg, 1200 μg, 1220 μg, 1240 μg, 1260 μg, 1280 μg, 1300 μg, 1320 μg, 1340 μg, 1360 μg, 1380 μg, 1400 μg, 1420 μg, or 1440 μg, 1460 μg, 1480 μg, 1500 μg at a frequency of once every four weeks.

In some embodiments, the binding protein is administered to the subject at a dose of 18 μg, 60 μg, 180 μg, 240 μg, 360 μg, 480 μg, 720 μg, 900 μg, 1080 μg, 1125 μg, or 1440 μg at a frequency of once every four weeks.

In some embodiments, the binding protein is administered to the subject at a dose of 10 μg to 1500 μg at a frequency of once every five weeks. In some embodiments, the binding protein is administered to the subject at a dose between about 10 μg to about 25 μg, about 20 μg to about 50 μg, about 40 μg to about 70 μg, about 60 μg to about 100 μg, about 90 μg to about 150 μg, about 140 μg to about 200 μg, about 190 μg to about 250 μg, about 240 μg to about 300 μg, about 290 μg to about 350 μg, about 340 μg to about 400 μg, about 390 μg to about 460 μg, about 450 μg to about 500 μg, about 490 μg to about 600 μg, about 590 μg to about 700 μg, about 690 μg to about 750 μg, about 740 μg to about 800 μg, about 790 μg to about 880 μg, about 870 μg to about 950 μg, about 940 μg to about 1000 μg, about 990 μg to about 1100 μg, about 1090 μg to about 1200 μg, about 1190 μg to about 1300 μg, about 1290 μg to about 1400 μg, or about 1390 μg to about 1500 μg at a frequency of once every five weeks. In some embodiments, the binding protein is administered to the subject at a dose between 10 μg to 25 μg, 20 μg to 50 μg, 40 μg to 70 μg, 60 μg to 100 μg, 90 μg to 150 μg, 140 μg to 200 μg, 190 μg to 250 μg, 240 μg to 300 μg, 290 μg to 350 μg, 340 μg to 400 μg, 390 μg to 460 μg, 450 μg to 500 μg, 490 μg to 600 μg, 590 μg to 700 μg, 690 μg to 750 μg, 740 μg to 800 μg, 790 μg to 880 μg, 870 μg to 950 μg, 940 μg to 1000 μg, 990 μg to 1100 μg, 1090 μg to 1200 μg, 1190 μg to 1300 μg, 1290 μg to 1400 μg, or 1390 μg to 1500 μg at a frequency of once every five weeks.

In some embodiments, the binding protein is administered to the subject at a dose of about 10 μg, about 11 μg, about 12 μg, about 13 μg, about 14 μg, about 15 μg, about 16 μg, about 17 μg, about 18 μg, about 19 μg, about 20 μg, about 30 μg, about 40 μg, about 50 μg, about 60 μg, about 70 μg, about 80 μg, about 90 μg, about 100 μg, about 110 μg, about 120 μg, about 130 μg, about 140 μg, about 150 μg, about 160 μg, about 170 μg, about 180 μg, about 190 μg, about 200 μg, about 220 μg, about 240 μg, about 260 μg, about 280 μg, about 300 μg, about 320 μg, about 340 μg, about 360 μg, about 380 μg, about 400 μg, about 420 μg, about 440 μg, about 460 μg, about 480 μg, about 500 μg, about 520 μg, about 540 μg, about 560 μg, about 580 μg, about 600 μg, about 620 μg, about 640 μg, about 660 μg, about 680 μg, about 700 μg, about 720 μg, about 740 μg, about 760 μg, about 780 μg, about 800 μg, about 820 μg, about 840 μg, about 860 μg, about 880 μg, about 900 μg, about 920 μg, about 940 μg, about 960 μg, about 980 μg, about 1000 μg, about 1020 μg, about 1040 μg, about 1060 μg, about 1080 μg, about 1100 μg, about 1120 μg, about 1125 μg, about 1140 μg, about 1160 μg, about 1180 μg, about 1200 μg, about 1220 μg, about 1240 μg, about 1260 μg, about 1280 μg, about 1300 μg, about 1320 μg, about 1340 μg, about 1360 μg, about 1380 μg, about 1400 μg, about 1420 μg, or about 1440 μg, about 1460 μg, about 1480 μg, about 1500 μg at a frequency of once every five weeks.

In some embodiments, the binding protein is administered to the subject at a dose of 10 μg, 11 μg, 12 μg, 13 μg, 14 μg, 15 μg, 16 μg, 17 μg, 18 μg, 19 μg, 20 μg, 30 μg, 40 μg, 50 μg, 60 μg, 70 μg, 80 μg, 90 μg, 100 μg, 110 μg, 120 μg, 130 μg, 140 μg, 150 μg, 160 μg, 170 μg, 180 μg, 190 μg, 200 μg, 220 μg, 240 μg, 260 μg, 280 μg, 300 μg, 320 μg, 340 μg, 360 μg, 380 μg, 400 μg, 420 μg, 440 μg, 460 μg, 480 μg, 500 μg, 520 μg, 540 μg, 560 μg, 580 μg, 600 μg, 620 μg, 640 μg, 660 μg, 680 μg, 700 μg, 720 μg, 740 μg, 760 μg, 780 μg, 800 μg, 820 μg, 840 μg, 860 μg, 880 μg, 900 μg, 920 μg, 940 μg, 960 μg, 980 μg, 1000 μg, 1020 μg, 1040 μg, 1060 μg, 1080 μg, 1100 μg, 1120 μg, 1125 μg, 1140 μg, 1160 μg, 1180 μg, 1200 μg, 1220 μg, 1240 μg, 1260 μg, 1280 μg, 1300 μg, 1320 μg, 1340 μg, 1360 μg, 1380 μg, 1400 μg, 1420 μg, or 1440 μg, 1460 μg, 1480 μg, 1500 μg at a frequency of once every five weeks.

In some embodiments, the binding protein is administered to the subject at a dose of 18 μg, 60 μg, 180 μg, 240 μg, 360 μg, 480 μg, 720 μg, 900 μg, 1080 μg, 1125 μg, or 1440 μg at a frequency of once every five weeks.

In some embodiments, the binding protein is administered to the subject at a dose of 10 μg to 1500 μg at a frequency of once every six weeks. In some embodiments, the binding protein is administered to the subject at a dose between about 10 μg to about 25 μg, about 20 μg to about 50 μg, about 40 μg to about 70 μg, about 60 μg to about 100 μg, about 90 μg to about 150 μg, about 140 μg to about 200 μg, about 190 μg to about 250 μg, about 240 μg to about 300 μg, about 290 μg to about 350 μg, about 340 μg to about 400 μg, about 390 μg to about 460 μg, about 450 μg to about 500 μg, about 490 μg to about 600 μg, about 590 μg to about 700 μg, about 690 μg to about 750 μg, about 740 μg to about 800 μg, about 790 μg to about 880 μg, about 870 μg to about 950 μg, about 940 μg to about 1000 μg, about 990 μg to about 1100 μg, about 1090 μg to about 1200 μg, about 1190 μg to about 1300 μg, about 1290 μg to about 1400 μg, or about 1390 μg to about 1500 μg at a frequency of once every six weeks. In some embodiments, the binding protein is administered to the subject at a dose between 10 μg to 25 μg, 20 μg to 50 μg, 40 μg to 70 μg, 60 μg to 100 μg, 90 μg to 150 μg, 140 μg to 200 μg, 190 μg to 250 μg, 240 μg to 300 μg, 290 μg to 350 μg, 340 μg to 400 μg, 390 μg to 460 μg, 450 μg to 500 μg, 490 μg to 600 μg, 590 μg to 700 μg, 690 μg to 750 μg, 740 μg to 800 μg, 790 μg to 880 μg, 870 μg to 950 μg, 940 μg to 1000 μg, 990 μg to 1100 μg, 1090 μg to 1200 μg, 1190 μg to 1300 μg, 1290 μg to 1400 μg, or 1390 μg to 1500 μg at a frequency of once every six weeks.

In some embodiments, the binding protein is administered to the subject at a dose of about 10 μg, about 11 μg, about 12 μg, about 13 μg, about 14 μg, about 15 μg, about 16 μg, about 17 μg, about 18 μg, about 19 μg, about 20 μg, about 30 μg, about 40 μg, about 50 μg, about 60 μg, about 70 μg, about 80 μg, about 90 μg, about 100 μg, about 110 μg, about 120 μg, about 130 μg, about 140 μg, about 150 μg, about 160 μg, about 170 μg, about 180 μg, about 190 μg, about 200 μg, about 220 μg, about 240 μg, about 260 μg, about 280 μg, about 300 μg, about 320 μg, about 340 μg, about 360 μg, about 380 μg, about 400 μg, about 420 μg, about 440 μg, about 460 μg, about 480 μg, about 500 μg, about 520 μg, about 540 μg, about 560 μg, about 580 μg, about 600 μg, about 620 μg, about 640 μg, about 660 μg, about 680 μg, about 700 μg, about 720 μg, about 740 μg, about 760 μg, about 780 μg, about 800 μg, about 820 μg, about 840 μg, about 860 μg, about 880 μg, about 900 μg, about 920 μg, about 940 μg, about 960 μg, about 980 μg, about 1000 μg, about 1020 μg, about 1040 μg, about 1060 μg, about 1080 μg, about 1100 μg, about 1120 μg, about 1125 μg, about 1140 μg, about 1160 μg, about 1180 μg, about 1200 μg, about 1220 μg, about 1240 μg, about 1260 μg, about 1280 μg, about 1300 μg, about 1320 μg, about 1340 μg, about 1360 μg, about 1380 μg, about 1400 μg, about 1420 μg, or about 1440 μg, about 1460 μg, about 1480 μg, about 1500 μg at a frequency of once every six weeks.

In some embodiments, the binding protein is administered to the subject at a dose of 10 μg, 11 μg, 12 μg, 13 μg, 14 μg, 15 μg, 16 μg, 17 μg, 18 μg, 19 μg, 20 μg, 30 μg, 40 μg, 50 μg, 60 μg, 70 μg, 80 μg, 90 μg, 100 μg, 110 μg, 120 μg, 130 μg, 140 μg, 150 μg, 160 μg, 170 μg, 180 μg, 190 μg, 200 μg, 220 μg, 240 μg, 260 μg, 280 μg, 300 μg, 320 μg, 340 μg, 360 μg, 380 μg, 400 μg, 420 μg, 440 μg, 460 μg, 480 μg, 500 μg, 520 μg, 540 μg, 560 μg, 580 μg, 600 μg, 620 μg, 640 μg, 660 μg, 680 μg, 700 μg, 720 μg, 740 μg, 760 μg, 780 μg, 800 μg, 820 μg, 840 μg, 860 μg, 880 μg, 900 μg, 920 μg, 940 μg, 960 μg, 980 μg, 1000 μg, 1020 μg, 1040 μg, 1060 μg, 1080 μg, 1100 μg, 1120 μg, 1125 μg, 1140 μg, 1160 μg, 1180 μg, 1200 μg, 1220 μg, 1240 μg, 1260 μg, 1280 μg, 1300 μg, 1320 μg, 1340 μg, 1360 μg, 1380 μg, 1400 μg, 1420 μg, or 1440 μg, 1460 μg, 1480 μg, 1500 μg at a frequency of once every six weeks.

In some embodiments, the binding protein is administered to the subject at a dose of 18 μg, 60 μg, 180 μg, 240 μg, 360 μg, 480 μg, 720 μg, 900 μg, 1080 μg, 1125 μg, or 1440 μg at a frequency of once every six weeks.

In some embodiments, the binding protein is administered to the subject at a dose of 10 μg to 1500 μg at a frequency of once every seven weeks. In some embodiments, the binding protein is administered to the subject at a dose between about 10 μg to about 25 μg, about 20 μg to about 50 μg, about 40 μg to about 70 μg, about 60 μg to about 100 μg, about 90 μg to about 150 μg, about 140 μg to about 200 μg, about 190 μg to about 250 μg, about 240 μg to about 300 μg, about 290 μg to about 350 μg, about 340 μg to about 400 μg, about 390 μg to about 460 μg, about 450 μg to about 500 μg, about 490 μg to about 600 μg, about 590 μg to about 700 μg, about 690 μg to about 750 μg, about 740 μg to about 800 μg, about 790 μg to about 880 μg, about 870 μg to about 950 μg, about 940 μg to about 1000 μg, about 990 μg to about 1100 μg, about 1090 μg to about 1200 μg, about 1190 μg to about 1300 μg, about 1290 μg to about 1400 μg, or about 1390 μg to about 1500 μg at a frequency of once every seven weeks. In some embodiments, the binding protein is administered to the subject at a dose between 10 μg to 25 μg, 20 μg to 50 μg, 40 μg to 70 μg, 60 μg to 100 μg, 90 μg to 150 μg, 140 μg to 200 μg, 190 μg to 250 μg, 240 μg to 300 μg, 290 μg to 350 μg, 340 μg to 400 μg, 390 μg to 460 μg, 450 μg to 500 μg, 490 μg to 600 μg, 590 μg to 700 μg, 690 μg to 750 μg, 740 μg to 800 μg, 790 μg to 880 μg, 870 μg to 950 μg, 940 μg to 1000 μg, 990 μg to 1100 μg, 1090 μg to 1200 μg, 1190 μg to 1300 μg, 1290 μg to 1400 μg, or 1390 μg to 1500 μg at a frequency of once every seven weeks.

In some embodiments, the binding protein is administered to the subject at a dose of about 10 μg, about 11 μg, about 12 μg, about 13 μg, about 14 μg, about 15 μg, about 16 μg, about 17 μg, about 18 μg, about 19 μg, about 20 μg, about 30 μg, about 40 μg, about 50 μg, about 60 μg, about 70 μg, about 80 μg, about 90 μg, about 100 μg, about 110 μg, about 120 μg, about 130 μg, about 140 μg, about 150 μg, about 160 μg, about 170 μg, about 180 μg, about 190 μg, about 200 μg, about 220 μg, about 240 μg, about 260 μg, about 280 μg, about 300 μg, about 320 μg, about 340 μg, about 360 μg, about 380 μg, about 400 μg, about 420 μg, about 440 μg, about 460 μg, about 480 μg, about 500 μg, about 520 μg, about 540 μg, about 560 μg, about 580 μg, about 600 μg, about 620 μg, about 640 μg, about 660 μg, about 680 μg, about 700 μg, about 720 μg, about 740 μg, about 760 μg, about 780 μg, about 800 μg, about 820 μg, about 840 μg, about 860 μg, about 880 μg, about 900 μg, about 920 μg, about 940 μg, about 960 μg, about 980 μg, about 1000 μg, about 1020 μg, about 1040 μg, about 1060 μg, about 1080 μg, about 1100 μg, about 1120 μg, about 1125 μg, about 1140 μg, about 1160 μg, about 1180 μg, about 1200 μg, about 1220 μg, about 1240 μg, about 1260 μg, about 1280 μg, about 1300 μg, about 1320 μg, about 1340 μg, about 1360 μg, about 1380 μg, about 1400 μg, about 1420 μg, or about 1440 μg, about 1460 μg, about 1480 μg, about 1500 μg at a frequency of once every seven weeks.

In some embodiments, the binding protein is administered to the subject at a dose of 10 μg, 11 μg, 12 μg, 13 μg, 14 μg, 15 μg, 16 μg, 17 μg, 18 μg, 19 μg, 20 μg, 30 μg, 40 μg, 50 μg, 60 μg, 70 μg, 80 μg, 90 μg, 100 μg, 110 μg, 120 μg, 130 μg, 140 μg, 150 μg, 160 μg, 170 μg, 180 μg, 190 μg, 200 μg, 220 μg, 240 μg, 260 μg, 280 μg, 300 μg, 320 μg, 340 μg, 360 μg, 380 μg, 400 μg, 420 μg, 440 μg, 460 μg, 480 μg, 500 μg, 520 μg, 540 μg, 560 μg, 580 μg, 600 μg, 620 μg, 640 μg, 660 μg, 680 μg, 700 μg, 720 μg, 740 μg, 760 μg, 780 μg, 800 μg, 820 μg, 840 μg, 860 μg, 880 μg, 900 μg, 920 μg, 940 μg, 960 μg, 980 μg, 1000 μg, 1020 μg, 1040 μg, 1060 μg, 1080 μg, 1100 μg, 1120 μg, 1125 μg, 1140 μg, 1160 μg, 1180 μg, 1200 μg, 1220 μg, 1240 μg, 1260 μg, 1280 μg, 1300 μg, 1320 μg, 1340 μg, 1360 μg, 1380 μg, 1400 μg, 1420 μg, or 1440 μg, 1460 μg, 1480 μg, 1500 μg at a frequency of once every seven weeks.

In some embodiments, the binding protein is administered to the subject at a dose of 18 μg, 60 μg, 180 μg, 240 μg, 360 μg, 480 μg, 720 μg, 900 μg, 1080 μg, 1125 μg, or 1440 μg at a frequency of once every seven weeks.

In some embodiments, the binding protein is administered to the subject at a dose of 10 μg to 1500 μg at a frequency of once every eight weeks. In some embodiments, the binding protein is administered to the subject at a dose between about 10 μg to about 25 μg, about 20 μg to about 50 μg, about 40 μg to about 70 μg, about 60 μg to about 100 μg, about 90 μg to about 150 μg, about 140 μg to about 200 μg, about 190 μg to about 250 μg, about 240 μg to about 300 μg, about 290 μg to about 350 μg, about 340 μg to about 400 μg, about 390 μg to about 460 μg, about 450 μg to about 500 μg, about 490 μg to about 600 μg, about 590 μg to about 700 μg, about 690 μg to about 750 μg, about 740 μg to about 800 μg, about 790 μg to about 880 μg, about 870 μg to about 950 μg, about 940 μg to about 1000 μg, about 990 μg to about 1100 μg, about 1090 μg to about 1200 μg, about 1190 μg to about 1300 μg, about 1290 μg to about 1400 μg, or about 1390 μg to about 1500 μg at a frequency of once every eight weeks. In some embodiments, the binding protein is administered to the subject at a dose between 10 μg to 25 μg, 20 μg to 50 μg, 40 μg to 70 μg, 60 μg to 100 μg, 90 μg to 150 μg, 140 μg to 200 μg, 190 μg to 250 μg, 240 μg to 300 μg, 290 μg to 350 μg, 340 μg to 400 μg, 390 μg to 460 μg, 450 μg to 500 μg, 490 μg to 600 μg, 590 μg to 700 μg, 690 μg to 750 μg, 740 μg to 800 μg, 790 μg to 880 μg, 870 μg to 950 μg, 940 μg to 1000 μg, 990 μg to 1100 μg, 1090 μg to 1200 μg, 1190 μg to 1300 μg, 1290 μg to 1400 μg, or 1390 μg to 1500 μg at a frequency of once every eight weeks.

In some embodiments, the binding protein is administered to the subject at a dose of about 10 μg, about 11 μg, about 12 μg, about 13 μg, about 14 μg, about 15 μg, about 16 μg, about 17 μg, about 18 μg, about 19 μg, about 20 μg, about 30 μg, about 40 μg, about 50 μg, about 60 μg, about 70 μg, about 80 μg, about 90 μg, about 100 μg, about 110 μg, about 120 μg, about 130 μg, about 140 μg, about 150 μg, about 160 μg, about 170 μg, about 180 μg, about 190 μg, about 200 μg, about 220 μg, about 240 μg, about 260 μg, about 280 μg, about 300 μg, about 320 μg, about 340 μg, about 360 μg, about 380 μg, about 400 μg, about 420 μg, about 440 μg, about 460 μg, about 480 μg, about 500 μg, about 520 μg, about 540 μg, about 560 μg, about 580 μg, about 600 μg, about 620 μg, about 640 μg, about 660 μg, about 680 μg, about 700 μg, about 720 μg, about 740 μg, about 760 μg, about 780 μg, about 800 μg, about 820 μg, about 840 μg, about 860 μg, about 880 μg, about 900 μg, about 920 μg, about 940 μg, about 960 μg, about 980 μg, about 1000 μg, about 1020 μg, about 1040 μg, about 1060 μg, about 1080 μg, about 1100 μg, about 1120 μg, about 1125 μg, about 1140 μg, about 1160 μg, about 1180 μg, about 1200 μg, about 1220 μg, about 1240 μg, about 1260 μg, about 1280 μg, about 1300 μg, about 1320 μg, about 1340 μg, about 1360 μg, about 1380 μg, about 1400 μg, about 1420 μg, or about 1440 μg, about 1460 μg, about 1480 μg, about 1500 μg at a frequency of once every eight weeks.

In some embodiments, the binding protein is administered to the subject at a dose of 10 μg, 11 μg, 12 μg, 13 μg, 14 μg, 15 μg, 16 μg, 17 μg, 18 μg, 19 μg, 20 μg, 30 μg, 40 μg, 50 μg, 60 μg, 70 μg, 80 μg, 90 μg, 100 μg, 110 μg, 120 μg, 130 μg, 140 μg, 150 μg, 160 μg, 170 μg, 180 μg, 190 μg, 200 μg, 220 μg, 240 μg, 260 μg, 280 μg, 300 μg, 320 μg, 340 μg, 360 μg, 380 μg, 400 μg, 420 μg, 440 μg, 460 μg, 480 μg, 500 μg, 520 μg, 540 μg, 560 μg, 580 μg, 600 μg, 620 μg, 640 μg, 660 μg, 680 μg, 700 μg, 720 μg, 740 μg, 760 μg, 780 μg, 800 μg, 820 μg, 840 μg, 860 μg, 880 μg, 900 μg, 920 μg, 940 μg, 960 μg, 980 μg, 1000 μg, 1020 μg, 1040 μg, 1060 μg, 1080 μg, 1100 μg, 1120 μg, 1125 μg, 1140 μg, 1160 μg, 1180 μg, 1200 μg, 1220 μg, 1240 μg, 1260 μg, 1280 μg, 1300 μg, 1320 μg, 1340 μg, 1360 μg, 1380 μg, 1400 μg, 1420 μg, or 1440 μg, 1460 μg, 1480 μg, 1500 μg at a frequency of once every eight weeks.

In some embodiments, the binding protein is administered to the subject at a dose of 18 μg, 60 μg, 180 μg, 240 μg, 360 μg, 480 μg, 720 μg, 900 μg, 1080 μg, 1125 μg, or 1440 μg at a frequency of once every eight weeks.

In some embodiments, at least 1 dose, at least 2 doses, at least 3 doses, at least 4 doses, at least 5 doses, at least 6 doses, at least 7 doses, at least 8 doses, at least 9 doses, at least 10 doses, at least 11 doses, at least 12 doses, at least 13 doses, at least 14 doses, at least 15 doses, at least 16 doses, at least 17 doses, at least 18 doses, at least 19 doses, or at least 20 doses of the binding protein provided herein is administered (e.g., subcutaneously or intravenously) to the subject.

In some embodiments, the subject is treated for a treatment period of up to 4 weeks, up to 5 weeks, up to 6 weeks, up to 7 weeks, up to 8 weeks, up to 9 weeks, up to 10 weeks, up to 11 weeks, up to 12 weeks, up to 13 weeks, up to 14 weeks, up to 15 weeks, up to 16 weeks, up to 17 weeks, up to 18 weeks, up to 19 weeks, up to 20 weeks, up to 21 weeks, up to 22 weeks, up to 23 weeks, up to 24 weeks, up to 25 weeks, up to 26 weeks, up to 27 weeks, up to 28 weeks, up to 29 weeks, up to 30 weeks, up to 31 weeks, up to 32 weeks, up to 33 weeks, up to 34 weeks, up to 35 weeks, up to 36 weeks, up to 37 weeks, up to 38 weeks, up to 39 weeks, up to 40 weeks, up to 41 weeks, up to 42 weeks, up to 43 weeks, up to 44 weeks, up to 45 weeks, up to 46 weeks, up to 47 weeks, up to 48 weeks, up to 49 weeks, up to 50 weeks, up to 51 weeks, or up to 52 weeks in length.

In some embodiments, the subject is treated for a treatment period of at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, at least 24 weeks, at least 25 weeks, at least 26 weeks, at least 27 weeks, at least 28 weeks, at least 29 weeks, at least 30 weeks, at least 31 weeks, at least 32 weeks, at least 33 weeks, at least 34 weeks, at least 35 weeks, at least 36 weeks, at least 37 weeks, at least 38 weeks, at least 39 weeks, at least 40 weeks, at least 41 weeks, at least 42 weeks, at least 43 weeks, at least 44 weeks, at least 45 weeks, at least 46 weeks, at least 47 weeks, at least 48 weeks, at least 49 weeks, at least 50 weeks, at least 51 weeks, or at least 52 weeks in length.

In some embodiments, the binding protein is administered to the subject intermittently over a cycling regimen. In some embodiments, the binding protein is administered to the subject for least 1 cycling regimen, at least 2 cycling regimens, at least 3 cycling regimens, at least 4 cycling regimens, at least 5 cycling regimens, at least 6 cycling regimens, at least 7 cycling regimens, at least 8 cycling regimens, at least 9 cycling regimens, at least 10 cycling regimens, at least 11 cycling regimens, at least 12 cycling regimens, at least 13 cycling regimens, at least 14 cycling regimens, at least 15 cycling regimens, at least 16 cycling regimens, at least 17 cycling regimens, at least 18 cycling regimens, at least 19 cycling regimens, or at least 20 cycling regimens. In some embodiments, the cycling regimen is a 28-day cycle. In some embodiments, the binding protein is administered to the subject for at least one 28-day cycle. In some embodiments, the binding protein is administered on at least day 1, 2, 3, 4, 5, 6, 7, 8, 9, 10, 11, 12, 13, 14, 15, 16, 17, 18, 19, 20, 21, 22, 23, 24, 25, 26, 27, or 28 of the 28-day cycle.

In some embodiments, the binding protein is administered to the subject weekly during the at least one 28-day cycle, wherein each dose of the binding protein is between about 10 μg to about 1500 μg. In some embodiments, each dose of the binding protein is 10 μg to 1500 μg. In some embodiments, each dose of the binding protein is between about 10 μg to about 25 μg, about 20 μg to about 50 μg, about 40 μg to about 70 μg, about 60 μg to about 100 μg, about 90 μg to about 150 μg, about 140 μg to about 200 μg, about 190 μg to about 250 μg, about 240 μg to about 300 μg, about 290 μg to about 350 μg, about 340 μg to about 400 μg, about 390 μg to about 460 μg, about 450 μg to about 500 μg, about 490 μg to about 600 μg, about 590 μg to about 700 μg, about 690 μg to about 750 μg, about 740 μg to about 800 μg, about 790 μg to about 880 μg, about 870 μg to about 950 μg, about 940 μg to about 1000 μg, about 990 μg to about 1100 μg, about 1090 μg to about 1200 μg, about 1190 μg to about 1300 μg, about 1290 μg to about 1400 μg, or about 1390 μg to about 1500 μg. In some embodiments, each dose of the binding protein is between 10 μg to 25 μg, 20 μg to 50 μg, 40 μg to 70 μg, 60 μg to 100 μg, 90 μg to 150 μg, 140 μg to 200 μg, 190 μg to 250 μg, 240 μg to 300 μg, 290 μg to 350 μg, 340 μg to 400 μg, 390 μg to 460 μg, 450 μg to 500 μg, 490 μg to 600 μg, 590 μg to 700 μg, 690 μg to 750 μg, 740 μg to 800 μg, 790 μg to 880 μg, 870 μg to 950 μg, 940 μg to 1000 μg, 990 μg to 1100 μg, 1090 μg to 1200 μg, 1190 μg to 1300 μg, 1290 μg to 1400 μg, or 1390 μg to 1500 μg. In some embodiments, each dose of the binding protein is about 18 μg, about 60 μg, about 180 μg, about 240 μg, about 360 μg, about 480 μg, about 720 μg, about 900 μg, about 1080 μg, about 1125 μg, or about 1440 μg. In some embodiments, each dose of the binding protein is 18 μg, 60 μg, 180 μg, 240 μg, 360 μg, 480 μg, 720 μg, 900 μg, 1080 μg, 1125 μg, or 1440 μg.

In some embodiments, the binding protein is administered to the subject weekly during at least one 28-day cycle, wherein the binding protein is administered to the subject on days 1, 8, 15, and 22 of the at least one 28-day cycle. In some embodiments, the binding protein is administered to the subject on days 1, 8, 15, and 22, wherein each dose of the binding protein is about 18 μg. In some embodiments, the binding protein is administered to the subject on days 1, 8, 15, and 22, wherein each dose of the binding protein is about 60 μg. In some embodiments, the binding protein is administered to the subject on days 1, 8, 15, and 22, wherein each dose of the binding protein is about 180 μg. In some embodiments, the binding protein is administered to the subject on days 1, 8, 15, and 22, wherein each dose of the binding protein is about 240 μg. In some embodiments, the binding protein is administered to the subject on days 1, 8, 15, and 22, wherein each dose of the binding protein is about 360 μg. In some embodiments, the binding protein is administered to the subject on days 1, 8, 15, and 22, wherein each dose of the binding protein is about 480 μg. In some embodiments, the binding protein is administered to the subject on days 1, 8, 15, and 22, wherein each dose of the binding protein is about 720 μg. In some embodiments, the binding protein is administered to the subject on days 1, 8, 15, and 22, wherein each dose of the binding protein is about 900 μg. In some embodiments, the binding protein is administered to the subject on days 1, 8, 15, and 22, wherein each dose of the binding protein is about 1080 μg. In some embodiments, the binding protein is administered to the subject on days 1, 8, 15, and 22, wherein each dose of the binding protein is about 1125 μg. In some embodiments, the binding protein is administered to the subject on days 1, 8, 15, and 22, wherein each dose of the binding protein is about 1440 μg.

In some embodiments, the binding protein is administered to the subject on days 1, 8, 15, and 22, wherein each dose of the binding protein is 18 μg. In some embodiments, the binding protein is administered to the subject on days 1, 8, 15, and 22, wherein each dose of the binding protein is 60 μg. In some embodiments, the binding protein is administered to the subject on days 1, 8, 15, and 22, wherein each dose of the binding protein is 180 μg. In some embodiments, the binding protein is administered to the subject on days 1, 8, 15, and 22, wherein each dose of the binding protein is 240 μg. In some embodiments, the binding protein is administered to the subject on days 1, 8, 15, and 22, wherein each dose of the binding protein is 360 μg. In some embodiments, the binding protein is administered to the subject on days 1, 8, 15, and 22, wherein each dose of the binding protein is 480 μg. In some embodiments, the binding protein is administered to the subject on days 1, 8, 15, and 22, wherein each dose of the binding protein is 720 μg. In some embodiments, the binding protein is administered to the subject on days 1, 8, 15, and 22, wherein each dose of the binding protein is 900 μg. In some embodiments, the binding protein is administered to the subject on days 1, 8, 15, and 22, wherein each dose of the binding protein is 1080 μg. In some embodiments, the binding protein is administered to the subject on days 1, 8, 15, and 22, wherein each dose of the binding protein is 1125 μg. In some embodiments, the binding protein is administered to the subject on days 1, 8, 15, and 22, wherein each dose of the binding protein is 1440 μg.

In some embodiments, the binding protein is administered to the subject during a lead-in phase prior to initiation of the first cycling regimen (e.g., a 28-day cycle). In some embodiments, the lead-in phase is performed to minimize the risk of severe toxicity (e.g., cytokine release syndrome (CRS)). In some embodiments, the lead-in phase comprises administration of the binding protein in an escalating treatment regimen.

In some embodiments, a target dose (e.g., the dose of the binding protein administered subsequently in a cycling regimen) is reached during the lead-in phase. In some embodiments, the lead-in phase is 28 days, and the target dose is reached during the third week (e.g., day 15) of the lead-in phase. In some embodiments, the lead-in phase is 28 days, and the target dose is reached during the fourth week (e.g., day 22) of the lead-in phase.

In some embodiments, the lead-in phase is 28 days, and each dose of the binding protein in the escalating treatment regimen is between about 0.2 μg to about 1500 μg. In some embodiments, each dose of the binding protein in the escalating treatment regimen is between 0.2 μg to 1500 μg.

In some embodiments, the escalating treatment regimen comprises administration of the binding protein to the subject at sequential doses of about 0.6 μg, about 1.8 μg, about 6.0 μg, about 18 μg, and about 18 μg. In some embodiments, the escalating treatment regimen comprises administration of the binding protein to the subject at sequential doses of about 1.8 μg, about 6.0 μg, about 18 μg, about 60 μg, and about 60 μg. In some embodiments, the escalating treatment regimen comprises administration of the binding protein to the subject at sequential doses of about 6.0 μg, about 18 μg, about 60 μg, about 180 μg, and about 180 μg. In some embodiments, the escalating treatment regimen comprises administration of the binding protein to the subject at sequential doses of about 9.0 μg, about 27 μg, about 90 μg, about 240 μg, and about 240 μg. In some embodiments, the escalating treatment regimen comprises administration of the binding protein to the subject at sequential doses of about 18 μg, about 60 μg, about 180 μg, about 360 μg, and about 360 μg. In some embodiments, the escalating treatment regimen comprises administration of the binding protein to the subject at sequential doses of about 18 μg, about 60 μg, about 180 μg, about 720 μg, and about 720 μg. In some embodiments, the escalating treatment regimen comprises administration of the binding protein to the subject at sequential doses of about 18 μg, about 60 μg, about 180 μg, about 1080 μg, and about 1080 μg. In some embodiments, the escalating treatment regimen comprises administration of the binding protein to the subject at sequential doses of about 18 μg, about 60 μg, about 180 μg, about 1440 μg, and about 1440 μg. In some embodiments, the escalating treatment regimen comprises administration of the binding protein to the subject at sequential doses of about 18 μg, about 60 μg, about 120 μg, about 240 μg, and about 480 μg. In some embodiments, the escalating treatment regimen comprises administration of the binding protein to the subject at sequential doses of about 18 μg, about 60 μg, about 120 μg, about 240 μg, and about 720 μg. In some embodiments, the escalating treatment regimen comprises administration of the binding protein to the subject at sequential doses of about 18 μg, about 60 μg, about 150 μg, about 300 μg, and about 900 μg. In some embodiments, the escalating treatment regimen comprises administration of the binding protein to the subject at sequential doses of about 18 μg, about 60 μg, about 150 μg, about 300 μg, and about 1125 μg.

In some embodiments, the escalating treatment regimen comprises administration of the binding protein to the subject at sequential doses of 0.6 μg, 1.8 μg, 6.0 μg, 18 μg, and 18 μg. In some embodiments, the escalating treatment regimen comprises administration of the binding protein to the subject at sequential doses of 1.8 μg, 6.0 μg, 18 μg, 60 μg, and 60 μg. In some embodiments, the escalating treatment regimen comprises administration of the binding protein to the subject at sequential doses of 6.0 μg, 18 μg, 60 μg, 180 μg, and 180 μg. In some embodiments, the escalating treatment regimen comprises administration of the binding protein to the subject at sequential doses of 9.0 μg, 27 μg, 90 μg, 240 μg, and 240 μg. In some embodiments, the escalating treatment regimen comprises administration of the binding protein to the subject at sequential doses of 18 μg, 60 μg, 180 μg, 360 μg, and 360 μg. In some embodiments, the escalating treatment regimen comprises administration of the binding protein to the subject at sequential doses of 18 μg, 60 μg, 180 μg, 720 μg, and 720 μg. In some embodiments, the escalating treatment regimen comprises administration of the binding protein to the subject at sequential doses of 18 μg, 60 μg, 180 μg, 1080 μg, and 1080 μg. In some embodiments, the escalating treatment regimen comprises administration of the binding protein to the subject at sequential doses of 18 μg, 60 μg, 180 μg, 1440 μg, and 1440 μg. In some embodiments, the escalating treatment regimen comprises administration of the binding protein to the subject at sequential doses of 18 μg, 60 μg, 120 μg, 240 μg, and 480 μg. In some embodiments, the escalating treatment regimen comprises administration of the binding protein to the subject at sequential doses of 18 μg, 60 μg, 120 μg, 240 μg, and 720 μg. In some embodiments, the escalating treatment regimen comprises administration of the binding protein to the subject at sequential doses of 18 μg, 60 μg, 150 μg, 300 μg, and 900 μg. In some embodiments, the escalating treatment regimen comprises administration of the binding protein to the subject at sequential doses of 18 μg, 60 μg, 150 μg, 300 μg, and 1125 μg.

In some embodiments, the lead-in phase is 28 days, wherein the lead-in phase comprises administration of the binding protein to the subject on days 1, 4, 8, 15, and 22 of the lead-in phase. In some embodiments, the escalating treatment regimen comprises administration of the binding protein to the subject at sequential doses of about 0.6 μg, about 1.8 μg, about 6.0 μg, about 18 μg, and about 18 μg on days 1, 4, 8, 15, and 22, respectively. In some embodiments, the escalating treatment regimen comprises administration of the binding protein to the subject at sequential doses of about 1.8 μg, about 6.0 μg, about 18 μg, about 60 μg, and about 60 μg on days 1, 4, 8, 15, and 22, respectively. In some embodiments, the escalating treatment regimen comprises administration of the binding protein to the subject at sequential doses of about 6.0 μg, about 18 μg, about 60 μg, about 180 μg, and about 180 μg on days 1, 4, 8, 15, and 22, respectively. In some embodiments, the escalating treatment regimen comprises administration of the binding protein to the subject at sequential doses of about 9.0 μg, about 27 μg, about 90 μg, about 240 μg, and about 240 μg on days 1, 4, 8, 15, and 22, respectively. In some embodiments, the escalating treatment regimen comprises administration of the binding protein to the subject at sequential doses of about 18 μg, about 60 μg, about 180 μg, about 360 μg, and about 360 μg on days 1, 4, 8, 15, and 22, respectively. In some embodiments, the escalating treatment regimen comprises administration of the binding protein to the subject at sequential doses of about 18 μg, about 60 μg, about 180 μg, about 720 μg, and about 720 μg on days 1, 4, 8, 15, and 22, respectively. In some embodiments, the escalating treatment regimen comprises administration of the binding protein to the subject at sequential doses of about 18 μg, about 60 μg, about 180 μg, about 1080 μg, and about 1080 μg on days 1, 4, 8, 15, and 22, respectively. In some embodiments, the escalating treatment regimen comprises administration of the binding protein to the subject at sequential doses of about 18 μg, about 60 μg, about 180 μg, about 1440 μg, and about 1440 μg on days 1, 4, 8, 15, and 22, respectively. In some embodiments, the escalating treatment regimen comprises administration of the binding protein to the subject at sequential doses of about 18 μg, about 60 μg, about 120 μg, about 240 μg, and about 480 μg on days 1, 4, 8, 15, and 22, respectively. In some embodiments, the escalating treatment regimen comprises administration of the binding protein to the subject at sequential doses of about 18 μg, about 60 μg, about 120 μg, about 240 μg, and about 720 μg on days 1, 4, 8, 15, and 22, respectively. In some embodiments, the escalating treatment regimen comprises administration of the binding protein to the subject at sequential doses of about 18 μg, about 60 μg, about 150 μg, about 300 μg, and about 900 μg on days 1, 4, 8, 15, and 22, respectively. In some embodiments, the escalating treatment regimen comprises administration of the binding protein to the subject at sequential doses of about 18 μg, about 60 μg, about 150 μg, about 300 μg, and about 1125 μg on days 1, 4, 8, 15, and 22, respectively.

In some embodiments, the escalating treatment regimen comprises administration of the binding protein to the subject at sequential doses of 0.6 μg, 1.8 μg, 6.0 μg, 18 μg, and 18 μg on days 1, 4, 8, 15, and 22, respectively. In some embodiments, the escalating treatment regimen comprises administration of the binding protein to the subject at sequential doses of 1.8 μg, 6.0 μg, 18 μg, 60 μg, and 60 μg on days 1, 4, 8, 15, and 22, respectively. In some embodiments, the escalating treatment regimen comprises administration of the binding protein to the subject at sequential doses of 6.0 μg, 18 μg, 60 μg, 180 μg, and 180 μg on days 1, 4, 8, 15, and 22, respectively. In some embodiments, the escalating treatment regimen comprises administration of the binding protein to the subject at sequential doses of 9.0 μg, 27 μg, 90 μg, 240 μg, and 240 μg on days 1, 4, 8, 15, and 22, respectively. In some embodiments, the escalating treatment regimen comprises administration of the binding protein to the subject at sequential doses of 18 μg, 60 μg, 180 μg, 360 μg, and 360 μg on days 1, 4, 8, 15, and 22, respectively. In some embodiments, the escalating treatment regimen comprises administration of the binding protein to the subject at sequential doses of 18 μg, 60 μg, 180 μg, 720 μg, and 720 μg on days 1, 4, 8, 15, and 22, respectively. In some embodiments, the escalating treatment regimen comprises administration of the binding protein to the subject at sequential doses of 18 μg, 60 μg, 180 μg, 1080 μg, and 1080 μg on days 1, 4, 8, 15, and 22, respectively. In some embodiments, the escalating treatment regimen comprises administration of the binding protein to the subject at sequential doses of 18 μg, 60 μg, 180 μg, 1440 μg, and 1440 μg on days 1, 4, 8, 15, and 22, respectively. In some embodiments, the escalating treatment regimen comprises administration of the binding protein to the subject at sequential doses of 18 μg, 60 μg, 120 μg, 240 μg, and 480 μg on days 1, 4, 8, 15, and 22, respectively. In some embodiments, the escalating treatment regimen comprises administration of the binding protein to the subject at sequential doses of 18 μg, 60 μg, 120 μg, 240 μg, and 720 μg on days 1, 4, 8, 15, and 22, respectively. In some embodiments, the escalating treatment regimen comprises administration of the binding protein to the subject at sequential doses of 18 μg, 60 μg, 150 μg, 300 μg, and 900 μg on days 1, 4, 8, 15, and 22, respectively. In some embodiments, the escalating treatment regimen comprises administration of the binding protein to the subject at sequential doses of 18 μg, 60 μg, 150 μg, 300 μg, and 1125 μg on days 1, 4, 8, 15, and 22, respectively.

In some embodiments, the binding protein is administered to the subject intravenously. Any suitable method for intravenous administration can be implemented for administration of the binding provided herein. In some embodiments, intravenous administration comprises injection of the binding protein provided herein using a needle or catheter directly into the vein of the subject. Exemplary injection sites include, but are not limited to, the vein of the wrist, elbow, or back of the hand. In some embodiments, the binding protein provided herein is administered through an intravenous “push” or “bolus” injection, wherein the binding protein is injected into the vein rapidly. In some embodiments, the binding protein provided herein is administered through an intravenous infusion (e.g., pump or drip infusion), wherein the binding protein is administered over time.

In some embodiments, the binding protein is administered to the subject weekly for at least one week, and wherein each dose of the binding protein is between about 300 μg to about 4500 μg. In some embodiments, each dose of the binding protein is between 300 μg to 4500 μg. In some embodiments, each dose of the binding protein is between about 100 μg to about 1000 μg, about 200 μg to about 500 μg, about 300 μg to about 400 μg, about 750 μg to about 1500 μg, about 1000 μg to about 1200 μg, about 1900 μg to about 2500 μg, about 2100 μg to about 2300 μg, about 3000 μg to about 3600 μg, about 3200 μg to about 3400 μg, about 3900 μg to about 4500 μg, or about 4100 μg to about 4300 μg. In some embodiments, each dose of the binding protein is between 100 μg to 1000 μg, 200 μg to 500 μg, 300 μg to 400 μg, 750 μg to 1500 μg, 1000 μg to 1200 μg, 1900 μg to 2500 μg, 2100 μg to 2300 μg, 3000 μg to 3600 μg, 3200 μg to 3400 μg, 3900 μg to 4500 μg, or 4100 μg to 4300 μg.

In some embodiments, each dose of the binding protein is about 300 μg, about 310 μg, about 330 μg, about 340 μg, about 350 μg, about 360 μg, about 370 μg, about 380 μg, about 390 μg, about 400 μg, about 450 μg, about 500 μg, about 550 μg, about 600 μg, about 650 μg, about 700 μg, about 750 μg, about 800 μg, about 850 μg, about 900 μg, about 950 μg, about 1000 μg, about 1050 μg, about 1100 μg, about 1150 μg, about 1200 μg, about 1250 μg, about 1300 μg, about 1350 μg, about 1400 μg, about 1450 μg, about 1500 μg, about 1550 μg, about 1600 μg, about 1650 μg, about 1700 μg, about 1750 μg, about 1800 μg, about 1850 μg, about 1900 μg, about 1950 μg, about 2000 μg, about 2050 μg, about 2100 μg, about 2150 μg, about 2200 μg, about 2300 μg, about 2400 μg, about 2500 μg, about 2600 μg, about 2700 μg, about 2800 μg, about 2900 μg, about 3000 μg, about 3100 μg, about 3200 μg, about 3300 μg, about 3400 μg, about 3500 μg, about 3600 μg, about 3700 μg, about 3800 μg, about 3900 μg, about 4000 μg, about 4100 μg, or about 4200 μg.

In some embodiments, each dose of the binding protein is 300 μg, 310 μg, 330 μg, 340 μg, 350 μg, 360 μg, 370 μg, 380 μg, 390 μg, 400 μg, 450 μg, 500 μg, 550 μg, 600 μg, 650 μg, 700 μg, 750 μg, 800 μg, 850 μg, 900 μg, 950 μg, 1000 μg, 1050 μg, 1100 μg, 1150 μg, 1200 μg, 1250 μg, 1300 μg, 1350 μg, 1400 μg, 1450 μg, 1500 μg, 1550 μg, 1600 μg, 1650 μg, 1700 μg, 1750 μg, 1800 μg, 1850 μg, 1900 μg, 1950 μg, 2000 μg, 2050 μg, 2100 μg, 2150 μg, 2200 μg, 2300 μg, 2400 μg, 2500 μg, 2600 μg, 2700 μg, 2800 μg, 2900 μg, 3000 μg, 3100 μg, 3200 μg, 3300 μg, 3400 μg, 3500 μg, 3600 μg, 3700 μg, 3800 μg, 3900 μg, 4000 μg, 4100 μg, or 4200 μg.

In some embodiments, each dose of the binding protein is about 360 μg, about 1100 μg, about 2200 μg, about 3300 μg, or about 4200 μg. In some embodiments, each dose of the binding protein is 360 μg, 1100 μg, 2200 μg, 3300 μg, or 4200 μg.

In some embodiments, the binding protein is administered to the subject during a lead-in phase prior to initiation of weekly administration of the binding protein, and wherein the lead-in phase comprises administration of the binding protein in an escalating treatment regimen. In some embodiments, the lead-in phase is 8 days, wherein each dose of the binding protein in the escalating treatment regimen is between about 10 μg to about 600 μg. In some embodiments, each dose of the binding protein in the escalating treatment regimen is between 10 μg to 600 μg.

In some embodiments, the escalating treatment regimen comprises administration of the binding protein to the subject at the sequential doses about 20 μg, about 60 μg, and about 180 μg. In some embodiments, the escalating treatment regimen comprises administration of the binding protein to the subject at the sequential doses about 60 μg, about 180 μg, and about 500 μg. In some embodiments, the escalating treatment regimen comprises administration of the binding protein to the subject at the sequential doses 20 μg, 60 μg, and 180 μg. In some embodiments, the escalating treatment regimen comprises administration of the binding protein to the subject at the sequential doses 60 μg, 180 μg, and 500 μg.

In some embodiments, the escalating treatment regimen comprises administration of the binding protein to the subject at the sequential doses about 20 μg, about 60 μg, and about 180 μg on days 1, 4, and 8 of the lead-in phase, respectively. In some embodiments, the escalating treatment regimen comprises administration of the binding protein to the subject at the sequential doses about 60 μg, about 180 μg, and about 500 μg on days 1, 4, and 8 of the lead-in phase, respectively.

In some embodiments, the escalating treatment regimen comprises administration of the binding protein to the subject at the sequential doses 20 μg, 60 μg, and 180 μg on days 1, 4, and 8 of the lead-in phase, respectively. In some embodiments, the escalating treatment regimen comprises administration of the binding protein to the subject at the sequential doses 60 μg, 180 μg, and 500 μg on days 1, 4, and 8 of the lead-in phase, respectively.

In some embodiments, the binding protein is administered to the subject subcutaneously. Any suitable method for subcutaneous administration can be implemented for administration of the binding provided herein. In some embodiments, subcutaneous administration comprises injection of the binding protein provided herein using a needle and syringe under the skin (e.g., the fatty tissue layer between the skin and the muscle). Exemplary injection sites include, but are not limited to, abdomen, arm, or thigh.

In some embodiments, the binding protein is administered to the subject subcutaneously and with a lead-in phase. In some embodiments, prior to initiation of the subcutaneous lead-in phase, the binding protein is administered to the subject intravenously in an additional escalating treatment regimen. In some embodiments, the additional escalating treatment regimen is administered to the subject within a 28-day period, wherein each dose of the binding protein in the additional escalating treatment regimen is between about 0.2 μg to about 1500 μg. In some embodiments, each dose of the binding protein in the additional escalating treatment regimen is between 0.2 μg to 1500 μg.

In some embodiments, administration of the binding protein does not comprise a lead-in phase.

In some embodiments, the binding protein is administered until disease progression.

In some embodiments, the cycling regimen or lead-in phase is interrupted for one or more times. In some embodiments, the cycling regimen is interrupted or modified if the subject develops one or more adverse event, dose-limiting toxicity (DLT), neutropenia or febrile neutropenia, thrombocytopenia, cytokine release syndrome (CRS), neurotoxicity (NT), etc. In some embodiments, the dose of the binding protein for each administration is altered after the subject develops one or more adverse event, dose-limiting toxicity (DLT), neutropenia or febrile neutropenia, thrombocytopenia, cytokine release syndrome (CRS), neurotoxicity (NT), etc.

In some embodiments, the pharmacokinetics (e.g., elimination half-life (ti/2)) of the binding protein are assessed after administration of the binding protein to the subject. Elimination half-life (ti/2) comprises the length of time required for the concentration of the binding protein of the present disclosure to decrease to half of its starting dose in the body (e.g., in the blood, serum, or plasma) after administration to the subject. The elimination half-life of the binding protein provided herein in the blood (e.g., serum) of an subject is determined using any method known in the art, such as immunoassays, immunoblots, and mass spectrometry. In certain embodiments, the half-life of the binding protein of the disclosure in the blood (e.g., serum) of an individual is determined using an ELISA assay.

### B. Pharmaceutical Compositions

In some embodiments, the binding protein is administered to the subject as a therapeutic or pharmaceutical composition. Such therapeutic or pharmaceutical compositions can comprise about 0.2 μg to about 4500 μg of the binding protein, or binding protein-drug conjugate, in admixture with a pharmaceutically or physiologically acceptable formulation agent selected for suitability with the mode of administration (e.g., intravenously or subcutaneously).

Acceptable formulation materials are nontoxic to recipients at the dosages and concentrations employed.

The pharmaceutical composition can contain formulation materials for modifying, maintaining, or preserving, for example, the pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton; tromethamine; lecithin; cholesterol or tyloxapal), stability enhancing agents (such as sucrose or sorbitol), tonicity enhancing agents (such as alkali metal halides—e.g., sodium or potassium chloride—or mannitol sorbitol), delivery vehicles, diluents, excipients and/or pharmaceutical adjuvants (see, e.g., REMINGTON'S PHARMACEUTICAL SCIENCES (18th Ed., A. R. Gennaro, ed., Mack Publishing Company 1990), and subsequent editions of the same, incorporated herein by reference for any purpose).

The optimal pharmaceutical composition will be determined by a skilled artisan depending upon, for example, the intended route of administration, delivery format, and desired dosage. Such compositions can influence the physical state, stability, rate of in vivo release, and rate of in vivo clearance of the binding protein.

The primary vehicle or carrier in a pharmaceutical composition can be either aqueous or non-aqueous in nature. For example, a suitable vehicle or carrier for injection can be water, physiological saline solution, or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which can further include sorbitol or a suitable substitute. In one embodiment of the disclosure, binding protein compositions can be prepared for storage by mixing the selected composition having the desired degree of purity with optional formulation agents in the form of a lyophilized cake or an aqueous solution. Further, the binding protein can be formulated as a lyophilizate using appropriate excipients such as sucrose.

The pharmaceutical compositions of the disclosure can be selected for parenteral delivery (e.g., intravenous or subcutaneous). The preparation of such pharmaceutically acceptable compositions is within the skill of the art.

The formulation components are present in concentrations that are acceptable to the site of administration. For example, buffers are used to maintain the composition at physiological pH or at a slightly lower pH, typically within a pH range of from about 5 to about 8.

When parenteral administration is contemplated, the therapeutic compositions for use can be in the form of a pyrogen-free, parenterally acceptable, aqueous solution comprising the desired binding protein in a pharmaceutically acceptable vehicle. A particularly suitable vehicle for parenteral injection is sterile distilled water in which a binding protein is formulated as a sterile, isotonic solution, properly preserved. Yet another preparation can involve the formulation of the desired molecule with an agent, such as injectable microspheres, bio-erodible particles, polymeric compounds (such as polylactic acid or polyglycolic acid), beads, or liposomes, that provides for the controlled or sustained release of the product which can then be delivered via a depot injection. Hyaluronic acid can also be used, and this can have the effect of promoting sustained duration in the circulation. Other suitable means for the introduction of the desired molecule include implantable drug delivery devices.

Additional pharmaceutical compositions of the disclosure will be evident to those skilled in the art, including formulations involving binding proteins in sustained- or controlled-delivery formulations. Techniques for formulating a variety of other sustained- or controlled-delivery means, such as liposome carriers, bio-erodible microparticles or porous beads and depot injections, are also known to those skilled in the art. Additional examples of sustained-release preparations include semipermeable polymer matrices in the form of shaped articles, e.g. films, or microcapsules. Sustained release matrices can include polyesters, hydrogels, polylactides, copolymers of L-glutamic acid and gamma ethyl-L-glutamate, poly(2-hydroxyethyl-methacrylate), ethylene vinyl acetate, or poly-D(−)-3-hydroxybutyric acid. Sustained-release compositions can also include liposomes, which can be prepared by any of several methods known in the art.

Pharmaceutical compositions to be used for in vivo administration typically must be sterile. This can be accomplished by filtration through sterile filtration membranes. Where the composition is lyophilized, sterilization using this method can be conducted either prior to, or following, lyophilization and reconstitution. The composition for parenteral administration can be stored in lyophilized form or in a solution. In addition, parenteral compositions generally are placed into a container having a sterile access port, for example, an intravenous solution bag or vial having a stopper pierceable by a hypodermic injection needle.

Once the pharmaceutical composition has been formulated, it can be stored in sterile vials as a solution, suspension, gel, emulsion, solid, or as a dehydrated or lyophilized powder. Such formulations can be stored either in a ready-to-use form or in a form (e.g., lyophilized) requiring reconstitution prior to administration.

In some embodiments, a kit is implemented for producing a single-dose administration unit. The kits can each contain both a first container having a dried protein and a second container having an aqueous formulation. In some embodiments, the kit contains single and multi-chambered pre-filled syringes (e.g., liquid syringes and lyosyringes).

The route of administration of the pharmaceutical composition is in accord with known methods, e.g., through injection by intravenous, subcutaneous; intraperitoneal, intracerebral (intraparenchymal), intracerebroventricular, intramuscular, intraocular, intraarterial, intraportal, or intralesional routes; by sustained release systems; or by implantation devices. Where desired, the compositions can be administered by bolus injection or continuously by infusion, or by implantation device.

The composition can also be administered locally via implantation of a membrane, sponge, or other appropriate material onto which the desired molecule has been absorbed or encapsulated. Where an implantation device is used, the device can be implanted into any suitable tissue or organ, and delivery of the desired molecule can be via diffusion, timed-release bolus, or continuous administration.

The pharmaceutical compositions can be used to prevent and/or treat HER2+ cancers. The pharmaceutical compositions can be used as a standalone therapy or in combination with standard anti-cancer therapy.

In some embodiments, the binding protein is provided in a kit comprising the binding protein. In some embodiments, the kit comprises other reagents such as a detectable label, blocking serum, positive and negative control samples, and detection reagents. In some embodiments, the kit comprises a composition comprising any binding protein described herein. In some embodiments, the kit comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing a cancer and may have a sterile access port (for example the container may be an intravenous solution bag or a vial having a stopper pierceable by a hypodermic injection needle). In some embodiments, the label or package insert indicates that the composition is used for preventing, diagnosing, and/or treating the cancer. Alternatively, or additionally, the article of manufacture or kit may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EXAMPLES

The presently disclosed subject matter will be better understood by reference to the following Examples, which are provided as exemplary of the invention, and not by way of limitation.

### Example 1: Phase 1/1b Open-Label Study of a HER2-Targeted T-Cell Engager (TCE) Binding Protein in Patients with Advanced Solid Tumors

### Objectives and Endpoints

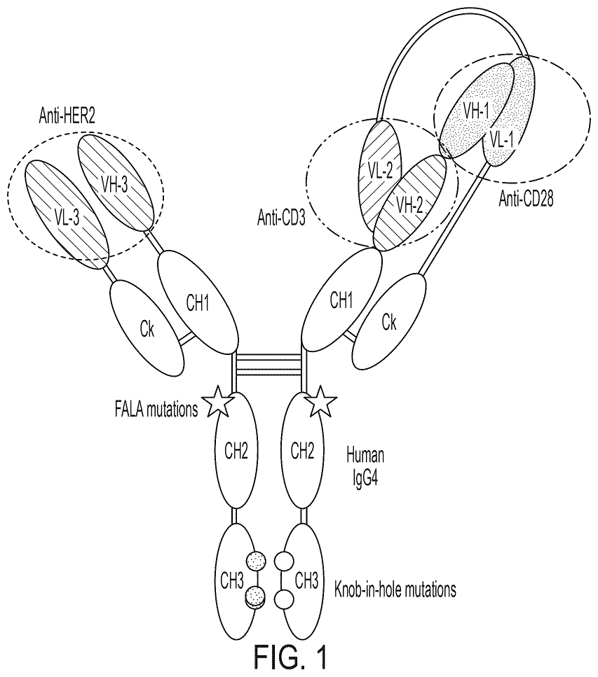

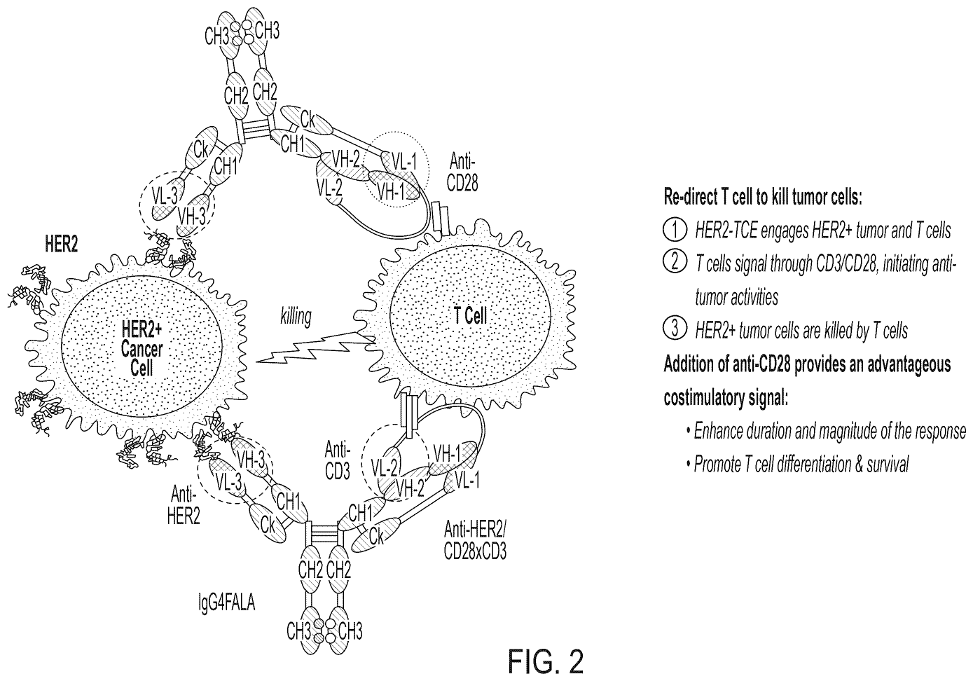

The binding proteins provided herein are trispecific antibodies capable of redirecting T cells to kill human epidermal growth factor receptor 2 (HER2)-expressing tumors, irrespective of T cell antigen receptor specificity. The binding proteins provided herein function by co-engaging tumor cells via HER2 and T cells via CD3 and CD28, resulting in T cell activation and cytolysis of the tumors (FIG. 2). HER2 is a tyrosine kinase receptor that is overexpressed in multiple cancers, including breast, gastric, lung, gastroesophageal, ovarian, bladder, colon, and others. It is a clinically validated target for cancer treatment, with currently approved HER2-targeting antibodies and small molecule inhibitors of its kinase activity. As a T cell engager (TCE), the binding proteins provided herein differentiate from available HER2 therapeutics by leveraging T cells of the adaptive immunity to eradicate HER2-expressing cancers, harnessing the power of the immune system. The aim of this first-in-human single agent study (ClinicalTrials.gov Identifier: NCT05013554) was to evaluate the safety, the maximum tolerated dose (MTD), the pharmacokinetics (PK) as well as recommended dose (s) (RD(s)), biomarker data, and preliminary clinical efficacy of Binding Protein #2 (corresponding polypeptide sequences are shown in Table 4, FIG. 1) in participants with solid tumors.

TABLE 5
Objectives and Endpoints

| Objectives | Endpoints |
|---|---|
| Primary | |
| Part 1 (Dose Escalation) | |
| To determine the MTD/maximum administered | Incidence of dose-limiting toxicities (DLTs) during |
| dose (MAD) of Binding Protein #2 administered | DLT observation period |
| as a single agent in participants with HER2 | Incidence of treatment-emergent adverse events |
| expressing solid tumors and determine the RD(s) | (TEAEs), serious adverse events (SAEs), and lab |
| for intravenous (IV) and subcutaneous (SC) | abnormalities according to the National Cancer |
| administration in the dose escalation part. | Institute (NCI) Common Terminology Criteria for |
| To determine the safety of Binding Protein #2 after | Adverse Events (CTCAE) Version 5.0 |
| IV and SC administration. | |
| Part 2 (Dose expansion) | |
| To assess preliminary clinical activity of single | Objective response rate (ORR) and duration of |
| agent Binding Protein #2 at the RD(s) in | response (DoR) of Binding Protein #2 in all |
| participants with HER2 expressing solid tumors, | participants. ORR of Binding Protein #2 treatment |
| with various levels of HER2 expression. | will be based on Response Evaluation Criteria in |
| | Solid Tumors (RECIST v1.1) |
| Secondary | |
| (Part 1) | |
| To assess preliminary clinical activity of single | Objective response rate (ORR) and DoR of Binding |
| agent Binding Protein #2 after IV and SC | Protein #2 in all participants. ORR of Binding |
| administration at the RD(s) in participants with | Protein #2 treatment was based on RECIST v1.1. |
| HER2 expressing solid tumors, with various levels | |
| of HER2 expression. | |
| (Part 2) | |
| To determine the safety of Binding Protein #2. | Incidence of TEAEs, SAEs, and lab abnormalities |
| | according to NCI CTCAE Version 5.0. |
| (Part 1 and Part 2) | |
| To characterize the PK profile of Binding Protein | Plasma concentrations and PK parameters (Cₘₐₓ, |
| #2 when administered as a single agent after IV and | C_{trough}, t_{1/2}, AUC_{0-τ}) of Binding Protein #2 |
| SC (Part 1 only) administration. | Incidence of antidrug antibodies (ADAs) against |
| To evaluate the potential immunogenicity of | Binding Protein #2 |
| Binding Protein #2 after IV and SC administration. | Progression free survival (PFS) of Binding Protein |
| To assess preliminary clinical activity of single | #2 in all participants based on RECIST v1.1 |
| agent Binding Protein #2 at the RD(s) in | |
| participants with HER2 expressing solid tumors, | |
| with various levels of HER2 expression. | |
| Tertiary | |
| (Part 1 and Part 2) | |
| To monitor immune cell activation, expansion, and | Assess changes from baseline and over time |
| kinetics in blood induced by Binding Protein #2 | following administration of Binding Protein #2 in T |
| To document the modulation of immune response | cell activation, T cell, and NK cell expansion (Ki67) |
| in the tumor microenvironment (TME). | in blood. |
| To monitor kinetics of cytokine production in blood | Programmed cell death-ligand 1 (PD-L1), |
| To evaluate HER2 expression in tumors (biopsies, | programmed cell death 1 (PD-1), CD8+/Ki67, |
| ctDNA, and soluble HER2). | CD3+, CD4+, FoxP3, Granzyme B, and |
| To explore the association between biomarkers and | T/NK/Treg/Myeloid cell abundance and activation |
| response | biomarkers in baseline, on treatment, and end of |
| | treatment (EOT) tumor tissue samples |
| | Assess change in cytokine levels following |
| | treatment administration and their relationship with |
| | exposure of Binding Protein #2 |
| | Evaluate HER2 expression and genomic aberration |
| | at baseline and modulation in tumors (biopsies) and |
| | in blood (ctDNA) over time with Binding Protein |
| | #2 administration |
| | Assess soluble HER2 at baseline and modulation |
| | over time with Binding Protein #2 administration |
| | HER expression in tumors, soluble HER2 at |
| | baseline and modulation over time, PD-L1, immune |
| | gene signatures, circulating tumor DNA in baseline |
| | samples (for both dose escalation and expansion) |
| | and modulation of genomic aberrations (including |
| | HER2 copy number variation and HER2 |
| | mutations). |

### Overall Design

This study was a Phase 1/1b open-label, first-in-human, multicenter, open-label, non-randomized, single agent study to evaluate the safety, pharmacokinetics, pharmacodynamics, and signs of clinical activity of Binding Protein #2 administered intravenously (IV) or subcutaneously (SC) in participants with solid tumors. The study included 2 parts: 1) a dose escalation part for safety and dose findings and 2) a dose expansion part for the assessment of safety and efficacy at RD(s) in various cohorts defined by HER2 expression and/or HER2 activating mutation.

The dose escalation was conducted in participants with advanced relapsed or refractory solid tumors that express HER2 as defined by an in situ validated HER2 diagnostic test or with detected HER2 activating mutations in tissue or blood using a validated test, and who have exhausted all standard of care therapy. Tumors eligible for the dose escalation of this study were characterized as immunohistochemistry (IHC) 3+, 2+, 1+ and/or carrying HER2 genomic aberration including amplification and activating mutations. The dose escalation part was to evaluate the safety, including potential dose-limiting toxicities (DLTs) and the maximum tolerated doses (MTDs) of Binding Protein #2, and was to determine the recommended dose (s) (RD(s) for IV and SC administration route). The goal of dose expansion was for the assessment of efficacy and safety at RD(s) in various cohorts defined by HER2 expression and/or genomic aberrations.

First week of Cycle 1 (lead-in phase) was bi-weekly administration on Day 1 and Day 4; subsequent weeks and subsequent cycles were weekly administration. Cycle length in this study was 4 weeks (28 days).

In the dose expansion part, participants with advanced solid tumors were enrolled in four cohorts. The cohorts were as follows:
Cohort A: Participants with metastatic breast cancers with HER2 IHC 3+ or 2+ (with HER2 amplification).
Cohort B: Participants with metastatic breast cancers with HER2 IHC 1+ or 2+ (without HER2 amplification) or HER2 activating mutation.
Cohort C: Participants with metastatic gastric cancers with HER2 IHC 1+ or 2+ (without HER2 amplification) or HER2 activating mutation.
Cohort D: Participants with metastatic non-small cell lung cancer (NSCLC) with HER2 IHC 1+, 2+, 3+ and/or HER2 amplification and/or HER2 activating mutation.

### Dosing Regimen

### Lead-In Dose and Dose Escalation

To minimize the risk of severe CRS, an intra-participant dose escalation (lead-in part) in Cycle 1 for each dose levels (DL1 to DL7) of the dose escalation part was proposed. Also, to allow slow increase while reaching more rapidly doses associated with pharmacological activity, a second dosing at Day 4 of Week 1 was planned. For subsequent weeks, weekly dosing was planned for all dose levels. The lead-in dose escalation did not exceed half-log increase (about 3-fold) during the first four dose levels (DL1 to DL4). More than 3-fold increase was planned for subsequent dose levels once the tolerability was confirmed at previous dose level. The dose escalation schema is shown in Table 6 and Table 7. The target dose was first administered on Cycle 1 Day 15 or for extended lead-in cohort on Cycle 1 Day 22. DLT observation period was completed after two consecutive target doses. Information of ongoing cohort was analyzed at the Study Committee review to finally decide on the next dose levels. Together with safety monitoring, pharmacokinetic and pharmacodynamic data were collected that was used to inform and to adapt the choice of subsequent dose levels for the Dose Escalation Part. There were at least 7 days between the first participant and the second participant enrolled and treated in each dose level. An optimal lead-in dosing was then proposed for the dose expansion part based on the data obtained in dose escalation part. If no DLT was observed, MAD was considered as MTD.

After Cycle 1, each participant received a fixed dose until the end of treatment, corresponding to the maximum weekly dose that they achieved in Cycle 1, unless the dose needed to be modified for safety reasons. For the Expansion Part, the final dosing scheme was defined based on the totality of the information available at all dose levels in dose escalation part without exceeding the maximum first applied dose, nor the maximum treatment dose.

Binding Protein #2 was intended to be administered by intravenous (IV) infusion with targeted total infusion duration of about 4 hours or by subcutaneous (SC) injection until 2 target doses were completed, and faster at the following cycles. The IV infusion rate could be increased progressively if previous administrations were well tolerated. For the subcutaneous (SC) injection, a maximum of 2 ml was administered in the abdominal area away from the belly button, in thighs, or upper arms. At higher SC dose levels (higher than 4000 μg), a second injection was sometimes needed, and was administered at a second site in the abdomen, thighs or upper arms.

### Flat Dosing Strategy

Studies have demonstrated that for most monoclonal antibodies, fixed doses generated similar exposures in participants when compared to body weight/body surface area-based doses due to interparticipant variability and non-linear relationship between body weight and blood volume (Wang D D et al. Fixed dosing versus body size-based dosing of monoclonal antibodies in adult clinical trials. J Clin Pharmacol. 2009; 49(9):1012-24; Feldschuh J et al. Prediction of the normal blood volume. Relation of blood volume to body habitus. Circulation. 1977; 56(4 Pt 1):605-12; and Lemmens H J M et al. Estimating blood volume in obese and morbidly obese patients. Obes Surg. 2006; 16(6):773-6). Binding Protein #2 was predicted to be essentially distributed in central compartment (blood). Thus, body weight was expected to have limited impact on its plasma exposure. Therefore, to follow approaches used by other TCEs (e.g., BLINCYTO) a flat dose strategy for Binding Protein #2 was used in this protocol. At first, only subjects≥45 kg and ≤150 kg were enrolled and dosed with Binding Protein #2. Ultimately, this strategy was thought to limit error of dose preparation and mitigate safety risk related to that.

Dose levels for Cycle 1 IV are provided in Table 6.

TABLE 6
Flat dosing scheme for dose escalation IV administration

| Dose level | | | | | | | | | | |
|---|---|---|---|---|---|---|---|---|---|---|
| (nominal dose | | Cycle 1 (lead-in phase) | | | | | Cycle 2 and beyond | | | |
| and total | | W 1 | | W 2 | W 3 | W 4 | W 1 | W 2 | W 3 | W 4 |
| dose) | | D 1 | D 4 | D 8 | D 15 | D 22 | D 1 | D 8 | D 15 | D 22 |
| DL1 | μg | 0.6 | 1.8 | 6.0 | 18 | 18 | 18 | 18 | 18 | 18 |
| DL2 | μg | 1.8 | 6.0 | 18 | 60 | 60 | 60 | 60 | 60 | 60 |
| DL3 | μg | 6.0 | 18 | 60 | 180 | 180 | 180 | 180 | 180 | 180 |
| DL3b | μg | 9.0 | 27 | 90 | 240 | 240 | 240 | 240 | 240 | 240 |
| DL4 | μg | 18 | 60 | 180 | 360 | 360 | 360 | 360 | 360 | 360 |
| DL5 | μg | 18 | 60 | 180 | 720 | 720 | 720 | 720 | 720 | 720 |
| DL6 | μg | 18 | 60 | 180 | 1080 | 1080 | 1080 | 1080 | 1080 | 1080 |
| DL7 | μg | 18 | 60 | 180 | 1440 | 1440 | 1440 | 1440 | 1440 | 1440 |

Abbreviations: D = Day, DL = dosing level, W = week.

TABLE 7
Flat dosing scheme for dose escalation IV administration with a 3-week lead-in

| Dose level | | Cycle 1 (lead-in phase)^{a} | | | | | Cycle 2 and beyond | | | |
|---|---|---|---|---|---|---|---|---|---|---|
| (nominal dose | | W 1 | | W 2 | W 3 | W 4 | W 1 | W 2 | W 3 | W 4 |
| and total dose) | | D 1 | D 4 | D 8 | D 15 | D 22 | D 1 | D 8 | D 15 | D 22 |
| DL1ext | μg | 18 | 60 | 120 | 240 | 480 | 480 | 480 | 480 | 480 |
| DL2ext | μg | 18 | 60 | 120 | 240 | 720 | 720 | 720 | 720 | 720 |
| DL3ext | μg | 18 | 60 | 150 | 300 | 900 | 900 | 900 | 900 | 900 |
| DL4ext | μg | 18 | 60 | 150 | 300 | 1125 | 1125 | 1125 | 1125 | 1125 |

Abbreviations: D = Day, DL = dosing level, W = week.
^{a}Lead-in-phase correspond to all dosing up to administration of the two consecutive target doses.
With 3-week lead-in schedule DLT period extends to 1 week from the C2D 1 administration.

Participants with low body weight (≤50 kg), in case of weight loss more than 1000 of their starting weight during the conduct of the study were considered on a case by case after the evaluation of the safety by both the Study Investigator and Study Medical Monitor.

### Subcutaneous Administration at Selected Dose Levels

To safeguard participant's safety, the SC starting dose level was cleared previously in IV escalation. This was considered a safe approach knowing that doses applied were already shown to be tolerated, and that the SC bioavailability anticipated to be limited based on the preclinical data further limited the actual exposure. On the other hand, based on the preclinical data in monkeys showing reduced bioavailability, SC dosing was required to start at dose levels which allowed to measure plasma concentrations and derive key PK parameters (ideally with a dose≥180 Ng).

After the first SC DL (DL1sc) completed, the following SC DL applied doses adjusted to achieve when possible similar plasma exposure as the leading safe DL of IV escalation, but without increasing SC doses by more than half-log (about 3-fold). The initial adjustment was done based on the bioavailability derived from DL1sc. This was calculated from at least 3 subjects using PK data collected up to at least Cycle 1—Day 22 compared to the exposure obtained after IV administration.

In this initial investigation of the SC route, the dosing regimen remained the same to that of the IV dosing (weekly dosing after Cycle 1 Day 8 (C1D8)). The dose escalation decisions during SC dose escalation were determined by the Study Committee based on evaluation of DLTs, the modified continual reassessment method (mCRM) recommendation, and other available relevant information.

TABLE 8
Flat dosing scheme for dose escalation SC administration
Following IV administration and assuming bioavailability of 30% ^{a, b, c}

| | | Lead-in doses | | | Maintenance dose |
|---|---|---|---|---|---|
| | | C1D1 | C1D4 | C1D8 | C1D15 onward |
| DL1sc | μg | 20 | 60 | 180 | 360 |
| DL2sc | μg | 60 | 180 | 500 | 1100 |
| DL3sc | μg | 60 | 180 | 500 | 2200 |
| DL4sc | μg | 60 | 180 | 500 | 3300 |
| DL5sc | μg | 60 | 180 | 500 | 4200 |

Abbreviations: D = Day, DLsc = subcutaneous dosing level, C = cycle.
^{a} If different IV doses were applied during conduct of the escalation, then SC doses were adapted accordingly. DL1sc doses were meant to be similar to last IV doses deemed safe. In the example, DL1sc = DL4 IV. DL1sc started with doses of 10 μg (C1D1), 27 μg (C1D4), 90 μg (C1D8) and 240 μg (C1D15 onwards) after DL3b IV cohort was deemed tolerable.
^{b} SC doses after DL1sc were adapted according to observed initial estimation of the SC bioavailability and plasma exposure observed after IV dosing. Proposed DL2sc to DL5sc doses were based on a potential SC bioavailability of approximately 30%. Further adaptation of DL3sc, DL4sc and DL5sc doses are possible in case of a lower bioavailability and if safety allowed. Maximum dose increase between in SC DLs did not exceed half-log (about 3-fold).
^{c} For SC application, doses were rounded according to anticipated volume of administration.

### Alternative Dose Levels for Dose Escalation

The following rules were applied in the specific situations described below (applicable to both IV and SC dose escalation).

DL(−)1 was initiated if any participant experienced DLTs at DL1 including Grade≥3 cytokine release syndrome (CRS) (per the NCI Consensus Guidelines) or neurotoxicity which precluded further dose escalation. DL (−)1 was 50% of the doses, including both lead-in and target doses, at DL1.

A maximum of 100% increment including both lead-in and target doses of the previous dose level was initiated instead of the planned next DL if participants of a given DL experienced Grade≥2 treatment-related adverse events (including but not limited to cytokine release syndrome and neurotoxicity) during the DLT period that could not resolve to Grade 1 or baseline within 7 days.

Grade 2 adverse events (AEs) that did not lead to subsequent dose modification, unless judged appropriate by the Investigators, included:
Grade 2 fatigue or asthenia.
Grade 2 nausea, vomiting or diarrhea if successfully managed with optimal medical support.
Grade≥2 isolated electrolyte abnormalities (i.e., those occurring without clinical consequences).

A maximum of 50% increment of the doses of the previous dose level, including both lead-in and target doses, was initiated instead of the planned next DL if participants of a given DL experienced two or more Grade≥2 treatment-related adverse events (including but not limited to cytokine release syndrome and neurotoxicity) during the DLT period that could not resolve to Grade 1 or baseline within 7 days. Grade 2 AEs that did not lead to subsequent dose modification, unless judged appropriate by the Investigators, included:
Grade 2 fatigue or asthenia.
Grade 2 nausea, vomiting or diarrhea if successfully managed with optimal medical support.
Grade≥2 isolated electrolyte abnormalities (i.e., those occurring without clinical consequences).

A maximum of 50% increment of the doses of the previous dose level, including both lead-in and target doses, was initiated instead of the planned next DL if participants of a given cohort experienced one DLT during the DLT period.

A maximum of 30% increment of the doses of the previous dose level, including both lead-in and target doses, was initiated instead of the planned next DL if participants of a given DL experienced two DLTs during the DLT period.

### Study Population

### A. Inclusion Criteria

Participants were eligible to be included in the study only if all of the following criteria applied:

### Age:

Participant was 18 or the legal age of consent in the jurisdiction in which the study was taking place, at the time of signing the informed consent.

### Type of Participant and Disease Characteristics:

Eastern Cooperative Oncology Group (ECOG) performance status 0-1.
All participants had at least 1 measurable disease per the Response Evaluation Criteria in Solid Tumors (RECIST v1.1). An irradiated lesion was considered measurable only if progression had been demonstrated on the irradiated lesion.
Cancer Diagnosis Dose Escalation:
   A) Participant had histologically or cytologically confirmed diagnosis of metastatic solid tumors who had exhausted all locally available and clinically appropriate standard of care therapies or for which no standard treatment was available, and,
   B) With HER2 expression in tumor tissue (IHC 3+, IHC 2+ or IHC 1+) and/or with HER2 activating mutations detected in tumor or blood by means of validated assay (s).
Cancer Diagnosis for Dose Expansion Cohort A:
   A) Participants had a histologically or cytologically confirmed diagnosis of metastatic Breast Cancers who had exhausted all locally available and clinically appropriate standard of care therapies, or for which no standard treatment was available, and,
   B) With HER2 expression in tumor tissue (IHC 3+ or IHC 2+ with [HER2 amplification]) detected in blood or tumor with validated assay(s).
Cancer Diagnosis for Dose Expansion Cohort B:
   A) Participants had a histologically or cytologically confirmed diagnosis of metastatic of Breast Cancer who had exhausted all standard of care therapies, or for which no standard treatment was available, and,
   B) With HER2 expression in tumor tissue (IHC 1+ or IHC 2+[without HER2 amplification]) or HER2 activating mutations detected in blood or tumor with validated assay(s).
Cancer Diagnosis for Dose Expansion Cohort C:
   A) Participant had a histologically or cytologically confirmed diagnosis of metastatic Gastric Cancer who had exhausted all standard of care therapies, or for which no standard treatment was available, and,
   B) With HER2 expression in tumor tissue (IHC 1+ or 2+[without HER2 amplification]) or HER2 activating mutations detected in blood or tumor with validated assay(s).
Cancer Diagnosis for Dose Expansion Cohort D:
   A) Participant had histologically or cytologically confirmed diagnosis of metastatic Non-Small Cell Lung Cancer who has exhausted all standard of care therapies, or for which no standard treatment was available, and,
   B) With HER2 expression in tumor tissue (IHC 1+, 2+, 3+) and/or HER2 amplification and/or HER2 activating mutations detected in tumor or blood using validated assay(s).

### Weight:

Body weight within [45-150 kg](inclusive).

### Sex, Contraceptive Barrier Method and Pregnancy Testing Requirements:

All Contraceptive use by men and women were consistent with local regulations regarding the methods of contraception for those participating in clinical studies.
Male participants were eligible to participate if they agreed to the following during the intervention period and for at least 30 days after the last administration of study intervention:
   Refrain from donating sperm, plus either:
   A) Be abstinent from heterosexual intercourse as their preferred and usual lifestyle (abstinent on a long term and persistent basis) and agree to remain abstinent; OR
   B) Must agree to use contraception/barrier.
A female participant was eligible to participate if she was not pregnant or breastfeeding, and one of the following conditions applies:
   A) Is a woman of non-childbearing potential (WONCBP), OR
   B) Is a woman of childbearing potential (WOCBP) and agrees to use a contraceptive method that is highly effective, with a failure rate of <1%, during the study intervention period and for at least 90 days after the last administration of study intervention.
A WOCBP has a negative highly sensitive serum pregnancy test within 72 hours before the first administration of study intervention.

### Informed Consent

Participant was capable of giving signed informed consent.

### B. Exclusion Criteria

Participants were excluded from the study if any of the following criteria apply:

### Medical Conditions

Diagnosed or treated for another malignancy within 3 years prior to enrollment, except for basal cell carcinoma or squamous cell carcinoma of the skin, an in-situ malignancy, superficial bladder carcinoma, or low risk prostate cancer.
Primary central nervous system (CNS) tumors.
Known active brain metastases or leptomeningeal metastases. Participants with previously treated brain metastases were able to participate provided they were clinically stable for at least 4 weeks and, had no evidence of new or enlarging brain metastases, and also were off steroids at least 2 weeks prior to dosing with study medication. Participants with asymptomatic brain metastases (i.e., no neurological symptoms, with no requirements for corticosteroids, and no lesion>1.5 cm) were able to participate but required regular imaging (i.e., every 8 weeks) of the brain as a site of disease.
Any of the following cardiovascular abnormalities:
   Symptomatic congestive heart failure, New York Heart Association Class II or higher.
   Acute coronary syndrome (e.g., unstable angina, myocardial infarction), percutaneous coronary intervention (e.g., coronary artery stenting, or angioplasty) or coronary artery bypass graft (CABG) surgery within 6 months prior to study entry
   History of myocarditis within 6 months prior to study entry.
   History or current uncontrolled clinically significant unstable arrhythmias.
   Participants who have pacemakers to control atrial arrhythmias are candidates for the study. Participants with medically controlled atrial fibrillation>1 month prior to C1D1 were eligible.
   History of congenital long QT syndrome or prolonged QTcF interval>480 msec using Fridericia's formula (unless a pacemaker is in place) or uncorrectable abnormalities in blood electrolytes (sodium, potassium, calcium, magnesium, phosphorus).
   Left ventricular ejection fraction (LVEF)<50%.
   Increased troponin (higher than ULN per local laboratory) at screening.
   Increased pro-BNP (higher than ULN per local laboratory) at screening.
   Moderate to large pericardial effusion (e.g., >approximately 100 mL) as measured by echocardiogram at screening/baseline.
Uncontrolled or unresolved acute renal failure, or significant pulmonary conditions such as the following:
   Uncontrolled chronic lung disease.
   A known history (past 5 years) of, or any evidence of, interstitial lung disease.
   Active, non-infectious pneumonitis.
History of allogeneic or solid organ transplant.
Active, known or suspected autoimmune disease that had required systemic treatment in the past 2 years (i.e., with use of disease modifying agents, corticosteroids or immunosuppressive drugs), except for replacement therapy (e.g., thyroxine, insulin, or physiologic corticosteroid replacement).
Clinically uncontrolled chronic or ongoing infectious disease requiring treatment at the time of first dose or within the 14 days before first dose.
Known active hepatitis A, B, and C as defined below:
   A) Active hepatitis A defined as positive IgM.
   B) Known uncontrolled hepatitis B virus (HBV) infection:
      1. Anti-HBV therapy started before initiation of IMP and HBV viral load<2000 IU/mL (104 copies/mL) were eligible. The anti-HBV therapy continued throughout the treatment period.
      2. Positive anti-HBc, positive anti-HBs, negative HBsAg, and no HBV virus load detectable without HBV therapy were eligible.
   C) Known untreated current HCV infection:
      1. Anti-HCV therapy started before initiation of IMP were eligible. The anti-HCV therapy continued throughout the treatment period until seroconversion.
      2. Positive HCV antibody and undetectable HCV RNA without anti-HCV therapy were eligible.
Known positivity for Human Immunodeficiency Virus (HIV).
Unresolved toxicities from prior anticancer therapy or other treatment, defined as not having resolved to CTCAE Version 5.0 Grade 1 or to levels dictated in the eligibility criteria with the exception of Grade 1 peripheral neuropathy, alopecia or toxicities from prior anticancer therapy that are considered irreversible (defined as having been present and stable for >4 weeks) which were allowed if they were not otherwise described in the exclusion criteria.
Sensitivity to any of the study interventions, or components thereof, or drug or other allergy that, in the opinion of the Investigator, contraindicates participation in the study.
Known contraindication to any of the non-investigational medicinal products (NIMPs) dexamethasone and tocilizumab or alternative therapies per site practice in CRS management, acetaminophen or equivalent, diphenhydramine or equivalent, ranitidine or equivalent, montelukast or similar agents.

### Prior/Concomitant Therapy

Systemic radiation therapy<28 days and focal radiotherapy<14 days prior to the first dose administration of Binding Protein #2.
Major surgery, defined as surgery requiring general anesthesia with endotracheal intubation within 28 days prior to the first dose administration of Binding Protein #2, unless discussed with and eligibility approved by sponsor medical monitor.
Participant received any anticancer therapy including any investigational drug within 5 half-lives (of the drug) or within 21 days of the first dose of Binding Protein #2, whichever was shorter.
Participants on Vitamin K antagonists were excluded but low-molecular-weight heparin (LMWH) and direct Factor Xa inhibitors were allowed. Low dose aspirin was also allowed.
Receipt of a live-virus vaccination within 28 days of planned treatment start. Receipt of viral vaccine that do not contain live virus within 7 days.

### Prior/Concurrent Clinical Study Experience

Participation in a concurrent clinical study in the treatment period.

### Diagnostic Assessments

Participant had screening laboratory results including one of following:
   Absolute neutrophil count (ANC)<1000 cells/μL (1.0×10⁹/L) (Growth factor was not used within 7 days prior to the ANC test).
   Platelet count<100×10³ counts/μL (after at least 3 days without platelet transfusion).
   Hemoglobin<9 g/dL or <5.6 mmol/L (without transfusions within 2 weeks of initiation of IMP).
   Total bilirubin>1.5× upper limit of normal (ULN) (unless the subject had documented Gilbert syndrome in which case direct bilirubin was not >2×ULN).
   Aspartate aminotransferase (AST/SGOT) or Alanine aminotransferase (ALT/SGPT)>2.5×ULN.
   Blood creatinine>1.5×ULN or creatinine clearance of <50 mL/min (using Cockcroft Gault formula or 24-hour urine collection).

### Other Exclusions

Individuals accommodated in an institution because of regulatory or legal order; prisoners or participants who were legally institutionalized.
Any country-related specific regulation that prevented the participant from entering the study.
Participant not suitable for participation, whatever the reason, as judged by the Investigator, including medical or clinical conditions, or participants potentially at risk of noncompliance to study procedures.
Participants were employees of the clinical study site or other individuals directly involved in the conduct of the study, or immediate family members of such individuals.
Any specific situation during study implementation/course that were to raise ethics considerations.

### Results for IV Dose-Escalation Cohorts

Binding Protein #2 was administered intravenously every week with a 2-week lead-in (2 LI; DL: 18-720 μg) or a 3-week lead-in (3 LI; DL: 480-900 μg) phase prior to administration of weekly target doses in heavily pre-treated adult patients with HER2+ (HER2-expression [1+, 2+, or 3+ by immunohistochemistry] and/or HER2-activating mutations) metastatic solid tumors, mostly including breast, ovarian, and gastric cancers. Patient baseline characteristics are presented in Table 9. The primary endpoints were dose-limiting toxicities (DLTs), treatment emergent adverse events (TEAEs), serious adverse events, and lab abnormalities.

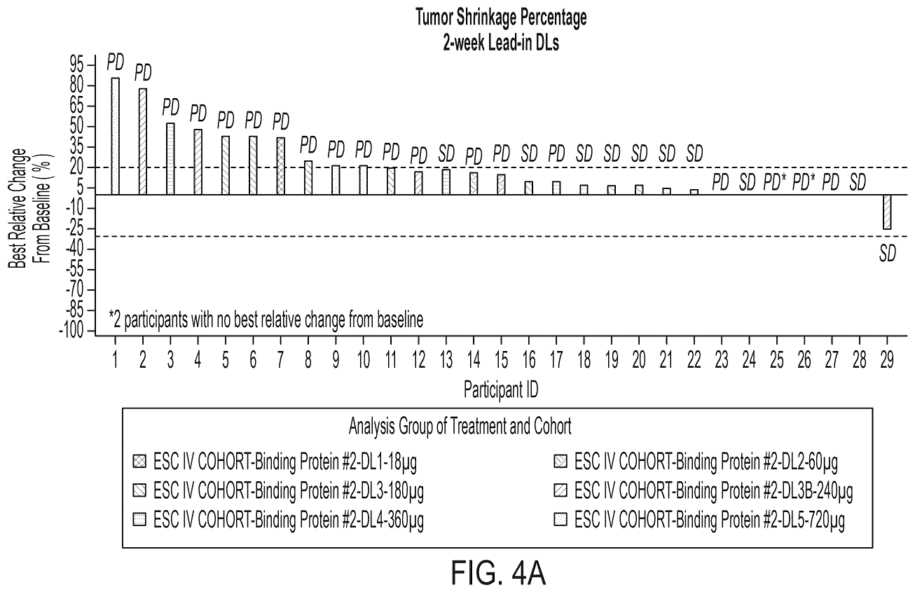

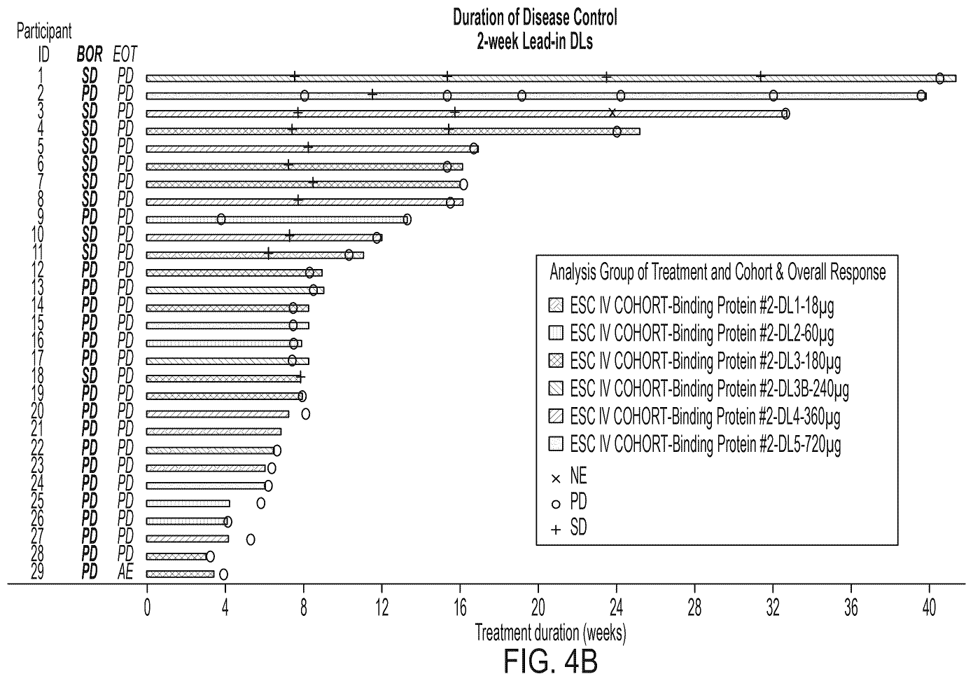

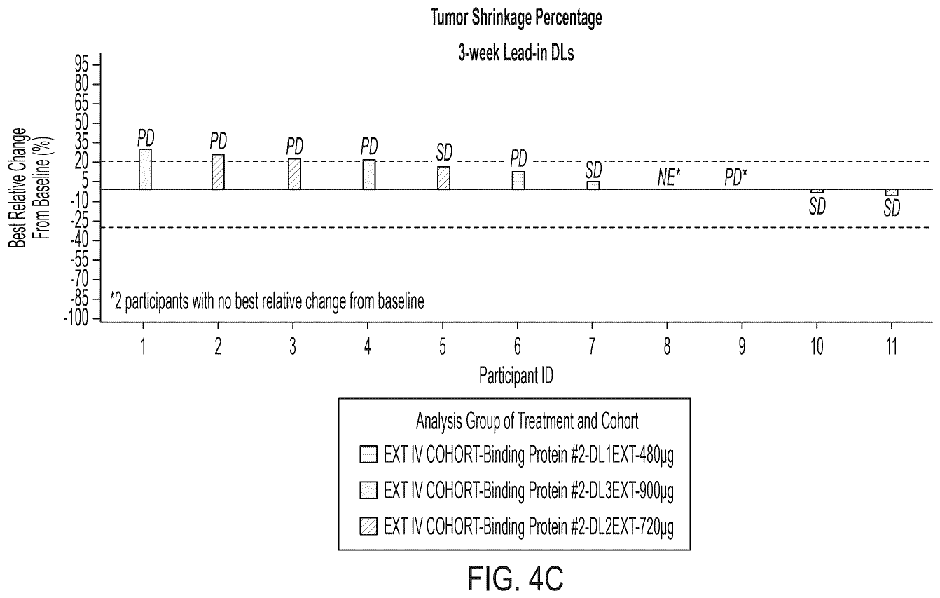

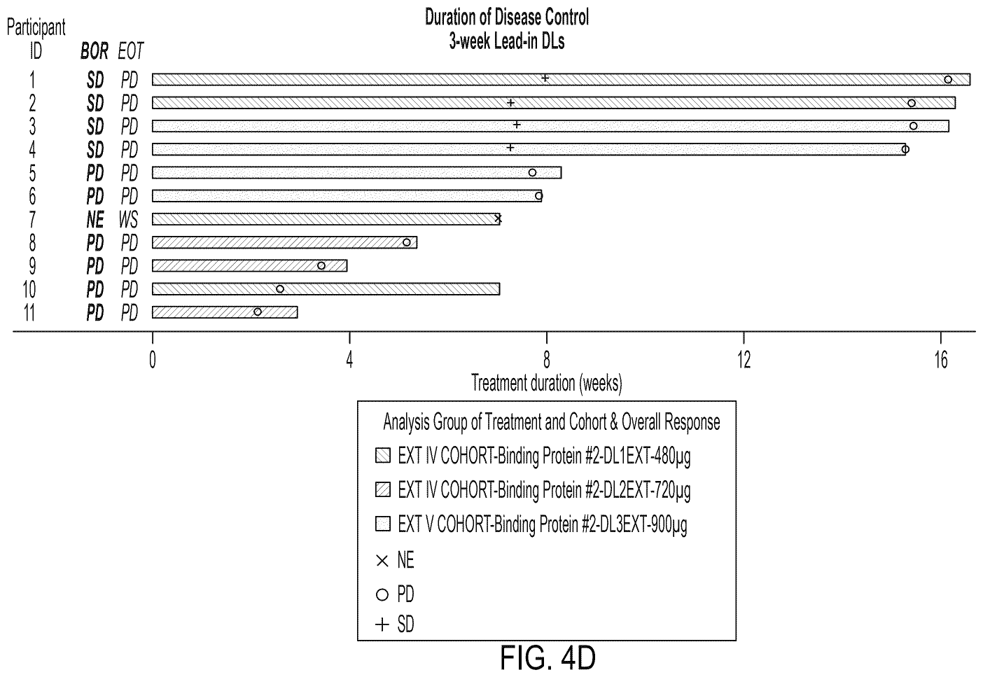

Forty patients received Binding Protein #2, with at least 3 patients in each DL. Three DLTs were observed in the 2 LI cohort (1 grade 3 cardiac failure at 180 μg and 2 maximum grade 4 alanine aminotransferase (ALT) elevations at 720 μg). No DLTs were reported in the IV 3-week lead-in group. Most frequent TEAEs in all DLs were cytokine release syndrome (CRS) (2 LI: 37.9%; 3 LI: 81.8%; all grade 1-2), ALT increase (2 LI: 34.5%; 3 LI: 27.3%), and pyrexia (2 LI: 31.0%; 3 LI: 45.5%; all grade 1-2) (Table 11). CRS events were grade 1-2, pyrexia events were grade 1-2, and ALT increase events were asymptomatic with maximum grade 4. No serious CRS was observed in the 3-week lead-in group. All CRS reactions were manageable, and patients were shown to have a quick recovery time with conventional treatment. Further, no grade 4 ALT or aspartate aminotransferase (AST) increase was observed in the 3-week lead-in group. Other than disease progression, no grade 5 TEAEs were observed. In the 2-week lead-in group, 7 deaths occurred, 3 during treatment due to disease progression and 4 during post-treatment. No deaths were reported in the 3-week lead-in group. Only one patient discontinued treatment due to adverse events (AE) (with DLT and cardiac failure) (Table 10). The maximum tolerated dose of Binding Protein #2 was not reached. No objective responses were observed, with a disease control rate of 35% (14/40) (FIGS. 4A-4D). Best overall responses are listed in Table 12.

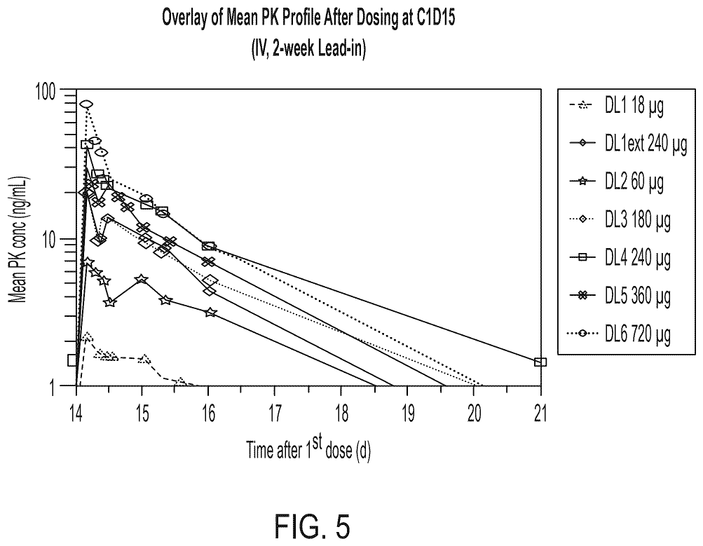

Pharmacokinetics (PK) was mostly dose-proportional in the explored dose range, with terminal half-life (T_{1/2}) generally between 20 and 50 hours. Mean maximum concentrations of Binding Protein #2 increased from 1.9 ng/mL for the 18 μg dose to 96 ng/mL for the 720 μg dose. The exposures were characterized by a moderate to high variability, with coefficient of variation (CV) % from 30% to >100% (FIG. 5).

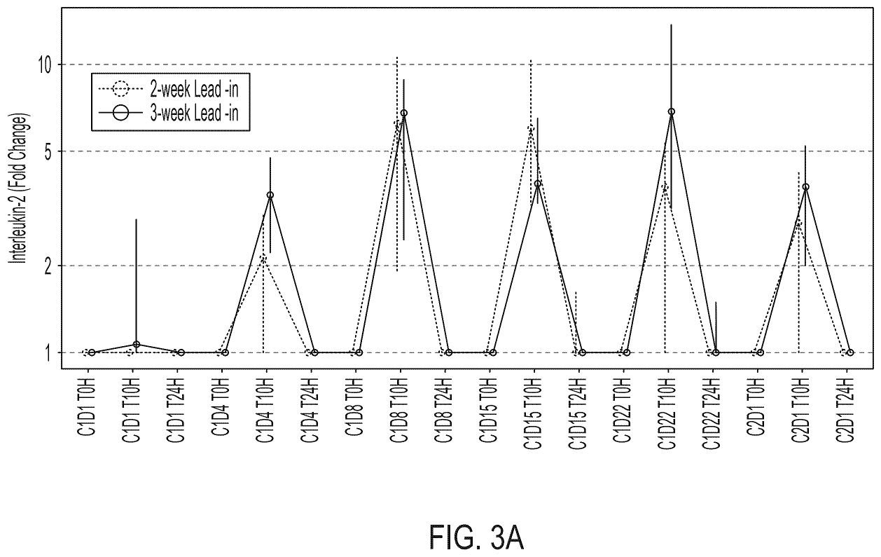

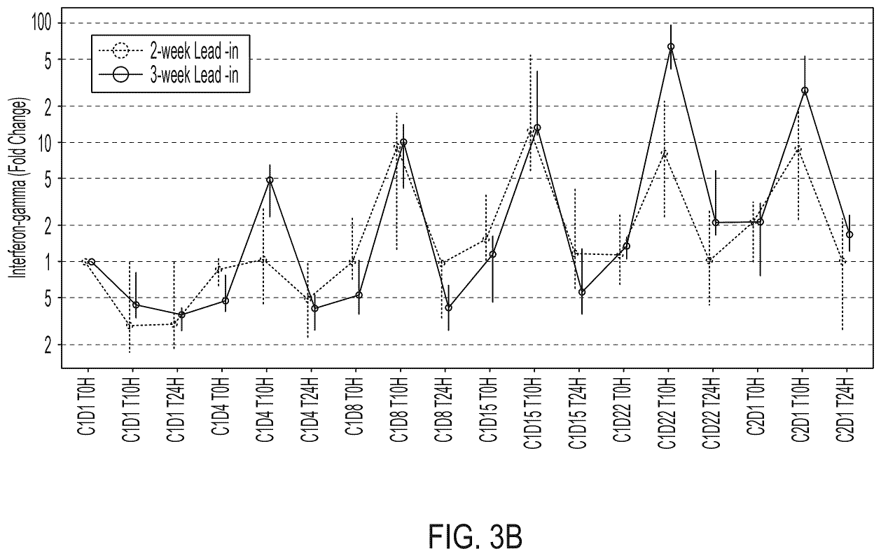

Moderate immunogenicity was observed after IV, with 8/29 (27.6%) treatment-emergent antidrug antibodies (ADAs) in the 2-week lead-in and 1/11 (9.1%) in the 3-week lead-in. Some participants that were ADA+ showed reduced PK exposure. The median time to ADA onset was 60.0 days in the 2-week lead-in cohort and 108 days in the 3-week lead-in cohort. Biomarker analysis revealed transient increase in the levels of serum pro-inflammatory cytokines after each infusion, which returned to near baseline levels before the next infusion. IL2 and IFNγ (starting at Cycle 1, Day 4 2^{nd} infusion) increased at 10-hour and returned to baseline at 24-hour after each infusion (median fold-change in all patients, inter quartile range (IQR); FIGS. 3A-3B), thus indicated T-cell activation and target engagement.

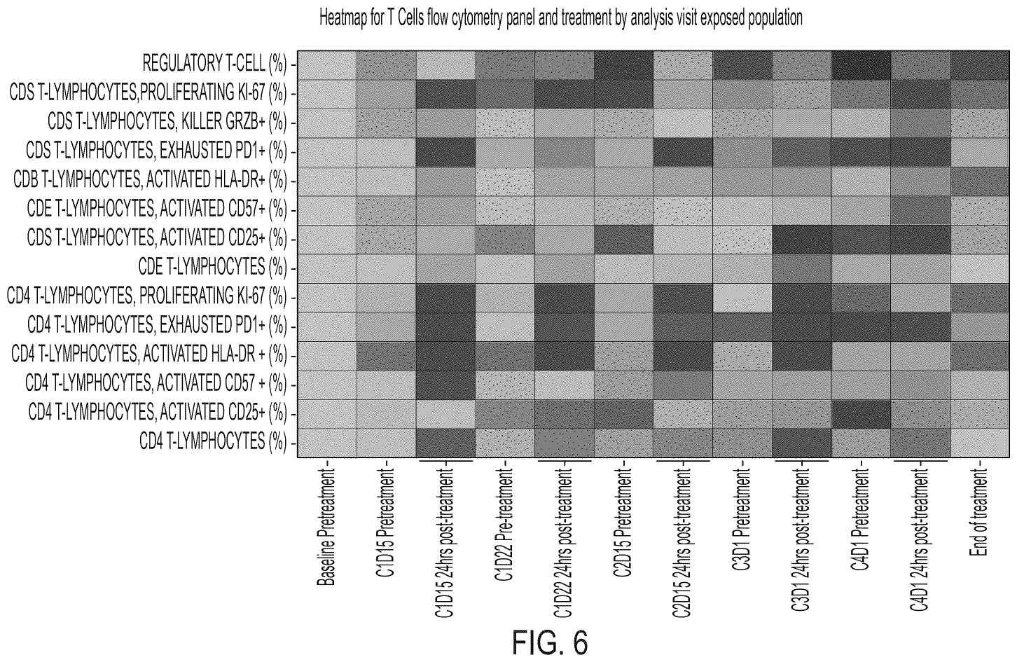

Binding Protein #2 infusion led to increases in T cell activation markers such as cluster of differentiation 57 (CD57), programmed cell death protein 1 (PD1), human leukocyte antigen-DR isotype (HLA-DR); the cytotoxicity biomarker granzyme B; and the proliferation biomarker Ki67; along with decreases in regulatory T cells (Tregs). After each infusion, a rapid decrease in peripheral lymphocyte counts was observed. This lymphocyte distribution was accompanied by an increase in the T cell activation markers (FIG. 6).

TABLE 9
Patient Baseline Characteristics

| | | 2-week Lead-in DLs^{a} | | | | | | 3-week Lead-in DLs^{a} | | | |
|---|---|---|---|---|---|---|---|---|---|---|---|
| | | 18 μg | 60 μg | 180 μg | 240 μg | 360 μg | 720 μg | 480 μg | 720 μg | 900 μg | All |
| | | (n = 4) | (n = 3) | (n = 9) | (n = 4) | (n = 6) | (n = 3) | (n = 4) | (n = 4) | (n = 3) | (N = 40) |
| Age (years), median | | 52.5 | 53 | 58 | 50.5 | 49.5 | 61 | 60.5 | 56 | 67 | 67.90 |
| (range) | | (43- | (30- | (41- | (47- | (32- | (44 | (46- | (53- | (46- | (17.09) |
| | | 77) | 71) | 68) | 79) | 62) | 75) | 77) | 61) | 68) | |
| Sex, | Male | 3 | 1 | 3 | 0 | 2 | 0 | 1 | 3 | 2 | 15 (37.5) |
| n (%) | | (75.0) | (33.3) | (33.3) | | (33.3 | | (25.0) | (75.0) | (66.7) | |
| | Female | 1 | 2 | 6 | 4 (100) | 4 | 3 (100) | 3 | 1 | 1 | 25 (62.5) |
| | | (25.0) | (66.7) | (66.7) | | (66.7) | | (75.0) | (25.0) | (33.3) | |
| ECOG | 0 | 2 | 1 | 4 | 2 | 4 | 2 | 0 | 3 | 1 | 19 (47.5) |
| PS, | | (50.0) | (33.3) | (44.4) | (50.0) | (66.7) | (66.7) | | (75.0) | (33.3) | |
| n (%) | 1 | 2 | 2 | 5 | 2 | 2 | 1 | 4 (100) | 1 | 2 | 21 (52.5) |
| | | (50.0) | (66.7) | (55.6) | (50.0) | (33.3) | (33.3) | | (25.0) | (66.7) | |
| Primary | Breast | 1 | 1 | 0 | 2 | 0 | 2 | 0 | 1 | 0 | 7 (17.5) |
| tumor | | (25.0) | (33.3) | | (50.0) | | (66.7) | | (25.0) | | |
| location, | Stomach | 2 | 0 | 0 | 0 | 1 | 1 | 1 | 0 | 0 | 5 (12.5) |
| n (%) | | (50.0) | | | | (16.7) | (33.3) | (25.0) | | | |
| | Lung | 0 | 0 | 1 | 0 | 1 | 0 | 0 | 1 | 0 | 3 (7.5) |
| | | | | (11.1) | | (16.7) | | | (25.0) | | |
| | Rectum | 0 | 0 | 1 | 0 | 1 | 0 | 1 | 0 | 0 | 3 (7.5) |
| | | | | (11.1) | | (16.7) | | (25.0) | | | |
| | Others | 1 | 2 | 7 | 2 | 3 | 0 | 2 | 2 | 3 (100) | 22 (55.0) |
| | | (25.0) | (66.7) | (77.8) | (50.0) | (50.0) | | (50.0) | (50.0) | | |
| No. of | ≥3 | 2 | 3 (100) | 5 (100) | 4 (100) | 5 | 2 | 2 | 4 (100) | 3 (100) | 30 (75) |
| prior | | | | | | | | | | | |
| regimens, | | (50.0) | | | | (83.3) | (66.7) | (50.0) | | | |
| n (%) | | | | | | | | | | | |
| Tumor | IHC3+ | 4 | 0 | 2 | 0 | 4 (66.7 | 2 | 2 | 1 | 2 | 17 (42.5) |
| Histology- | | (100) | | (22.2) | | | (66.7) | (50.0) | (25.0) | (66.7) | |
| IHC, | IHC2+ | 0 | 2 | 2 | 2 | 0 | 1 | 1 | 1 | 1 | 10 (25.0) |
| n(%) | | | (66.7) | (22.2) | (50.0) | | (33.3) | (25.0) | (25.0) | (33.3) | |
| | IHC1+ | 0 | 1 | 0 | 1 | 1 | 0 | 1 | 0 | 0 | 4 (10.0) |
| | | | (33.3) | | (25.0) | (16.7) | | (25.0) | | | |
| HER2 | Abnormality/ | 3 | 1 | 6 | 2 | 3 | 0 | 1 | 3 | 3 (100) | 22 (55.0) |
| mutation | alteration | (75.0) | (33.3) | (66.7) | (50.0) | (50.0) | | (25.0) | (75.0) | | |
| status^{b}, n | | | | | | | | | | | |
| (%) | detected | | | | | | | | | | |

^{a}Fixed dose;
^{b}Nine participants who didn't have IHC result were included in the study with HER2 mutation/aberration.
Abbreviations: DL, dose levels; ECOG PS, Eastern Cooperative Oncology Group Performance Status; HER2, human epidermal growth factor receptor 2; IV, intravenous.

TABLE 10
Overview of adverse events

| | 2-week Lead-in DLs | | | | | | 3-week Lead-in DLs | | | |
|---|---|---|---|---|---|---|---|---|---|---|
| | DL1 | DL2 | DL3 | DL3b | DL4 | DL5 | DL1ext | DL2ext | DL3ext | |
| Incidence, n | 18 μg | 60 μg | 180 μg | 240 μg | 360 μg | 720 μg | 480 μg | 720 μg | 900 μg | All |
| (%) | (n = 4) | (n = 3) | (n = 9) | (n = 4) | (n = 6) | (n = 3) | (n = 4) | (n = 4) | (n = 3) | (N = 40) |
| DLTs | 0 | 0 | 1 | 0 | 0 | 2 | 0 | 0 | 0 | 3 (7.5) |
| | | | (11.1) | | | (66.7) | | | | |
| Any TEAE | 3 | 3 | 9 | 4 | 6 | 3 | 4 (100) | 4 (100) | 3 (100) | 39 |
| | (75.0) | (100) | (100) | (100) | (100) | (100) | | | | (97.5) |
| Grade ≥3 | 3 | 1 | 4 | 2 | 2 | 3 | 1 (25.0) | 1 (25.0) | 1 (33.3) | 18 (45) |
| TEAE | (75.0) | (33.3) | (44.4) | (50.0) | (33.3) | (100) | | | | |
| Any treatment- | 3 | 2 | 3 | 2 | 1 | 3 | 0 | 1 (25.0) | 1 (33.3) | 16 (40) |
| emergent SAE | (75.0) | (66.7) | (33.3) | (50.0) | (16.7) | (100) | | | | |
| Any TEAE | 0 | 0 | 1 | 0 | 0 | 0 | 0 | 0 | 0 | 1 (2.5) |
| leading to | | | (11.1) | | | | | | | |
| permanent | | | | | | | | | | |
| discontinuation | | | | | | | | | | |
| Any treatment- | 1 | 0 | 2 | 2 | 1 | 3 | 2 (50.0) | 1 (25.0) | | 12 (30) |
| emergent | (25.0) | | (22.2) | (50.0) | (16.7) | (100) | | | | |
| AESI^{a} | | | | | | | | | | |
| Any TEAE | 2 | 1 | 8 | 4 | 6 | 3 | 4 (100) | 4 (100) | 3 (100) | 35 |
| related to | (50.0) | (33.3) | (88.9) | (100) | (100) | (100) | | | | (87.5) |
| Binding | | | | | | | | | | |
| Protein #2 | | | | | | | | | | |

^{a}Cytokine release syndrome (grade 2), cardiac failure (grade 3), and infusion-related reaction (grade 2).
AESI, adverse event of special interest; DL, dose level; DLT, dose-limiting toxicities; TEAE, treatment-emergent adverse event.

TABLE 11
Most frequent TEAEs of any grade (≥15%)

| | 2-week Lead-in DLs | | | | | | 3-week Lead-in DLs | | | |
|---|---|---|---|---|---|---|---|---|---|---|
| | DL1 | DL2 | DL3 | DL3b | DL4 | DL5 | DL1ext | DL2ext | DL3ext | |
| Incidence, | 18 μg | 60 μg | 180 μg | 240 μg | 360 μg | 720 μg | 480 μg | 720 μg | 900 μg | All |
| n (%) | (n = 4) | (n = 3) | (n = 9) | (n = 4) | (n = 6) | (n = 3) | (n = 4) | (n = 4) | (n = 3) | (N = 40) |
| CRS | 1 | 0 | 4 | 1 | 3 | 2 | 3 (75.0) | 3 (75.0) | 3 (100) | 20 (50) |
| | (25.0) | | (44.4) | (25.0) | (50.0) | (66.7) | | | | |
| Pyrexia | 0 | 1 | 4 | 2 | 2 | 0 | 1 (25.0) | 2 (50.0) | 2 (66.7) | 14 (35) |
| | | (33.3) | (44.4) | (50.0) | (33.3) | | | | | |
| Increased | 0 | 1 | 5 | 1 | 1 | 2 | 1 (25.0) | 1 (25.0) | 1 (33.3) | 13 (32.5) |
| ALT levels | | (33.3) | (55.6) | (25.0) | (16.7) | (66.7) | | | | |
| Infusion | 0 | 0 | 0 | 3 | 1 | 3 | 2 (50.0) | 2 (50.0) | 0 | 11 (27.5) |
| related | | | | (75.0) | (16.7) | (100) | | | | |
| reaction | | | | | | | | | | |
| AST | 0 | 0 | 4 | 1 | 1 | 2 | 1 (25.0) | 1 (25.0) | 1 (33.3) | 10 (25) |
| increased | | | (44.4) | (25.0) | (16.7) | (66.7) | | | | |
| Fatigue | 3 | 0 | 1 | 1 | 0 | 2 | 0 | 0 | 2 (66.7) | 9 (22.5) |
| | (75.0) | | (11.1) | (25.0) | | (66.7) | | | | |
| Dyspnoea | 1 | 1 | 2 | 1 | 1 | 0 | 0 | 0 | 2 (66.7) | 8 (20) |
| | (25.0) | (33.3) | (22.2) | (25.0) | (16.7) | | | | | |
| Headache | 0 | 1 | 3 | 1 | 0 | 1 | 0 | 0 | 1 (33.3) | 7 (17.5) |
| | | (33.3) | (33.3) | (25.0) | | (33.3) | | | | |
| Nausea | 0 | 0 | 2 | 1 | 2 | 1 | 0 | 0 | 1 (33.3) | 7 (17.5) |
| | | | (22.2) | (25.0) | (33.3) | (33.3) | | | | |
| Anemia | 1 | 0 | 1 | 1 | 2 | 2 | 0 | 0 | 0 | 7 (17.5) |
| | (25.0) | | (11.1) | (25.0) | (33.3) | (66.7) | | | | |
| Decreased | 1 | 2 | 0 | 1 | 1 | 0 | 1 (25.0) | 0 | 0 | 6 (15) |
| appetite | (25.0) | (66.7) | | (25.0) | (16.7) | | | | | |
| Asthenia | 0 | 1 | 0 | 2 | 1 | 1 | 1 (25.0) | 0 | 0 | 6 (15) |
| | | (33.3) | | (50.0) | (16.7) | (33.3) | | | | |
| Chills | 0 | 0 | 2 | 1 | 2 | 0 | 0 | 1 (25.0) | 0 | 6 (15) |
| | | | (22.2) | (25.0) | (33.3) | | | | | |

ALT, alanine aminotransferase; AST, aspartate aminotransferase; CRS, Cytokine release
syndrome; DL, dose level; TEAE, treatment-emergent adverse event.

TABLE 12
Best overall response

| | 2-week Lead-in DLs | | | | | | 3-week Lead-in DLs | | | |
|---|---|---|---|---|---|---|---|---|---|---|
| | DL1 | DL2 | DL3 | DL3b | DL4 | DL5 | DL1ext | DL2ext | DL3ext | |
| | 18 μg | 60 μg | 180 μg | 240 μg | 360 μg | 720 μg | 480 μg | 720 μg | 900 μg | All |
| | (n = 4) | (n = 3) | (n = 9) | (n = 4) | (n = 6) | (n = 3) | (n = 4) | (n = 4) | (n = 3) | (N = 40) |
| Complete | 0 | 0 | 0 | 0 | 0 | 0 | 0 | 0 | 0 | 0 |
| response or | | | | | | | | | | |
| partial | | | | | | | | | | |
| response | | | | | | | | | | |
| Stable | 1 | 0 | 4 | 1 | 4 | 0 | 2 (50.0) | 2 (50.0) | 0 | 14 |
| disease | (25.0) | | (44.4) | (25.0) | (66.7) | | | | | (35.0) |
| Progressive | 3 | 3 | 5 | 3 | 2 | 3 | 1 (25.0) | 2 (50.0) | 3 (100) | 25 |
| disease | (75.0) | (100) | (55.6) | (75.0) | (33.3) | (100) | | | | (62.5) |
| Not | 0 | 0 | 0 | 0 | 0 | 0 | 1 (25.0) | 0 | 0 | 1 |
| evaluable | | | | | | | | | | (2.5) |

DL, dose level.

### Conclusion for IV Dose-Escalation Cohorts

Treatment with Binding Protein #2 in advanced HER2+ solid tumors is feasible with manageable toxicities. During the treatment-emergent period, no fatal or related TEAEs were observed in any cohort or dose groups. Binding Protein #2 induced serum IFNγ and IL2 post drug administration, indicating T-cell activation and target engagement. In the dose escalation, treatment with Binding Protein #2 resulted in a disease control rate of 35% (14/40 patients).

### Example 2: a Quantitative Systems Pharmacology Framework for Tri-Specific T Cell Engagers: Application to HER2×CD3×CD28 Molecules

### Abstract

Binding Protein #2 is a tri-specific antibody (TsAb) for treating solid tumors that express human epidermal growth factor receptor 2 (HER2). Binding Protein #2 functions as a T cell engager (TCE) with anti-CD3, -CD28, and -HER2 sites capable of engaging respective cell-surface receptor proteins. The molecule mediates pharmacological actions by forming synaptic bridges between CD3+/CD28+ T cells and HER2+ tumor cells. Similar to other TsAb-based TCEs, numerous biological variables at the molecular and cellular level govern the immunomodulatory action of Binding Protein #2. Therefore, interpreting the pharmacological behaviors of the compound is challenging by experiment alone. This work introduces a quantitative systems pharmacology (QSP) framework to understand the pharmacological behaviors of Binding Protein #2. The QSP model was used to analyze ex vivo cytotoxicity and cytokine release profiles of Binding Protein #2. The model was also applied to mechanistically understand interleukin 6 (IL-6) responses in vivo based on data from preclinical studies. The analysis provided insights into the pharmacologically active concentration range of the drug at variable expression of the target protein HER2 in tumor cells. In addition, the model provided a possible explanation for in vivo IL-6 dynamics distinct from the TCE's mechanism of action. The QSP model predictions from this study may extend the understanding of other emerging TCE molecules with a similar mechanism of action.

### 1. Introduction

Recently, quantitative systems pharmacology (QSP) approaches have been successfully used to gain insights into several T cell engager (TCE) functions. Driven by the knowledge of the drug mechanism of action (MoA) and disease biology, a QSP model can serve as a predictive tool when informed by limited data from preclinical studies. However, most of the QSP platforms developed to date are for the BsAb molecules only. There has been a recent effort to build a QSP model for a CD38×CD28×CD3 tri-specific TCE targeting multiple myeloma. However, this model was purposed for an in vitro system only. An in vivo system-scale model for a tri-specific TCE is currently lacking.

In this study, a QSP model for Binding Protein #2, a CD3×CD28×HER2 tri-specific TCE was developed. The tumor-associated antigen (TAA)-binding arm of Binding Protein #2 targets the human epidermal growth factor 2 (HER2) protein of tumor cells. The molecule contains two T cell antigen-binding arms, one is specific for the CD3 protein and the other for CD28, a co-stimulatory receptor protein of T cells. Unlike a BsAb, Binding Protein #2 is designed with this added capability to bind CD28 besides CD3 to induce more potent stimulation of the cytotoxic T lymphocytes (CTLs) and elicit stronger anti-tumor effects.

The QSP framework for Binding Protein #2 was developed by considering essential biology at the cellular and molecular scale that describes the MoA of this tri-specific antibody (TsAb) was developed herein. The model accounts for the molecular interactions between the three binding arms of Binding Protein #2 and its target antigens that drive synapse formation between the CD8/CD4 T lymphocytes and HER2-expressing tumor cells. It also incorporates the activation and proliferation of both T lymphocytes and their cytolytic actions on the tumor cells upon synapse formation. In addition, the model includes activated T cell release of four different cytokines which are important biomarkers of safety risks associated with TCE treatment. The QSP model reproduced several in vitro datasets and predicted pharmacologically active concentrations of Binding Protein #2. Based on cynomolgus monkey studies, the model also provided mechanistic insights into in vivo response profiles of interleukin-6 (IL-6), a key biomarker for cytokine release syndrome (CRS) associated with TCE therapies.

### 2. Materials and Methods

The multiscale QSP model was scoped for 1) an in vitro system, and 2) a whole-body system corresponding to the physiology of a cynomolgus monkey. Below these two forms of the model are explained. Then experimental materials and methods related to different datasets used in this work are provided.

### 2.1 In Vitro System Model Development

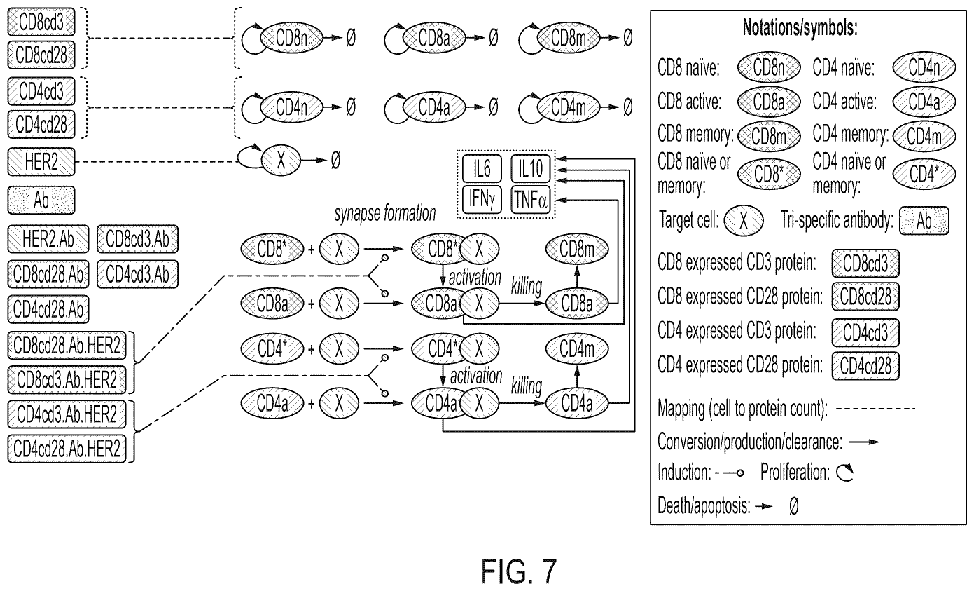

FIG. 7 provides a schematic of the model showing the cellular and molecular-scale interactions and processes, which are detailed below.

### 2.1.1 Compartment, Cells and Protein Molecules

The in vitro system was defined by a one-compartment model, where the compartment represents a cell culture microplate well. The volume of the compartment (microplate well) corresponds to a co-culture solution of CD8 and CD4 T cells and a tumor cell of interest. Each cell type is associated with a copy number for corresponding surface antigen molecules. Both CD8 and CD4 T cells are assigned a copy number of CD3 and CD28 molecules, while the tumor cell is assigned a copy number of HER2 molecule. Each of these cell-specific protein copy numbers is a fixed parameter obtained from direct measurements.

The initial condition of the system was defined by a baseline number of each cell type based on the experimental assay condition. During the course of a simulation, the number of each cell type evolves by their defined rate of proliferation, apoptosis, and drug-mediated killing (if the drug is added). The growth of each cell type is capped by the maximum possible number (carrying capacity of the in vitro system). The model updates the number of each cell-surface antigen molecule with any changes in the corresponding cell type during the course of a simulation. This allows the copy number (per cell basis) of each protein to be defined as a fixed parameter.

### 2.1.2 Drug-Protein Interactions

Binding Protein #2 addition to the system at time zero triggers the interactions illustrated in FIG. 7. The antibody binds to CD3, CD8, and HER2 to form possible bi-molecular and trimeric antibody-antigen complexes (FIG. 7). The stability of each of these complexes depends on the affinities (equilibrium dissociation constant K_{D}) for associated antibody-antigen bonds. The model permits the antibody to bind either CD3 or CD28 but not both proteins simultaneously. This constraint accounts for possible steric hindrance arising from the location of the CD3 and CD28 binding arms in Binding Protein #2. However, each antibody molecule bound to HER2 and CD3 or CD28 contributes to synapse formation between a T cell and a tumor cell in the model.

### 2.1.3 Implementation of Cytokine Release In Vitro

The model includes four cytokines: IL-6, IL-10, IFN-γ, and TNF-α. The following equation describes activated T cell release of a cytokine with rate constant kᵢ:

$${r}_{i , j} = \frac{d {S}_{i}}{{d}_{t}} = {k}_{i} {T}^{*} + {k}_{i} ( 1 + δ ) {T}_{j}^{*} \qquad ( 1 )$$

Above, Sᵢ stands for the concentration of a cytokine i∈{IL-6, IL-10, IFN-γ, TNF-α}. The variable Tⱼ* represents activated T cell j∈{CD8, CD4}. Parameter δ takes is zero or positive depending on whether the activated T cell is free or in a synapse with a tumor cell.

### 2.2 In Vivo System Model Development

Below, features of the in vivo animal model parameterized for cynomolgus monkeys are provided.

### 2.2.1 In Vivo System: Cells and Molecules

The animal model is described as a one-compartment system. The volume of the compartment represents the typical plasma volume of an adult cynomolgus monkey. Similar to the in vitro system model, the volume contains species representing CD8 and CD4 T cells and target cells. The T cell densities are assigned based on CD8 and CD4 counts in the peripheral blood mononuclear cells (PBMCs). Each cell type is assigned CD3 and CD28 protein copy numbers (the same values used for the in vitro system). Because the animals lack tumors, they are unlikely to have high plasma levels of HER2 protein. Nevertheless, to account for non-specific HER2 expression, plasma HER2 is considered as well to perform the IL-6 response analysis detailed in the Results section.

### 2.2.2 Implementation of Binding Protein #2 Pharmacokinetics (PK)

First-order clearance of Binding Protein #2 from the (blood) compartment was considered. The PK parameters (volume of distribution and rate of clearance) of Binding Protein #2 are derived by fitting the PK data. The temporal concentration of the drug in plasma, determined by the PK, drives the drug-protein binding interactions (FIG. 7).

### 2.2.3 Implementation of T Cell Dynamics In Vivo

In vivo T cell dynamics was modeled following an earlier model by De Boer et al (De Boer, R. J., Homann, D., Perelson, A. S.: Different dynamics of cd4+ and cd8+t cell responses during and after acute lymphocytic choriomeningitis virus infection. The Journal of Immunology 171(8), 3928-3935 (2003)). In the absence of the drug, CD4 and CD8 cells in the compartment remain in the resting or naive form. The resting T cell populations maintain a steady-state balance between death and proliferation to represent their peripheral blood densities. The addition of the drug triggers the drug-antigen binding interactions (FIG. 7) driving the synapse formation, T cell activation, target cell killing, and cytokine release. Although these biological interactions are the same as the in vitro model, the T cell dynamics to follow an in vivo condition based on De Boer et al was considered. Accordingly, upon drug infusion, the activated CD8 and CD4 proliferate following a logistic growth for the first λ=7 days. For subsequent Δ=8 days, activated T cells go through a rapid contraction at a rate α. Thereafter, activated T cells decay at a basic turnover rate δ_{A}. The following equations describe this proliferation rule for activated CD8 or CD4.

$$0 \leq t \leq λ : \frac{d {T}^{*}}{dt} = {k}_{p} ( ( 1 - \frac{{T}_{tot}}{{T}_{max}} ) {T}^{*} \qquad ( 2 )$$

$$λ < t \leq λ + Δ : \frac{d {T}^{*}}{dt} = - ( r + α + {δ}_{A} ) \qquad ( 3 )$$

$$t > λ + Δ : \frac{d {T}^{*}}{dt} = - ( r + δ ) {T}^{*} \qquad ( 4 )$$

In the above equations, a single notation T* is used to represent both CD8 and CD4 density in the blood (the same notation is used, as the equations apply to both cell types). Tₜₒₜ represents total T cell density (CD8 and CD4 combined), and Tₘₐₓ represents the carrying capacity of blood for total T cells (CD8 and CD4 combined).

Tₘₐₓ is assigned twice the normal T cell density. This assumption is based on the observations that the average T cell count in blood rarely exceeded twofold of normal density even under PD-1-targeted treatments or viral infections. Parameter represents the first-order transition of an activated T cell into a memory T cell. In repeat dosing cases, this scenario of activated T cell proliferation is renewed after each dosing. Unlike the activated T cells, resting cells (naive and memory) maintain a base rate of proliferation and death.

### 2.2.4 Implementation of IL6 Dynamics In Vivo

In the in vivo model, two distinct sources for IL-6 release in plasma are considered (please see the Result section for further details). One source is directly linked to T-cell activation by the drug's MoA. The other source is a non-specific (generic) response that could be triggered in response to the infused drug. This latter source represents components of the immune system other than T cells can also recognize the drug as an antigenic species and may respond by generating IL-6 and other pro-inflammatory cytokines. The following equation describes contributions from the two sources in the model.

$${r}_{IL 6} = {r}_{IL 6}^{s} + {r}_{IL 6}^{ns} \qquad ( 5 )$$

Here,

$${r}_{IL 6}^{s}$$

represents the contribution from the drug's MoA and described by Eq. 1, and

$${r}_{IL 6}^{ns}$$

represents the non-specific source described by the following equation:

$${r}_{IL 6}^{ns} = \{ \begin{matrix}0 ; & 0 < ( t - {t}_{k} ) < θ \\ ( \frac{C}{{E}_{50} + C} ) {I}_{max} {e}^{- γ ( t - {t}_{j} ) - {k}_{I} , t} ; & ( t - {t}_{j} ) \geq θ\end{matrix} \qquad ( 6 )$$

In Eq. 6, the system responds with a time lag θ after infusion at time tₖ (subscript k represents the kth infusion in a repeat dosing scheme). The IL-6 release rate is a saturating function of the plasma drug concentration (C) with maximum possible release intensity Imax. The decay term e−^{K}^{_{I}}^{t} describes diminishing sensitivity to the drug over time leading to smaller peak responses in subsequent dosing. The additional decay term e^{-γ(t-t}^{ⱼ}⁾ with tⱼ≤t<tⱼ₊₁ allows the model to consider a decaying capacity for this non-specific release after an infusion (based on the observed dynamics). IL-6 is eliminated from plasma following a first-order rate law in the model.

### 2.3 Experimental Methods

### 2.3.1 T Cell Activation and Cytokine Release Assays

Co-cultures of primary T cells and HER-2 expressing target cells (HCC-1954) were used to determine Binding Protein #2 ability to activate human T cells (CD8 or CD4) and cytokine release (IL-6, IL-10, IFN-γ, and TNF-α). CD8 and CD4 T cells from normal donors were added to wells containing HCC-1954 at a 5:1 ratio (five T cells per target cell). The co-cultures were incubated with Binding Protein #2 or control antibody IgG4-FALA at four concentrations in 10-fold serial dilutions: 10, 100, 1,000, and 10,000 pM. T-cell activation and cytokine release were examined after 48 hours of incubation. T cell activation was measured by flow cytometric assessment of CD69 expression in CD8 and CD4 T cells. Activation was quantified as CD69 expression in the presence of Binding Protein #2 relative to the control condition (IgG4-FALA treatment). Cytokine release was measured by collecting supernatants from the co-cultures for all four doses tested. The assays were conducted with T cells from five different donors and in duplicate at each antibody treatment condition.

### 2.3.2 Binding Protein #2 Cytotoxicity Assay

The ability of Binding Protein #2 to kill tumor cells was examined in TDCC (T-cell dependent cellular cytotoxicity). Primary T cells derived from peripheral blood mononuclear cells of normal donors were added to wells containing the tumor cells (HCC-1954) at 5:1 effector-to-target ratio. The co-culture was incubated in the presence of Binding Protein #2 or control (IgG4-FALA) for 48 hours. Tumor cell lysis in the co-culture was monitored using xCELLigence® real-time cell analysis (RTCA) system (Acea Biosciences). Post-incubation cytotoxicity was quantified in percent cell killing in the presence of Binding Protein #2 relative to the control condition.

### 2.4 Binding Protein #2 and Cytokine Assays in Cynomolgus Monkeys

Two groups of cynomolgus macaques were used to generate data presented in this study. There were three animals per group (two males and one female in each group). The first group received four repeat doses of 10 μg/kg Binding Protein #2 on days 1, 4, 8, and 11. The second group received four repeat doses of 30 μg/kg Binding Protein #2 injected intravenously on days 1, 4, 8, and 11. The drug was infused intravenously in an aqueous solution (control article). Each intravenous infusion was carried out over one hour. IL-6 plasma concentration was examined on each day of administration once before the start of treatment and at 4 and 24 hours post-treatment.

### 3. Results

As described in Materials and Methods, under this QSP framework, two models have been developed to simulate in vitro and in vivo system conditions. Key results from these model-based analyses are discussed.

### 3.1 In Vitro System Model: Analysis of Binding Protein #2 Pharmacological Action

The in vitro system model was scoped to analyze the pharmacological behaviors of Binding Protein #2, as discussed below.

### 3.1.1 Binding Protein #2-Induced T Cell Activation and Tumor Cell Killing

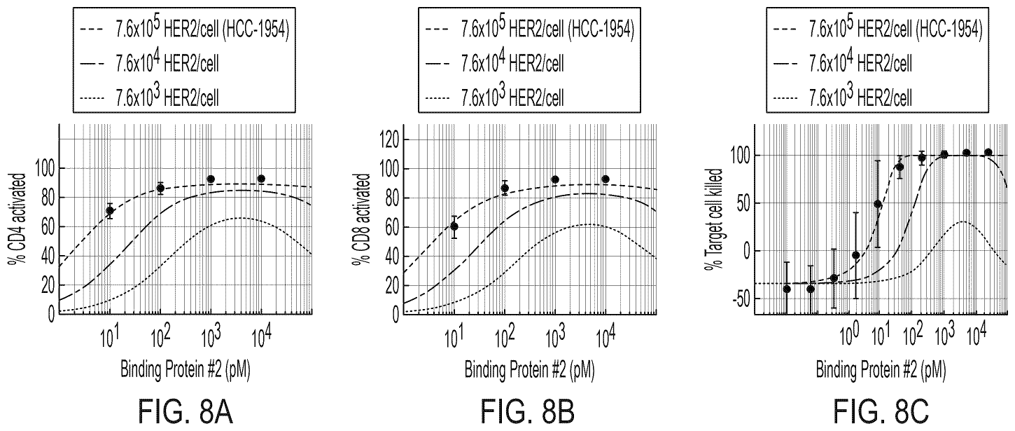

Whether the biological details and drug MoA included in the in vitro system model could correctly reproduce the observed ex vivo pharmacological behavior of Binding Protein #2 was first tested. The model was calibrated with data from in vitro studies that examined Binding Protein #2-induced T cell activation and tumor cell killing. As shown in FIGS. 8A-8B, the model was able to reproduce the activation profiles of CD8 T cells (FIG. 8B) and CD4 T cells (FIG. 8A) at various doses of Binding Protein #2. It also captured the corresponding lysis of HCC-1954 (FIG. 8C).

The calibrated model was then used to predict the effects of HER2 expression on the drug's ability to activate T cells and mediate tumor cell killing. The HCC-1954 cell mentioned above is a high HER2-expressing breast tumor-derived cell line with around 740,000 HER2 molecules per cell. Two additional scenarios representing moderate and low HER2 conditions were considered, which were assumed to be 10- and 100-fold reduced HER2 levels relative to HCC-1954. Under these conditions, simulations revealed a bell-shaped profile with optimal Binding Protein #2 concentration between 1-10 nM. For HCC-1954 (high HER2), neither the model nor the data indicated this feature for the Binding Protein #2 concentrations studied. Nonetheless, when simulations were performed for a dose range beyond 10⁵ nM Binding Protein #2, the model indicated a bell-shaped profile with high-dose inhibition for HCC-1954 (results not presented).

### 3.1.2 Trimeric Complexes Driving Pharmacological Action of Binding Protein #2

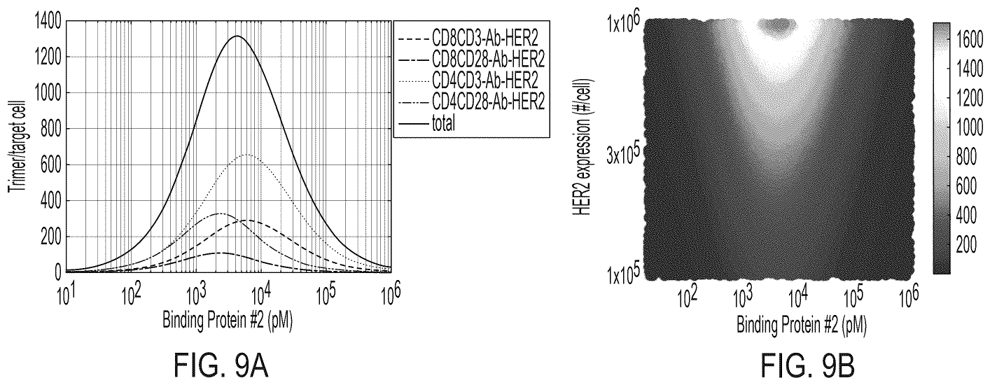

The bell-shaped response profile can inform the optimal dose range for TCEs. Theoretically, such profiles can arise from the binding competition among the drug molecules for the target antigens leading to high-dose inhibition, as discussed in other modeling works for the bi-specific TCEs. To further examine this mechanistic explanation, the model was applied to interrogate the dose-response relationship of the four trimeric complexes contributing to synapse formation (FIG. 7). FIG. 9A shows the model-predicted profiles of these four complexes. At high Binding Protein #2 concentrations, all four complexes were compromised because competition for the HER2 molecules favored the unproductive 1:1 drug-protein complexes (FIG. 7) at the expense of the trimers. The peak of each trimer occurred at different concentration ranges determined by their distinct stabilities. All these four complexes were considered to be equally capable of synapse formation. Therefore, the sum of these four complexes determines the optimal concentration range, as depicted in FIG. 9A.

To understand how HER2 level might impact the formation of these trimers, simulations considering various combinations of the drug concentration and HER2 expression were performed. The colormap in FIG. 9B shows the predicted pharmacologically active region corresponding to these two variables. However, whether this predicted HER2 dependency of the optimal region is relevant for clinical dose selection is debatable because intra-tumor TCE concentrations are unlikely to reach the concentration range of the high-dose inhibition.

### 3.1.3 Proinflammatory Cytokine Responses In Vitro

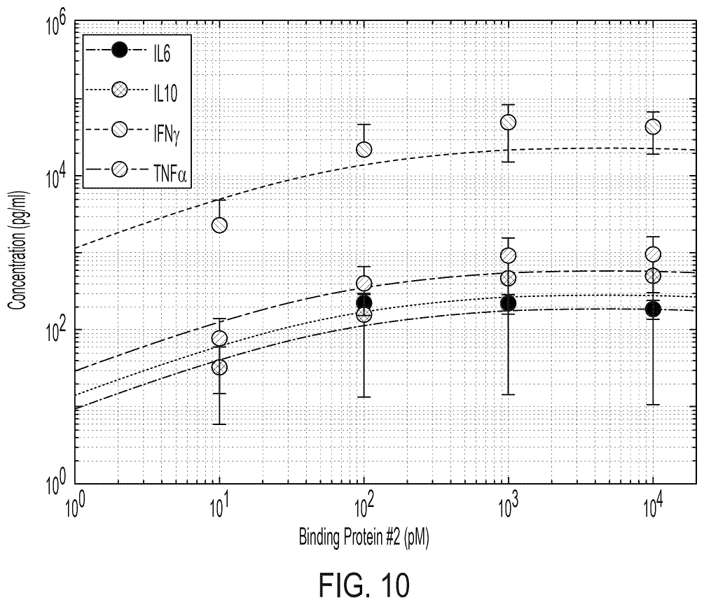

While keeping the T-cell activation and cytotoxicity profiles of HCC-1954 unchanged from the previous calibrations (FIGS. 9A-9B), the model was further calibrated to reproduce the IL-6, IL-10, IFN-γ and TNF-α responses observed in the same set of in vitro studies. The model could explain the dose-response profiles of each cytokine, as shown in FIG. 10. These calibrations provided quantitative estimates for activated T cell release of each cytokine. However, the calibrations led us to consider distinct rates of cytokine release depending on whether the activated T cells were in synapse or free state. The model indicated approximately three-fold higher cytokine release from T cells in synapse than their free states (δ=3 in Eq.).

### 3.2 In Vivo System Model: Analysis of IL-6 Responses in Cynomolgus Monkeys

The in vivo system model was used to understand possible sources of IL-6 in cynomolgus monkeys. The animals, despite being devoid of tumors (and hence the target protein HER2), displayed dose-dependent IL-6 responses. A possible explanation was that there could be a nominal expression of HER2 by epithelial or other non-tumor cells in the systemic circulation to generate the IL-6 responses. The other possibility is that the immune system recognized the infused drug as an antigenic substance that stimulated various cells capable of releasing IL-6. These two possibilities were systematically interrogated. The former possibility is directly linked to the drug's MoA, while the latter represents a non-specific antigenic response to the drug molecule.

3.2.1 Binding Protein #2 MoA Vs. IL-6 Response In Vivo

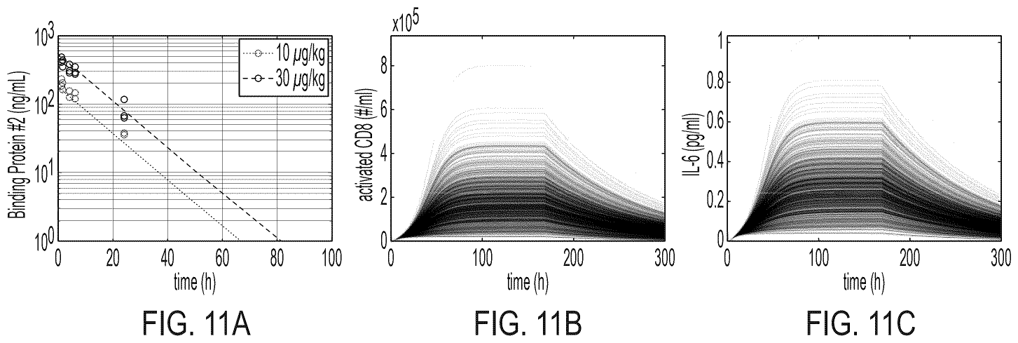

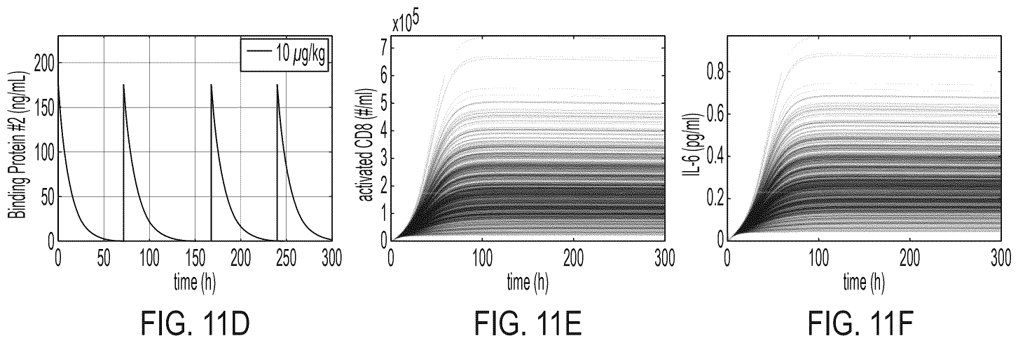

To investigate if the drug's MoA was linked to the observed IL-6 responses, the mode to the PK of Binding Protein #2 (FIG. 11A) was first calibrated to estimate the PK parameters. With the estimated PK parameters, the model was then used to simulate the four repeat dose conditions (FIG. 11B used to study IL-6 responses in the animals (FIG. 11C). Clearly, the predicted serum level of IL-6 was too small to explain the responses observed. Note that these predictions were made considering plasma HER2 density similar to the in vitro system for HCC-1954 cells. Such a high abundance of HER2 is unlikely for animals devoid of tumors. However, this consideration was to favor the contribution from the drug's MoA.

The reason for the model to predict such low blood concentration of IL-6 despite high HER2 abundance turned out to be the fast clearance of IL-6 due to its short half-life (˜4.3 minutes in cynomolgus monkeys). The relatively slow process of the drug-induced T cell activation and consequent IL-6 release could not make up for this fast disappearance to cause noticeable elevation of IL-6 concentration in blood. This in vivo scenario is quite different from the in vitro case where IL-6 produced by the drug's MoA only accumulated over time with no mechanism for its clearance.

### 3.2.2 Non-Specific Source of IL-6 Response In Vivo

The above results led us to investigate the second possibility for IL-6 release independent of the TCE's MoA. To model this non-specific response (Eq. 6), several assumptions were made based on certain characteristics of the observed IL-6 dynamics. These assumptions that serve as the rationale of Eq. 6 and may apply to other TCEs showing similar responses are explained below.

It was noticed that the IL-6 response followed the PK profile closely. This indicates that IL-6 release was sensitive to instantaneous changes of Binding Protein #2 concentration in the blood rather than the slow process of the TCE-mediated T cell activation and proliferation. Therefore, the IL-6 release rate was related directly to plasma Binding Protein #2 concentration using an Emax function. Secondly, a lag time was introduced to account for the observed time delay (˜4 hours) between drug infusion and induction of the IL-6 response. Thirdly, peak IL-6 responses became smaller in each subsequent infusion, suggesting possible adaptation of the immune system to the drug over time. Therefore, a decaying sensitivity was introduced to account for this loss of responsiveness. Finally, IL-6 concentration fell sharply after reaching each peak which could not be explained by the IL-6's half-life alone with continual synthesis at a constant rate. This effect of diminishing releasing capacity was accounted for by introducing an additional exponential term in Eq. 6. These observations together included in Eq. 6 allowed the model to capture all aspects of IL-6 dynamics noted in response to the repeat dose conditions. In summary, the model suggests this drug MoA-independent route of IL-6 release alone could account for the magnitude and dynamics of IL-6 responses in vivo, thus discounting possible contributions from the TCE's MoA.

### 3.2.3 A Simple Explanation for the IL-6 Responses In Vivo

To further interrogate why the drug MoA led to insignificant IL-6 responses, even under high HER2 abundance, a simple model was adopted that could explain the QSP predictions:

$$\frac{dS ( t )}{dt} = p N ( t ) - c S \qquad ( 7 )$$

In this simple description, the plasma concentration of IL-6, S(t), is governed by its rate of production (rate parameter p times activated T cell density N) and clearance (parameter c times S). To simplify, let us assume that, under sufficiently high doses (in this case 10 μg/kg), all T cells in the blood could have been activated before generating the first peak. This assumption is rather unlikely. Nevertheless, this hypothetical but unlikely scenario favors the first possibility that the response was coming from T-cell activation mediated by the drug. With this assumption, N(t) can be replaced with the baseline T cell count N₀:

$$\frac{dS ( t )}{dt} = p {N}_{0} - c S ( t ) \qquad ( 8 )$$

The solution to the above equation is

$$S ( t ) = \frac{p {N}_{0}}{c} ( 1 - {e}^{- ct} ) \qquad ( 9 )$$

A question is whether Eq. 3.2.3 can explain the observed IL-6 level with a reasonable estimate for N₀, p, and c. A high estimate of N₀ is about 10⁶ T cells/ml. The value of p can be estimated from the vitro system, where 100 pg/ml IL-6 was reached by the end of 48 hours. For the in vitro system, c=0 and N₀=5×10⁵/ml, therefore p≈S(t)/N₀=4×10⁻⁶ pg h⁻¹ per activated T cell. Based on the 4.3 min half-life of IL-6, c=9.24 h⁻¹. It took about 0.5 hours to reach the peak response from baseline IL-6 once the system started responding after the lag time, i.e., t=0.5 hour in. Plugging in these values for N₀, p, c, and t in Eq. 3.2.3 yields S(t)=0.43 pg/ml. This value is comparable to the QSP prediction of maximum IL-6 level from the drug's MoA alone and much smaller than the observed first peak occurring at ˜300 pg/ml. This analysis further confirms that the drug's MoA is inadequate to explain the observed IL-6 responses given the short half-life of IL-6.

### 4. Conclusion

This study has introduced a multiscale QSP framework for a tri-specific TCE Binding Protein #2. The QSP model recapitulated the response profiles of several biological mediators and cytokine biomarkers that reflect the pharmacological activity of Binding Protein #2. The model predicted the ex-vivo optimal concentration range of the compound and revealed the impacts of target expression on its pharmacological actions. The model-based analyses of this study suggest that in vivo IL-6 responses may arise from generic antigenic recognition of a TCE in contrast to the common idea that a TCE's mechanism of action underly cytokine responses in vivo.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1. A method of treating a subject having a cancer comprising administering to the subject about 0.2 μg to about 4500 μg of a trispecific binding protein, wherein the trispecific binding protein comprises a first antigen binding site that binds a CD28 polypeptide, a second antigen binding site that binds a CD3 polypeptide, and a third antigen binding site that binds a HER2 polypeptide.

2. The method of claim 1, wherein the trispecific binding protein comprises four polypeptide chains that form three antigen binding sites, wherein a first polypeptide chain comprises a structure represented by the formula: 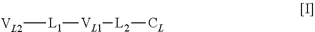
and a second polypeptide chain comprises a structure represented by the formula: 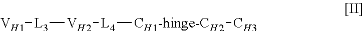
and a third polypeptide chain comprises a structure represented by the formula: 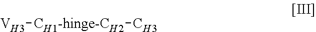
and a fourth polypeptide chain comprises a structure represented by the formula: 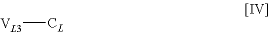
wherein:
V_{L1} is a first immunoglobulin light chain variable domain;
V_{L2} is a second immunoglobulin light chain variable domain;
V_{L3} is a third immunoglobulin light chain variable domain;
V_{H1} is a first immunoglobulin heavy chain variable domain;
V_{H2} is a second immunoglobulin heavy chain variable domain;
V_{H3} is a third immunoglobulin heavy chain variable domain;
C_{L} is an immunoglobulin light chain constant domain;
C_{H1} is an immunoglobulin C_{H1} heavy chain constant domain;
C_{H2} is an immunoglobulin C_{H2} heavy chain constant domain;
C_{H3} is an immunoglobulin C_{H3} heavy chain constant domain;
hinge is an immunoglobulin hinge region connecting the C_{H1} and C_{H2} domains; and
L₁, L₂, L₃ and L₄ are amino acid linkers;
wherein the polypeptide of formula I and the polypeptide of formula II form a cross-over light chain-heavy chain pair; and
wherein V_{H1} and V_{L1} form the first antigen binding site that binds a CD28 polypeptide, wherein the V_{H1} domain comprises a CDR-H1 sequence comprising the amino acid sequence of GYTFTSYY (SEQ ID NO: 49), a CDR-H2 sequence comprising the amino acid sequence of IYPGNVNT (SEQ ID NO:50), and a CDR-H3 sequence comprising the amino acid sequence of TRSHYGLDWNFDV (SEQ ID NO:51), and the V_{L1} domain comprises a CDR-L1 sequence comprising the amino acid sequence of QNIYVW (SEQ ID NO:52), a CDR-L2 sequence comprising the amino acid sequence of KAS (SEQ ID NO:53), and a CDR-L3 sequence comprising the amino acid sequence of QQGQTYPY (SEQ ID NO:54);
wherein V_{H2} and V_{L2} form the second antigen binding site that binds a CD3 polypeptide, wherein the V_{H2} domain comprises a CDR-H1 sequence comprising the amino acid sequence of GFTFTKAW (SEQ ID NO:55), a CDR-H2 sequence comprising the amino acid sequence of IKDKSNSYAT (SEQ ID NO:56), and a CDR-H3 sequence comprising the amino acid sequence of RGVYYALSPFDY (SEQ ID NO:57), and the V_{L2} domain comprises a CDR-L1 sequence comprising the amino acid sequence of QSLVHQNAQTY (SEQ ID NO:59), a CDR-L2 sequence comprising the amino acid sequence of KVS (SEQ ID NO:64), and a CDR-L3 sequence comprising the amino acid sequence of GQGTQYPFT (SEQ ID NO:65); and
wherein V_{H3} and V_{L3} form the third antigen binding site that binds a HER2 polypeptide, wherein the V_{H3} domain comprises a CDR-H1 sequence comprising the amino acid sequence of GFNIRDTY (SEQ ID NO:2), a CDR-H2 sequence comprising the amino acid sequence of IYPTQGYT (SEQ ID NO:4), and a CDR-H3 sequence comprising the amino acid sequence of SRWGGEGFYAMDY (SEQ ID NO:7), and the V_{L3} domain comprises a CDR-L1 sequence comprising the amino acid sequence of QDVNTA (SEQ ID NO:9), a CDR-L2 sequence comprising the amino acid sequence of SAS (SEQ ID NO:11), and a CDR-L3 sequence comprising the amino acid sequence of QQHYTTP (SEQ ID NO:12).

3. The method of claim 2, wherein the V_{H1} domain comprises the amino acid sequence of QVQLVQSGAEVVKPGASVKVSCKASGYTFTSYYIHWVRQAPGQGLEWIGSIYPGNVNT NYAQKFQGRATLTVDTSISTAYMELSRLRSDDTAVYYCTRSHYGLDWNFDVWGKGTT VTVSS (SEQ ID NO:91), and the V_{L1} domain comprises the amino acid sequence of DIQMTQSPSSLSASVGDRVTITCQASQNIYVWLNWYQQKPGKAPKLLIYKASNLHTGVP SRFSGSGSGTDFTLTISSLQPEDIATYYCQQGQTYPYTFGQGTKLEIK (SEQ ID NO:92).

4. The method of claim 2, wherein the V_{H2} domain comprises the amino acid sequence of QVQLVESGGGVVQPGRSLRLSCAASGFTFTKAWMHWVRQAPGKQLEWVAQIKDKSNS YATYYADSVKGRFTISRDDSKNTLYLQMNSLRAEDTAVYYCRGVYYALSPFDYWGQG TLVTVSS (SEQ ID NO:93), and the V_{L2} domain comprises the amino acid sequence of DIVMTQTPLSLSVTPGQPASISCKSSQSLVHQNAQTYLSWYLQKPGQSPQSLIYKVSNRF SGVPDRFSGSGSGTDFTLKISRVEAEDVGVYYCGQGTQYPFTFGSGTKVEIK (SEQ ID NO:95).

5. The method of claim 2, wherein the V_{H3} domain comprises the amino acid sequence of EVQLVESGGGLVQPGGSLRLSCAASGFNIRDTYIHWVRQAPGKGLEWVARIYPTQGYTR YADSVKGRFTISADTSKNTAYLQMNSLRAEDTAVYYCSRWGGEGFYAMDYWGQGTL VTVSS (SEQ ID NO:73), and the V_{L3} domain comprises the amino acid sequence of DIQMTQSPSSLSASVGDRVTITCRASQDVNTAVAWYQQKPGKAPKLLIYSASFLYSGVP SRFSGSRSGTDFTLTISSLQPEDFATYYCQQHYTTPPTFGQGTKVEIK (SEQ ID NO:77).

6. The method of claim 2, wherein the first polypeptide chain comprises the amino acid sequence of SEQ ID NO:104 or an amino acid sequence that is at least 95% identical to the amino acid sequence of SEQ ID NO:104; the second polypeptide chain comprises the amino acid sequence of SEQ ID NO:105 or an amino acid sequence that is at least 95% identical to the amino acid sequence of SEQ ID NO:105; the third polypeptide chain comprises the amino acid sequence of SEQ ID NO:106 or an amino acid sequence that is at least 95% identical to the amino acid sequence of SEQ ID NO:106; and the fourth polypeptide chain comprises the amino acid sequence of SEQ ID NO:107 or an amino acid sequence that is at least 95% identical to the amino acid sequence of SEQ ID NO:107.

7. The method of claim 2, wherein L₁, L₂, L₃ and L₄ each independently comprise the sequence DKTHT (SEQ ID NO:66).

8. The method of claim 2, wherein the hinge-C_{H2}-C_{H3} domains of the second and the third polypeptide chains are human IgG4 hinge-C_{H2}-C_{H3} domains, and wherein the hinge-C_{H2}-C_{H3} domains each comprise amino acid substitutions at positions corresponding to positions 234 and 235 of human IgG4 according to EU Index, wherein the amino acid substitutions are F234A and L235A.

9. The method of claim 2, wherein the hinge-C_{H2}-C_{H3} domains of the second and the third polypeptide chains are human IgG4 hinge-C_{H2}-C_{H3} domains, and wherein the hinge-C_{H2}-C_{H3} domains each comprise amino acid substitutions at positions corresponding to positions 228 and 409 of human IgG4 according to EU Index, wherein the amino acid substitutions are S228P and R409K.

10. The method of claim 2, wherein the hinge-C_{H2}-C_{H3} domain of the second polypeptide chain comprises amino acid substitutions at positions corresponding to positions 349, 366, 368, and 407 of human IgG1 or IgG4 according to EU Index, wherein the amino acid substitutions are Y349C, T366S, L368A, and Y407V; and wherein the hinge-C_{H2}-C_{H3} domain of the third polypeptide chain comprises amino acid substitutions at positions corresponding to positions 354 and 366 of human IgG1 or IgG4 according to EU Index, wherein the amino acid substitutions are S354C and T366W.

11. The method of claim 2, wherein the hinge-C_{H2}-C_{H3} domain of the second polypeptide chain comprises amino acid substitutions at positions corresponding to positions 354 and 366 of human IgG1 or IgG4 according to EU Index, wherein the amino acid substitutions are S354C and T366W; and wherein the hinge-C_{H2}-C_{H3} domain of the third polypeptide chain comprises amino acid substitutions at positions corresponding to positions 349, 366, 368, and 407 of human IgG1 or IgG4 according to EU Index, wherein the amino acid substitutions are Y349C, T366S, L368A, and Y407V.

12. The method of claim 1, wherein the trispecific binding protein is administered to the subject once per week, once every two weeks, once every three weeks, or once every four weeks.

13. The method of claim 1, wherein the trispecific binding protein
(a) is administered to the subject for at least one 28-day cycle, and/or
(b) is administered to the subject weekly at a dose of between about 10 μg to about 1500 μg for at least one 28-day cycle.

14-16. (canceled)

17. The method of claim 13, wherein the trispecific binding protein
(a) is administered to the subject during a lead-in phase prior to initiation of the at least one 28-day cycle, optionally wherein the lead-in phase is 28 days, and/or
(b) is administered to the subject at a dose of between about 0.2 μg to about 1500 μg during a lead-in phase prior to initiation of the at least one 28-day cycle.

18-21. (canceled)

22. The method of claim 1, wherein the trispecific binding protein is administered to the subject weekly at a dose of between about 300 μg to about 4500 μg.

23. (canceled)

24. The method of claim 22, wherein the trispecific binding protein is administered to the subject during a lead-in phase prior to initiation of weekly administration of the trispecific binding protein.

25-28. (canceled)

29. The method of claim 24, wherein prior to initiation of the lead-in phase, the trispecific binding protein is administered to the subject intravenously in an additional escalating treatment regimen.

30-31. (canceled)

32. The method of claim 1, wherein the cancer is a HER2-positive cancer.

33-45. (canceled)

46. The method of claim 1, wherein administration of the trispecific binding protein to the subject results in
(a) an increase in the level of at least one cytokine in the peripheral blood of the subject compared to the level of the at least one cytokine in the peripheral blood of the subject prior to the administration of the trispecific binding protein,
(b) an increase in the level of at least one T cell activation marker in the peripheral blood of the subject compared to the level of the at least one T cell activation marker in the peripheral blood of the subject prior to the administration of the trispecific binding protein,
(c) an increase in the level of a cytotoxicity biomarker in the peripheral blood of the subject compared to the level of the cytotoxicity biomarker in the peripheral blood of the subject prior to the administration of the trispecific binding protein, and/or
(d) an increase in the level of a proliferation biomarker in the peripheral blood of the subject compared to the level of the proliferation biomarker in the peripheral blood of the subject prior to the administration of the trispecific binding protein.

47-54. (canceled)

55. The method of claim 1, wherein the method
(a) delays progression of the cancer in the subject,
(b) reduces the tumor size in the subject, and/or
(c) reduces the number and/or size of metastatic lesions in the subject.

56-62. (canceled)


SEQUENCE LISTING (302 sequences)

SEQ ID NO: 1 - protein, 8 aa, synthetic construct
GFNIKDTY

SEQ ID NO: 2 - protein, 8 aa, synthetic construct
GFNIRDTY

SEQ ID NO: 3 - protein, 8 aa, synthetic construct
IYPTNGYT

SEQ ID NO: 4 - protein, 8 aa, synthetic construct
IYPTQGYT

SEQ ID NO: 5 - protein, 8 aa, synthetic construct
IYPTNAYT

SEQ ID NO: 6 - protein, 13 aa, synthetic construct
SRWGGDGFYAMDY

SEQ ID NO: 7 - protein, 13 aa, synthetic construct
SRWGGEGFYAMDY

SEQ ID NO: 8 - protein, 13 aa, synthetic construct
SRWGGSGFYAMDY

SEQ ID NO: 9 - protein, 6 aa, synthetic construct
QDVNTA

SEQ ID NO: 10 - protein, 6 aa, synthetic construct
QDVQTA

SEQ ID NO: 11 - skipped

SEQ ID NO: 12 - protein, 7 aa, synthetic construct
QQHYTTP

SEQ ID NO: 13-48 - skipped (36 entries)

SEQ ID NO: 49 - protein, 8 aa, synthetic construct
GYTFTSYY

SEQ ID NO: 50 - protein, 8 aa, synthetic construct
IYPGNVNT

SEQ ID NO: 51 - protein, 13 aa, synthetic construct
TRSHYGLDWNFDV

SEQ ID NO: 52 - protein, 6 aa, synthetic construct
QNIYVW

SEQ ID NO: 53 - skipped

SEQ ID NO: 54 - protein, 8 aa, synthetic construct
QQGQTYPY

SEQ ID NO: 55 - protein, 8 aa, synthetic construct
GFTFTKAW

SEQ ID NO: 56 - protein, 10 aa, synthetic construct
IKDKSNSYAT

SEQ ID NO: 57 - protein, 12 aa, synthetic construct
RGVYYALSPFDY

SEQ ID NO: 58 - protein, 11 aa, synthetic construct
QSLVHNNANTY

SEQ ID NO: 59 - protein, 11 aa, synthetic construct
QSLVHQNAQTY

SEQ ID NO: 60 - protein, 11 aa, synthetic construct
QSLVHENLQTY

SEQ ID NO: 61 - protein, 11 aa, synthetic construct
QSLVHENLFTY

SEQ ID NO: 62 - protein, 11 aa, synthetic construct
QSLVHENLRTY

SEQ ID NO: 63 - protein, 11 aa, synthetic construct
QSLVHDNAQTY

SEQ ID NO: 64 - skipped

SEQ ID NO: 65 - protein, 9 aa, synthetic construct
GQGTQYPFT

SEQ ID NO: 66 - protein, 5 aa, synthetic construct
DKTHT

SEQ ID NO: 67 - protein, 7 aa, synthetic construct
GQPKAAP

SEQ ID NO: 68 - protein, 5 aa, synthetic construct
TKGPS

SEQ ID NO: 69 - protein, 10 aa, synthetic construct
GGGGSGGGGS

SEQ ID NO: 70 - protein, 15 aa, synthetic construct
GGGGSGGGGSGGGGS

SEQ ID NO: 71 - protein, 10 aa, synthetic construct
GGSGSSGSGG

SEQ ID NO: 72 - protein, 120 aa, synthetic construct [sequence omitted]

SEQ ID NO: 73 - protein, 120 aa, synthetic construct
EVQLVESGGGLVQPGGSLRLSCAASGFNIRDTYIHWVRQAPGKGLEWVARIYPTQGYTRYADSVKGRFTISADTSKNTAYLQMNSLRAEDTAVYYCSRWGGEGFYAMDYWGQGTLVTVSS

SEQ ID NO: 74 - protein, 120 aa, synthetic construct
EVQLVESGGGLVQPGGSLRLSCAASGFNIRDTYIHWVRQAPGKGLEWVARIYPTQGYTRYADSVKGRFTISADTSKNTAYLQMNSLRAEDTAVYYCSRWGGSGFYAMDYWGQGTLVTVSS

SEQ ID NO: 75 - protein, 120 aa, synthetic construct
EVQLVESGGGLVQPGGSLRLSCAASGFNIRDTYIHWVRQAPGKGLEWVARIYPTNAYTRYADSVKGRFTISADTSKNTAYLQMNSLRAEDTAVYYCSRWGGSGFYAMDYWGQGTLVTVSS

SEQ ID NO: 76 - protein, 120 aa, synthetic construct
EVQLVESGGGLVQPGGSLRLSCAASGFNIRDTYIHWVRQAPGKGLEWVARIYPTNAYTRYADSVKGRFTISADTSKNTAYLQMNSLRAEDTAVYYCSRWGGEGFYAMDYWGQGTLVTVSS

SEQ ID NO: 77 - protein, 107 aa, synthetic construct [sequence omitted]

SEQ ID NO: 78 - protein, 107 aa, synthetic construct
DIQMTQSPSSLSASVGDRVTITCRASQDVQTAVAWYQQKPGKAPKLLIYSASFLYSGVPSRFSGSRSGTDFTLTISSLQPEDFATYYCQQHYTTPPTFGQGTKVEIK

SEQ ID NO: 79-90 - skipped (12 entries)

SEQ ID NO: 91 - protein, 120 aa, synthetic construct
QVQLVQSGAEVVKPGASVKVSCKASGYTFTSYYIHWVRQAPGQGLEWIGSIYPGNVNTNYAQKFQGRATLTVDTSISTAYMELSRLRSDDTAVYYCTRSHYGLDWNFDVWGKGTTVTVSS

SEQ ID NO: 92 - protein, 107 aa, synthetic construct
DIQMTQSPSSLSASVGDRVTITCQASQNIYVWLNWYQQKPGKAPKLLIYKASNLHTGVPSRFSGSGSGTDFTLTISSLQPEDIATYYCQQGQTYPYTFGQGTKLEIK

SEQ ID NO: 93 - protein, 121 aa, synthetic construct
QVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSS

SEQ ID NO: 94 - protein, 112 aa, synthetic construct
DIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGSGTKVEIK

SEQ ID NO: 95 - protein, 112 aa, synthetic construct
DIVMTQTPLSLSVTPGQPASISCKSSQSLVHQNAQTYLSWYLQKPGQSPQSLIYKVSNRFSGVPDRFSGSGSGTDFTLKISRVEAEDVGVYYCGQGTQYPFTFGSGTKVEIK

SEQ ID NO: 96 - protein, 112 aa, synthetic construct
DIVMTQTPLSLSVTPGQPASISCKSSQSLVHENLQTYLSWYLQKPGQSPQSLIYKVSNRFSGVPDRFSGSGSGTDFTLKISRVEAEDVGVYYCGQGTQYPFTFGSGTKVEIK

SEQ ID NO: 97 - protein, 112 aa, synthetic construct
DIVMTQTPLSLSVTPGQPASISCKSSQSLVHENLFTYLSWYLQKPGQSPQSLIYKVSNRFSGVPDRFSGSGSGTDFTLKISRVEAEDVGVYYCGQGTQYPFTFGSGTKVEIK

SEQ ID NO: 98 - protein, 112 aa, synthetic construct
DIVMTQTPLSLSVTPGQPASISCKSSQSLVHENLRTYLSWYLQKPGQSPQSLIYKVSNRFSGVPDRFSGSGSGTDFTLKISRVEAEDVGVYYCGQGTQYPFTFGSGTKVEIK

SEQ ID NO: 99 - protein, 112 aa, synthetic construct
DIVMTQTPLSLSVTPGQPASISCKSSQSLVHDNAQTYLSWYLQKPGQSPQSLIYKVSNRFSGVPDRFSGSGSGTDFTLKISRVEAEDVGVYYCGQGTQYPFTFGSGTKVEIK

SEQ ID NO: 100 - protein, 336 aa, synthetic construct
DIVMTQTPLSLSVTPGQPASISCKSSQSLVHQNAQTYLSWYLQKPGQSPQSLIYKVSNRFSGVPDRFSGSGSGTDFTLKISRVEAEDVGVYYCGQGTQYPFTFGSGTKVEIKDKTHTDIQMTQSPSSLSASVGDRVTITCQASQNIYVWLNWYQQKPGKAPKLLIYKASNLHTGVPSRFSGSGSGTDFTLTISSLQPEDIATYYCQQGQTYPYTFGQGTKLEIKDKTHTRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 577 aa, synthetic construct
QVQLVQSGAEVVKPGASVKVSCKASGYTFTSYYIHWVRQAPGQGLEWIGSIYPGNVNTNYAQKFQGRATLTVDTSISTAYMELSRLRSDDTAVYYCTRSHYGLDWNFDVWGKGTTVTVSSDKTHTQVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSSDKTHTASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSKLTVDKSRWQEGNVFSCSVMHEALHNHYTQKSLSLSLG

SEQ ID NO: 102 - protein, 446 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNHYTQKSLSLSLG

SEQ ID NO: 103 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 336 aa, synthetic construct; identical to SEQ ID NO: 100

SEQ ID NO: 105 - protein, 577 aa, synthetic construct; identical to SEQ ID NO: 101

SEQ ID NO: 106 - protein, 446 aa, synthetic construct
EVQLVESGGGLVQPGGSLRLSCAASGFNIRDTYIHWVRQAPGKGLEWVARIYPTQGYTRYADSVKGRFTISADTSKNTAYLQMNSLRAEDTAVYYCSRWGGEGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNHYTQKSLSLSLG

SEQ ID NO: 107 - protein, 214 aa, synthetic construct; identical to SEQ ID NO: 103

SEQ ID NO: 108 - protein, 336 aa, synthetic construct
DIVMTQTPLSLSVTPGQPASISCKSSQSLVHDNAQTYLSWYLQKPGQSPQSLIYKVSNRFSGVPDRFSGSGSGTDFTLKISRVEAEDVGVYYCGQGTQYPFTFGSGTKVEIKDKTHTDIQMTQSPSSLSASVGDRVTITCQASQNIYVWLNWYQQKPGKAPKLLIYKASNLHTGVPSRFSGSGSGTDFTLTISSLQPEDIATYYCQQGQTYPYTFGQGTKLEIKDKTHTRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 577 aa, synthetic construct; identical to SEQ ID NO: 101

SEQ ID NO: 110 - protein, 446 aa, synthetic construct; identical to SEQ ID NO: 106

SEQ ID NO: 111 - protein, 214 aa, synthetic construct; identical to SEQ ID NO: 103

SEQ ID NO: 112 - protein, 338 aa, synthetic construct
DIVMTQTPLSLSVTPGQPASISCKSSQSLVHQNAQTYLSWYLQKPGQSPQSLIYKVSNRFSGVPDRFSGSGSGTDFTLKISRVEAEDVGVYYCGQGTQYPFTFGSGTKVEIKGQPKAAPDIQMTQSPSSLSASVGDRVTITCQASQNIYVWLNWYQQKPGKAPKLLIYKASNLHTGVPSRFSGSGSGTDFTLTISSLQPEDIATYYCQQGQTYPYTFGQGTKLEIKTKGPSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 570 aa, synthetic construct
QVQLVQSGAEVVKPGASVKVSCKASGYTFTSYYIHWVRQAPGQGLEWIGSIYPGNVNTNYAQKFQGRATLTVDTSISTAYMELSRLRSDDTAVYYCTRSHYGLDWNFDVWGKGTTVTVSSSQVQLVESGGGVVQPGRSLRLSCAASGFTFTKAWMHWVRQAPGKQLEWVAQIKDKSNSYATYYADSVKGRFTISRDDSKNTLYLQMNSLRAEDTAVYYCRGVYYALSPFDYWGQGTLVTVSSRTASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSKLTVDKSRWQEGNVFSCSVMHEALHNHYTQKSLSLSLG

SEQ ID NO: 114 - protein, 446 aa, synthetic construct; identical to SEQ ID NO: 106

SEQ ID NO: 115 - protein, 214 aa, synthetic construct; identical to SEQ ID NO: 103

SEQ ID NO: 116 - protein, 338 aa, synthetic construct
DIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGSGTKVEIKGQPKAAPDIQMTQSPSSLSASVGDRVTITCQASQNIYVWLNWYQQKPGKAPKLLIYKASNLHTGVPSRFSGSGSGTDFTLTISSLQPEDIATYYCQQGQTYPYTFGQGTKLEIKTKGPSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 570 aa, synthetic construct; identical to SEQ ID NO: 113

SEQ ID NO: 118 - protein, 446 aa, synthetic construct
EVQLVESGGGLVQPGGSLRLSCAASGFNIRDTYIHWVRQAPGKGLEWVARIYPTQGYTRYADSVKGRFTISADTSKNTAYLQMNSLRAEDTAVYYCSRWGGSGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNHYTQKSLSLSLG

SEQ ID NO: 119 - protein, 214 aa, synthetic construct; identical to SEQ ID NO: 103

SEQ ID NO: 120 - protein, 338 aa, synthetic construct; identical to SEQ ID NO: 116

SEQ ID NO: 121 - protein, 570 aa, synthetic construct; identical to SEQ ID NO: 113

SEQ ID NO: 122 - protein, 446 aa, synthetic construct
EVQLVESGGGLVQPGGSLRLSCAASGFNIRDTYIHWVRQAPGKGLEWVARIYPTNAYTRYADSVKGRFTISADTSKNTAYLQMNSLRAEDTAVYYCSRWGGSGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNHYTQKSLSLSLG

SEQ ID NO: 123 - protein, 214 aa, synthetic construct; identical to SEQ ID NO: 103

SEQ ID NO: 124 - protein, 338 aa, synthetic construct; identical to SEQ ID NO: 116

SEQ ID NO: 125 - protein, 570 aa, synthetic construct; identical to SEQ ID NO: 113

SEQ ID NO: 126 - protein, 446 aa, synthetic construct
EVQLVESGGGLVQPGGSLRLSCAASGFNIRDTYIHWVRQAPGKGLEWVARIYPTNAYTRYADSVKGRFTISADTSKNTAYLQMNSLRAEDTAVYYCSRWGGEGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSKLTVDKSRWQEGNVFSCSVMHEALHNHYTQKSLSLSLG

SEQ ID NO: 127 - protein, 214 aa, synthetic construct; identical to SEQ ID NO: 103

SEQ ID NO: 128 - protein, 338 aa, synthetic construct; identical to SEQ ID NO: 112

SEQ ID NO: 129 - protein, 570 aa, synthetic construct; identical to SEQ ID NO: 113

SEQ ID NO: 130 - protein, 446 aa, synthetic construct; identical to SEQ ID NO: 102

SEQ ID NO: 131 - protein, 214 aa, synthetic construct; identical to SEQ ID NO: 103

SEQ ID NO: 132 - protein, 336 aa, synthetic construct
DIVMTQTPLSLSVTPGQPASISCKSSQSLVHNNANTYLSWYLQKPGQSPQSLIYKVSNRFSGVPDRFSGSGSGTDFTLKISRVEAEDVGVYYCGQGTQYPFTFGSGTKVEIKDKTHTDIQMTQSPSSLSASVGDRVTITCQASQNIYVWLNWYQQKPGKAPKLLIYKASNLHTGVPSRFSGSGSGTDFTLTISSLQPEDIATYYCQQGQTYPYTFGQGTKLEIKDKTHTRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 577 aa, synthetic construct; identical to SEQ ID NO: 101

SEQ ID NO: 134 - protein, 446 aa, synthetic construct; identical to SEQ ID NO: 102

SEQ ID NO: 135 - protein, 214 aa, synthetic construct; identical to SEQ ID NO: 103

SEQ ID NO: 136 - protein, 336 aa, synthetic construct
DIVMTQTPLSLSVTPGQPASISCKSSQSLVHENLRTYLSWYLQKPGQSPQSLIYKVSNRFSGVPDRFSGSGSGTDFTLKISRVEAEDVGVYYCGQGTQYPFTFGSGTKVEIKDKTHTDIQMTQSPSSLSASVGDRVTITCQASQNIYVWLNWYQQKPGKAPKLLIYKASNLHTGVPSRFSGSGSGTDFTLTISSLQPEDIATYYCQQGQTYPYTFGQGTKLEIKDKTHTRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 577 aa, synthetic construct; identical to SEQ ID NO: 101

SEQ ID NO: 138 - protein, 446 aa, synthetic construct; identical to SEQ ID NO: 102

SEQ ID NO: 139 - protein, 214 aa, synthetic construct; identical to SEQ ID NO: 103

SEQ ID NO: 140 - protein, 336 aa, synthetic construct
DIVMTQTPLSLSVTPGQPASISCKSSQSLVHENLQTYLSWYLQKPGQSPQSLIYKVSNRFSGVPDRFSGSGSGTDFTLKISRVEAEDVGVYYCGQGTQYPFTFGSGTKVEIKDKTHTDIQMTQSPSSLSASVGDRVTITCQASQNIYVWLNWYQQKPGKAPKLLIYKASNLHTGVPSRFSGSGSGTDFTLTISSLQPEDIATYYCQQGQTYPYTFGQGTKLEIKDKTHTRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - protein, 577 aa, synthetic construct; identical to SEQ ID NO: 101

SEQ ID NO: 142 - protein, 446 aa, synthetic construct; identical to SEQ ID NO: 102

SEQ ID NO: 143 - protein, 214 aa, synthetic construct; identical to SEQ ID NO: 103

SEQ ID NO: 144 - protein, 336 aa, synthetic construct
DIVMTQTPLSLSVTPGQPASISCKSSQSLVHENLFTYLSWYLQKPGQSPQSLIYKVSNRFSGVPDRFSGSGSGTDFTLKISRVEAEDVGVYYCGQGTQYPFTFGSGTKVEIKDKTHTDIQMTQSPSSLSASVGDRVTITCQASQNIYVWLNWYQQKPGKAPKLLIYKASNLHTGVPSRFSGSGSGTDFTLTISSLQPEDIATYYCQQGQTYPYTFGQGTKLEIKDKTHTRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 577 aa, synthetic construct; identical to SEQ ID NO: 101

SEQ ID NO: 146 - protein, 446 aa, synthetic construct; identical to SEQ ID NO: 102

SEQ ID NO: 147 - protein, 214 aa, synthetic construct; identical to SEQ ID NO: 103

SEQ ID NO: 148 - protein, 338 aa, synthetic construct; identical to SEQ ID NO: 116

SEQ ID NO: 149 - protein, 570 aa, synthetic construct; identical to SEQ ID NO: 113

SEQ ID NO: 150 - protein, 446 aa, synthetic construct; identical to SEQ ID NO: 102

SEQ ID NO: 151 - protein, 214 aa, synthetic construct; identical to SEQ ID NO: 103

SEQ ID NO: 152 - protein, 336 aa, synthetic construct; identical to SEQ ID NO: 136

SEQ ID NO: 153 - protein, 577 aa, synthetic construct; identical to SEQ ID NO: 101

SEQ ID NO: 154 - protein, 446 aa, synthetic construct; identical to SEQ ID NO: 106

SEQ ID NO: 155 - protein, 214 aa, synthetic construct; identical to SEQ ID NO: 103

SEQ ID NO: 156-179 - skipped (24 entries)

SEQ ID NO: 180 - protein, 11 aa, synthetic construct
  VARIANT 6: note = X = E or Q
  VARIANT 8: note = X = A or L
  VARIANT 9: note = X = Q, R, or F
QSLVHXNXXTY

SEQ ID NO: 181-285 - skipped (105 entries)

SEQ ID NO: 286 - protein, 336 aa, synthetic construct; identical to SEQ ID NO: 100

SEQ ID NO: 287 - protein, 577 aa, synthetic construct; identical to SEQ ID NO: 101

SEQ ID NO: 288 - protein, 446 aa, synthetic construct; identical to SEQ ID NO: 122

SEQ ID NO: 289 - protein, 214 aa, synthetic construct; identical to SEQ ID NO: 103

SEQ ID NO: 290 - protein, 338 aa, synthetic construct; identical to SEQ ID NO: 112

SEQ ID NO: 291 - protein, 577 aa, synthetic construct
QVQLVQSGAEVVKPGASVKVSCKASGYTFTSYYIHWVRQAPGQGLEWIGSIYPGNVNTNYAQKFQGRATLTVDTSISTAYMELSRLRSDDTAVYYCTRSHYGLDWNFDVWGKGTTVTVSSDKTHTQVQLVESGGGVVQPGRSLRLSCAASGFTFTKAWMHWVRQAPGKQLEWVAQIKDKSNSYATYYAESVKGRFTISRDDSKNTLYLQMNSLRAEDTAVYYCRGVYYALSPFDYWGQGTLVTVSSDKTHTASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSKLTVDKSRWQEGNVFSCSVMHEALHNHYTQKSLSLSLG

SEQ ID NO: 292 - protein, 446 aa, synthetic construct; identical to SEQ ID NO: 106

SEQ ID NO: 293 - protein, 214 aa, synthetic construct; identical to SEQ ID NO: 103

SEQ ID NO: 294 - protein, 338 aa, synthetic construct; identical to SEQ ID NO: 112

SEQ ID NO: 295 - protein, 570 aa, synthetic construct
QVQLVQSGAEVVKPGASVKVSCKASGYTFTSYYIHWVRQAPGQGLEWIGSIYPGNVNTNYAQKFQGRATLTVDTSISTAYMELSRLRSDDTAVYYCTRSHYGLDWNFDVWGKGTTVTVSSSQVQLVESGGGVVQPGRSLRLSCAASGFTFTKAWMHWVRQAPGKQLEWVAQIKDKSNSYATYYASSVKGRFTISRDDSKNTLYLQMNSLRAEDTAVYYCRGVYYALSPFDYWGQGTLVTVSSRTASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSKLTVDKSRWQEGNVFSCSVMHEALHNHYTQKSLSLSLG

SEQ ID NO: 296 - protein, 446 aa, synthetic construct; identical to SEQ ID NO: 106

SEQ ID NO: 297 - protein, 214 aa, synthetic construct; identical to SEQ ID NO: 103

SEQ ID NO: 298 - protein, 338 aa, synthetic construct
DIVMTQTPLSLSVTPGQPASISCKSSQSLVHQSAQTYLSWYLQKPGQSPQSLIYKVSNRFSGVPDRFSGSGSGTDFTLKISRVEAEDVGVYYCGQGTQYPFTFGSGTKVEIKGQPKAAPDIQMTQSPSSLSASVGDRVTITCQASQNIYVWLNWYQQKPGKAPKLLIYKASNLHTGVPSRFSGSGSGTDFTLTISSLQPEDIATYYCQQGQTYPYTFGQGTKLEIKTKGPSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 299 - protein, 570 aa, synthetic construct; identical to SEQ ID NO: 295

SEQ ID NO: 300 - protein, 446 aa, synthetic construct; identical to SEQ ID NO: 106

SEQ ID NO: 301 - protein, 214 aa, synthetic construct; identical to SEQ ID NO: 103

SEQ ID NO: 302 - protein, 121 aa, synthetic construct
QVQLVESGGGVVQPGRSLRLSCAASGFTFTKAWMHWVRQAPGKQLEWVAQIKDKSNSYATYYASSVKGRFTISRDDSKNTLYLQMNSLRAEDTAVYYCRGVYYALSPFDYWGQGTLVTVSS